SEQUENCE LISTING (108 sequences)

SEQ ID NO: 1 - protein, 29 aa, synthetic construct
AALEALAEALEALAEALEALAEAAAAGGC

SEQ ID NO: 2 - protein, 30 aa, synthetic construct
AALAEALAEALAEALAEALAEALAAAAGGC

SEQ ID NO: 3 - protein, 15 aa, synthetic construct
ALEALAEALEALAEA

SEQ ID NO: 4 - protein, 22 aa, synthetic construct
GLFEAIEGFIENGWEGMIWDYG

SEQ ID NO: 5 - protein, 23 aa, synthetic construct
GLFGAIAGFIENGWEGMIDGWYG

SEQ ID NO: 6 - protein, 48 aa, synthetic construct
GLFEAIEGFIENGWEGMIDGWYGCGLFEAIEGFIENGWEGMIDGWYGC

SEQ ID NO: 7 - protein, 44 aa, synthetic construct
GLFEAIEGFIENGWEGMIDGGCGLFEAIEGFIENGWEGMIDGGC

SEQ ID NO: 8 - protein, 35 aa, synthetic construct
GLFGALAEALAEALAEHLAEALAEALEALAAGGSC

SEQ ID NO: 9 - protein, 34 aa, synthetic construct
GLFEAIEGFIENGWEGLAEALAEALEALAAGGSC

SEQ ID NO: 10 - protein, 41 aa, synthetic construct
  SITE 17: note = Nle
  SITE 38: note = Nle
GLFEAIEGFIENGWEGXIDGKGLFEAIEGFIENGWEGXIDG

SEQ ID NO: 11 - protein, 19 aa, synthetic construct
LFEALLELLESLWELLLEA

SEQ ID NO: 12 - protein, 20 aa, synthetic construct
GLFKALLKLLKSLWKLLLKA

SEQ ID NO: 13 - protein, 20 aa, synthetic construct
GLFRALLRLLRSLWRLLLRA

SEQ ID NO: 14 - protein, 30 aa, synthetic construct
WEAKLAKALAKALAKHLAKALAKALKACEA

SEQ ID NO: 15 - protein, 22 aa, synthetic construct
GLFFEAIAEFIEGGWEGLIEGC

SEQ ID NO: 16 - protein, 26 aa, synthetic construct
GIGAVLKVLTTGLPALISWIKRKRQQ

SEQ ID NO: 17 - protein, 8 aa, synthetic construct
HHHHHWYG

SEQ ID NO: 18 - protein, 10 aa, synthetic construct
CHKKKKKKHC

SEQ ID NO: 19 - protein, 16 aa, synthetic construct
RQIKIWFQNRRMKWKK

SEQ ID NO: 20 - protein, 14 aa, synthetic construct
GRKKRRQRRRPPQC

SEQ ID NO: 21 - protein, 27 aa, synthetic construct
GALFLGWLGAAGSTMGAWSQPKKKRKV

SEQ ID NO: 22 - protein, 18 aa, synthetic construct
LLIILRRRIRKQAHAHSK

SEQ ID NO: 23 - protein, 26 aa, synthetic construct
GWTLNSAGYLLKINLKALAALAKKIL

SEQ ID NO: 24 - protein, 18 aa, synthetic construct
KLALKLALKALKAALKLA

SEQ ID NO: 25 - protein, 9 aa, synthetic construct
RRRRRRRRR

SEQ ID NO: 26 - protein, 10 aa, synthetic construct
KFFKFFKFFK

SEQ ID NO: 27 - protein, 37 aa, synthetic construct
LLGDFFRKSKEKIGKEFKRIVQRIKDFLRNLVPRTES

SEQ ID NO: 28 - protein, 31 aa, synthetic construct
SWLSKTAKKLENSAKKRISEGIAIAIQGGPR

SEQ ID NO: 29 - protein, 30 aa, synthetic construct
ACYCRIPACIAGERRYGTCIYQGRLWAFCC

SEQ ID NO: 30 - protein, 36 aa, synthetic construct
DHYNCVSSGGQCLYSACPIFTKIQGTCYRGKAKCCK

SEQ ID NO: 31 - protein, 42 aa, synthetic construct
RRRPRPPYLPRPRPPPFFPPRLPPRIPPGFPPRFPPRFPGKR

SEQ ID NO: 32 - protein, 13 aa, synthetic construct
ILPWKWPWWPWRR

SEQ ID NO: 33 - protein, 16 aa, synthetic construct
AAVALLPAVLLALLAP

SEQ ID NO: 34 - protein, 11 aa, synthetic construct
AALLPVLLAAP

SEQ ID NO: 35 - protein, 12 aa, synthetic construct
RKCRIVVIRVCR

SEQ ID NO: 36 - RNA, 12 nt, synthetic construct
  source 1..12: mol_type = other RNA
tacagtctatgt

SEQ ID NO: 37 - RNA, 12 nt, synthetic construct
  source 1..12: mol_type = other RNA
acatagactgta

SEQ ID NO: 38 - RNA, 12 nt, synthetic construct
  source 1..12: mol_type = other RNA
  modified_base 6: mod_base = OTHER; note = 2-prime-deoxy-2-prime-fluoronucleotide-3-prime-phos phate
tacagtctatgt

SEQ ID NO: 39 - RNA, 12 nt, synthetic construct
  source 1..12: mol_type = other RNA
  modified_base 6: mod_base = OTHER; note = 2-prime-F/Me nucleotide
tacagtctatgt

SEQ ID NO: 40 - RNA, 12 nt, synthetic construct
  source 1..12: mol_type = other RNA
  modified_base 7: mod_base = OTHER; note = 2-prime-deoxy-2-prime-fluoronucleotide-3-prime-phos phate
tacagtctatgt

SEQ ID NO: 41 - RNA, 12 nt, synthetic construct
  source 1..12: mol_type = other RNA
  modified_base 7: mod_base = OTHER; note = 2-prime-F/Me nucleotide
tacagtctatgt

SEQ ID NO: 42 - RNA, 21 nt, synthetic construct
  source 1..21: mol_type = other RNA
  ncRNA 1..21: ncRNA_class = siRNA
  modified_base order(1,3,5,7,9..11,13,15,17,19,21): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base order(2,4,6,8,12,14,16,18,20): mod_base = OTHER; note = 2-prime-O-methylnucleotides-3-prime-phosphate
aacagtgttcttgctctataa

SEQ ID NO: 43 - RNA, 21 nt, synthetic construct
  source 1..21: mol_type = other RNA
  ncRNA 1..21: ncRNA_class = siRNA
  modified_base order(1,3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
aagatgatgtttgaaactatt

SEQ ID NO: 44 - RNA, 21 nt, synthetic construct
  source 1..21: mol_type = other RNA
  ncRNA 1..21: ncRNA_class = siRNA
  modified_base order(1,3,5,7,9..11,13,15,17,19,21): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base order(2,4,6,8,12,14,16,18,20): mod_base = OTHER; note = 2-prime-O-methylnucleotides-3-prime-phosphate
caggatcatctcaagtcttaa

SEQ ID NO: 45 - RNA, 21 nt, synthetic construct
  source 1..21: mol_type = other RNA
  ncRNA 1..21: ncRNA_class = siRNA
  modified_base order(3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1: mod_base = OTHER; note = 2-prime-F/Me nucleotide
  modified_base 1^2: mod_base = OTHER; note = Phosphorothioate linkage
  modified_base 2^3: mod_base = OTHER; note = Phosphorothioate linkage
caggatcatctcaagtcttaa

SEQ ID NO: 46 - RNA, 21 nt, synthetic construct
  source 1..21: mol_type = other RNA
  ncRNA 1..21: ncRNA_class = siRNA
  modified_base order(1..6,9,12..21): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
  modified_base order(7,9..11): mod_base = OTHER; note = 2-prime-deoxy-2-prime-fluoronucleotides-3-prime-pho sphate
agtgttcttgctctataaaca

SEQ ID NO: 47 - RNA, 23 nt, synthetic construct
  source 1..23: mol_type = other RNA
  ncRNA 1..23: ncRNA_class = siRNA
  modified_base order(2,4,6,8,10,14,16,18,20): mod_base = OTHER; note = 2-prime-deoxy-2-prime-fluoronucleotides-3-prime-pho sphate
  modified_base order(1,3,5,7,9,11..13,15,17,19,21..23): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttatagagcaagaacactgtttt

SEQ ID NO: 48 - RNA, 23 nt, synthetic construct
  source 1..23: mol_type = other RNA
  ncRNA 1..23: ncRNA_class = siRNA
  modified_base 1: mod_base = OTHER; note = 2-prime-F/Me nucleotide
  modified_base order(2,4,6,8,10,14,16,18,20): mod_base = OTHER; note = 2-prime-deoxy-2-prime-fluoronucleotides-3-prime-pho sphate
  modified_base order(3,5,7,9,11..13,15,17,19,21..23): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttatagagcaagaacactgtttt

SEQ ID NO: 49 - RNA, 23 nt, synthetic construct
  source 1..23: mol_type = other RNA
  ncRNA 1..23: ncRNA_class = siRNA
  modified_base order(1,3,5,7,9,11..13,15,17,19,21,23): mod_base = OTHER; note = 2-prime-O-methylnucleotides-3-prime-phosphate
  modified_base order(2,4,6,8,10,14,16,18,20): mod_base = OTHER; note = 2-prime-deoxy-2-prime-fluoronucleotides-3-prime-pho sphate
  modified_base 22: mod_base = OTHER; note = 2-prime-F/Me nucleotide
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttatagagcaagaacactgtttt

SEQ ID NO: 50 - RNA, 23 nt, synthetic construct
  source 1..23: mol_type = other RNA
  ncRNA 1..23: ncRNA_class = siRNA
  modified_base order(1,3,5,7,9,11..13,15,17,19,21): mod_base = OTHER; note = 2-prime-O-methylnucleotides-3-prime-phosphate
  modified_base order(2,4,6,8,10,14,16,18,20): mod_base = OTHER; note = 2-prime-deoxy-2-prime-fluoronucleotides-3-prime-pho sphate
  modified_base 22..23: mod_base = OTHER; note = 2-prime-F/Me nucleotide
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttatagagcaagaacactgtttt

SEQ ID NO: 51 - RNA, 23 nt, synthetic construct
  source 1..23: mol_type = other RNA
  ncRNA 1..23: ncRNA_class = siRNA
  modified_base order(1,3,5,7,9,11..13,15,17,19,21..23): mod_base = OTHER; note = 2-prime-O-methylnucleotides-3-prime-phosphate
  modified_base order(2,4,6,8,10,14,16,18,20): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
aatagtttcaaacatcatcttgt

SEQ ID NO: 52 - RNA, 23 nt, synthetic construct
  source 1..23: mol_type = other RNA
  ncRNA 1..23: ncRNA_class = siRNA
  modified_base order(1,3,5,7,9,11..13,15,17,19,21..23): mod_base = OTHER; note = 2-prime-O-methylnucleotides-3-prime-phosphate
  modified_base order(2,4,8,10,14,16,18,20): mod_base = OTHER; note = 2-prime-deoxy-2-prime-fluoronucleotides-3-prime-pho sphate
  modified_base 6: mod_base = OTHER; note = 2-prime-F/Me nucleotide
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
aatagtttcaaacatcatcttgt

SEQ ID NO: 53 - RNA, 23 nt, synthetic construct
  source 1..23: mol_type = other RNA
  ncRNA 1..23: ncRNA_class = siRNA
  modified_base order(1,3,5,9,11..13,15,17,19,21..23): mod_base = OTHER; note = 2-prime-O-methylnucleotides-3-prime-phosphate
  modified_base order(2,4,6,8,10,14,16,18,20): mod_base = OTHER; note = 2-prime-deoxy-2-prime-fluoronucleotides-3-prime-pho sphate
  modified_base 7: mod_base = OTHER; note = 2-prime-F/Me nucleotide
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
aatagtttcaaacatcatcttgt

SEQ ID NO: 54 - RNA, 23 nt, synthetic construct
  source 1..23: mol_type = other RNA
  ncRNA 1..23: ncRNA_class = siRNA
  modified_base order(1,3,5,9,11..13,15,17,19,21..23): mod_base = OTHER; note = 2-prime-O-methylnucleotides-3-prime-phosphate
  modified_base order(2,4,8,10,14,16,18,20): mod_base = OTHER; note = 2-prime-deoxy-2-prime-fluoronucleotides-3-prime-pho sphate
  modified_base 6..7: mod_base = OTHER; note = 2-prime-F/Me nucleotides
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
aatagtttcaaacatcatcttgt

SEQ ID NO: 55 - RNA, 23 nt, synthetic construct
  source 1..23: mol_type = other RNA
  ncRNA 1..23: ncRNA_class = siRNA
  modified_base order(1,3,5,7,9,11..13,15,17,19,21..23): mod_base = OTHER; note = 2-prime-O-methylnucleotides-3-prime-phosphate
  modified_base order(2,4,6,8,10,14,16,18,20): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttaagacttgagatgatcctggc

SEQ ID NO: 56 - RNA, 23 nt, synthetic construct
  source 1..23: mol_type = other RNA
  ncRNA 1..23: ncRNA_class = siRNA
  modified_base 1: mod_base = OTHER; note = 2-prime-F/Me nucleotide
  modified_base order(2,4,6,8,10,14,16,18,20): mod_base = OTHER; note = 2-prime-deoxy-2-prime-fluoronucleotides-3-prime-pho sphate
  modified_base order(3,5,7,9,11..13,15,17,19,21..23): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttaagacttgagatgatcctggc

SEQ ID NO: 57 - RNA, 23 nt, synthetic construct
  source 1..23: mol_type = other RNA
  ncRNA 1..23: ncRNA_class = siRNA
  modified_base 7: mod_base = OTHER; note = 2-prime-F/Me nucleotide
  modified_base order(1,3,5,9,11..13,15,17,19,21..23): mod_base = OTHER; note = 2-prime-O-methylnucleotides-3-prime-phosphate
  modified_base order(2,4,6,8,10,14,16,18,20): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttaagacttgagatgatcctggc

SEQ ID NO: 58 - RNA, 23 nt, synthetic construct
  source 1..23: mol_type = other RNA
  ncRNA 1..23: ncRNA_class = siRNA
  modified_base order(1,3..13,15,17..23): mod_base = OTHER; note = 2-prime-O-methylnucleotides-3-prime-phosphate
  modified_base order(2,14,16): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
tgtttatagagcaagaacactgt

SEQ ID NO: 59 - RNA, 23 nt, synthetic construct
  source 1..23: mol_type = other RNA
  ncRNA 1..23: ncRNA_class = siRNA
  modified_base 7: mod_base = OTHER; note = 2-prime-F/Me nucleotide
  modified_base order(1,3..6,8..13,15,17..23): mod_base = OTHER; note = 2-prime-O-methylnucleotides-3-prime-phosphate
  modified_base order(2,14,16): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
tgtttatagagcaagaacactgt

SEQ ID NO: 60 - RNA, 23 nt, synthetic construct
  source 1..23: mol_type = other RNA
  modified_base order(1,2,4,6,8,10,14,16,18,20): mod_base = OTHER; note = 2-prime-deoxy-2-prime-fluoronucleotides-3-prime-pho sphate
  modified_base order(3,5,7,9,11..13,15,17,19,21..23): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttatagagcaagaacactgtttt

SEQ ID NO: 61 - RNA, 23 nt, synthetic construct
  source 1..23: mol_type = other RNA
  modified_base 1: mod_base = OTHER; note = 2-prime-F/Me nucleotide
  modified_base order(2,4,6,8,10,14,16,18,20): mod_base = OTHER; note = 2-prime-deoxy-2-prime-fluoronucleotides-3-prime-pho sphate
  modified_base order(3,5,7,9,11..13,15,17,19,21..23): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttatagagcaagaacactgtttt

SEQ ID NO: 62 - DNA, 21 bp, synthetic construct
  misc_feature 20..21: note = DNA
  misc_feature 1..19: note = RNA
  source 1..21: mol_type = other DNA
  modified_base order(4,5,7..10,14..17,19): mod_base = OTHER; note = 2-prime-deoxy-2-prime-fluoronucleotides-3-prime-pho sphate
  modified_base 20..21: mod_base = OTHER; note = 2-prime-deoxynucleotides-3-prime-phosphate
  modified_base 20^21: mod_base = OTHER; note = Phosphorothioate linkage
ggatcatctcaagtcttactt

SEQ ID NO: 63 - DNA, 21 bp, synthetic construct
  source 1..21: mol_type = other DNA
  modified_base order(2,7..9,14,17..19): mod_base = OTHER; note = 2-prime-deoxy-2-prime-fluoronucleotides-3-prime-pho sphate
  misc_feature 1..19: note = RNA
  misc_feature 20..21: note = DNA
  modified_base 20..21: mod_base = OTHER; note = 2-prime-deoxynucleotides-3-prime-phosphate
  modified_base 20^21: mod_base = OTHER; note = Phosphorothioate linkage
gtaagacttgagatgatcctt

SEQ ID NO: 64 - DNA, 21 bp, synthetic construct
  misc_feature 20..21: note = DNA
  misc_feature 1..19: note = RNA
  source 1..21: mol_type = other DNA
  modified_base 20..21: mod_base = OTHER; note = 2-prime-deoxynucleotides-3-prime-phosphate
  modified_base 2: mod_base = OTHER; note = 2-prime-F/Me nucleotide
  modified_base order(7..9,14,17..19): mod_base = OTHER; note = 2-prime-deoxy-2-prime-fluoronucleotides-3-prime-pho sphate
  modified_base 20^21: mod_base = OTHER; note = Phosphorothioate linkage
gtaagacttgagatgatcctt

SEQ ID NO: 65 - DNA, 21 bp, synthetic construct
  misc_feature 20..21: note = DNA
  misc_feature 1..19: note = RNA
  source 1..21: mol_type = other DNA
  modified_base order(2,14): mod_base = OTHER; note = 2-prime-deoxy-2-prime-fluoronucleotides-3-prime-pho sphate
  modified_base order(7..9,17..19): mod_base = OTHER; note = 2-prime-F/Me nucleotides
  modified_base 20..21: mod_base = OTHER; note = 2-prime-deoxynucleotides-3-prime-phosphate
  modified_base 20^21: mod_base = OTHER; note = Phosphorothioate linkage
gtaagacttgagatgatcctt

SEQ ID NO: 66 - DNA, 21 bp, synthetic construct
  source 1..21: mol_type = other DNA
  misc_feature 1..19: note = RNA
  misc_feature 20..21: note = DNA
  modified_base 20..21: mod_base = OTHER; note = 2-prime-deoxynucleotides-3-prime-phosphate
  modified_base order(2,7..9,14,17..19): mod_base = OTHER; note = 2-prime-F/Me nucleotides
  modified_base 20^21: mod_base = OTHER; note = Phosphorothioate linkage
gtaagacttgagatgatcctt

SEQ ID NO: 67 - DNA, 21 bp, synthetic construct
  misc_feature 20..21: note = DNA
  misc_feature 1..19: note = RNA
  source 1..21: mol_type = other DNA
  modified_base order(4,5,7..10,14..17,19): mod_base = OTHER; note = 2-prime-F/Me nucleotides
  modified_base 20..21: mod_base = OTHER; note = 2-prime-deoxynucleotides-3-prime-phosphate
  modified_base 20^21: mod_base = OTHER; note = Phosphorothioate linkage
ggatcatctcaagtcttactt

SEQ ID NO: 68 - RNA, 21 nt, synthetic construct
  source 1..21: mol_type = other RNA
  modified_base order(1,3,5,7,9..11,13,15,17): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
caggatcatctcaagtcttac

SEQ ID NO: 69 - RNA, 23 nt, synthetic construct
  source 1..23: mol_type = other RNA
  modified_base order(1,3,5,7,9,11..13,15,17,19,21..23): mod_base = OTHER; note = 2-prime-O-methylnucleotides-3-prime-phosphate
  modified_base order(2,4,6,8,10,14,16,18,20): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
gtaagacttgagatgatcctggc

SEQ ID NO: 70 - RNA, 23 nt, synthetic construct
  source 1..23: mol_type = other RNA
  modified_base order(1,3..5,7,9,11..13,15,17,19,21..23): mod_base = OTHER; note = 2-prime-O-methylnucleotides-3-prime-phosphate
  modified_base order(2,4,6,8,10,14,16,18): mod_base = OTHER; note = 2-prime-deoxy-2-prime-fluoronucleotides-3-prime-pho sphate
  modified_base 20: mod_base = OTHER; note = 2-prime-F/Me nucleotide
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
gtaagacttgagatgatcctggc

SEQ ID NO: 71 - RNA, 23 nt, synthetic construct
  source 1..23: mol_type = other RNA
  modified_base order(1,3,5,7,9,11..13,15,17,19,21..23): mod_base = OTHER; note = 2-prime-O-methylnucleotides-3-prime-phosphate
  modified_base order(2,4,6,8,10,14,16): mod_base = OTHER; note = 2-prime-deoxy-2-prime-fluoronucleotides-3-prime-pho sphate
  modified_base order(18,20): mod_base = OTHER; note = 2-prime-F/Me nucleotides
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
gtaagacttgagatgatcctggc

SEQ ID NO: 72 - RNA, 23 nt, synthetic construct
  source 1..23: mol_type = other RNA
  modified_base order(1,3,5,7,9,11..13,15,17,19,21..23): mod_base = OTHER; note = 2-prime-O-methylnucleotides-3-prime-phosphate
  modified_base order(2,4,6,10,14,16): mod_base = OTHER; note = 2-prime-deoxy-2-prime-fluoronucleotides-3-prime-pho sphate
  modified_base order(8,18,20): mod_base = OTHER; note = 2-prime-F/Me nucleotides
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
gtaagacttgagatgatcctggc

SEQ ID NO: 73 - RNA, 21 nt, synthetic construct
  source 1..21: mol_type = other RNA
  modified_base order(1,3,5,7,13,15,17,19,21): mod_base = OTHER; note = 2-prime-deoxy-2-prime-fluoronucleotides-3-prime-pho sphate
  modified_base order(9,10,11): mod_base = OTHER; note = 2-prime-F/Me nucleotides
caggatcatctcaagtcttac

SEQ ID NO: 74 - DNA, 12 bp, synthetic construct
  misc_feature order(1..5,7..12): note = DNA
  misc_feature 6: note = RNA
  source 1..12: mol_type = other DNA
  modified_base order(1..5,7..12): mod_base = OTHER; note = 2-prime-deoxynucleotides-3-prime-phosphate
  modified_base 6: mod_base = OTHER; note = 2-prime-deoxy-2-prime-fluoronucleotide-3-prime-phos phate
tacagtctatgt

SEQ ID NO: 75 - DNA, 12 bp, synthetic construct
  misc_feature order(1..5,7..12): note = DNA
  misc_feature 6: note = RNA
  source 1..12: mol_type = other DNA
  modified_base order(1..5,7..12): mod_base = OTHER; note = 2-prime-deoxynucleotides-3-prime-phosphate
  modified_base 6: mod_base = OTHER; note = 2-prime-F/Me nucleotide
tacagtctatgt

SEQ ID NO: 76 - DNA, 12 bp, synthetic construct
  misc_feature order(1..6,8..12): note = DNA
  misc_feature 7: note = RNA
  source 1..12: mol_type = other DNA
  modified_base order(1..6,8..12): mod_base = OTHER; note = 2-prime-deoxynucleotides-3-prime-phosphate
  modified_base 7: mod_base = OTHER; note = 2-prime-deoxy-2-prime-fluoronucleotide-3-prime-phos phate
tacagtctatgt

SEQ ID NO: 77 - DNA, 12 bp, synthetic construct
  misc_feature order(1..5,7..12): note = DNA
  misc_feature 6: note = RNA
  source 1..12: mol_type = other DNA
  modified_base order(1..5,7..12): mod_base = OTHER; note = 2-prime-deoxynucleotides-3-prime-phosphate
  modified_base 6: mod_base = OTHER; note = 2-prime-F/Me nucleotide
tacagcctatgt

SEQ ID NO: 78 - DNA, 12 bp, synthetic construct
  source 1..12: mol_type = other DNA
  modified_base 1..12: mod_base = OTHER; note = 2-prime-deoxynucleotides-3-prime-phosphate
tacagtctatgt

SEQ ID NO: 79 - DNA, 12 bp, synthetic construct
  source 1..12: mol_type = other DNA
  modified_base order(1..5,7..12): mod_base = OTHER; note = 2-prime-deoxynucleotides-3-prime-phosphate
  modified_base 6: mod_base = OTHER; note = 2-prime-deoxy-2-prime-fluorodeoxyribosenucleotide-3 -prime-phosphate
tacagtctatgt

SEQ ID NO: 80 - DNA, 12 bp, synthetic construct
  source 1..12: mol_type = other DNA
  modified_base order(1..5,7..12): mod_base = OTHER; note = 2-prime-deoxynucleotides-3-prime-phosphate
  modified_base 6: mod_base = OTHER; note = 2-prime-F/Me deoxyribose nucleotide
tacagtctatgt

SEQ ID NO: 81 - DNA, 12 bp, synthetic construct
  source 1..12: mol_type = other DNA
  modified_base order(1..6,8..12): mod_base = OTHER; note = 2-prime-deoxynucleotides-3-prime-phosphate
  modified_base 7: mod_base = OTHER; note = 2-prime-deoxy-2-prime-fluorodeoxyribosenucleotide-3 -prime-phosphate
tacagtctatgt

SEQ ID NO: 82 - DNA, 12 bp, synthetic construct
  source 1..12: mol_type = other DNA
  modified_base order(1..6,8..12): mod_base = OTHER; note = 2-prime-deoxynucleotides-3-prime-phosphate
  modified_base 7: mod_base = OTHER; note = 2-prime-F/Me deoxyribose nucleotide
tacagtctatgt

SEQ ID NO: 83 - DNA, 19 bp, synthetic construct
  source 1..19: mol_type = other DNA
taatattacataaataaaa

SEQ ID NO: 84 - RNA, 12 nt, synthetic construct
  source 1..12: mol_type = other RNA
acatagactgta

SEQ ID NO: 85 - DNA, 12 bp, synthetic construct
  source 1..12: mol_type = other DNA
  modified_base 1..12: mod_base = OTHER; note = 2-prime-deoxynucleotides-3-prime-phosphate
acatacactgta

SEQ ID NO: 86 - RNA, 21 nt, synthetic construct
  source 1..21: mol_type = other RNA
  ncRNA 1..21: ncRNA_class = siRNA
  modified_base order(1,3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
atctgtgttcttgctctataa

SEQ ID NO: 87 - RNA, 23 nt, synthetic construct
  source 1..23: mol_type = other RNA
  ncRNA 1..23: ncRNA_class = siRNA
  modified_base order(1..3,5,7,9,11..13,15,17,19,21,23): mod_base = OTHER; note = 2-prime-O-methylnucleotides-3-prime-phosphate
  modified_base order(4,6,8,10,14,16,18,20,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
ttttgtcacaagaacgagatatt

SEQ ID NO: 88 - RNA, 21 nt, synthetic construct
  source 1..21: mol_type = other RNA
  ncRNA 1..21: ncRNA_class = siRNA
  modified_base order(1,3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
aagatgatgtttgaaactatt

SEQ ID NO: 89 - RNA, 23 nt, synthetic construct
  source 1..23: mol_type = other RNA
  ncRNA 1..23: ncRNA_class = siRNA
  modified_base order(1..3,5,7,9,11..13,15,17,19,21,23): mod_base = OTHER; note = 2-prime-O-methylnucleotides-3-prime-phosphate
  modified_base order(4,6,8,10,14,16,18,20,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
tgttctactacaaactttgataa

SEQ ID NO: 90 - RNA, 21 nt, synthetic construct
  source 1..21: mol_type = other RNA
  ncRNA 1..21: ncRNA_class = siRNA
  modified_base order(1,3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
caggatcatctcaagtcttaa

SEQ ID NO: 91 - RNA, 23 nt, synthetic construct
  source 1..23: mol_type = other RNA
  ncRNA 1..23: ncRNA_class = siRNA
  modified_base order(1..3,5,7,9,11..13,15,17,19,21,23): mod_base = OTHER; note = 2-prime-O-methylnucleotides-3-prime-phosphate
  modified_base order(4,6,8,10,14,16,18,20,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
cggtcctagtagagttcagaatt

SEQ ID NO: 92 - DNA, 21 bp, synthetic construct
  misc_feature 20..21: note = DNA
  misc_feature 1..19: note = RNA
  source 1..21: mol_type = other DNA
  modified_base 20..21: mod_base = OTHER; note = 2-prime-deoxynucleotides-3-prime-phosphate
  modified_base order(4,5,7..10,14..17,19): mod_base = OTHER; note = 2-prime-deoxy-2-prime-fluoronucleotides-3-prime-pho sphate
  modified_base 20^21: mod_base = OTHER; note = Phosphorothioate linkage
ggatcatctcaagtcttactt

SEQ ID NO: 93 - DNA, 21 bp, synthetic construct
  misc_feature 1..2: note = DNA
  misc_feature 3..21: note = RNA
  source 1..21: mol_type = other DNA
  modified_base 1..2: mod_base = OTHER; note = 2-prime-deoxynucleotides-3-prime-phosphate
  modified_base order(3..5,8,13..15,20): mod_base = OTHER; note = 2-prime-deoxy-2-prime-fluoronucleotides-3-prime-pho sphate
  modified_base 1^2: mod_base = OTHER; note = Phosphorothioate linkage
ttcctagtagagttcagaatg

SEQ ID NO: 94 - RNA, 21 nt, synthetic construct
  source 1..21: mol_type = other RNA
  modified_base order(1,3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
  ncRNA 1..21: ncRNA_class = siRNA
caggatcatctcaagtcttac

SEQ ID NO: 95 - RNA, 23 nt, synthetic construct
  source 1..23: mol_type = other RNA
  ncRNA 1..23: ncRNA_class = siRNA
  modified_base order(1..3,5,7,9,11..13,15,17,19,21,23): mod_base = OTHER; note = 2-prime-O-methylnucleotides-3-prime-phosphate
  modified_base order(4,6,8,10,14,16,18,20,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
cggtcctagtagagttcagaatg

SEQ ID NO: 96 - RNA, 21 nt, synthetic construct
  source 1..21: mol_type = other RNA
  ncRNA 1..21: ncRNA_class = siRNA
  modified_base order(1..6,8,12..21): mod_base = OTHER; note = 2-prime-O-methylnucleotides-3-prime-phosphate
  modified_base order(7,9..11): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
cctctgatggtcaaagtccta

SEQ ID NO: 97 - RNA, 23 nt, synthetic construct
  source 1..23: mol_type = other RNA
  ncRNA 1..23: ncRNA_class = siRNA
  modified_base order(1..7,9,11..21,23): mod_base = OTHER; note = 2-prime-O-methylnucleotides-3-prime-phosphate
  modified_base order(8,10,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
caggagactaccagtttcaggat

SEQ ID NO: 98 - RNA, 21 nt, synthetic construct
  source 1..21: mol_type = other RNA
  modified_base order(1,3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
aacagtgttcttgctctataa

SEQ ID NO: 99 - RNA, 23 nt, synthetic construct
  source 1..23: mol_type = other RNA
  modified_base order(1..3,5,7,9,11..13,15,17,19,21): mod_base = OTHER; note = 2-prime-O-methylnucleotides-3-prime-phosphate
  modified_base order(4,6,8,10,14,16,18,20,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 23: mod_base = OTHER; note = 2-prime-F/Me nucleotide
  modified_base 21^22: mod_base = OTHER; note = Phosphorothioate linkage
  modified_base 22^23: mod_base = OTHER; note = Phosphorothioate linkage
ttttgtcacaagaacgagatatt

SEQ ID NO: 100 - RNA, 21 nt, synthetic construct
  source 1..21: mol_type = other RNA
  modified_base order(1..6,8,12..21): mod_base = OTHER; note = 2-prime-O-methylnucleotides-3-prime-phosphate
  modified_base order(7,9..11): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
agtgttcttgctctataaaca

SEQ ID NO: 101 - RNA, 23 nt, synthetic construct
  source 1..23: mol_type = other RNA
  modified_base order(1..7,9,11..21,23): mod_base = OTHER; note = 2-prime-O-methylnucleotides-3-prime-phosphate
  modified_base order(8,10,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
tgtcacaagaacgagatatttgt

SEQ ID NO: 102 - RNA, 23 nt, synthetic construct
  source 1..23: mol_type = other RNA
  modified_base order(1..7,9,11..16,18..21,23): mod_base = OTHER; note = 2-prime-O-methylnucleotides-3-prime-phosphate
  modified_base 17: mod_base = OTHER; note = 2-prime-F/Me nucleotide
  modified_base order(8,10,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
tgtcacaagaacgagatatttgt

SEQ ID NO: 103 - RNA, 21 nt, synthetic construct
  source 1..21: mol_type = other RNA
  modified_base order(1,3,5,7,9..11,13,15,17,19,21): mod_base = OTHER; note = 2-prime-deoxy-2-prime-fluoronucleotides-3-prime-pho sphate
  modified_base order(2,4,6,8,12,14,16,18,20): mod_base = OTHER; note = 2-prime-O-methylnucleotides-3-prime-phosphate
  modified_base 1^2: mod_base = OTHER; note = Phosphorothioate linkage
  modified_base 2^3: mod_base = OTHER; note = Phosphorothioate linkage
cctggacattcagaacaagaa

SEQ ID NO: 104 - RNA, 23 nt, synthetic construct
  source 1..23: mol_type = other RNA
  modified_base order(1..3,5,7,9,11..13,15,17,19,21,23): mod_base = OTHER; note = 2-prime-O-methylnucleotides-3-prime-phosphate
  modified_base order(4,6,8,10,14,16,18,20,22): mod_base = OTHER; note = 2-prime-deoxy-2-prime-fluoronucleotides-3-prime-pho sphate
  modified_base 1^2: mod_base = OTHER; note = Phosphorothioate linkage
  modified_base 2^3: mod_base = OTHER; note = Phosphorothioate linkage
  modified_base 21^22: mod_base = OTHER; note = Phosphorothioate linkage
  modified_base 22^23: mod_base = OTHER; note = Phosphorothioate linkage
tgggacctgtaagtcttgttctt

SEQ ID NO: 105 - RNA, 18 nt, synthetic construct
  source 1..18: mol_type = other RNA
  modified_base 1^2: mod_base = OTHER; note = Phosphorothioate linkage
  modified_base 2^3: mod_base = OTHER; note = Phosphorothioate linkage
cccccccccccccccccc

SEQ ID NO: 106 - RNA, 18 nt, synthetic construct
  source 1..18: mol_type = other RNA
cccccccccccccccccc

SEQ ID NO: 107 - RNA, 12 nt, synthetic construct
  source 1..12: mol_type = other RNA
acatagbctgta

SEQ ID NO: 108 - RNA, 12 nt, synthetic construct
  source 1..12: mol_type = other RNA
acatabactgta

(19) United States
(12) Patent Application Publication
(10) Pub. No.: US 2025/0223588 A1
(43) Pub. Date: Jul. 10, 2025

(54) OLIGONUCLEOTIDES WITH 2'-DEOXY-2'-F-2'-C-METHYL NUCLEOTIDES

(71) Applicant: ALNYLAM PHARMACEUTICALS, INC., Cambridge, MA (US)

(72) Inventors: Muthiah MANOHARAN, Cambridge, MA (US); Shigeo MATSUDA, Cambridge, MA (US); Dhrubajyoti DATTA, Cambridge, MA (US)

(73) Assignee: ALNYLAM PHARMACEUTICALS, INC., Cambridge, MA (US)

(21) Appl. No.: 18/702,640

(22) PCT Filed: Oct. 19, 2022
(86) PCT No.: PCT/US2022/047102

§ 371 Date: Apr. 18, 2024

Related U.S. Application Data
(60) Provisional application No. 63/257,289, filed on Oct. 19, 2021.

(51) Int. Cl. C12N 15/113

(52) U.S. Cl. CPC C12N 15/113; C12N 2310/11; C12N 2310/14; C12N 2310/315; C12N 2310/321; C12N 2310/322; C12N 2310/3231; C12N 2310/344; C12N 2310/351

(57) ABSTRACT

The present disclosure relates generally to 2′-geminal substituted nucleosides and nucleotides, and oligonucleotides and dsRNA molecules comprising such 2′-geminal substituted nucleosides and nucleotides.

### CROSS-REFERENCE TO RELATED APPLICATIONS

This application is a 35 U.S.C. § 371 National Phase Entry application of International Application No. PCT/US2022/047102 filed Oct. 19, 2022, which designates the U.S. and claims benefit under § 119(e) of U.S. Provisional Application No. 63/257,289 filed Oct. 19, 2021, contents of all of which are incorporated herein by reference in their entireties.

### SEQUENCE LISTING

The instant application contains a Sequence Listing which has been submitted electronically in XML format and is hereby incorporated by reference in its entirety. Said XML copy, created on Dec. 22, 2022, is named 051058-190210WOPT.xml and is 282,484 bytes in size.

### TECHNICAL FIELD

The present disclosure relates generally to 2′-geminal-substituted nucleosides, oligonucleotides and dsRNA comprising same and uses thereof.

### BACKGROUND

RNA interference or “RNAi” is a term initially coined by Fire and co-workers to describe the observation that double-stranded RNAi (dsRNA) can block gene expression (Fire et al. (1998) Nature 391, 806-811; Elbashir et al. (2001) Genes Dev. 15, 188-200). Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, but the protein components of this activity remained unknown.

There remains a need in the art for effective nucleotides or modifications for dsRNA molecules, which are advantageous for inhibition of target gene expression. This invention is directed to that effort.

### SUMMARY

In one aspect, provided herein is an oligonucleotide comprising: (i) at least one 2′-geminal-substituted nucleoside of formula (I) or (I′):

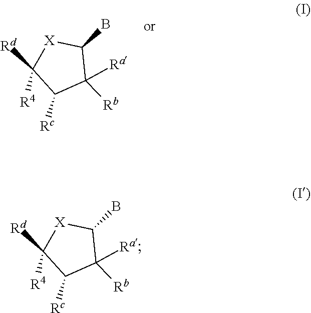

and/or (ii) a 2′-geminal-substituted nucleoside of formula (II) or (II′) at the 5′-terminal nucleotide:

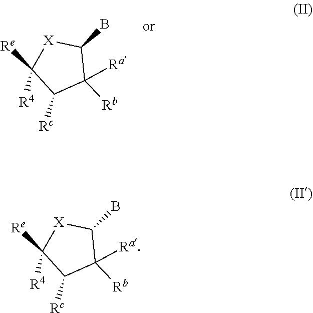

In formulae (I), (I′), (II), and (II′):
X is O, S, C(R^{X})₂, or N(R^{XN});
   each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{N}), or =CH₂;
   R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
B is an optionally modified nucleobase;
R^{a′} is hydrogen, halogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a bond to an internucleoside linkage to a subsequent nucleotide;
   R^{a2} is hydrogen or hydroxyl protecting group;
   R^{a3} is hydrogen or sulfur protecting group;
   R^{a4} is independently for each occurrence H, C₁-C₃₀alkyl, cyclyl, heterocyclyl, aryl, heteroaryl, aralkyl, sugar or R^{a5};
   R^{a5} is independently for each occurrence amino (NH₂), alkylamino, dialkylamino, arylamino, diarylamino, heteroarylamino, or diheteroaryl amino;
   m is 1-50;
   n is 1-50;
R^{b} is optionally substituted C₁₋₃₀alkyl, optionally substituted C₂₋₃₀alkenyl, optionally substituted C₂₋₃₀alkynyl, or halogen;
R^{c} is a bond to an internucleoside linkage to a subsequent nucleotide, hydrogen, halogen, —OR^{c2}, —SR^{c3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, or a linker covalently attached to a solid support, and where, optionally, at least R^{c} or R^{b} is a bond to \n internucleoside linkage to a subsequent nucleotide;
   R^{c2} is hydrogen or hydroxyl protecting group;
   R^{c3} is hydrogen or sulfur protecting group;
   R^{c4} is independently for each occurrence H, C₁-C₃₀alkyl, cyclyl, heterocyclyl, aryl, heteroaryl, aralkyl, sugar or R^{c5};
   R^{c5} is independently for each occurrence amino (NH₂), alkylamino, dialkylamino, arylamino, diarylamino, heteroarylamino, or diheteroaryl amino;
   r is 1-50;
   s is 1-50;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R^{a} taken together are 4′-C(R^{a11}R^{a12})ᵥY-2′ or 4′-Y—C(R^{a11}R^{a12})ᵥ-2′;
   Y is —O—, —CH₂—, —CH(Me)-, —C(CH₃)₂—, —S—, —N(R^{a13})—, —C(O)—, —C(S)—, —S(O)—, —S(O)₂—, —OC(O)—, —C(O)O—, —N(R^{a13})C(O)—, or —C(O)N(R^{a13})—;
   R^{a11} and R^{a12} independently are H, optionally substituted C₁-C₆alkyl, optionally substituted C₂-C₆alkenyl or optionally substituted C₂-C₆alkynyl;
   R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
   v is 1, 2 or 3;
or R⁴ and R^{c} taken together with the atoms to which they are attached form an optionally substituted C₃₋₈cycloalkyl, optionally substituted C₃₋₈cycloalkenyl, or optionally substituted 3-8 membered heterocyclyl;
R^{d} is —CH(R^{d1})—R^{d2} or —C(R^{d1})=CHR^{d2};
   R^{d1} is hydrogen, optionally substituted C₁₋₃₀alkyl, optionally substituted —C₂₋₃₀alkenyl, or optionally substituted —C₂₋₃₀alkynyl;
   R^{d2} is a bond to an internucleoside linkage to the preceding nucleotide;
R^{e} is optionally substituted C₁₋₆alkyl-R^{e1}, optionally substituted —C₂₋₆alkenyl-R^{e1}, or optionally substituted —C₂₋₆alkynyl-R^{e1};
   R^{e1} is —OR^{e2}, —SR^{e3}, —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂;
   R^{e2} is hydrogen or oxygen protecting group;
   R^{e3} is hydrogen or sulfur protecting group;
   each R^{e4} is independently hydrogen, optionally substituted C₁₋₃₀alkyl, optionally substituted C₂₋₃₀alkenyl, or optionally substituted C₂₋₃₀alkynyl, or an oxygen-protecting group;
      and each R^{e5} is independently hydrogen, optionally substituted C₁₋₃₀alkyl, optionally substituted C₂₋₃₀alkenyl, or optionally substituted C₂₋₃₀alkynyl, or a sulfur-protecting group.

In another aspect, provided herein is a double-stranded nucleic acid (e.g., dsRNA) comprising a first strand and a second strand substantially complementary to the first strand, and wherein at least one of the first or second strand is an oligonucleotide described herein, e.g., an oligonucleotide comprising: (i) at least one 2′-geminal-substituted nucleoside of formula (I) or (I′); and/or (ii) a 2′-geminal-substituted nucleoside of formula (II) or (II′) at the 5′-terminal nucleotide. In some embodiments, the first strand is an oligonucleotide described herein. In some embodiments of any one of the aspects described herein, the oligonucleotide comprises: (i) at least one 2′-geminal-substituted nucleoside of formula (I); and/or (ii) a 2′-geminal-substituted nucleoside of formula (II) at the 5′-terminal nucleotide.

In some embodiments of any one of the aspects described herein, the double-stranded nucleic acid (e.g., dsRNA) comprises an antisense strand and a sense strand, wherein the antisense strand and the sense strand are complementary to each other and form a double-stranded region, e.g., a double-stranded region of at least 15 base-pairs. The antisense strand comprises one or both of: (a) a 5′-terminal nucleoside that is a 2′-geminal-substituted nucleoside of formula (II) or (II′); and (b) a 2′-geminal-substituted nucleoside according to formula (I) or (I′) at least at one of positions 2-9 (e.g., at position 2, 3, 4, 5, 6, 7, 8 and/or 9), counting from the 5′-end of the antisense strand.

In some embodiments of any one of the aspects described herein, the antisense strand comprises: (i) a 5′-terminal nucleoside that is a 2′-geminal-substituted nucleoside of formula (II); and/or (ii) a 2′-geminal-substituted nucleoside according to formula (I) at least at one of positions 2-9 (e.g., at position 2, 3, 4, 5, 6, 7, 8 and/or 9), counting from the 5′-end of the antisense strand.

In some embodiments of any one of the aspects described herein, the 5′-terminal nucleotide of the antisense strand is a 2′-geminal-substituted nucleotide of formula (II) or (II′). For example, the 5′-terminal nucleotide of the antisense strand is a 2′-geminal-substituted nucleotide of formula (II).

In some embodiments of any one of the aspects described herein, the antisense strand comprises a vinylphosphonate (e.g., E-vinylphosphonate) group at its 5′-end. For example, the 5′-terminal nucleotide of the antisense strand is a 2′-geminal-substituted nucleotide of formula (II) or (II′), and wherein R^{e} is vinyl phosphonate (e.g., R^{e} is R^{e} is —CH═CHR^{e} and R^{e1} is —P(O)(OR^{e4})₂). In some embodiments of any one of the aspects described herein, the 5′-terminal nucleotide of the antisense strand is a 2′-geminal-substituted nucleotide of formula (II), and wherein R^{e} is vinyl phosphonate (e.g., R^{e} is R^{e} is —CH═CHR^{e} and R^{e1} is —P(O)(OR^{e4})₂)

In some embodiments of any one of the aspects described herein, the antisense strand comprises a nucleoside of Formula (I) or (I′) at least at position 3, 4, 5, 6, 7, 8 or 9, counting from the 5′-end of the antisense strand. For example, the antisense strand comprises a nucleoside of Formula (I) or (I′) at least at position 4, counting from the 5′-end of the antisense strand. In another example, the antisense strand comprises a nucleoside of Formula (I) or (I′) at least at least at position 5, counting from the 5′-end of the antisense strand. In yet another example, the antisense strand comprises a nucleoside of Formula (I) or (I′) at least at least at position 6, counting from the 5′-end of the antisense strand. In still another example, the antisense strand comprises a nucleoside of Formula (I) or (I′) at least at least at position 7, counting from the 5′-end of the antisense strand. In yet still another example, the antisense strand comprises a nucleoside of Formula (I) or (I′) at least at least at position 8, counting from the 5′-end of the antisense strand. In one example, the antisense strand comprises a nucleoside of Formula (I) or (I′) at least at least at position 9, counting from the 5′-end of the antisense strand.

In some embodiments of any one of the aspects described herein, the antisense strand comprises a nucleoside of Formula (I) at least at position 3, 4, 5, 6, 7, 8 or 9, counting from the 5′-end of the antisense strand. For example, the antisense strand comprises a nucleoside of Formula (I) at least at position 4, counting from the 5′-end of the antisense strand. In another example, the antisense strand comprises a nucleoside of Formula (I) at least at least at position 5, counting from the 5′-end of the antisense strand. In yet another example, the antisense strand comprises a nucleoside of Formula (I) at least at least at position 6, counting from the 5′-end of the antisense strand. In still another example, the antisense strand comprises a nucleoside of Formula (I) at least at least at position 7, counting from the 5′-end of the antisense strand. In yet still another example, the antisense strand comprises a nucleoside of Formula (I) at least at least at position 8, counting from the 5′-end of the antisense strand. In one example, the antisense strand comprises a nucleoside of Formula (I) at least at least at position 9, counting from the 5′-end of the antisense strand.

In some embodiments of any one of the aspects described herein, a nucleoside of formula (I) is of formula (IA):

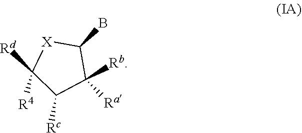

In some embodiments of any one of the aspects described herein, a nucleoside of formula (I) is of formula (IB):

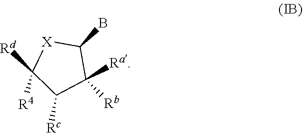

In some embodiments of any one of the aspects described herein, a nucleoside of formula (II) is of formula (IIA):

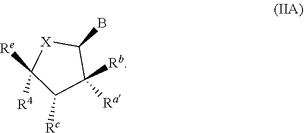

In some embodiments of any one of the aspects described herein, a nucleoside of formula (II) is of formula (IIB):

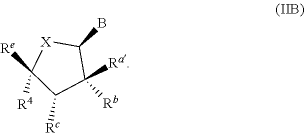

In some embodiments of any one of the aspects described herein, a nucleoside of formula (I′) is of formula (IA′):

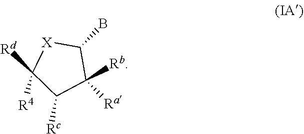

In some embodiments of any one of the aspects described herein, a nucleoside of formula (I′) is of formula (IB′):

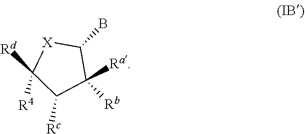

In some embodiments of any one of the aspects described herein, a nucleoside of formula (II′) is of formula (IIA′):

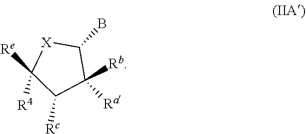

In some embodiments of any one of the aspects described herein, a nucleoside of formula (II′) is of formula (IIB′):

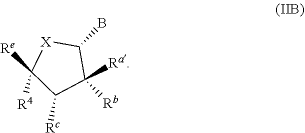

In some embodiments of any one of the aspects described herein, a nucleoside of formula (II) is of formula (IIA) or (IIB):
wherein:
X is O;
R^{a′} is halogen (e.g., F or Cl), hydroxyl, optionally substituted C₁₋₃₀alkoxy (e.g., —(CH-2₂)ₙCH3, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{e} is —CH═CHR^{e1}, where R^{e1} is —P(O)(OR^{e4})₂.

In some embodiments of any one of the aspects described herein, a nucleoside of formula (II) is of formula (IIA) or (IIB):
wherein:
X is O;
R^{a′} is F, Cl or optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2);
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R^{c} is a bond to an internucleoside linkage to a subsequent nucleoside;
R⁴ is hydrogen; and
R^{e} is —CH═CHR^{e}, where R^{e1} is —P(O)(OR^{e4})₂.

In some embodiments of any one of the aspects described herein, a nucleoside of formula (II) is of formula (IIA) or (IIB):
wherein:
X is O;
R^{a′} is Cl or optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2);
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R^{c} is a bond to an internucleoside linkage to a subsequent nucleoside;
R⁴ is hydrogen; and
R^{e} is —CH₂—O—R^{e2} or —CH═CHR^{e1}, where R^{e2} is hydrogen or oxygen protecting group and
R^{e1} is —P(O)(OR^{e4})₂.

In some embodiments of any one of the aspects described herein, a nucleoside of formula (II) is of formula (IIA) or (IIB):
wherein:
X is O;
R^{a′} is halogen (e.g., F, Br or Cl);
R^{b} is R^{a′} is halogen (e.g., F, Br or Cl);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{e} is —CH₂—O—R^{e2} or —CH═CHR^{e1}, where R^{e2} is hydrogen or oxygen protecting group and
R^{e1} is —P(O)(OR^{e4})₂.

In some embodiments of any one of the aspects described herein, a nucleoside of formula (II′) is of formula (IIA′) or (IIB′):
wherein:
X is O;
R^{a′} is halogen (e.g., F, Br or Cl), hydroxyl, optionally substituted C₁₋₃₀alkoxy (e.g., —(CH2₂)ₙCH3, where n is 1-21, e.g., 1, 16; or —(CH2₂)ₘ-NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Br or Cl);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{e} is —CH₂—O—R^{e2} or —CH═CHR^{e1}, where R^{e2} is hydrogen or oxygen protecting group and
R^{e1} is —P(O)(OR^{e4})₂.

In some embodiments of any one of the aspects described herein, a nucleoside of formula (I) is of formula (IA) or (IB):
wherein:
X is O;
R^{a′} is halogen (e.g., F or Cl), hydroxyl, optionally substituted C₁₋₃₀alkoxy (e.g., —(CH-2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R^{e} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{d} is a bond to an internucleoside linkage to a preceding nucleoside.

In some embodiments of any one of the aspects described herein, a nucleoside of formula (I) is of formula (IA) or (IB
wherein:
X is O;
R^{a′} is F, Cl or optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2);
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R^{c} is a bond to an internucleoside linkage to a subsequent nucleoside or hydroxyl;
R⁴ is hydrogen; and
R^{d} is a bond to an internucleoside linkage to a preceding nucleoside.

In some embodiments of any one of the aspects described herein, a nucleoside of formula (I) is of formula (IA) or (IB
wherein:
X is O;
R^{a′} Cl or optionally substituted C₁₋₃₀alkoxy (e.g., —(CH2₂)ₙCH3, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2);
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R^{c} is a bond to an internucleoside linkage to a subsequent nucleoside or hydroxyl;
R⁴ is hydrogen; and
R^{d} is a bond to an internucleoside linkage to a preceding nucleoside.

In some embodiments of any one of the aspects described herein, a nucleoside of formula (I) is of formula (IA) or (IB):
wherein:
X is O;
R^{a′} is halogen (e.g., F, Br or Cl);
R^{b} is halogen (e.g., F, Br or Cl);
R^{c} is a bond to an internucleoside linkage to a subsequent nucleoside or hydroxyl;
R⁴ is hydrogen; and
R^{d} is a bond to an internucleoside linkage to a preceding nucleoside.

In some embodiments of any one of the aspects described herein, a nucleoside of formula (I) is of formula (IA) or (IB):
wherein:
X is O;
R^{a′} is halogen (e.g., F, Br or Cl), hydroxyl, optionally substituted C₁₋₃₀alkoxy (e.g., —(CH2₂)ₙCH3, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Br or Cl);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{d} is a bond to an internucleoside linkage to a preceding nucleoside

In some embodiments of any one of the aspects described herein, the antisense strand can be about 17-42 nucleotides in length. For example, the antisense strand is at least about 17, e.g., about 18, about 19, about 20, about 21, about 22, about 23, about 24, about 25, about 26, about 27, about 28, about 29, about 30 or more nucleotides in length. In some embodiments of any one of the aspects described herein, the antisense strand is about 19, about 20, about 21, about 22, about 23, about 24, about 25 or about 26 nucleotides in length. For example, the antisense strand is about 22, about 23, about 24, or about 25 nucleotides in length.

In some embodiments of any one of the aspects described herein, the sense strand can be about 15-40 nucleotides in length. For example, the sense strand is at least about 15, about 16, e.g., about 17, about 18, about 19, about 20, about 21, about 22, about 23, about 24, about 25, about 26, or more nucleotides in length. In some embodiments of any one of the aspects described herein, the sense strand is about 19, about 20, about 21, about 22, about 23, about 24 or about 25 nucleotides in length. For example, the sense strand is about 21 nucleotides in length.

In some embodiments of any one of the aspects described herein, the sense strand is 15 nucleotides in length and the antisense strand is 18, 19, 20, 21, or 22 (e.g., 20) nucleotides in length. In some embodiments of any one of the aspects described herein, the sense strand is 19 nucleotides in length and the antisense strand is 19, 20, or 21 nucleotides in length. In some embodiments of any one of the aspects described herein, the sense strand is 20 nucleotides in length and the antisense strand is 20, 21, or 22 nucleotides in length. In some embodiments of any one of the aspects described herein, the sense strand is 21 nucleotides in length and the antisense strand is 21, 22, or 23 nucleotides in length. In some embodiments of any one of the aspects described herein, the sense strand is 20-24 (e.g., 22) nucleotides in length and the antisense strand is 34-38 (e.g. 36) nucleotides in length.

In some embodiments of any one of the aspects described herein, the sense strand is 21 nucleotides in length and the antisense strand is 22, 23 or 25 nucleotides in length.

In some embodiments of the various aspects described herein, the double-stranded region of the double-stranded nucleic acid (e.g., dsRNA) can be at least about 18, e.g., about 19, about 20, about 21, about 22, about 23, about 24, about 25 or more base-pairs, for example, a double-stranded region of about 21 base-pairs.

In some embodiments of any one of the aspects described herein, the antisense strand is about 21, about 22, about 23, about 24 or about 25 nucleotides in length, the sense strand is about 21 nucleotides in length, and the dsRNA comprises a double-stranded region of at least 18, e.g., 19, 20 or 21 base-pairs, such as 21 base-pairs.

The double-stranded nucleic acid (e.g., dsRNA) can comprise blunt ends and/or single-stranded overhangs at the end. For example, the double-stranded nucleic acid (e.g., dsRNA) can comprise comprises a blunt end at 5′-end of the antisense strand. In another example, the double-stranded nucleic acid (e.g., dsRNA) can comprise comprises a 1-5 (e.g., 1 or 2) nucleotide single-stranded overhang at 3′-end of the antisense strand, e.g., the 3′-end of the antisense strand extends past the 5′-end of the sense strand.

In some embodiments of any one of the aspects described herein, the double-stranded nucleic acid (e.g., dsRNA) comprises a blunt end at 5′-end of the antisense strand and a 1-5 (e.g., 1 or 2) nucleotide single-stranded overhang at 3′-end of the antisense strand.

In some embodiments of any one of the aspects described herein, the double-stranded nucleic acid (e.g., dsRNA) comprises at least one, e.g., 2, 3, 4, 5, 6, 7, 8, 9, 10 or more phosphorothioate internucleoside linkages. For example, the double-stranded nucleic acid (e.g., dsRNA) comprises at least 4 phosphorothioate internucleoside linkages, such as at least 6 phosphorothioate internucleoside linkages or at least 8 phosphorothioate internucleoside linkages.

It is noted that the phosphorothioate internucleoside linkages can be present in one strand or both strand. Further, the phosphorothioate internucleoside linkages can be present anywhere in the strand. For example, the phosphorothioate internucleoside linkages can be present at one end of the strand, at both ends of the strand, both at one end and at internal positions of the strand, or at both ends and at internal positions of the strand. Preferably, the phosphorothioate internucleoside linkages are present at both ends of the strand.

In some embodiments, the antisense strand comprises at least one, e.g., two, three, four or more phosphorothioate internucleoside linkages. For example, the antisense strand comprises 4 or more phosphorothioate internucleoside linkages. In some embodiments of any one of the aspects described herein, the antisense strand comprises a phosphorothioate internucleoside linkage between positions 1 and 2, counting from the 3′-end of the strand, and a phosphorothioate internucleoside linkage between positions 1 and 2, counting from the 5′-end of the strand. In yet some other embodiments of any one of the aspects described herein, the antisense strand comprises a phosphorothioate internucleoside linkage between positions 1 and 2, and between positions 2 and 3, counting from the 3′-end of the strand, and a phosphorothioate internucleoside linkage between positions 1 and 2, counting from the 5′-end of the strand. In still some other embodiments of any one of the aspects described herein, the antisense strand comprises a phosphorothioate internucleoside linkage between positions 1 and 2, and between positions 2 and 3, counting from the 3′-end of the strand, and a phosphorothioate internucleoside linkage between positions 1 and 2, and between positions 2 and 3, counting from the 5′-end of the strand. In yet still some other embodiments of any one of the aspects described herein, the antisense strand comprises a phosphorothioate internucleoside linkage between positions 1 and 2, between positions 2 and 3, and between positions 3 and 4, counting from the 3′-end of the strand, and a phosphorothioate internucleoside linkage between positions 1 and 2, counting from the 5′-end of the strand. In some embodiments of any one of the aspects described herein, the antisense strand comprises a phosphorothioate internucleoside linkage between positions 1 and 2, counting from the 3′-end of the strand, and a phosphorothioate internucleoside linkage between positions 1 and 2, and between positions 2 and 3, counting from the 5′-end of the strand. In yet other embodiments of any one of the aspects described herein, the antisense strand comprises a phosphorothioate internucleoside linkage between positions 1 and 2, counting from the 3′-end of the strand, and a phosphorothioate internucleoside linkage between positions 1 and 2, between positions 2 and 3, and between positions 3 and 4, counting from the 5′-end of the strand.

Like the antisense strand, the sense strand can also comprise one or more, e.g., two, three, four or more phosphorothioate internucleoside linkages. For example, the sense strand comprises a phosphorothioate internucleoside linkage between positions 1 and 2, counting from 5′-end of the strand. In some embodiments of any one of the aspects described herein, the sense strand comprises a phosphorothioate internucleoside linkage between positions 1 and 2, counting from 5′-end of the strand, and between positions 1 and 2, counting from 3′-end of the strand.

In yet some embodiments of any one of the aspects described herein, the sense strand comprises a phosphorothioate internucleoside linkage between positions 1 and 2, and between positions 2 and 3, counting from 5′-end of the strand. For example, the sense strand comprises a phosphorothioate internucleoside linkage between positions 1 and 2, and between positions 2 and 3, counting from 5′-end of the strand, and between positions 1 and 2, and between positions 2 and 3, counting from 3′-end of the strand.

In some embodiments of any one of the aspects described herein, the double-stranded nucleic acid (e.g., dsRNA) comprises a ligand. For example, the sense strand comprises a ligand linked thereto. It is noted that the ligand can be linked to any available position of the nucleotide at the 3′-end, i.e., nucleotide at position 1 (counting 3′-end) or at the 5′-end, i.e., nucleotide at position 1 (counting 5′-end) of the sense strand.

Embodiments of the various aspects described herein include a ligand. It is noted that the ligand can be selected from the group consisting of peptides, centyrins, antibodies (e.g., antiCD-4 antibodies and antiCD-117 antibodies), antibody fragments, T-cell targeting ligands, B-cell targeting ligands, cancer cell targeting ligands (e.g., DUPA, folate, and RGD), spleen targeting functionalities, lung targeting functionalities, bone marrow targeting functionalities, phage display peptides, cell permeation peptides (CPPs), integrin ligands, multianionic ligands, multicationic ligands, monovalent and multivalent carbohydrates (e.g., GalNAc, mannose, mannose-6 phosphate, mucose, and mlucose), kidney targeting ligands, BBB penetration ligands, lipids, and amino acids (e.g., L-amino acids, D-amino acids, ando-amino acids). In some embodiments of any one of the aspects described herein, the ligand is a mono- or multi-valent N-acetylgalactosamine (GalNac).

It is noted that the double-stranded nucleic acid (e.g., dsRNA) described herein can comprise one or more additional nucleic acid modifications such as nucleobase modifications, sugar modifications, inter-sugar linkage modifications, or any combination thereof. Accordingly, in some embodiments of any one of the aspects described herein, double-stranded nucleic acid (e.g., dsRNA) comprises at least one, e.g., 2, 3, 4, 5, 6, 7, 8, 9, 10 or more 2′-fluoro nucleotide. For example, the antisense strand and/or the sense stand comprises independently at least one, e.g., 2, 3, 4, 5 or more 2′-fluoro nucleotides.

In some embodiments of any one of the aspects described herein, the antisense strand comprises a 2′-fluoro nucleotide at positions 2, 14 and 16, counting from the 5′-end of the antisense strand. For example, the antisense strand comprises a 2′-fluoro nucleotide at positions 2, 6, 14 and 16, counting from the 5′-end of the antisense strand. In another non-limiting example, the antisense strand comprises a 2′-fluoro nucleotide at positions 2, 6, 9, 14 and 16, counting from the 5′-end of the antisense strand. In some further examples, the antisense strand comprises a 2′-fluoro nucleotide at positions 2, 6, 8, 9, 14 and 16, counting from the 5′-end of the antisense strand.

In some embodiments of any one of the aspects described herein, the antisense strand comprises a 2′-fluoro nucleotide at positions 2, 5, 7, 12, 14 and 16 counting from the 5′-end of the antisense strand.

In some embodiments of any one of the aspects described herein, the sense strand comprises a 2′-fluoro nucleotide at positions 7, 9 and 11, counting from the 5′-end of the sense strand or at positions 11, 13 and 15, counting from the 3′-end of the sense strand. For example, the sense strand comprises a 2′-fluoro nucleotide at positions 7, 9, 10 and 11, counting from the 5′-end of the sense strand or at positions 11, 12, 13 and 15, counting from the 3′-end of the sense strand.

In some embodiments of any one of the aspects described herein, the sense strand comprises a 2′-fluoro nucleotide at positions 9, 10, and 11, counting from the 5′-end of the sense strand or at positions 11, 12, and 13 counting from the 3′-end of the sense strand.

In some embodiments of any one of the aspects described herein, the antisense strand comprises a 2′-fluoro nucleotide at least at positions 2, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand. For example, the antisense strand comprises a 2′-fluoro nucleotide at least at positions 2, 6, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand. In another example, the antisense strand comprises a 2′-fluoro nucleotide at least at positions 2, 6, 9, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand. In yet another example, the antisense strand comprises a 2′-fluoro nucleotide at least at positions 2, 6, 8, 9, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand.

In some further non-limiting example, the antisense strand comprises a 2′-fluoro nucleotide at least at positions 2, 14 and 16, counting from the 5′-end of the antisense strand, and the sense strand comprises a 2′-fluoro nucleotide at least at positions 7, 9 and 11, counting from the 5′-end of the sense strand or at least at positions 11, 12, 13 and 15, counting from the 3′-end of the sense strand. For example, the antisense strand comprises a 2′-fluoro nucleotide at least at positions 2, 6, 14 and 16, counting from the 5′-end of the antisense strand, and the sense strand comprises a 2′-fluoro nucleotide at least at positions 7, 9, 10 and 11, counting from the 5′-end of the sense strand or at least at positions 11, 12, 13 and 15, counting from the 3′-end of the sense strand. In another example, the antisense strand comprises a 2′-fluoro nucleotide at least at positions 2, 6, 9, 14 and 16, counting from the 5′-end of the antisense strand, and the sense strand comprises a 2′-fluoro nucleotide at least at positions 7, 9, 10 and 11, counting from the 5′-end of the sense strand or at least at positions 11, 12, 13 and 15, counting from the 3′-end of the sense strand. In yet another example, the antisense strand comprises a 2′-fluoro nucleotide at least at positions 2, 6, 8, 9, 14 and 16, counting from the 5′-end of the antisense strand, and the sense strand comprises a 2′-fluoro nucleotide at least at positions 7, 9, 10 and 11, counting from the 5′-end of the sense strand or at least at positions 11, 12, 13 and 15, counting from the 3′-end of the sense strand.

The dsRNAs described herein can comprise one or more, e.g., 2, 3, 4, 5, 6, 7, 8, 9, 10 or more 2′-deoxy (i.e., 2′-H or DNA) nucleotides. For example, the antisense strand and/or the sense stand comprises independently at least one, e.g., 2, 3, 4, 5 or more 2′-deoxy (i.e., 2′-H or DNA) nucleotides.

In some embodiments of any one of the aspects described herein, the antisense strand comprises a DNA nucleotide at positions 2, 5, 7, and 12 counting from the 5′-end of the antisense strand. In some embodiments of any one of the aspects described herein, the antisense strand comprises a DNA nucleotide at positions 2, 5, 7, 12, and 14 counting from the 5′-end of the antisense strand. In some embodiments of any one of the aspects described herein, the antisense strand comprises a DNA nucleotide at positions 2, 5, 7, 12, 14 and 16 counting from the 5′-end of the antisense strand.

In some embodiments of any one of the aspects described herein, the antisense strand comprises a DNA nucleotide at positions 2, 5, 7 and 12, counting from the 5′-end of the antisense strand; and a 2′-fluoro nucleotide at position 14 of the antisense strand.

The dsRNAs described herein can comprise one or more, e.g., 2, 3, 4, 5, 6, 7, 8, 9, 10 or more 2′-OMe nucleotides. For example, the antisense strand and/or the sense stand comprises independently at least one, e.g., 2, 3, 4, 5 or more 2′-OMe nucleotides. In some embodiments of any one of the aspects described herein, all remaining nucleotides, i.e., other than modifications specified herein, in the antisense strand are 2′-OMe nucleotides. Similarly, in some embodiments of any one of the aspects described herein, all remaining nucleotides, i.e., other than modifications specified herein, in the antisense strand are 2′-OMe nucleotides.

In some embodiments of any one of the aspects described herein, the antisense strand comprises aphosphate group or aphosphate analog or derivative thereof at its 5′-end. For example, the antisense strand comprises a 5′-vinylphosphonate nucleotide at its 5′-end. For example, the antisense strand comprises a 5′-E-vinylphosphanate nucleotide at its 5′-end.

In some embodiments of any one of the aspects described herein, the dsRNA comprises at least one, e.g., 2, 3, 4, 5, 6, 7, 8, 9, 10 or more locked nucleic acid (LNA) or bridged nucleic acid (BNA) nucleotides. For example, the antisense and/or the sense strand comprises independently at least one, e.g., 2, 3, 4, 5 or more LNA or BNA nucleotides.

In some embodiments of any one of the aspects described herein, the dsRNA comprises at least one, e.g., 2, 3, 4, 5, 6, 7, 8, 9, 10 or more cyclohexene nucleic acid (CeNA) nucleotides. For example, the antisense and/or the sense strand comprises independently at least one, e.g., 2, 3, 4, 5 or more CeNA nucleotides.

In some embodiments of any one of the aspects described herein, the dsRNA comprises at least one, e.g., 2, 3, 4, 5, 6, 7, 8, 9, 10 or more thermally stabilizing modification. For example, the antisense and/or the sense strand comprises independently at least one, e.g., 2, 3, 4, 5 or more thermally stabilizing modification.

In some embodiments of any one of the aspects described herein, the dsRNA comprises at least one, e.g., 2, 3, 4, 5, 6, 7, 8, 9, 10 or more abasic nucleotides. For example, the antisense and/or the sense strand comprises independently at least one, e.g., 2, 3, 4, 5 or more abasic nucleotides.

In some embodiments of any one of the aspects described herein, the dsRNA comprises at least one, e.g., 2, 3, 4, 5, 6, 7, 8, 9, 10 or more 2′-deoxy nucleotides. For example, the antisense and/or the sense strand comprises independently at least one, e.g., 2, 3, 4, 5 or more 2′-deoxy nucleotides. In some embodiments, the antisense strand comprises one or more, e.g., one, two or more 2′-deoxy nucleotides in the single-stranded overhang.

In some embodiments of any one of the aspects described herein, the dsRNA comprises at least one, e.g., 2, 3, 4, 5, 6, 7, 8, 9, 10 or acyclic (e.g., unlocked nucleic acid (UNA), glycol nucleic acid (GNA) or (S)-glycol nucleic acid (S-GNA)) nucleotides. For example, the antisense and/or the sense strand comprises independently at least one, e.g., 2, 3, 4, 5 or more UNA and/or GNA nucleotides.

In some embodiments of any one of the aspects described herein, the dsRNA comprises at least one, e.g., 2, 3, 4, 5, 6, 7, 8, 9, 10 or thermally destabilizing modifications. For example, the antisense and/or the sense strand comprises independently at least one, e.g., 2, 3, 4, 5 or more thermally destabilizing modifications. Some exemplary thermally destabilizing modifications include, but are not limited to, abasic nucleotides, 2′-deoxy nucleotides, acyclic nucleotides (e.g., UNA, GNA and (S)-GNA), 2′-5′ linked nucleotides (3′-RNA), threose nucleotides (TNA), 2′ gem Me/F nucleotides, and a mismatch with the opposing nucleotide in the other strand.

In some embodiments of any one of the aspects described herein, the antisense strand comprises at least one thermally destabilizing modification in the seed region (i.e., positions 2-9 from the 5′-end) of the antisense strand. For example, the antisense strand comprises a thermally destabilizing modification at least at one of positions 6, 7 or 8, counting from the 5′-end of the strand. In some embodiments of any one of the aspects described herein, the antisense strand comprises a thermally destabilizing modification at position 7, counting from the 5′-end of the strand.

In some embodiments of any one of the aspects described herein, an oligonucleotide described herein solely comprises 2′-geminal-substituted nucleotides of formulae (I) and (II).

In some embodiments of any one of the aspects described herein, the oligonucleotide solely comprises 2′-geminal-substituted nucleotides of formulae (I) and (II), and the oligonucleotide further comprises a ligand, e.g., a mono- or multi-valent N-acetylgalactosamine (GalNac) linked to the oligonucleotide. For example, the oligonucleotide solely comprises 2′-geminal-substituted nucleotides of formulae (I) and (II), and the oligonucleotide further comprises a ligand, e.g., a mono- or multi-valent N-acetylgalactosamine (GalNac) linked to its 3′-end.

In yet another aspect, provided herein is a 2′-geminal-substituted nucleotide or monomer of formula (III) or (III′):

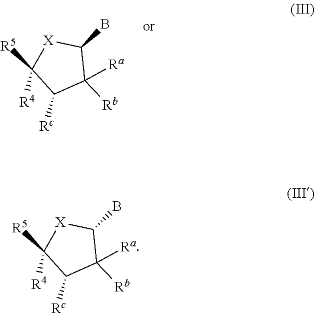

In formula (III) and (III′):
X is O, S, C(R^{X})₂, or N(R^{XN});
   each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{N}), or =CH₂;
   R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
B is an optionally modified nucleobase;
R^{a} is hydrogen, halogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a reactive phosphorus group;
   R^{a2} is hydrogen or hydroxyl protecting group;
   R^{a3} is hydrogen or sulfur protecting group;
   R^{a4} is independently for each occurrence H, C₁-C₃₀alkyl, cyclyl, heterocyclyl, aryl, heteroaryl, aralkyl, sugar or R^{a5};
   R^{a5} is independently for each occurrence amino (NH₂), alkylamino, dialkylamino, arylamino, diarylamino, heteroarylamino, or diheteroaryl amino;
   m is 1-50;
   n is 1-50;
R^{b} is optionally substituted C₁₋₃₀alkyl, optionally substituted C₂₋₃₀alkenyl, optionally substituted C₂₋₃₀alkynyl, or halogen;
R³ is hydrogen, halogen, —OR^{e2}, —SR^{e3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, a linker covalently attached to a solid support, or a reactive phosphorus group;
   R^{c2} is hydrogen or hydroxyl protecting group;
   R^{c3} is hydrogen or sulfur protecting group;
   R^{c4} is independently for each occurrence H, C₁-C₃₀alkyl, cyclyl, heterocyclyl, aryl, heteroaryl, aralkyl, sugar or R^{c5};
   R^{c5} is independently for each occurrence amino (NH₂), alkylamino, dialkylamino, arylamino, diarylamino, heteroarylamino, or diheteroaryl amino;
   r is 1-50;
   s is 1-50;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R^{c} taken together are 4′-C(R^{a11}R^{a12})ᵥ—Y-2′ or 4′-Y—C(R^{a11}R^{a12})ᵥ-2′;
   Y is —O—, —CH₂—, —CH(Me)-, —C(CH₃)₂—, —S—, —N(R^{a13})—, —C(O)—, —C(S)—, —S(O)—, —S(O)₂—, —OC(O)—, —C(O)O—, —N(R^{a13})C(O)—, or —C(O)N(R^{a13})—;
   R^{a11} and R^{a12} independently are H, optionally substituted C₁-C₆alkyl, optionally substituted C₂-C₆alkenyl or optionally substituted C₂-C₆alkynyl;
   R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
   v is 1, 2 or 3;
   or R⁴ and R^{c} taken together with the atoms to which they are attached form an optionally substituted C₃₋₈cycloalkyl, optionally substituted C₃₋₈cycloalkenyl, or optionally substituted 3-8 membered heterocyclyl;
R⁵ is optionally substituted C₁₋₆alkyl-R^{5a}, optionally substituted —C₂₋₆alkenyl-R^{5a}, or optionally substituted —C₂₋₆alkynyl-R^{5a};
   R^{5a} is —OR^{5b}, —SR^{5c}, hydrogen, a phosphorus group, a phosphorous group, a solid support or a linker to a solid support, provided that only one of R^{3a}, R³ and R⁵ is a linkage to a solid support;
   R^{5b} is H or hydroxyl protecting group; and
   R^{5c} is H or sulfur protecting group.

In some embodiments of any one of the aspects described herein, a compound of Formula (III) is of Formula (IIIA):

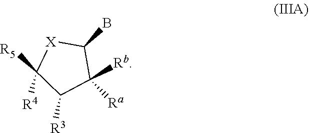

In some embodiments of any one of the aspects described herein, a compound of Formula (III) is of Formula (IIIB′):

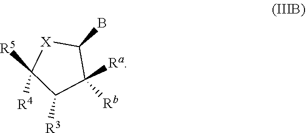

In some embodiments of any one of the aspects described herein, a compound of Formula (III′) is of Formula (IIIA′):

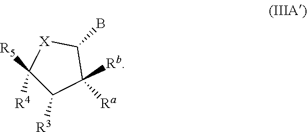

In some embodiments of any one of the aspects described herein, a compound of Formula (III′) is of Formula (IIIB′):

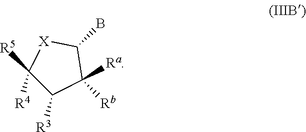

In some embodiments of any one of the aspects described herein, a compound of Formula (III) is of Formula (IIIA) or (IIIB), and wherein:
X is O;
R^{a} is halogen (e.g., F or Cl), hydroxyl, protected hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), a reactive phosphorous group (e.g., —OP(OR^{P})N(R^{P2})₂ (such as —OP(OCH₂CH₂CN)N(iPr)₂), —OP(SR^{P}) N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support, or a linker covalently attached to a solid support;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R⁴ is hydrogen;
R³ is a a reactive phosphorous group (e.g., —OP(OR^{P})N(R^{P2})₂ (such as —OP(OCH₂CH₂CN)N(iPr)₂), —OP(SR^{P}) N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3},
or —OP(O)(SR^{P})R^{P3}), a solid support, a linker covalently attached to a solid support, hydroxyl, or protected hydroxyl, provided that only one of R^{a} and R³ is reactive phosphorous group a solid support, or a linker covalently attached to a solid support; and
R⁵ is —CH═CHR^{5a}, where R^{5a} is —P(O)(OR^{5e})₂ and each R^{5e} is independently hydrogen, or optionally substituted C₁₋₃₀alkyl.

In some embodiments of any one of the aspects described herein, a compound of Formula (III) is of Formula (IIIA) or (IIIB), and wherein:
X is O;
R^{a} is F, Cl or optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where
p is 1 to 21, e.g., 1 or 2);
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R⁴ is hydrogen;
R³ is a reactive phosphorous group (e.g., —OP(OR^{P})N(R^{P2})₂ (such as —OP(OCH₂CH₂CN)N(iPr)₂), —OP(SR^{P}) N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3},
or —OP(O)(SR^{P})R^{P3}), a solid support, a linker covalently attached to a solid support, hydroxyl, or protected hydroxyl; and
R⁵ is —CH═CHR^{5a}, where R^{5a} is —P(O)(OR^{5e})₂ and each R^{5e} is independently hydrogen, or optionally substituted C₁₋₃₀alkyl.

In some embodiments of any one of the aspects described herein, a compound of Formula (III) is of Formula (IIIA) or (IIIB), and wherein:
X is O;
R^{a} is Cl or optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2);
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R⁴ is hydrogen;
R³ is a reactive phosphorous group (e.g., —OP(OR^{P})N(R^{P2})₂ (such as —OP(OCH₂CH₂CN)N(iPr)₂), —OP(SR^{P}) N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support, a linker covalently attached to a solid support, hydroxyl, or protected hydroxyl; and
R⁵ is —CH₂OR^{5b} or —CH═CHR^{5a}, where R^{5b} is H or hydroxyl protecting group and ^{5a} is —P(O)(OR^{5e})₂, and where each R^{5e} is independently hydrogen, or optionally substituted C₁₋₃₀alkyl.

In some embodiments of any one of the aspects described herein, a compound of Formula (III) is of Formula (IIIA) or (IIIB), and wherein
X is O;
R^{a} is halogen (e.g., Cl, Br or F);
R^{b} is halogen (e.g., Cl, Br or F);
R⁴ is hydrogen;
R³ is a reactive phosphorous group (e.g., —OP(OR^{P})N(R^{P2})₂ (such as —OP(OCH₂CH₂CN)N(iPr)₂), —OP(SR^{P}) N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support, a linker covalently attached to a solid support, hydroxyl, or protected hydroxyl; and
R⁵ is —CH₂OR^{5b} or —CH═CHR^{5a}, where R^{5b} is H or hydroxyl protecting group and ^{5a} is —P(O)(OR^{5e})₂, and where each R^{5e} is independently hydrogen, or optionally substituted C₁₋₃₀alkyl, and
provided that when R⁵ is —CH₂OR^{5b}, then (a) the nucleobase B is not uracil or (b) both of R^{a} and R^{b} are not F.

In some embodiments of any one of the aspects described herein, a compound of Formula (III′) is of Formula (IIIA′) or (IIIB′), and wherein:
X is O;
R^{a} is halogen (e.g., F, Br or Cl), hydroxyl, protected hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), a reactive phosphorous group (e.g., —OP(OR^{P})N(R^{P2})₂ (such as —OP(OCH₂CH₂CN)N(iPr)₂), —OP(SR^{P}) N(R²)₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support, a linker covalently attached to a solid support;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl), or halogen (e.g., F, Br or Cl);
R⁴ is hydrogen;
R³ is a reactive phosphorous group (e.g., —OP(OR^{P})N(R^{P2})₂ (such as —OP(OCH₂CH₂CN)N(iPr)₂), —OP(SR^{P}) N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support, a linker covalently attached to a solid support, hydroxyl, or protected hydroxyl, provided that only one of R^{a} and R³ is a reactive phosphorous group, a solid support, or a linker covalently attached to a solid support; and
R⁵ is —CH₂OR^{5b} or —CH═CHR^{5a}, where R^{5b} is H or hydroxyl protecting group and ^{5a} is —P(O)(OR^{5e})₂, and where each R^{5e} is independently hydrogen, or optionally substituted C₁₋₃₀alkyl.

In another aspect, provided herein is a method for inhibiting or reducing the expression of a target gene in a subject. The method comprises administering to the subject: (i) a double-stranded nucleic acid (e.g. dsRNA) described herein, wherein the first strand is complementary to a target gene; and/or (ii) an oligonucleotide described herein, wherein the oligonucleotide is complementary to a target gene.

### BRIEF DESCRIPTION OF THE DRAWINGS

FIGS. 1-15 are synthesis schemes for some exemplary compounds of formula (III).

FIG. 16 shows structures of the antiviral HCV drug Sofosbuvir and the active metabolite, which inspired the modifications 2′-F/Me uridine (U_{F/Me}) and cytidine (C_{F/Me}), and the corresponding 5′-vinyl phosphonate isomers (E-VP-U_{F/Me} and Z-VP-U_{F/Me}) studied herein.

FIGS. 17A-17D show in vitro potency of fully 2′-modified siRNA targeting (FIG. 17A) Ttr, (FIG. 17B) Pten, (FIG. 17C) F7. For experimental conditions, see Table 8. 2′—F and 2′-OMe nucleotides are represented as green or black circles, respectively. A yellow bar represents a PS linkage, VP is 5′-(E)-vinyl phosphonate, and zVP is 5′-(Z)-vinyl phosphonate. Error bars show standard deviations from mean

FIGS. 18A and 18B show mitigation of seed-mediated off-target activity by incorporation at AS7. FIG. 18A) On- and off-target effects were evaluated in a dual luciferase reporter assay. Luciferase reporter plasmids were co-transfected with indicated siRNAs into COS-7 cells. The cells were harvested 48 h, and luciferase activity was assayed. Percent target remaining was calculated by dividing the ratio of Renilla to firefly luciferase signal at each siRNA concentration by the ratio in the absence of siRNA. FIG. 18B) Transcriptional dysregulation in primary rat hepatocytes. Primary rat hepatocytes were transfected with 50 nM of the indicated siRNA. The upper panel is the parent (si19) and the lower one the modified (si20). After 48 h, total RNA was isolated for RNA-seq analysis. Dots represent individual rat transcripts, their average read count, and the level of change in expression compared to the mock transfection control. Grey dots represent genes not differentially expressed after siRNA treatment relative to the control, and the blue and red dots represent differentially expressed genes (false discovery rate <0.05) with or without a canonical miRNA match (8mer, 7mer-A1, 7mer-m8)⁵⁴ to the seed region, respectively. On-target knockdown of Ttr is indicated by the circled dot. Log 2 fold change and cumulative distribution plots are shown on the left and right, respectively.

FIGS. 19A-19D show impact of 2′-F/Me modifications on in vivo activity. FIGS. 19A and 19B) C57BL/6 mice (n=3) received a single dose of either 1 mg/kg (pink) or 3 mg/kg (grey) of FIG. 19A) F7-targeted siRNA as an LNP formulation intravenously or FIG. 1B) F7-targeted GalNAc-conjugated siRNA subcutaneously. Control animals received PBS. Serum F7 protein levels were measured at parent nadir: 48 h for LNP formulations and 10 days for GalNAc conjugates. FIGS. 19C and 19D) C57BL/6 mice (n=3) received a single dose of 1 mg/kg of indicated TTR-targeted siRNA, and serum protein levels were monitored until day 28. 2′—F, 2′-OMe, deoxyribonucleotides, and ribonucleotides are represented as green, black, blue, and red circles, respectively. A yellow bar represents a PS linkage. Data points were normalized to pre-dose F7 or TTR levels, and values are group means±SD.

FIG. 20A illustrating steric clashes as a consequence of the introduction of a 2′-β-C-methyl group on a single nucleotide in a 2′-F-modified RNA A-form duplex (PDB ID 3P4A).⁶³

FIG. 20B after molecular mechanics minimization lacks clashes between the methyl group and its nearest neighbors, but stacking is lost between uridines. Methyl carbon and hydrogen atoms are colored in yellow and white, respectively, fluorine atoms are light green. Short contacts are indicated with arrows. Watson-Crick hydrogen bonds and additional selected distances are shown with thin solid lines, and backbone torsion angle ranges are depicted in FIG. 20A.

FIGS. 21A-21D are modeled conformations of 2′-F/Me nucleotides incorporated into the siRNA guide strand bound to human Ago2 at positions (FIG. 21A) 1, (FIG. 21B) 2, (FIG. 21C) 6, and (FIG. 21D) 7 of the antisense strand incorporated into the siRNA antisense strand bound to human Ago2. Methyl carbon and hydrogen atoms are colored in yellow and white, respectively, fluorine atoms are light green. Short contacts are indicated with arrows. The initial conformation of the antisense strand as seen in the crystal structure of the human Ago2:miR-20a complex (PDB ID 4F3T)⁵³ is shown with thin black lines. A potentially favorable contact is indicated by a dashed line in FIG. 21D.

FIGS. 22A-22C are models of the VP-2′-F/Me-modified nucleotides at AS1 bound to the Ago2 MID domain. (FIG. 22A) E-VP with the C2′-endo sugar conformation; carbon atoms colored in purple. (FIG. 22B) Z—VP with the O4′-endo sugar conformation; carbon atoms colored in light blue. (FIG. 22C) Overlay of the E-VP and Z—VP nucleotides. The distance between the two phosphorus atoms (1.85 Å) is indicated with a double arrow. VP moieties, 2′-F (light green) and 2′-Me carbon (yellow) are highlighted in ball-and-stick mode, salt bridges and hydrogen bonds are drawn with thin solid lines, and selected Ago2 side chains are labeled.

FIGS. 23A and 23B shows origins of the improved resistance to exonuclease degradation by 2′-F/Me-modified oligonucleotides. (FIG. 23A) Model of oligo(dT) (yellow carbons) with two 5′-terminal 2′-F/Me-U residues (cyan carbons) bound to the active site of D. melanogaster Xrn1 5′-exoribonuclease. (FIG. 23B) Model of oligo(dT) (yellow carbons) with two 3′-terminal 2′-F/Me-U residues (cyan carbons) bound to the active site of E. coli DNA polymerase I Klenow fragment 3′-exonuclease. Distances between the 2′-Me carbon and phosphorus atoms are indicated with orange arrows. Distances between 2′-Me carbon and selected protein and DNA atoms are indicated with black arrows. 2′-F (light green), 2′-Me carbon (yellow), phosphorus (orange), non-bridging phosphate oxygens (red) and metal ions are highlighted in ball-and-stick mode. Salt bridges and hydrogen bonds are drawn with thin solid lines, metal ion coordination spheres are drawn with dashed lines, and selected Xrna1 and Klenow fragment side chains are labeled. All water molecules except those coordinated to catalytic metal ions were omitted.

FIGS. 24A-24D are thermal denaturation (Tm) curves of modified duplexes (2.5 uM) in 6.8×PBS ([NaCl]=931.6 mM, [KCl]=18.4 mM, [Na₂HPO₄]=68 mM, [KH₂PO₄]=12.2 mM, pH 7.4).

FIGS. 25A-25D are curves of modified oligonucleotides in the presence of 3′ exonuclease. Oligonucleotides (0.1 mg/mL) were incubated with 150 mU/mL SVPD in 50 mM Tris, pH 7.2, 10 mM MgCl₂, and full-length product was monitored via IEX-HPLC. ON13(X)=dT₁₉X-3′; ON14(X)=dT₁₈XdT; ON15(X)=dT₁₈X₂; ON16(X)=dT₁₈X•dT-3′; ON17(X)=dT₁₈•X-3′; ON18(X)=dT₁₈X•X-3′; PS linkage=•.

FIGS. 26A-26D are degradation curves of modified oligonucleotides in the presence of 5′ exonuclease. Oligonucleotides (0.1 mg/mL) were incubated with PD II (500 mU/mL) in 50 mM sodium acetate buffer (pH 6.5) with 10 mM MgCl₂ and monitored via IEX-HPLC. ON19(X) =5′-XdT₁₉; ON20(X)=5′-dTXdT₁₈; ON21(X)=5′-X₂dT₁₈; ON16(X)=dT₁₈X•dT-3′; ON17(X)=dT₁₈•X-3′; ON18(X)=dT₁₈X•X-3′; PS linkage=•.

FIG. 27 shows in vitro metabolic stability in rat liver homogenate after 24 h incubation at 37° C. Arrows represent the percent of the strand observed via LC-MS, where the tail direction depicts the observed fragment. Green balls=2′-F nucleotides, black balls=2′-OMe nucleotides, and pink ball=2′-F/Me uridine, VP=5′-(E)-vinyl phosphonate, zVP=5′-(Z)-vinyl phosphonate.

FIG. 28 are fitted dose response curves for IC₅₀-value determination of siRNA targeting TTR mRNA.

FIG. 29 are fitted dose response curves for IC₅₀-value determination of siRNA targeting PTEN mRNA.

FIG. 30 are fitted dose response curves for IC₅₀-value determination of siRNA targeting FVII mRNA.

FIG. 31 shows impact of 2′-F/Me modifications on in vivo activity. ApoB-targeting siRNA were dosed at 10 mg/kg, and relative mRNA expression of the target gene was calculated by qPCR from liver tissue at day 7. 2′—F, 2′-OMe, deoxyribonucleotides, and ribonucleotides are represented as green, black, blue, and red circles, respectively. Yellow bar represents a PS linkage and VP is 5′-(E)-vinyl phosphonate. Data points were normalized to pre-dose ApoB levels and value represents the group mean±SD.

FIG. 32 is a schematic reporesentation showing origin of the inability of POLG to incorporate a 2′-F/Me-modified nucleotide. The active site in the crystal structure of a ternary POLG•DNA•dCTP Mg²⁺ complex (PDB ID 4ZTZ) is shown. The view is into the minor groove of the duplex formed by the template (pink carbon atoms) and the primer (cyan carbon atoms). The 2′-F/Me CMP (purple carbon atoms) is superimposed on the incoming dCTP (gold carbon atoms). Two Mg²⁺ ions are visible in the background as gray spheres. The distance of 2.78 Å between the 2′-F/Me methyl carbon (highlighted in yellow) and the center of mass of the Tyr-951 ring (black dot) is indicative of a short contact (the sum of vdW radii for the methyl group, 2 Å, and phenyl carbons, 1.5 Å, is 3.5 Å). Selected distances are shown with thin solid lines.

FIGS. 33A and 33B are synthesis schemes for some exemplary compounds.

FIG. 34 is a schematic representation of p-oligonucleotide for therapeutic utility involving exemplary nucleoside building blocks. As shown, any number of building blocks can be assembled as an oligonucleotide and attached to a ligand of choice (e.g. TriGalNAc). For example, 2′-gem Me/F compound with cytosine nucleobase in the delivery to liver hepatocytes for HCV. Similarly, Gemcitabine can be delivered to e.g. hepatocellular carcinoma.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### X

In some embodiments of any one of the aspects described herein, X is O, S, C(R^{X})₂, or N(R^{XN}). When X is C(R^{X})₂, each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{XN}), or =CH₂. For example, X is O. When X is N(R^{XN}), R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects described herein, X is O.

### Nucleobases

In some embodiments of any one of the aspects described herein, B is H or a nucleobase. It is noted that the nucleobase can be a natural, non-natural and/or modified nucleobase. Exemplary natural nucleobases include, but are not limited to, adenine, cytosine, guanine, thymine, and uracil. By a “non-natural nucleobase” is meant a nucleobase other than adenine, guanine, cytosine, uracil, or thymine. Exemplary non-natural nucleobases include, but are not limited to, inosine, xanthine, hypoxanthine, nubularine, isoguanisine, tubercidine, and substituted or modified analogs of adenine, guanine, cytosine and uracil, such as 2-aminoadenine and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613, content of all which is incorporated herein by reference.

In some embodiments, the non-natural nucleobase can be selected from the group consisting of inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, nucleobase is a modified nucleobase, i.e., the nucleobase comprises a nucleobase modification described herein, e.g., the nucleobase is a substituted or modified analog of any of the natural nucleobases. Examples of the nucleobase modifications include, but not limited to: C-5 pyrimidine with an alkyl group or aminoalkyls and other cationic groups such as guanidinium and amidine functionalities, N²- and N⁶- with an alkyl group or aminoalkyls and other cationic groups such as guanidinium and amidine functionalities of purines, G-clamps, guanidinium G-clamps, and pseudouridine known in the art.

In some embodiments of any one of the aspects, the nucleobase is a universal nucleobase. As used herein, a universal nucleobase is any modified or unmodified natural or non-natural nucleobase that can base pair with all of adenine, cytosine, guanine and uracil without substantially affecting the melting behavior, recognition by intracellular enzymes or activity of the oligonucleotide comprising the universal nucleobase. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof.

In some embodiments of any one of the aspects described herein, the nucleobase (e.g., B) is a protected nucleobase. As used herein, a “protected nucleobase” referes to a nucleobase comprising a nitrogen protecting group, and/or an oxygen protecting group, and/or a sulfur protecting group.

In some embodiments of any one of the aspects described herein, the nucleobase (e.g., B) is a nucleobase selected from adenine, cytosine, guanine, thymine, uracil, and any modified, protected or substituted analogs thereof.

### R^{a′}

In some embodiments of any one of the aspects described herein, R^{a′} is halogen, hydrogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a bond to an internucleoside linkage to a subsequent nucleoside.

In some embodiments of any one of the aspects described herein, R_{a′} is a halogen. For example, R^{a′} is fluoro (F). In some examples, R^{a′} is chloro (Cl).

In some embodiments of any one of the aspects, when R^{a′} is —OR^{a2}, R^{a2} can be hydrogen or a hydroxyl protecting group. For example, R^{a2} can be hydrogen in some embodiments of any one of the aspects described herein.

When R^{a′} is —SR^{a3}, R^{a3} can be hydrogen or a sulfur protecting group. Accordingly, in some embodiments of any one of the aspects, R^{a3} is hydrogen.

When R^{a′} is —O(CH₂CH₂O)ₘCH₂CH₂OR^{a4}, m is 1-50; R^{a4} is independently for each occurrence H, C₁-C₃₀alkyl, cyclyl, heterocyclyl, aryl, heteroaryl, aralkyl, sugar or R^{a5}; and R^{a5} is independently for each occurrence amino (NH₂), alkylamino, dialkylamino, arylamino, diarylamino, heteroarylamino, or diheteroaryl amino.

When R^{a′} is —NH(CH₂CH₂NH)ₙCH₂CH₂—R^{a5}, n is 1-50 and R⁵ is independently for each occurrence amino (NH₂), alkylamino, dialkylamino, arylamino, diarylamino, heteroarylamino, or diheteroaryl amino.

In some embodiments of any one of the aspects described herein, R^{a′} is hydrogen, halogen, —OR^{a2}, or optionally substituted C₁-C₃₀alkoxy. For example, R^{a} is halogen, —OR^{a2}, or optionally substituted C₁-C₃₀alkoxy. In some embodiments of any one of the aspects described herein, R^{a} is F, Cl, OH or optionally substituted C₁-C₃₀alkoxy.

In some embodiments of any one of the aspects described herein, R^{a} is a halogen. For example, R^{a} is fluoro (F). In some examples, R^{a} is chloro (Cl).

In some embodiments of any one of the aspects described herein, R^{a′} is C₁-C₃₀alkoxy optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ(CH₂)ₚ—NH₂ or CH₂-aryl-alkoxy, where “m” and “p” are independently 1, 2, 3, 4, 5 or 6. For example, R_{a′} is C₁-C₃₀alkoxy optionally substituted with a NH₂, OH, C(O)NH₂, COOH, halo, SH, or C₁-C₆alkoxy. In some embodiments of any one of the aspects described herein, R^{a′} is —O(CH₂)ₚCH₃, where p is 1-21. For example, p is 14, 15, 16, 17 or 18. In one non-limiting example, p is 16.

In some embodiments of any one of the aspects, R^{a′} is —O(CH₂)_{q}R^{a7}, where q is 2-10; R^{a7} is C₁-C₆alkoxy, amino (NH₂), CO₂H, OH or halo. For example, R^{a7} is —CH₃ or —NH₂. Accordingly, in some embodiments of any one of the aspects, R^{a} is —O(CH₂)_{q}—OMe or R^{a} is —O(CH₂)_{q}—NH₂.

In some embodiments of any one of the aspects described herein, q is 2, 3, 4, 5 or 6. For example, q is 2, 3 or 6. In one non-limiting example, q is 2. In another non-limiting example, q is 3 or 6.

In some embodiments of any one of the aspects described herein, R^{a′} is a C₁-C₆haloalkyl. For example, R^{a′} is a C₁-C₄haloalkyl. In some embodiments of any one of the aspects described herein, R^{a} is —CF₃, —CF₂CF₃, —CF₂CF₂CF₃ or —CF₂(CF₃)₂.

In some embodiments of any one of the aspects described herein, R^{a′} is —OCH(CH₂OR^{a8})CH₂OR^{a9}, where R^{a8} and R^{a9} independently are H, optionally substituted C₁-C₃₀alkyl, optionally substituted C₂-C₃₀alkenyl or optionally substituted C₂-C₃₀alkynyl. For example, R^{a8} and R^{a9} independently are optionally substituted C₁-C₃₀alkyl.

In some embodiments of any one of the aspects described herein, R^{a′} is —CH₂C(O)NHR^{a10}, where R^{a10} is H, optionally substituted C₁-C₃₀alkyl, optionally substituted C₂-C₃₀alkenyl or optionally substituted C₂-C₃₀alkynyl. For example, R^{a10} is H or optionally substituted C₁-C₃₀alkyl. In some embodiments, R^{a10} is optionally substituted C₁-C₆alkyl.

In some embodiments of any one of the aspects described herein, R^{a′} can be a bond to an internucleoside linkage to a subsequent nucleoside.

In some embodiments of any one of the aspects descried herein, R^{a′} can be a linker to a solid support.

### R^{b}

In some embodiments of any one of the aspects described herein, R^{b} is optionally substituted C₁₋₃₀alkyl, optionally substituted C₂₋₃₀alkenyl, optionally substituted C₂₋₃₀alkynyl, or halogen. For example, R^{b} is C₁₋₃₀alkyl, optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ-NH₂ or CH₂-aryl-alkoxy, where “m” and “p” are independently 1, 2, 3, 4, 5 or 6. For example, R^{b} is C₁-C₃₀alkyl optionally substituted with a NH₂, OH, C(O)NH₂, COOH, halo, SH, or C₁-C₆alkoxy.

In some embodiments, R^{b} is C₁₋₃₀alkyl, optionally substituted with a NH₂, OH, C(O)NH₂, COOH, halo, SH, or C₁-C₆alkoxy. In some embodiments, R^{b} is methyl, ethyl, propyl, isopropyl, butyl, iso-butyl, t-butyl, vinyl, ethynyl, allyl or propargyl. For example, R^{b} is methyl, vinyl, ethynyl, allyl or propargyl. In some embodiments of any one of the aspects described herein, R^{b} is methyl.

In some embodiments of any one of the aspects described herein, R^{a′} is halogen and R^{b} is optionally substituted C₁₋₃₀alkyl. For example, R^{a′} is F and R^{b} is methyl, ethyl, propyl, isopropyl, butyl, iso-butyl, t-butyl, vinyl, ethynyl, allyl or propargyl. In some embodiments of any one of the aspects described herein, R^{a′} is F and R^{b} is methyl.

In some embodiments of any one of the aspects described herein, R^{a} is halogen and R^{b} is optionally substituted C₁₋₃₀alkyl. For example, R^{a} is F and R^{b} is methyl, ethyl, propyl, isopropyl, butyl, iso-butyl, t-butyl, vinyl, ethynyl, allyl or propargyl. In some embodiments of any one of the aspects described herein, R^{a} is F and R^{b} is methyl.

In some embodiments of any one of the aspects described herein, R^{a′} and R^{b} are halogen. For example, R^{a′} and R^{b} are independently F, Cl, Br or I. It is noted that the R^{a′} and R^{b}can be same or different. In some embodiments of any of the aspects described herein, R^{a′} and R^{b}are F. In some embodiments of any one of the aspects described herein, R^{a′} and R^{b} are not F at the same time. In some embodiments of any one of the aspects described herein, R^{a′} and R^{b} are Cl or Br.

In some embodiments of any one of the aspects described herein, R^{a} and R^{b} are halogen. For example, R^{a} and R^{b} are independently F, Cl, Br or I. It is noted that the R^{a} and R^{b} can be same or different. In some embodiments of any of the aspects described herein, R^{a} and R^{b} are F. In some embodiments of any one of the aspects described herein, R^{a} and R^{b} are not F at the same time. In some embodiments of any one of the aspects described herein, R^{a} and R^{b} are Cl or Br.

### R^{c}

In some embodiments of any one of the aspects described herein, R^{c} is a bond to an internucleoside linkage to a subsequent nucleoside, hydrogen, halogen, —OR^{c2}, —SR^{c3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH2CH₂—R^{c5}, NHC(O)R^{c4}, a lipid, a linker covalently attached to a lipid, a ligand, a linker covalently attached to a ligand, a solid support, or a linker covalently attached to a solid support.

In some embodiments of any one of the aspects, one of R^{b} and R^{c} is a bond to an internucleoside linkage to a subsequent nucleoside. For example, R^{c} is a bond to an internucleoside linkage to a subsequent nucleoside.

In some embodiments of any one of the aspects, when R^{c} is —OR^{c2}, R^{c2} can be hydrogen or a hydroxyl protecting group. For example, R^{c2} can be hydrogen in some embodiments of any one of the aspects described herein.

When R^{c} is —SR^{c3}, R^{c3} can be hydrogen or a sulfur protecting group. Accordingly, in some embodiments of any one of the aspects, R^{c3} is hydrogen.

When R^{c} is —O(CH₂CH₂O)ᵣCH₂CH₂OR^{c4}, r can be 1-50; R^{c4} is independently for each occurrence H, C₁-C₃₀alkyl, cyclyl, heterocyclyl, aryl, heteroaryl, aralkyl, sugar or R^{c5}; and R^{c5} is independently for each occurrence amino (NH₂), alkylamino, dialkylamino, arylamino, diarylamino, heteroarylamino, or diheteroaryl amino.

When R^{c} is —NH(CH₂CH₂NH)ₛCH₂CH₂—R^{c5}, s can be 1-50 and R^{c5} can be independently for each occurrence amino (NH₂), alkylamino, dialkylamino, arylamino, diarylamino, heteroarylamino, or diheteroaryl amino.

In some embodiments of any one of the aspects described herein, R^{c} is hydrogen, halogen, —OR^{c2}, or optionally substituted C₁-C₃₀alkoxy. For example, R^{c} is halogen, —OR^{c2}, or optionally substituted C₁-C₃₀alkoxy. In some embodiments of any one of the aspects described herein, R^{c} is F, OH or optionally substituted C₁-C₃₀alkoxy.

In some embodiments of any one of the aspects described herein, R^{c} is C₁-C₃₀alkoxy optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{c} is C₁-C₃₀alkyl optionally substituted with a NH₂, OH, C(O)NH₂, COOH, halo, SH, or C₁-C₆alkoxy. For example, R^{c} is C₁-C₃₀alkoxy optionally substituted with a NH₂ or C₁-C₆alkoxy.

In some embodiments of any one of the aspects described herein, R^{c} is —O(CH₂)ₜCH₃, where t is 1-21. For example, t is 14, 15, 16, 17 or 18. In one non-limiting example, t is 16.

In some embodiments of any one of the aspects, R^{c} is —O(CH₂)ᵤR^{c7}, where u is 2-10; R^{a7} is C₁-C₆alkoxy, amino (NH₂), CO₂H, OH or halo. For example, R^{c7} is —CH₃ or NH₂. Accordingly, in some embodiments of any one of the aspects described herein, R^{c} is —O(CH₂)ᵤ—OMe or R^{c} is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R^{c} is a C₁-C₆haloalkyl. For example, R^{c} is a C₁-C₄haloalkyl. In some embodiments of any one of the aspects described herein, R^{c} is —CF₃, —CF₂CF₃, —CF₂CF₂CF₃ or —CF₂(CF₃)₂.

In some embodiments of any one of the aspects described herein, R^{c} is —OCH(CH₂OR^{c8})CH₂OR^{c8}, where R^{c8} and R^{c9} independently are H, optionally substituted C₁-C₃₀alkyl, optionally substituted C₂-C₃₀alkenyl or optionally substituted C₂-C₃₀alkynyl. For example, R^{c8} and R^{c9} independently are optionally substituted C₁-C₃₀alkyl.

In some embodiments of any one of the aspects described herein, R^{c} is —CH₂C(O)NHR^{c10}, where R^{c10} is H, optionally substituted C₁-C₃₀alkyl, optionally substituted C₂-C₃₀alkenyl or optionally substituted C₂-C₃₀alkynyl. For example, R^{c10} is H or optionally substituted C₁-C₃₀alkyl. In some embodiments, R^{a10} is optionally substituted C₁-C₆alkyl.

In some embodiments of any one of the aspects descried herein, R^{c} is solid support or a linker covalently attached to a solid support.

In some embodiments of any one of the aspects described herein, R^{a′} is halogen, R^{b} is optionally substituted C₁₋₃₀alkyl, and R^{c} is a bond to an internucleoside linkage to a subsequent nucleoside. For example, R^{a′} is F; R^{b} is methyl, ethyl, propyl, isopropyl, butyl, iso-butyl, t-butyl, vinyl, ethynyl, allyl or propargyl; and R^{c} is a bond to an internucleoside linkage to a subsequent nucleoside. In some embodiments of any one of the aspects described herein, R^{a′} is F, R^{b} is methyl, and R^{c} is a bond to an internucleoside linkage to a subsequent nucleoside.

### R⁴

In some embodiments of any one of the aspects described herein, R⁴ can be hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ can be hydrogen, methyl, ethyl, propyl, isopropyl, butyl, iso-butyl, t-butyl, vinyl, ethynyl, allyl or propargyl. For example, R^{b} is methyl, vinyl, ethynyl, allyl or propargyl.

In some embodiments of any one of the aspects described herein, R⁴ is H.

In some embodiments of any one of the aspects described herein, R^{a} and R⁴ taken together are 4′-C(R^{a11}R^{a12})ᵥ—Y-2′ or 4′-Y—C(R^{a11}R^{a12})ᵥ-2′; v is 1, 2 or 3; where Y is —O—, —CH₂—, —CH(Me)-, —C(CH₃)₂—, —S—, —N(R^{a13}), —C(O)—, —C(S)—, —S(O)—, —S(O)₂—, —OC(O)—, —C(O)O—, —N(R^{a13})C(O)—, or —C(O)N(R^{a13}); R^{a11} and R^{a12} independently are H, optionally substituted C₁-C₆alkyl, optionally substituted C₂-C₆alkenyl or optionally substituted C₂-C₆alkynyl; R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, v is 1. In some other embodiments of any one of the aspects, v is 2.

In some embodiments, Y is 0. For example, R^{a} and R⁴ taken together are 4′-C(R^{a11}R^{a12})ᵥ—O-2′.

It is noted that R^{a11} and R^{a12} attached to the same carbon can be same or different. For example, one of R^{a11} and R^{a12} can be H and the other of the R^{a11} and R^{a12} can be an optionally substituted C₁-C₆alkyl. In one non-limiting example, one of R^{a11} and R^{a12} can be H and the other can be C₁-C₆alkyl, optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{a11} and R^{a12} independently are H or C₁-C₃₀alkyl optionally substituted with a NH₂, OH, C(O)NH₂, COOH, halo, SH, or C₁-C₆alkoxy. In some embodiments of any one of the aspects, one of Rai and R^{a12} is H and the other is C₁-C₆alkyl, optionally substituted with a C₁-C₆alkoxy. For example, one of R^{a11} and R^{a12} is H and the other is —CH₃ or CH₂OCH₃.

In some embodiments of any one of the aspects, Rai and R^{a12} attached to the same C are the same. For example, Rai and R^{a12} attached to the same C are H.

In some embodiments of any one of the aspects, R^{a} and R⁴ taken together are 4′-CH₂₋₀-2′, 4′—CH(CH₃)—O-2′, 4′—CH(CH₂OCH₃)—O-2′, or 4′-CH₂CH₂—O-2′.

In some embodiments of any one of the aspects described herein, R⁴ is H.

In some embodiments of any one of the aspects described herein, or R^{c} and R⁴ taken together with the atoms to which they are attached form an optionally substituted C₃₋₈cycloalkyl, optionally substituted C₃₋₈cycloalkenyl, or optionally substituted 3-8 membered heterocyclyl.

In some embodiments of any one of the aspects described herein, R^{a′} is halogen, R^{b} is optionally substituted C₁₋₃₀alkyl, R^{c} is a bond to an internucleoside linkage to a subsequent nucleoside, and R⁴ is H. For example, R^{a′} is F; R is methyl, ethyl, propyl, isopropyl, butyl, iso-butyl, t-butyl, vinyl, ethynyl, allyl or propargyl; R^{c} is a bond to an internucleoside linkage to a subsequent nucleoside; and R⁴ is H. In some embodiments of any one of the aspects described herein, R^{a′} is F, R^{b} is methyl, R^{c} is a bond to an internucleoside linkage to a subsequent nucleoside, and R⁴ is H.

### R^{d}

In embodiments of the various aspects described herein, R^{d} can be —CH(R^{d1})—R^{d2} or —C(R^{d1})=CHR^{d2}, where R^{d1} is hydrogen, optionally substituted C₁₋₃₀alkyl, optionally substituted —C₂₋₃₀alkenyl, or optionally substituted —C₂₋₃₀alkynyl, and R^{d2} is a bond to an internucleoside linkage to the preceding nucleotide; and R^{d3} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects described herein, X^{d} is O or a bond. For example, X^{d} is O.

In some embodiments of the various aspects described herein, R^{d} is —CH(R^{d1})—X^{d}—R^{d2}.

In some embodiments of the various aspects described herein, R^{d} is —CH(R^{d1})—X^{d}—R^{d2} and where R^{d1} is H or C₁-C₃₀alkyl optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{d1} is H. In some other non-limiting examples, R^{d1} is C₁-C₃₀alkyl optionally substituted with a NH₂, OH, C(O)NH₂, COOH, halo, SH, or C₁-C₆alkoxy.

In some embodiments of the various aspects described herein, R^{d} is —CH(R^{d1})—O—R^{d2}, where R^{d1} is H or C₁-C₃₀alkyl optionally substituted with 1, 2, 3, 4 or 5 substituents independently selected from 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “in” and “p” are independently 1, 2, 3, 4, 5 or 6. For example, R^{d1} is H. In some other non-limiting examples, R^{d1} is C₁-C₃₀alkyl optionally substituted with a NH₂, OH, C(O)NH₂, COOH, halo, SH, or C₁-C₆alkoxy.

In some embodiments of any one of the aspects described herein, R^{d} is —CH₂—O—R^{d2}.

In some embodiments of any one of the aspects described herein, R^{d} is —C(R^{d1})=CHR^{d2}. It is noted that the double bond in —C(R^{d1})=CHR^{d2} can be in the cis or trans configuration. Accordingly, in some embodiments of any one of the aspects, R^{d} is —C(R^{d1})=CHR^{d2} and wherein the double bond is in the cis configuration. In some other embodiments of any one of the aspects, R^{d} is —C(R^{d1})=CHR^{d2} and wherein the double bond is in the trans configuration. In some embodiments of any one of the aspects described herein, R^{d} is —CH═CHR^{d2}.

In some embodiments of any one of the aspects described herein, R^{a′} is halogen, R^{b} is optionally substituted C₁₋₃₀alkyl, R^{c} is a bond to an internucleoside linkage to a subsequent nucleoside, R⁴ is H, and R^{d} is a bond to an internucleoside linkage to the preceding nucleotide. For example, R^{a′} is F; R^{b} is methyl, ethyl, propyl, isopropyl, butyl, iso-butyl, t-butyl, vinyl, ethynyl, allyl or propargyl; R^{c} is a bond to an internucleoside linkage to a subsequent nucleoside; R⁴ is H; and R^{d} is a bond to an internucleoside linkage to the preceding nucleotide. In some embodiments of any one of the aspects described herein, R^{a′} is F, R^{b} is methyl, R^{c} is a bond to an internucleoside linkage to a subsequent nucleoside, R⁴ is H; and R^{d} is a bond to an internucleoside linkage to the preceding nucleotide.

In some embodiments of any one of the aspects described herein, R^{a′} is halogen, R^{b} is optionally substituted C₁₋₃₀alkyl; R^{c} is hydroxyl, solid support or a linker covalently linked to a solid support; R⁴ is H; and R^{d} is a bond to an internucleoside linkage to the preceding nucleotide. For example, R^{a′} is F; R^{b} is methyl, ethyl, propyl, isopropyl, butyl, iso-butyl, t-butyl, vinyl, ethynyl, allyl or propargyl; R^{c} is hydroxyl, solid support or a linker covalently linked to a solid support; R⁴ is H; and R^{d} is a bond to an internucleoside linkage to the preceding nucleotide. In some embodiments of any one of the aspects described herein, R^{a′} is F; R^{b} is methyl; R^{c} is hydroxyl, solid support or a linker covalently linked to a solid support; R⁴ is H; and R^{d} is a bond to an internucleoside linkage to the preceding nucleotide.

In some embodiments of any one of the aspects described herein, R^{a′} is halogen, R^{b} is optionally substituted C₁₋₃₀alkyl, R^{c} is a bond to an internucleoside linkage to a subsequent nucleoside, R⁴ is H, and R^{d} is —CH₂—O—R^{d2}. For example, R^{a′} is F; R^{b} is methyl, ethyl, propyl, isopropyl, butyl, iso-butyl, t-butyl, vinyl, ethynyl, allyl or propargyl; R^{c} is a bond to an internucleoside linkage to a subsequent nucleoside; R⁴ is H; and R^{d} is —CH₂—O—R^{d2}. In some embodiments of any one of the aspects described herein, R^{a′} is F, R^{b} is methyl, R^{c} is a bond to an internucleoside linkage to a subsequent nucleoside, R⁴ is H; and R^{d} is —CH₂—O—R^{d2}.

In some embodiments of any one of the aspects described herein, R^{a′} is halogen, R^{b} is optionally substituted C₁₋₃₀alkyl; R^{c} is hydroxyl, solid support or a linker covalently linked to a solid support; R⁴ is H; and R^{d} is —CH₂—O—R^{d2}. For example, R^{a′} is F; R^{b} is methyl, ethyl, propyl, isopropyl, butyl, iso-butyl, t-butyl, vinyl, ethynyl, allyl or propargyl; R^{c} is hydroxyl, solid support or a linker covalently linked to a solid support; R⁴ is H; and R^{d} is —CH₂—O—R^{d2}. In some embodiments of any one of the aspects described herein, R^{a′} is F; R^{b} is methyl; R^{c} is hydroxyl, solid support or a linker covalently linked to a solid support; R⁴ is H; and R^{d} is —CH₂—O—R^{d2}.

### R^{e}

In embodiments of the various aspects described herein, R^{e} is optionally substituted —C₂₋₆alkenyl-R^{e1}, optionally substituted C₁₋₆alkyl-R^{e1}, or optionally substituted —C₂₋₆alkynyl-R^{e1}. In embodiments of the various aspects described herein, R^{e1} can be —OR^{e2}, —SR^{e3}, —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂; where R^{e2} is hydrogen or oxygen protecting group; R^{e3} is hydrogen or sulfur protecting group; each R^{e4} is independently hydrogen, optionally substituted C₁₋₃₀alkyl, optionally substituted C₂₋₃₀alkenyl, optionally substituted C₂₋₃₀alkynyl, an oxygen-protecting group or an alkali metal or a transition metal with an overall charge of +1; and each R^{e5} is independently hydrogen, optionally substituted C₁₋₃₀alkyl, optionally substituted C₂₋₃₀alkenyl, or optionally substituted C₂₋₃₀alkynyl, or a sulfur-protecting group. It is noted that a double bond in the optionally substituted —C₂₋₆alkenyl-R^{e1} can be in the cis or trans configuration.

In some embodiments of any one of the aspects, at least one R^{e4} in —P(O)(OR^{e4})₂, —P(S)(OR^{e4})₂, —P(S)(SR^{e5})(OR^{e4}), —OP(O)(OR^{e4})₂, —OP(S)(OR^{e4})₂, —OP(S)(SR^{e5})(OR^{e4}), SP(O)(OR^{e4})₂, —SP(S)(OR^{e4})₂, and —SP(S)(SR^{e5})(OR^{e4}) is hydrogen.

In some embodiments of any one of the aspects, at least one R^{e4} in —P(O)(OR^{e4})₂, —P(S)(OR^{e4})₂, —P(S)(SR^{e5})(OR^{e4}), —OP(O)(OR^{e4})₂, —OP(S)(OR^{e4})₂, —OP(S)(SR^{e5})(OR^{e4}), SP(O)(OR^{e4})₂, —SP(S)(OR^{e4})₂, and —SP(S)(SR^{e5})(OR^{e4}) is an alkali metal or a transition metal with an overall charge of +1

In some other embodiments of any one of the aspects, at least one R^{e4} in —P(O)(OR^{e4})₂, —P(S)(OR^{e4})₂, —P(S)(SR^{e5})(OR^{e4}), —OP(O)(OR^{e4})₂, —OP(S)(OR^{e4})₂, —OP(S)(SR^{e5})(OR^{e4}), SP(O)(OR^{e4})₂, —SP(S)(OR^{e4})₂, or —SP(S)(SR^{e5})(OR^{e4}) is not hydrogen. For example, at least one at least one R^{e4} in P(O)(OR^{e4})₂, —P(S)(OR^{e4})₂, —P(S)(SR^{e5})(OR^{e4}), —OP(O)(OR^{e4})₂, —OP(S)(OR^{e4})₂, —OP(S)(SR^{e5})(OR^{e4}), SP(O)(OR^{e4})₂, —SP(S)(OR^{e4})₂, and —SP(S)(SR^{e5})(OR^{e4}) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R^{e4} is H and at least one R^{e4} is other than H in —P(O)(OR^{e4})₂, —P(S)(OR^{e4})₂, —P(S)(SR^{e5})(OR^{e4}), —OP(O)(OR^{e4})₂, —OP(S)(OR^{e4})₂, —OP(S)(SR^{e5})(OR^{e4}), SP(O)(OR^{e4})₂, —SP(S)(OR^{e4})₂, and —SP(S)(SR^{e5})(OR^{e4}).

In some embodiments of any one of the aspects, all R^{e4} are H in —P(O)(OR^{e4})₂, —P(S)(OR^{e4})₂, —P(S)(SR^{e5})(OR^{e4}), —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), and —SP(S)(SR^{e5})₂.

In some embodiments of any one of the aspects, all R^{e4} are other than H in in —P(O)(OR^{e4})₂, —P(S)(OR^{e4})₂, —P(S)(SR^{e5})(OR^{e4}), —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), and —SP(S)(SR^{e5})₂.

In some embodiments of any one of the aspects, at least one R^{e5} in —P(S)(SR^{e5})(OR^{e4}), —P(S)(SR^{e5})₂, —OP(S)(OR^{e4})₂, —OP(S)(SR^{e5})(OR^{e4}), —OP(S)(SR^{e5})₂, —SP(S)(SR^{e5})(OR^{e4}), and —SP(S)(SR^{e5})₂ is H.

In some embodiments of any one of the aspects, at least one R^{e5} in —P(S)(SR^{e5})(OR^{e4}), —P(S)(SR^{e5})₂, —OP(S)(OR^{e4})₂, —OP(S)(SR^{e5})(OR^{e4}), —OP(S)(SR^{e5})₂, —SP(S)(SR^{e5})(OR^{e4}), and —SP(S)(SR^{e5})₂ is other than H. For example, at least one R^{e5} in —P(S)(SR^{e5})(OR^{e4}), —P(S)(SR^{e5})₂, —OP(S)(OR^{e4})₂, —OP(S)(SR^{e5})(OR^{e4}), —OP(S)(SR^{e5})₂, —SP(S)(SR^{e5})(OR^{e4}), and —SP(S)(SR^{e5})₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R^{e5} is H and at least one R^{e5} is other than H in —P(S)(SR^{e5})₂, —OP(S)(SR^{e5})₂ and —SP(S)(SR^{e5})₂.

In some embodiments, all R^{e5} are H in —P(S)(SR^{e5})(OR^{e4}), —P(S)(SR^{e5})₂, —OP(S)(OR^{e4})₂, —OP(S)(SR^{e5})(OR^{e4}), —OP(S)(SR^{e5})₂, —SP(S)(SR^{e5})(OR^{e4}), and —SP(S)(SR^{e5})₂.

In some embodiments, all R^{e5} are other than H in —P(S)(SR^{e5})(OR^{e4}), —P(S)(SR^{e5})₂, —OP(S)(OR^{e4})₂, —OP(S)(SR^{e5})(OR^{e4}), —OP(S)(SR^{e5})₂, —SP(S)(SR^{e5})(OR^{e4}), and —SP(S)(SR^{e5})₂.

In some embodiments of any one of the aspects described herein, R^{e} is optionally substituted —C₂₋₆alkenyl-R^{e1}. For example, R^{e} is —C₂₋₆alkenyl-R^{e1}, where C₂₋₆alkenyl is optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R^{e1} is —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂.

In some embodiments of the various aspects described herein, R^{e} can be —CH(R^{d1})—R^{e1} or —C(R^{d1})=CHR^{e1}, where R^{d1} is hydrogen, optionally substituted C₁₋₃₀alkyl, optionally substituted —C₂₋₃₀alkenyl, or optionally substituted —C₂₋₃₀alkynyl, and R^{e1} is —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), —SP(S)(SR^{e5})₂, —OR^{e2} or —SR^{e3}.

In some embodiments of any one of the aspects described herein, R^{e} is —C(R^{d1})=CHR^{e1}. It is noted that the double bond in —C(R^{d1})=CHR^{e1} can be in the cis or trans configuration. Accordingly, in some embodiments of any one of the aspects, R^{e} is —C(R^{d1})=CHR^{e1} and wherein the double bond is in the cis configuration. In some other embodiments of any one of the aspects, R^{e} is —C(R^{d1})=CHR^{e1} and wherein the double bond is in the trans configuration.

In some embodiments of any one of the aspects, R^{e} is —CH═CHR^{e1}. For example, R^{e} is —CH═CHR^{e1} and wherein the double bond is in the trans configuration. In some other examples, R^{e} is —CH═CHR^{e1} and wherein the double bond is in the cis configuration.

In some embodiments of any one of the aspects, R^{e} is —CH═CH—P(O)(OR^{e4})₂, —CH═CH—P(S)(OR^{e4})₂, —CH═CH—P(S)(SR^{e5})(OR^{e4}), —CH═CH—P(S)(SR^{e5})₂, —CH═CH—OP(O)(OR^{e4})₂, —CH═CH—OP(S)(OR^{e4})₂, —CH═CH—OP(S)(SR^{e5})(OR^{e4}), —CH═CH—OP(S)(SR^{e5})₂, —CH═CH—SP(O)(OR^{e4})₂, —CH═CH—SP(S)(OR^{e4})₂, —CH═CH—SP(S)(SR^{e5})(OR^{e4}), or —CH═CH—SP(S)(SR^{e5})₂. For example, R^{c} is —CH═CH—P(O)(OR^{e4})₂.

In some embodiments, of any one of the aspects, R^{e2} is hydrogen or an oxygen protecting group. For example, R^{e2} is hydrogen or 4,4′-dimethoxytrityl (DMT). In some preferred embodiments, R^{e2} is H.

In some embodiments of any one of the aspects described herein, R^{e} is optionally substituted —C₁₋₆alkenyl-R^{e1}. For example, R^{e} is —C₁₋₆alkenyl-R^{e1}, where C₁₋₆alkenyl is optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ(CH₂)ₚ—OH, CH₂—[CH(OH)]ₘ(CH₂)ₚ—NH₂ or CH₂-aryl-alkoxy, where “m” and “p” are independently 1, 2, 3, 4, 5 or 6; and R^{e1} is —OR^{e2}, —SR^{e3}, —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂.

In some embodiments of any one of the aspects described herein, R^{e} can be —CH(R^{e6})—R^{e1}, where R^{e1} is —OR^{e2}, —SR^{e3}, —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂; and R^{e6} is H, optionally substituted C₁₋₃₀alkyl, optionally substituted C₂₋₃₀alkenyl, or optionally substituted C₂₋₃₀alkynyl.

In some embodiments of any one of the aspects described herein, R⁶ is H or C₁-C₃₀alkyl optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In one non-limiting example, R^{e6} is H. In some other non-limiting examples, R^{e6} is C₁-C₃₀alkyl optionally substituted with a substituent selected from NH₂, OH, C(O)NH₂, COOH, halo, SH, and C₁-C₆alkoxy.

In some embodiments of any one of the aspects described herein, R^{e} is —CH(R^{e6})—O—R^{e7}, where R^{e7} is H, —P(O)(OR^{e4})₂, —P(S)(OR^{e4})₂, —P(S)(SR^{e5})(OR^{e4}), —P(S)(SR^{e5})₂, —OP(O)(OR^{e4})₂. For example, R^{e} is —CH(R^{e6})—O—R^{e7}, where R^{e6} is H or optionally substituted C₁-C₃₀alkyl and R^{e7} is H or —P(O)(OR^{e4})₂.

In some embodiments of any one of the aspects described herein R^{e} is —CH₂—O—R^{e2}, where R^{e2} is hydrogen or oxygen protecting group.

In some embodiments of any one of the aspects described herein, R^{e} is —CH(R^{e6})—S—R^{e8}, where R^{e8} is H, —P(O)(OR^{e4})₂, —P(S)(OR^{e4})₂, —P(S)(SR^{e5})(OR^{e4}), —P(S)(SR^{e5})₂, —OP(O)(OR^{e4})₂.

In some embodiments of any one of the aspects described herein, R^{a′} is halogen, R is optionally substituted C₁₋₃₀alkyl, R^{c} is a bond to an internucleoside linkage to a subsequent nucleoside, R⁴ is H, and R^{e} is —C(R^{d1})=CHR^{e1}. For example, R^{a′} is F; R is methyl, ethyl, propyl, isopropyl, butyl, iso-butyl, t-butyl, vinyl, ethynyl, allyl or propargyl; R^{c} is a bond to an internucleoside linkage to a subsequent nucleoside; R⁴ is H; and R^{e} is —C(R^{d1})=CHR^{e1}. In some embodiments of any one of the aspects described herein, R^{a′} is F, R is methyl, R^{c} is a bond to an internucleoside linkage to a subsequent nucleoside, R⁴ is H; and R^{e} is —C(R^{d1})=CHR^{e1}.

In some embodiments of any one of the aspects described herein, R^{a′} is halogen, R is optionally substituted C₁₋₃₀alkyl, R^{c} is a bond to an internucleoside linkage to a subsequent nucleoside, R⁴ is H, and R^{c} is —C(R^{d1})=CHR^{e1}, and where R^{e1} is —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), —SP(S)(SR^{e5})₂, —OR^{e2} or —SR^{e3}. For example, R^{a′} is F; R^{b} is methyl, ethyl, propyl, isopropyl, butyl, iso-butyl, t-butyl, vinyl, ethynyl, allyl or propargyl; R^{c} is a bond to an internucleoside linkage to a subsequent nucleoside; R⁴ is H; and R^{e} is —C(R^{d1})=CHR^{e1}, and where R^{e1} is —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂. In some embodiments of any one of the aspects described herein, R^{a′} is F, R^{b} is methyl, R^{c} is a bond to an internucleoside linkage to a subsequent nucleoside, R⁴ is H; and R^{e} is —C(R^{d1})=CHR^{e1}, where R^{e1} is —P(O)(OR^{e4})₂.

### R^{a}

In some embodiments of any one of the aspects described herein, R^{a} is halogen, hydrogen, —OR^{c2}, —SR^{c3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, a linker covalently attached to a solid support, or a reactive phosphorus group.

In some embodiments of any one of the aspects, when R^{a′} is —OR^{a2}, R^{a2} can be hydrogen or a hydroxyl protecting group. For example, R^{a2} can be hydrogen in some embodiments of any one of the aspects described herein.

When R^{a} is —SR^{a3}, R^{a3} can be hydrogen or a sulfur protecting group. Accordingly, in some embodiments of any one of the aspects, R^{a3} is hydrogen.

When R^{a} is —O(CH₂CH₂O)ₘCH₂CH₂OR^{a4}, m is 1-50; R^{a4} is independently for each occurrence H, C₁-C₃₀alkyl, cyclyl, heterocyclyl, aryl, heteroaryl, aralkyl, sugar or R^{a5}; and R^{a5} is independently for each occurrence amino (NH₂), alkylamino, dialkylamino, arylamino, diarylamino, heteroarylamino, or diheteroaryl amino.

When R^{a} is —NH(CH₂CH₂NH)ₙCH₂CH₂—R^{a5}, n is 1-50 and R^{a5} is independently for each occurrence amino (NH₂), alkylamino, dialkylamino, arylamino, diarylamino, heteroarylamino, or diheteroaryl amino.

In some embodiments of any one of the aspects described herein, R^{a} is hydrogen, halogen, —OR^{a2}, or optionally substituted C₁-C₃₀alkoxy. For example, R^{a} is halogen, —OR^{a2}, or optionally substituted C₁-C₃₀alkoxy. In some embodiments of any one of the aspects described herein, R^{a} is F, OH or optionally substituted C₁-C₃₀alkoxy.

In some embodiments of any one of the aspects described herein, R^{a} is a halogen. For example, R^{a} is fluoro (F).

In some embodiments of any one of the aspects described herein, R^{a} is C₁-C₃₀alkoxy optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH2-[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R^{a} is C₁-C₃₀alkoxy optionally substituted with a NH₂, OH, C(O)NH₂, COOH, halo, SH, or C₁-C₆alkoxy. In some embodiments of any one of the aspects described herein, R^{a} is —O(CH₂)ₚCH₃, where p is 1-21. For example, p is 14, 15, 16, 17 or 18. In one non-limiting example, p is 16.

In some embodiments of any one of the aspects, R^{a} is —O(CH₂)_{q}R^{a7}, where q is 2-10; R^{a7} is C₁-C₆alkoxy, amino (NH₂), CO₂H, OH or halo. For example, R^{a7} is —CH₃ or —NH₂. Accordingly, in some embodiments of any one of the aspects, R^{a} is —O(CH₂)_{q}—OMe or R^{a} is —O(CH₂)_{q}—NH₂.

In some embodiments of any one of the aspects described herein, q is 2, 3, 4, 5 or 6. For example, q is 2, 3 or 6. In one non-limiting example, q is 2. In another non-limiting example, q is 3 or 6.

In some embodiments of any one of the aspects described herein, R^{a} is a C₁-C₆haloalkyl. For example, R^{a} is a C₁-C₄haloalkyl. In some embodiments of any one of the aspects described herein, R^{a} is —CF₃, —CF₂CF₃, —CF₂CF₂CF₃ or —CF₂(CF₃)₂.

In some embodiments of any one of the aspects described herein, R^{a} is —OCH(CH₂OR^{a8})CH₂OR^{a9}, where R^{a8} and R^{a9} independently are H, optionally substituted C₁-C₃₀alkyl, optionally substituted C₂-C₃₀alkenyl or optionally substituted C₂-C₃₀alkynyl. For example, R^{a8} and R^{a9} independently are optionally substituted C₁-C₃₀alkyl.

In some embodiments of any one of the aspects described herein, R^{a} is —CH₂C(O)NHR^{a10}, where R^{a10} is H, optionally substituted C₁-C₃₀alkyl, optionally substituted C₂-C₃₀alkenyl or optionally substituted C₂-C₃₀alkynyl. For example, R^{a10} is H or optionally substituted C₁-C₃₀alkyl. In some embodiments, R^{a10} is optionally substituted C₁-C₆alkyl.

In some embodiments of any one of the aspects, R^{a} is a phosphorous group. For example, R^{a} is a reactive phosphorous group.

Without wishing to be bound by a theory, reactive phosphorus groups are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. Reactive phosphorous group in the form of phosphoramidites (P^{III} chemistry) as reactive phosphites are a preferred reactive phosphorous group for solid phase oligonucleotide synthesis. The intermediate phosphite compounds are subsequently oxidized to the Pv state using known methods to yield phosphodiester or phosphorothioate internucleoside linkages.

In some embodiments of any one of the aspects described herein, the reactive phosphate group is —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}. For example, the reactive phosphorous group is —OP(OR^{P})N(R^{P2})₂.

In some embodiments of any one of the aspects, R_{P} is an optionally substituted C₁₋₆alkyl. For example, R is a C₁₋₆alkyl, optionally substituted with

1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “in” and “p” are independently 1, 2, 3, 4, 5 or 6. In some embodiments, R^{P} is a C₁₋₆alkyl, optionally substituted with a CN or —SC(O)Ph. For example, R^{P} is cyanoethyl (—CH₂CH₂CN).

In the reactive phosphorous groups, each R^{P2} is independently optionally substituted C₁₋₆alkyl. For example, each R^{P2} can be independently selected from methyl, ethyl, propyl, isopropyl, n-butyl, iso-butyl, pentyl or hexyl. It is noted that when two or more R^{P2} groups are present in the reactive phosphorous group, they can be same or different. Thus, in some none-limiting examples, when two or more R^{P2} groups are present, the R^{P2} groups are different. In some other non-limiting examples, when two or more R^{P2} groups are present, the R^{P2} groups are same. In some embodiments of any one of the aspects, each R^{P2} is isopropyl.

In some embodiments of any one of the aspects, both R^{P2} taken together with the nitrogen atom to which they are attached form an optionally substituted 3-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6.

In some embodiments of any one of the aspects, R^{P} and one of R^{P2} taken together with the atoms to which they are attached form an optionally substituted 4-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH2-[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6.

In the reactive phosphorous groups, each R^{P3} is independently optionally substituted C₁₋₆alkyl. For example, R^{P3} can be a C₁₋₆alkyl, optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R^{P3} is methyl, ethyl, propyl, isopropyl, n-butyl, iso-butyl, pentyl or hexyl, each of which can be optionally substituted with a NH₂, OH, C(O)NH₂, COOH, halo, SH, or C₁-C₆alkoxy.

In some embodiments of any one of the aspects, the reactive phosphorous group is —OP(OR^{P})N(R^{P2})₂. For example, the reactive phosphorous group is —OP(OR^{P})N(R^{P2})₂, where R^{P} is cyanoethyl (—CH₂CH₂CN) and each R^{P2} is isopropyl.

In some embodiments of any one of the aspects described herein, R^{a} is a reactive phosphorus group. For example, R^{a} is —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

In some embodiments of any one of the aspects, R³ is —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}, where R is an optionally substituted C₁₋₆alkyl, each R² is independently optionally substituted C₁₋₆alkyl; and each R^{P3} is independently optionally substituted C₁₋₆alkyl.

In some embodiments of any one of the aspects, R^{a} is —OP(OR^{P})N(R^{P2})₂. For example, R^{a} is —OP(OR^{P})N(R^{P2})₂, where R is cyanoethyl (—CH₂CH₂CN) and each R² is isopropyl.

In some embodiments of any one of the aspects descried herein, R^{a}can be a linker to a solid support.

In some embodiments of any one of the aspects described herein, R^{a} is halogen and R^{b} is optionally substituted C₁₋₃₀alkyl. For example, R^{a} is F and R^{b} is methyl, ethyl, propyl, isopropyl, butyl, iso-butyl, t-butyl, vinyl, ethynyl, allyl or propargyl. In some embodiments of any one of the aspects described herein, R^{a} is F and R is methyl.

### R³

In some embodiments of any one of the aspects described herein, R³ is hydrogen, halogen, —OR^{e2}, —SR^{e3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, a linker covalently attached to a solid support, or a reactive phosphorus group.

In some embodiments of any one of the aspects described herein, R³ is a reactive phosphorus group. For example, R³ is —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

In some embodiments of any one of the aspects, R³ is —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P}) an optionally substituted C₁₋₆alkyl, each R² is independently optionally substituted C₁₋₆alkyl; and each R^{P3} is independently optionally substituted C₁₋₆alkyl.

In some embodiments of any one of the aspects, R³ is —OP(OR^{P})N(R^{P2})₂. For example, the R³ is —OP(OR^{P})N(R^{P2})₂, where R is cyanoethyl (—CH₂CH₂CN) and each R^{P2} is isopropyl.

In some embodiments of any one of the aspects descried herein, R³ is solid support or a linker covalently attached to a solid support.

In some embodiments of any one of the aspects, when R³ is —OR^{c2}, R^{c2} can be hydrogen or a hydroxyl protecting group. For example, R^{c2} can be hydrogen in some embodiments of any one of the aspects described herein.

When R³ is —SR^{c3}, R^{c3} can be hydrogen or a sulfur protecting group. Accordingly, in some embodiments of any one of the aspects, R^{c3} is hydrogen.

When R³ is —O(CH₂CH₂O)ᵣCH₂CH₂OR^{c4}, r can be 1-50; R^{c4} is independently for each occurrence H, C₁-C₃₀alkyl, cyclyl, heterocyclyl, aryl, heteroaryl, aralkyl, sugar or R^{c5}; and R^{c5} is independently for each occurrence amino (NH₂), alkylamino, dialkylamino, arylamino, diarylamino, heteroarylamino, or diheteroaryl amino.

When R³ is —NH(CH₂CH₂NH)ₛCH₂CH₂—R^{e5}, s can be 1-50 and R^{c5} can be independently for each occurrence amino (NH₂), alkylamino, dialkylamino, arylamino, diarylamino, heteroarylamino, or diheteroaryl amino.

In some embodiments of any one of the aspects described herein, R³ is hydrogen, halogen, —OR^{c2}, or optionally substituted C₁-C₃₀alkoxy. For example, R³ is halogen, —OR^{c2}, or optionally substituted C₁-C₃₀alkoxy. In some embodiments of any one of the aspects described herein, R³ is F, OH or optionally substituted C₁-C₃₀alkoxy.

In some embodiments of any one of the aspects described herein, R³ is C₁-C₃₀alkoxy optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R³ is C₁-C₃₀alkoxy optionally substituted with a NH₂, OH, C(O)NH₂, COOH, halo, SH, or C₁-C₆alkoxy. In some embodiments of any one of the aspects described herein, R³ is —O(CH₂)ₜCH₃, where t is 1-21. For example, t is 14, 15, 16, 17 or 18. In one non-limiting example, t is 16.

In some embodiments of any one of the aspects, R³ is —O(CH₂)ᵤR^{c7}, where u is 2-10; R^{a7} is C₁-C₆alkoxy, amino (NH₂), CO₂H, OH or halo. For example, R^{c7} is —CH₃ or NH₂. Accordingly, in some embodiments of any one of the aspects described herein, R³ is —O(CH₂)ᵤ—OMe or R^{c} is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R³ is a C₁-C₆haloalkyl. For example, R³ is a C₁-C₄haloalkyl. In some embodiments of any one of the aspects described herein, R³ is —CF₃, —CF₂CF₃, —CF₂CF₂CF₃ or —CF₂(CF₃)₂.

In some embodiments of any one of the aspects described herein, R³ is —OCH(CH₂OR^{c8})CH₂OR^{c8}, where R^{c8} and R^{c9} independently are H, optionally substituted C₁-C₃₀alkyl, optionally substituted C₂-C₃₀alkenyl or optionally substituted C₂-C₃₀alkynyl. For example, R^{c8} and R^{c9} independently are optionally substituted C₁-C₃₀alkyl.

In some embodiments of any one of the aspects described herein, R^{c} is —CH₂C(O)NHR^{c10}, where R^{c10} is H, optionally substituted C₁-C₃₀alkyl, optionally substituted C₂-C₃₀alkenyl or optionally substituted C₂-C₃₀alkynyl. For example, R^{c10} is H or optionally substituted C₁-C₃₀alkyl. In some embodiments, R^{a10} is optionally substituted C₁-C₆alkyl.

In some embodiments of any one of the aspects described herein, R^{a} is halogen; R is optionally substituted C₁₋₃₀alkyl; and R³ is hydroxyl, protected hydroxyl, a reactive phosphorus group (e.g., —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support or a linker covalently linked to a solid support. For example, R^{a′} is F; R is methyl, ethyl, propyl, isopropyl, butyl, iso-butyl, t-butyl, vinyl, ethynyl, allyl or propargyl; and R³ is hydroxyl, protected hydroxyl, a reactive phosphorus group (e.g., —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support or a linker covalently linked to a solid support. In some embodiments of any one of the aspects described herein, R^{a′} is F; R^{b} is methyl; and R³ is hydroxyl, protected hydroxyl, a reactive phosphorus group (e.g., —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support or a linker covalently linked to a solid support.

In some embodiments of any one of the aspects described herein, R^{a} is halogen; R^{b} is optionally substituted C₁₋₃₀alkyl; and R³ is hydroxyl, protected hydroxyl, —OP(OR^{P})N(R^{P2})₂, or a linker covalently linked to a solid support. For example, R^{a′} is F; R^{b} is methyl, ethyl, propyl, isopropyl, butyl, iso-butyl, t-butyl, vinyl, ethynyl, allyl or propargyl; and R³ is hydroxyl, protected hydroxyl, —OP(OR^{P})N(R^{P2})₂, or a linker covalently linked to a solid support. In some embodiments of any one of the aspects described herein, R^{a′} is F; R^{b} is methyl; and R³ is hydroxyl, protected hydroxyl, —OP(OR^{P})N(R^{P2})₂, or a linker covalently linked to a solid support.

### R⁵

In some embodiments of the various aspects described herein, R⁵ is optionally substituted C₁₋₆alkyl-R^{5a}, optionally substituted —C₂₋₆alkenyl-R^{5a}, or optionally substituted —C₂₋₆alkynyl-R^{5a}, where R^{5a} can be —OR^{5b}, —SR^{5c}, hydrogen, a phosphorous group, a solid support or a linker to a solid support. When R^{5a} is —OR^{5b}, R^{5b} can be H or a hydroxyl protecting group. Similarly, when R^{5a} is —SR^{5c}, R^{5c} can be H or a sulfur protecting group.

In some embodiments of any one of the aspects described herein, R⁵ is —CH(R^{5d})—R^{5a}, where R^{5d} is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R⁵ is —CH(R^{5d})—R^{5a}, R^{5d} is H or C₁-C₃₀alkyl optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH2-[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R^{5d} is H. In some other non-limiting examples, R^{5d} is C₁-C₃₀alkyl optionally substituted with a NH₂, OH, C(O)NH₂, COOH, halo, SH, or C₁-C₆alkoxy.

In some embodiments of the various aspects described herein, R⁵ is —CH(R^{5d})—O—R^{5b}, where R^{5d} is H or C₁-C₃₀alkyl optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R^{5d} is H. In some other non-limiting examples, R^{5d} is C₁-C₃₀alkyl optionally substituted with a NH₂, OH, C(O)NH₂, COOH, halo, SH, or C₁-C₆alkoxy.

In some embodiments of the various aspects described herein, R⁵ is optionally substituted C₁₋₆alkyl-R^{5a} or optionally substituted —C₂₋₆alkenyl-R^{5a},

In some embodiments of any one of the aspects described herein, R⁵ is —C(R^{5d})=CHR^{5a}, It is noted that the double bond in —C(R^{5d})=CHR^{5a} can be in the cis or trans configuration. Accordingly, in some embodiments of any one of the aspects, R^{d} is —C(R^{5d})=CHR^{5a} and wherein the double bond is in the cis configuration. In some other embodiments of any one of the aspects, R^{d} is —C(R^{5d})=CHR^{5a} and wherein the double bond is in the trans configuration.

In some embodiments of any one of the aspects described herein, R⁵ is —CH═CHR^{5a}. For example, R⁵ is —CH═CHR^{5a} and wherein the double bond is in the trans configuration. In some other non-limiting example, R⁵ is —CH═CHR^{5a} and wherein the double bond is in the cis configuration.

In some embodiments of any one of the aspects, when R⁵ is —C(R^{5d})=CHR⁵a, R^{5d} is H or C₁-C₃₀alkyl optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6; and R^{5a} is a phosphorous group. For example, R⁵ is —CH═CHR^{5a}.

In some embodiments of any one of the aspects described herein, R^{5a} is a reactive phosphorous group.

In some embodiments of any one of the aspects, R^{5a} is —P(O)(OR^{5e})₂, —P(S)(OR^{5e})₂, —P(S)(SR^{5f})(OR^{5e}), —P(S)(SR^{5f})₂, —OP(O)(OR^{5e})₂, —OP(S)(OR^{5e})₂, —OP(S)(SR^{5f})(OR^{5e}), —OP(S)(SR^{5f})₂, —SP(O)(OR^{5e})₂, —SP(S)(OR^{5e})₂, —SP(S)(SR^{5f})(OR^{5e}), or —SP(S)(SR^{5f})₂, where each R^{5e} is independently hydrogen, optionally substituted C₁₋₃₀alkyl, optionally substituted C₂₋₃₀alkenyl, optionally substituted C₂₋₃₀alkynyl, an oxygen-protecting group or an alkali metal or a transition metal with an overall charge of +1; and each R^{5f} is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, R⁵ is —CH═CH—P(O)(OR^{5e})₂, —CH═CH—P(S)(OR^{5e})₂, —CH═CH—P(S)(SR^{5f})(OR^{5e}), —CH═CH—P(S)(SR^{5f})₂, —CH═CH—OP(O)(OR^{5e})₂, —CH═CH—OP(S)(OR^{5e})₂, —CH═CH—OP(S)(SR^{5f})(OR^{5e}), —CH═CH—OP(S)(SR^{5f})₂, —CH═CH—SP(O)(OR^{5e})₂, —CH═CH—SP(S)(OR^{5e})₂, —CH═CH—SP(S)(SR^{5f})(OR^{5e}), or —CH═CH—SP(S)(SR^{5f})₂, where each R^{5e} is independently hydrogen, optionally substituted C₁₋₃₀alkyl, optionally substituted C₂₋₃₀alkenyl, optionally substituted C₂₋₃₀alkynyl, an oxygen-protecting group or an alkali metal or a transition metal with an overall charge of +1; and each R^{5f} is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R^{5e} in —P(O)(OR^{5e})₂, —P(S)(OR^{5e})₂, —P(S)(SR^{5f})(OR^{5e}), —OP(O)(OR^{5e})₂, —OP(S)(OR^{5e})₂, —OP(S)(SR^{5f})(OR^{5e}), SP(O)(OR^{5e})₂, —SP(S)(OR^{5e})₂, and —SP(S)(SR^{5f})(OR^{5e}) is hydrogen.

In some other embodiments of any one of the aspects, at least one R^{5e} in —P(O)(OR^{5e})₂, —P(S)(OR^{5e})₂, —P(S)(SR^{5f})(OR^{5e}), —OP(O)(OR^{5e})₂, —OP(S)(OR^{5e})₂, —OP(S)(SR^{5f})(OR^{5e}), SP(O)(OR^{5e})₂, —SP(S)(OR^{5e})₂, or —SP(S)(SR^{5f})(OR^{5e}) is not hydrogen. For example, at least one at least one R^{5e} in P(O)(OR^{5e})₂, —P(S)(OR^{5e})₂, —P(S)(SR^{5f})(OR^{5e}), —OP(O)(OR^{5e})₂, —OP(S)(OR^{5e})₂, —OP(S)(SR^{5f})(OR^{5e}), SP(O)(OR^{5e})₂, —SP(S)(OR^{5e})₂, and —SP(S)(SR^{5f})(OR^{5e}) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R^{5e} is H and at least one R^{5e} is other than H in —P(O)(OR^{5e})₂, —P(S)(OR^{5e})₂, —P(S)(SR^{5f})(OR^{5e}), —OP(O)(OR^{5e})₂, —OP(S)(OR^{5e})₂, —OP(S)(SR^{5f})(OR^{5e}), SP(O)(OR^{5e})₂, —SP(S)(OR^{5e})₂, and —SP(S)(SR^{5f})(OR^{5e}).

In some embodiments of any one of the aspects, all R^{5e} are H in —P(O)(OR^{5e})₂, —P(S)(OR^{5e})₂, —P(S)(SR^{5f})(OR^{5e}), —OP(O)(OR^{5e})₂, —OP(S)(OR^{5e})₂, —OP(S)(SR^{5f})(OR^{5e}), —OP(S)(SR^{5f})₂, —SP(O)(OR^{5e})₂, —SP(S)(OR^{5e})₂, —SP(S)(SR^{5f})(OR^{5e}), and —SP(S)(SR^{5f})₂.

In some embodiments of any one of the aspects, all R^{5e} are other than H in in —P(O)(OR^{5e})₂, —P(S)(OR^{5e})₂, —P(S)(SR^{5f})(OR^{5e}), —OP(O)(OR^{5e})₂, —OP(S)(OR^{5e})₂, —OP(S)(SR^{5f})(OR^{5e}), —OP(S)(SR^{5f})₂, —SP(O)(OR^{5e})₂, —SP(S)(OR^{5e})₂, —SP(S)(SR^{5f})(OR^{5e}), and —SP(S)(SR^{5f})₂.

In some embodiments of any one of the aspects, at least one R^{5f} in —P(S)(SR^{5f})(OR^{5e}), —P(S)(SR^{5f})₂, —OP(S)(OR^{5e})₂, —OP(S)(SR^{5f})(OR^{5e}), —OP(S)(SR^{5f})₂, —SP(S)(SR^{5f})(OR^{5e}), and —SP(S)(SR^{5f})₂ is H.

In some embodiments of any one of the aspects, at least one R^{5f} in —P(S)(SR^{5f})(OR^{5e}), —P(S)(SR^{5f})₂, —OP(S)(OR^{5e})₂, —OP(S)(SR^{5f})(OR^{5e}), —OP(S)(SR^{5f})₂, —SP(S)(SR^{5f})(OR^{5e}), and —SP(S)(SR^{5f})₂ is other than H. For example, at least one R^{5f} in —P(S)(SR^{5f})(OR^{5e}), —P(S)(SR^{5f})₂, —OP(S)(OR^{5e})₂, —OP(S)(SR^{5f})(OR^{5e}), —OP(S)(SR^{5f})₂, —SP(S)(SR^{5f})(OR^{5e}), and —SP(S)(SR^{5f})₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R^{5f} is H and at least one R^{5f} is other than H in —P(S)(SR^{5f})₂, —OP(S)(SR^{5f})₂ and —SP(S)(SR⁵)₂.

In some embodiments, all R^{5f} are H in —P(S)(SR^{5f})(OR^{5e}), —P(S)(SR^{5f})₂, —OP(S)(OR^{5e})₂, —OP(S)(SR^{5f})(OR^{5e}), —OP(S)(SR^{5f})₂, —SP(S)(SR^{5f})(OR^{5e}), and —SP(S)(SR^{5f})₂.

In some embodiments, all R^{5f} are other than H in —P(S)(SR^{5f})(OR^{5e}), —P(S)(SR^{5f})₂, —OP(S)(OR^{5e})₂, —OP(S)(SR^{5f})(OR^{5e}), —OP(S)(SR^{5f})₂, —SP(S)(SR^{5f})(OR^{5e}), and —SP(S)(SR^{5f})₂.

In some embodiments of any one of the aspects, R⁵ is —CH═CH—P(O)(OR^{5e})₂, where each R^{5e} is H or an oxygen protecting group.

In some embodiments of any one of the aspects, R⁵ is —CH═CH—P(O)(OR^{5e})₂ and where the double bond is in the trans configuration. In some other embodiments of any one of the aspects, R⁵ is —CH═CH—P(O)(OR^{5e})₂ and where the double bond is in the cis configuration.

In some embodiments of any one of the aspects described herein, R^{a} is halogen; R is optionally substituted C₁₋₃₀alkyl; R³ is hydroxyl, protected hydroxyl, a reactive phosphorus group (e.g., —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}), a solid support or a linker covalently linked to a solid support; R⁴ is H; and R⁵ is —CH═CH—P(O)(OR^{5e})₂. For example, R^{a′} is F; R is methyl, ethyl, propyl, isopropyl, butyl, iso-butyl, t-butyl, vinyl, ethynyl, allyl or propargyl; R³ is hydroxyl, protected hydroxyl, a reactive phosphorus group (e.g., —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3} or —OP(O)(SR^{P})R^{P3}), a solid support or a linker covalently linked to a solid support; R⁴ is H; and R⁵ is —CH═CH—P(O)(OR^{5e})₂. In some embodiments of any one of the aspects described herein, R^{a′} is F; R is methyl; R³ is hydroxyl, protected hydroxyl, a reactive phosphorus group (e.g., —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})₂, —OP(O)(OR^{P})R^{P3}, —OP(S)(OR^{P})R^{P3}, or —OP(O)(SR^{P})R^{P3}), a solid support or a linker covalently linked to a solid support; R⁴ is H; and R⁵ is —CH═CH—P(O)(OR^{5e})₂.

In some embodiments of any one of the aspects described herein, R^{a} is halogen; R^{b} is optionally substituted C₁₋₃₀alkyl; R³ is hydroxyl, protected hydroxyl, —OP(OR^{P})N(R^{P2})₂, or a linker covalently linked to a solid support; R⁴ is H; and R⁵ is —CH═CH—P(O)(OR^{5e})₂. For example, R^{a′} is F; R^{b} is methyl, ethyl, propyl, isopropyl, butyl, iso-butyl, t-butyl, vinyl, ethynyl, allyl or propargyl; R³ is hydroxyl, protected hydroxyl, —OP(OR^{P})N(R^{P2})₂, or a linker covalently linked to a solid support; R⁴ is H; and R⁵ is —CH═CH—P(O)(OR^{5e})₂. In some embodiments of any one of the aspects described herein, R^{a′} is F; R is methyl; R³ is hydroxyl, protected hydroxyl, —OP(OR^{P})N(R^{P2})₂, or a linker covalently linked to a solid support; R⁴ is H; and R⁵ is —CH═CH—P(O)(OR^{5e})₂.

### Internucleoside Linkages

As used herein, “internucleoside linkage” refers to a covalent linkage between adjacent nucleosides. The two main classes of internucleoside linkages are defined by the presence or absence of a phosphorus atom. Representative phosphorus containing linkages include, but are not limited to, phosphodiesters (P=O), phosphotriesters, methylphosphonates, phosphoramidate, and phosphorothioates (P=S). Representative non-phosphorus containing linking groups include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Modified internucleoside linkages, compared to natural phosphodiester linkages, can be used to alter, typically increase, nuclease resistance of the oligonucleotide compound. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the internucleoside linkage can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphodiester internucleoside linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral. In other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. The non-bridging oxygens can be independently any one of O, S, Se, B, C, H, N, or OR (R is alkyl or aryl).

A phosphodiester internucleoside linkage can also be modified by replacement of bridging oxygen, (i.e. oxygen that links the phosphate to the sugar of the nucleosides), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH₂—S—C5′, C3′—O—P(O)—O—SS—C5′, C3′—CH₂—NH—NH—C5′, 3′—NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester internucleoside linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Additional exemplary non-phosphorus containing internucleoside linking groups are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, content of each of which is incorporated herein by reference.

In some embodiments of any one of the aspects, the oligonucleotides of the invention comprise one or more neutral internucleoside linkages that are non-ionic. Suitable neutral internucleoside linkages include, but are not limited to, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′); nonionic linkages containing siloxane (dialkylsiloxane), carboxylate ester, carboxamide, sulfide, sulfonate ester and/or amides (See for example: Carbohydrate Modifications in Antisense Research; Y. S. Sanghvi and P. D. Cook Eds. ACS Symposium Series 580; Chapters 3 and 4, (pp. 40-65)); and nonionic linkages containing mixed N, O, S and CH₂ component parts.

In one embodiment, the non-phosphodiester backbone linkage is selected from the group consisting of phosphorothioate, phosphorodithioate, alkyl-phosphonate and phosphoramidate backbone linkages.

In some embodiments of any one of the aspects described herein, the internucleoside linkage is

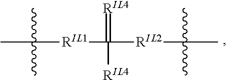

R^{IL4}, where R^{IL1} and R^{IL2} are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and R^{IL3} and R^{IL4} are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), alkyl or aryl. It is understood that one of R^{IL1} and R^{IL2} is replacing the oxygen linked to 5′ carbon of a first nucleoside sugar and the other of R^{IL1} and R^{IL2} is replacing the oxygen linked to 3′ (or 2′) carbon of a second nucleoside sugar.

In some embodiments of any one of the aspects, R^{IL1}, R^{IL2}, R^{IL1} and R^{IL2} all are 0.

In some embodiments, R^{IL1} and R^{IL2} are 0 and at least one of R^{IL3} and R^{IL4}. For example, one of R^{IL3} and R^{IL4} is S and the other is 0 or both of R^{IL3} and R^{IL4} are S.

In some embodiments of any one of the aspects, one of R^{a′} or R^{c} is a bond to R^{IL1} or R^{IL2}. For example, R^{c} is a bond to R^{IL1}.

In some embodiments of any one of the aspects, one of R^{a′} or R^{c} is a bond to R^{IL1} or R^{IL2} and R^{d} is a bond to the other of R^{IL1} or R^{IL2}. For example, R^{c} is a bond to R^{IL1} and R^{d} is a bond to R^{IL2}.

### Nitrogen Protecting Groups

Some embodiments of the various aspects described herein include a nitrogen protecting group (also referred to as an amino protecting group herein). Nitrogen protecting groups include, but are not limited to, —OH, —OR^{NP1}, —N(R^{N2})₂, —C(═O)R^{N1}, —C(═O)N(R^{NP2})₂, —CO₂R^{NP1}, —SO₂R^{NP1}, —C(═NR^{NP2})R^{NP1}, —C(═NR^{NP2})OR^{NP1}, —C(═NR^{NP2})N(R^{NP2})₂, —SO₂N(R^{NP2})₂, —SO₂R^{NP2}, —SO₂OR^{NP2}, —SOR^{NP1}, —C(═S)N(R^{NP2})₂, —C(═O)SR^{NP2}, —C(═S)SR^{NP2}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, where each R^{NP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{NP1} groups are joined to form a 3-14 membered heterocyclyl or 5-14 membered heteroaryl ring; and each R^{NP2} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring, and wherein each alkyl, alkenyl, alkynyl, carbocyclyl, heterocyclyl, aralkyl, aryl, and heteroaryl of R^{NP1} and R^{NP2} can be optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6.

Nitrogen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary amide (e.g., —C(═O)R^{NP1}) nitrogen protecting groups include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxy acylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Exemplary carbamate (e.g., —C(═O)OR^{NP1}) nitrogen protecting groups include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Exemplary sulfonamide (e.g., —S(═O)₂R^{NP1}) nitrogen protecting groups include, but are not limited to, such as p-toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Additional exemplary nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuNP2inimide (Dts), N—2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N—S-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane and N-diphenylborinic acid derivative, N-[phenyl(pentNP1cylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

### Oxygen Protecting Groups

Some embodiments of the various aspects described herein include an oxygen protecting group (also referred to as an hydroxyl protecting group herein). Oxygen protecting groups include, but are not limited to, —R^{OP1}, —N(R^{OP2})₂, —C(═O)SR^{OP1}, —C(═O)R^{OP1}, —CO₂R^{OP1}, —C(═O)N(R^{OP2})₂, —C(═NR^{OP2})R^{OP1}, —C(═NR^{OP2})OR^{OP1}, —C(═NR^{OP2})N(R^{OP2})₂, —S(═O)R^{OP1}, —SO⁺₂R^{OP1}, —Si(R^{OP1})₃, —P(R^{OP3})₂, —P(R^{OP3})⁺₃ X⁻, —P(OR^{OP3})₂, —P(OR^{OP3})₃ X⁻, —P(═O)(R^{OP1})₂, —P(═O)(OR^{OP3})₂, and —P(═O)(N(R^{OP2})₂)₂; wherein each X is a counterion; each R^{OP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{OP1} groups are joined to form a 3-14 membered heterocyclyl or 5-14 membered heteroaryl ring; each R^{OP2} is hydrogen, —OH, —OR^{OP1}, —N(R^{OP3})₂, —CN, —C(═O)R^{OP1}, —C(═O)N(R^{OP3})₂, —CO₂R^{OP1}, —SO₂R^{OP1}, —C(═NR^{OP3})OR^{OP1}, —C(═NR^{OP3})N(R^{OP3})₂, —SO₂N(R^{OP3})₂, —SO₂R^{OP3}, —SO₂OR^{OP3}, —SOR^{OP1}, —C(═S)N(R^{OP3})₂, —C(═O)SR^{OP3}, —C(═S)SR^{OP3}, —P(═O)(R^{OP1})₂, —P(═O)(OR^{OP3})₂, —P(═O)(N(R^{OP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{OP2} groups are joined to form a 3-14 membered heterocyclyl or 5-14 membered heteroaryl ring; and each R^{OP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{OP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{OP1}, R^{OP2} and R^{OP3} can be optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6.

Oxygen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, u-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuSP3inoate, (E)-2-methyl-2-butenoate, o-(methoxyacyl)benzoate, α-naphthoate, nitrate, alkylN,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In some embodiments of any one of the aspects described herein, oxygen protecting group is benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In certain embodiments, T1 is a hydroxyl protecting group selected from acetyl, benzyl, t-butyldimethylsilyl, t-butyldiphenylsilyl and dimethoxytrityl wherein a more preferred hydroxyl protecting group is T1 is 4,4′-dimethoxytrityl.

### Sulfur Protecting Groups

Some embodiments of the various aspects described herein include sulfur protecting group (also referred to as a thiol protecting group herein). Sulfur protecting groups include, but are not limited to, —R^{SP1}, —N(R^{SP2})₂, —C(═O)SR^{SP1}, —C(═O)R^{SP1}, —CO₂R^{SP1}, —C(═O)N(R^{SP2})₂, —C(═NR^{SP2})R^{SP1}, —C(═NR^{SP2})OR^{SP1}, —C(═NR^{SP2})N(R^{SP2})₂, —S(═O)R^{SP1}, —SO₂R^{SP1}, —Si(R^{SP1})₃, —P(R^{SP3})₂, —P(R^{SP3})⁺₃ X⁻, —P(OR^{SP3})₂, —P(OR^{SP3})⁺₃ X⁻, —P(═O)(R^{SP1})₂, —P(═O)(OR^{SP3})₂, and —P(═O)(N(R^{SP2})₂)₂, wherein

X⁻ is a counterion; each R^{SP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{SP1} groups are joined to form a 3-14 membered heterocyclyl or 5-14 membered heteroaryl ring; each R^{SP2} is hydrogen, —OH, —OR^{SP1}, —N(R^{SP3})₂, —CN, —C(═O)R^{SP1}, —C(═O)N(R^{SP3})₂, —CO₂R^{SP1}, —SO₂R^{SP1}, —C(═NR^{SP3})OR^{SP1}, —C(═NR^{SP3})N(R^{SP3})₂, —SO₂N(R^{SP3})₂, —SO₂R^{SP3}, —SO₂OR^{SP3}, —SOR^{SP1}, —C(═S)N(R^{SP3})₂, —C(═O)SR^{SP3}, —C(═S)SR^{SP3}, —P(═O)(R^{SP1})₂, —P(═O)(OR^{SP3})₂, —P(═O)(N(R^{SP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{SP2} groups are joined to form a 3-14 membered heterocyclyl or 5-14 membered heteroaryl ring; and each R^{SP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{SP1}, R^{SP2} and R^{SP3} can be optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6.

Sulfur protecting groups are well known in the art and include those described in detail in Greene's Protecting Groups in Organic Synthesis, P. G. M. Wuts, 5^{th} Edition, John Wiley & Sons, 2014, incorporated herein by reference.

### Compounds/Monomers

In one aspect, provided herein are compounds/monomers, i.e., 2′-geminal-substituted nucleosides and nucleotides of formula (III) or (III′). In some embodiments, the compound of formula (III) is selected from the compounds shown in Table 1.

TABLE 1
Exemplary compounds of formula (III)

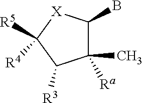

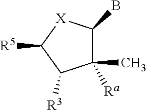

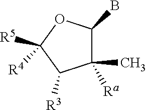

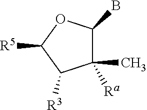

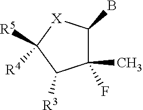

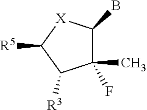

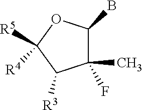

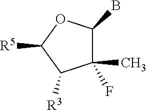

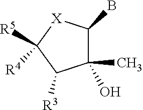

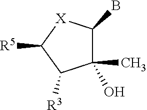

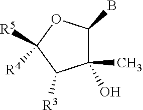

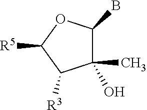

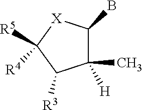

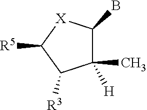

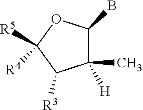

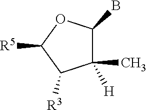

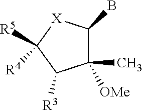

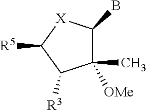

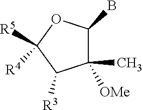

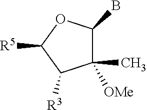

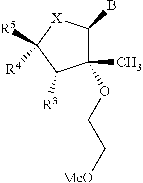

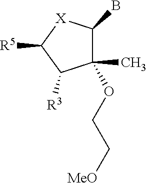

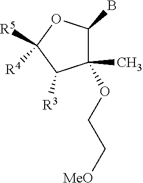

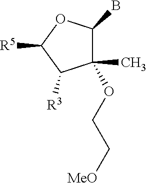

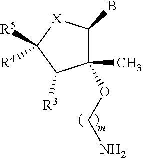

(m is 2-10, e.g., 3, 6)

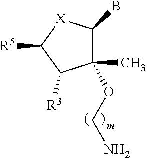

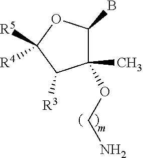

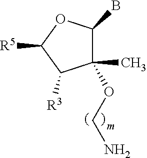

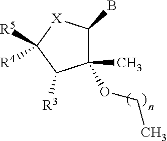

(n is 1-21, e.g., 16)

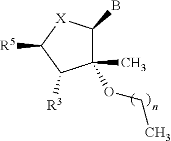

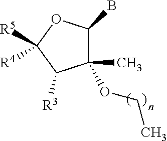

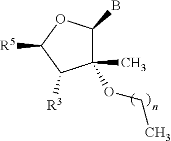

In some embodiments, the compound of formula (III) is selected from the compounds shown in Tables 2-3.

TABLE 2
Exemplary compounds of formula (III)

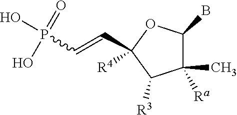

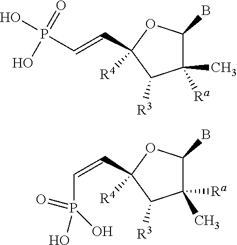

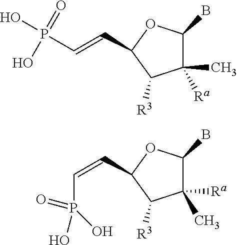

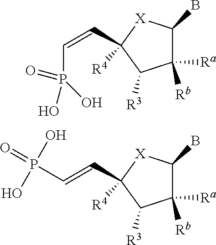

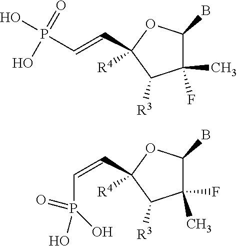

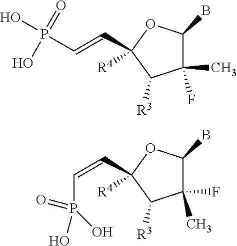

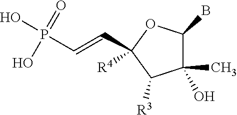

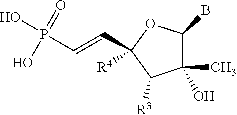

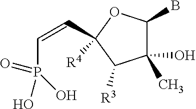

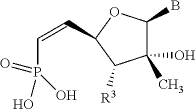

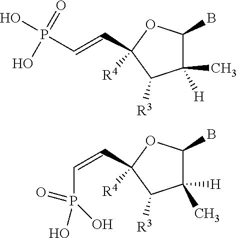

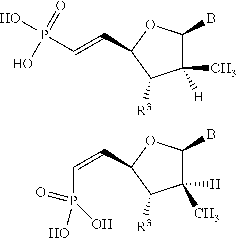

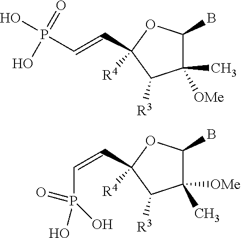

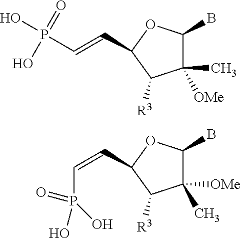

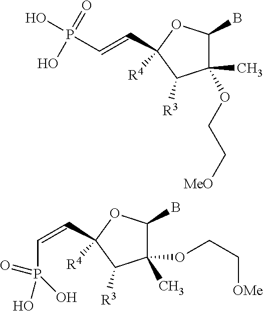

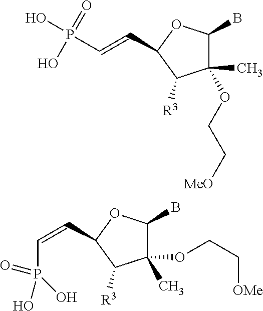

TABLE 3
Exemplary compounds of formula (III)

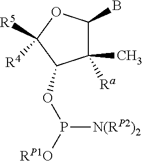

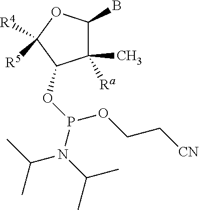

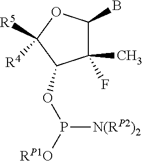

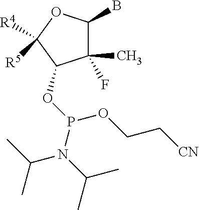

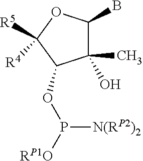

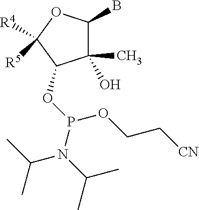

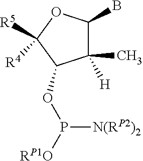

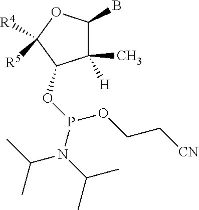

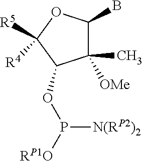

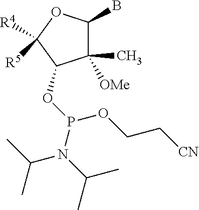

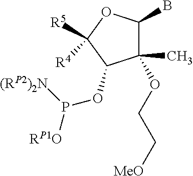

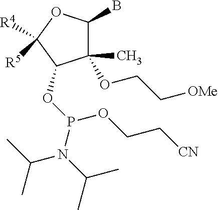

In the compounds of Table 3, one or both hydroxyl groups attached to the phosphorous can be replaced independently with an optionally substituted C₁-C₃₀alkyl, optionally substituted C₂-C₃₀alkenyl or optionally substituted C₂-C₃₀alkynyl. For example, one of the hydroxyl groups attached to the phosphorous can be replaced independently with an an optionally substituted C₁-C₆alkyl, optionally substituted C₂-C₆alkenyl or optionally substituted C₂-C₆₀alkynyl.

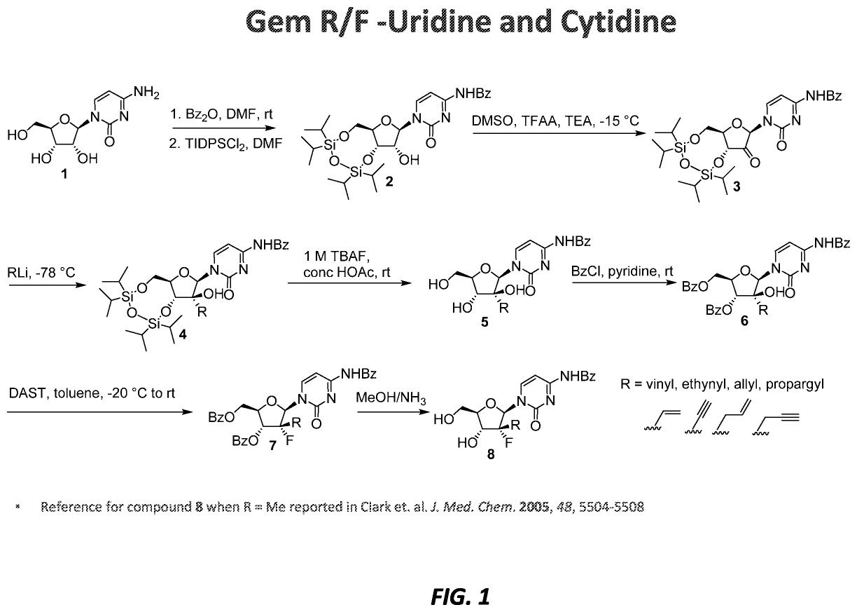

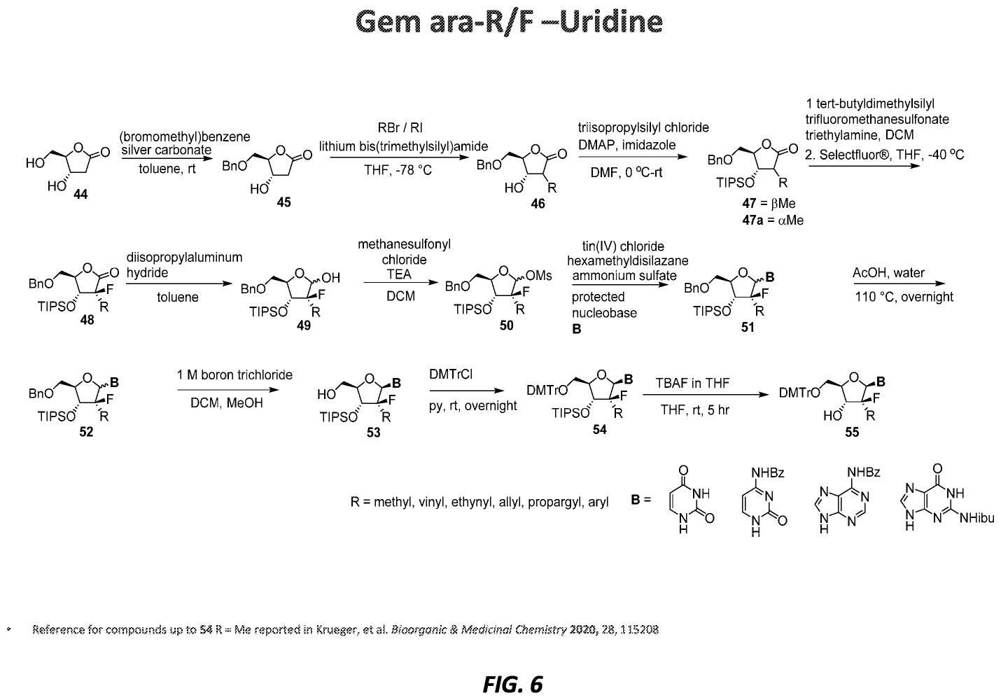

Exemplary 2′-geminal-substituted nucleosides and nucleotides of formula (III) can be prepared according to the synthetic schemes shown in FIGS. 1-15.

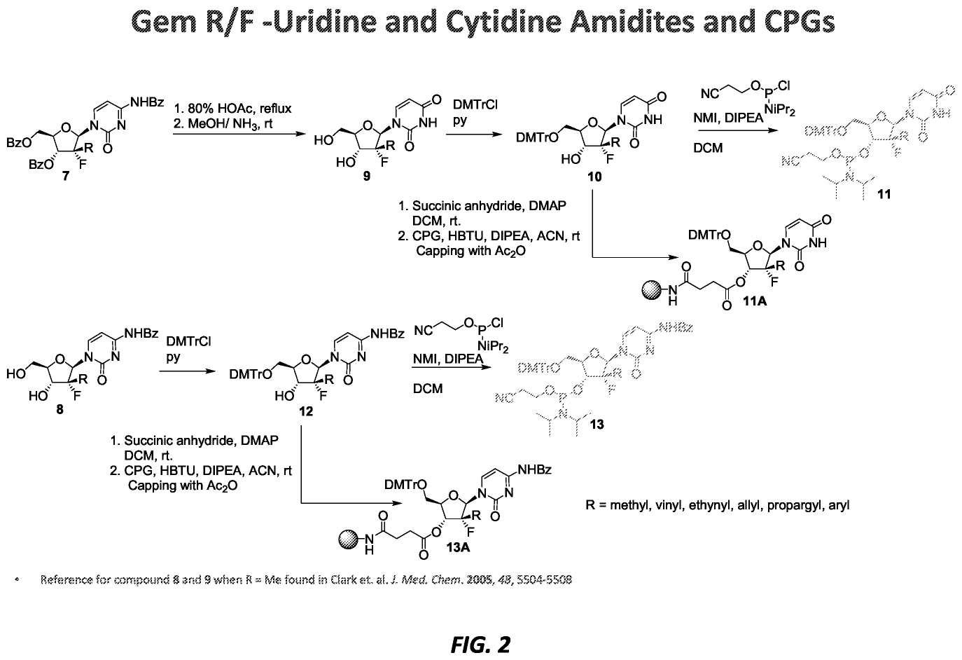

In some embodiments of any one of the aspects, a compound of formula (III) is Compound 10, 11, 11A, 12, 13 or 13A as shown in FIG. 2.

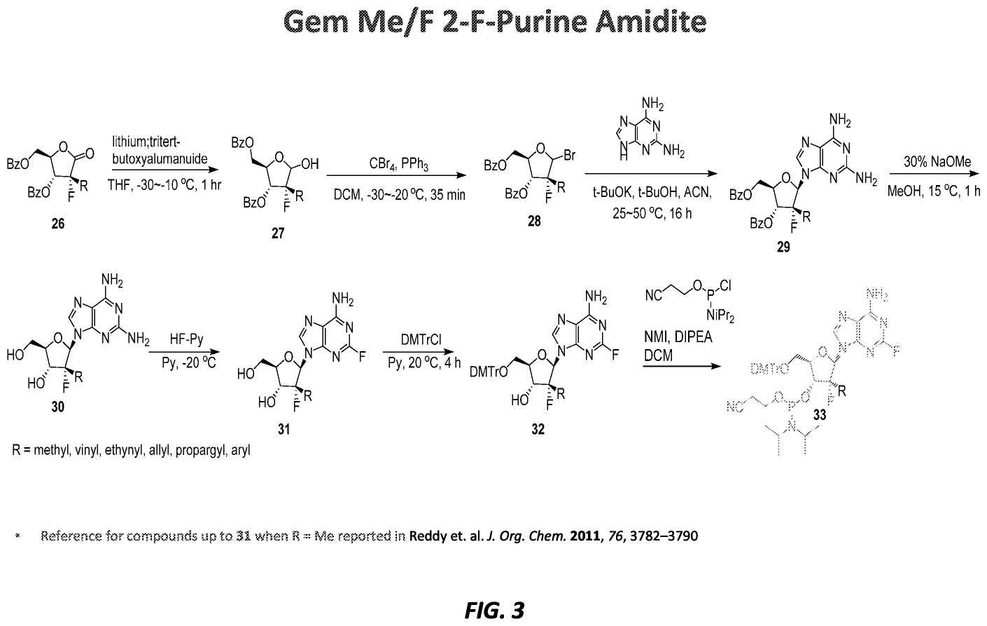

In some embodiments of any one of the aspects, a compound of formula (III) is Compound 32 or 33 as shown in FIG. 3.

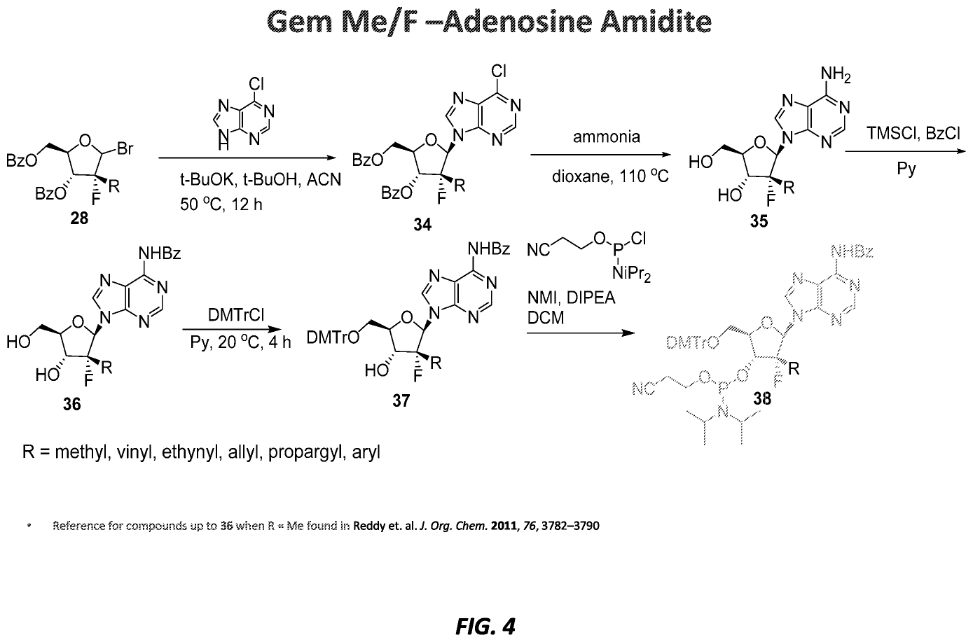

In some embodiments of any one of the aspects, a compound of formula (III) is Compound 37 or 38 as shown in FIG. 4.

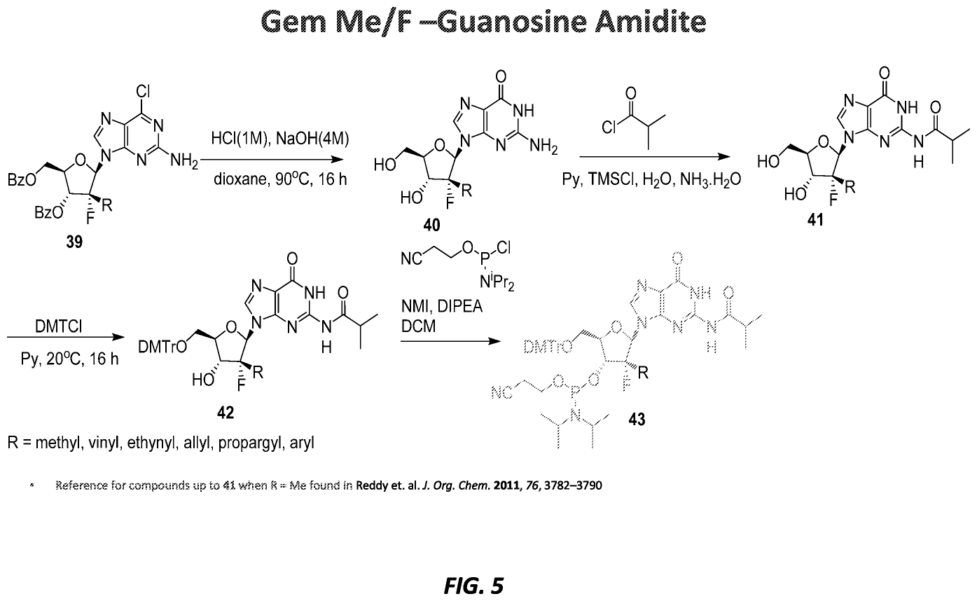

In some embodiments of any one of the aspects, a compound of formula (III) is Compound 42 or 43 as shown in FIG. 5.

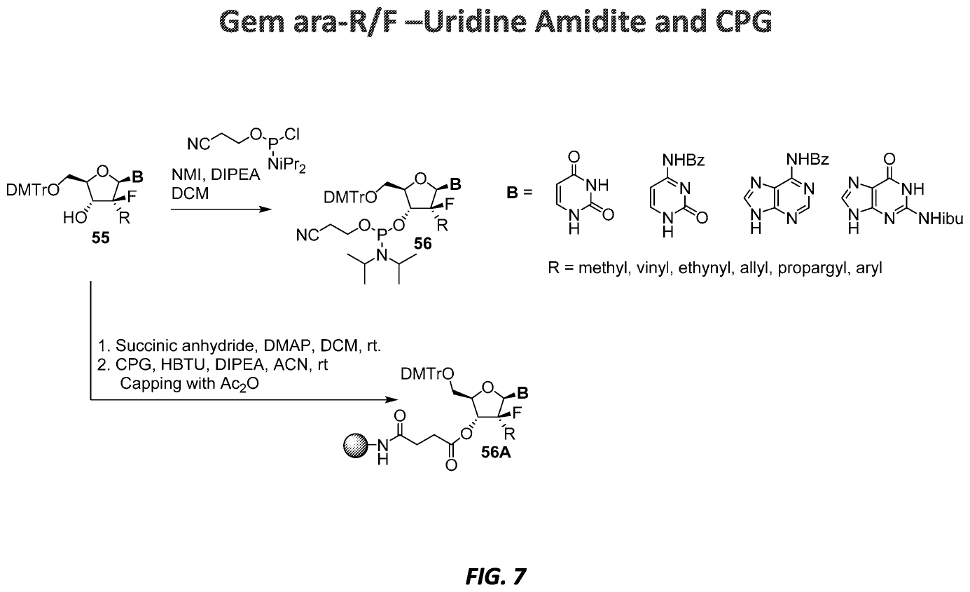

In some embodiments of any one of the aspects, a compound of formula (III) is Compound 55, 56 or 56A as shown in FIG. 7.

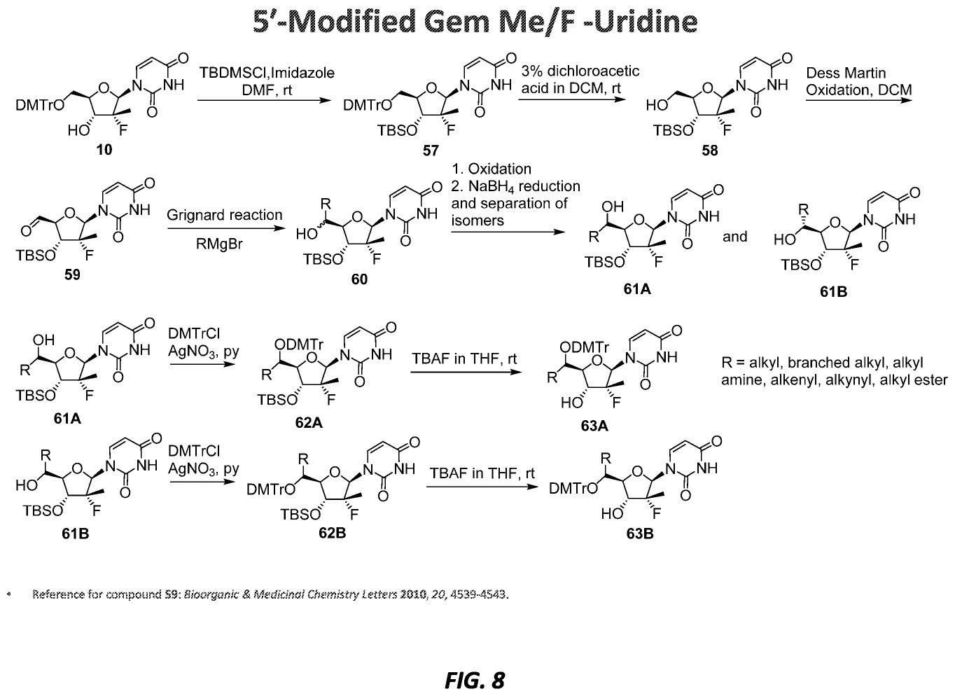

In some embodiments of any one of the aspects, a compound of formula (III) is Compound 60, 61A, 61B, 62A, 62B, 63A or 63B as shown in FIG. 8.

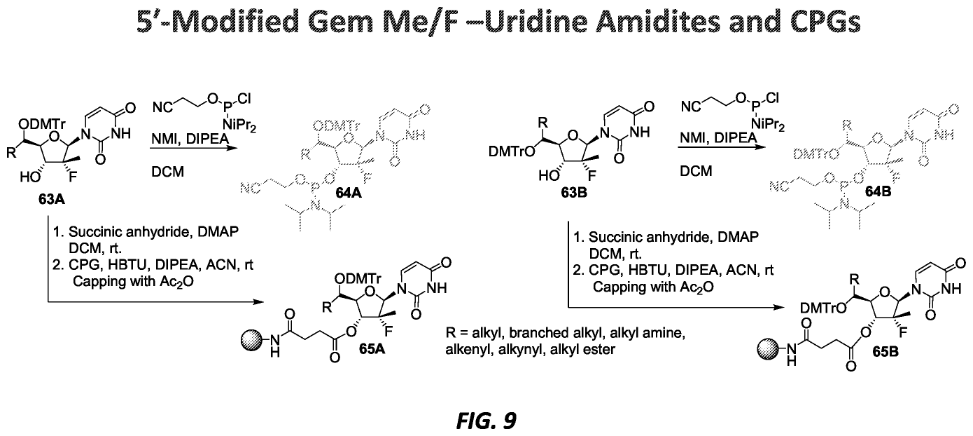

In some embodiments of any one of the aspects, a compound of formula (III) is Compound 64A, 64B, 64A or 65B as shown in FIG. 9.

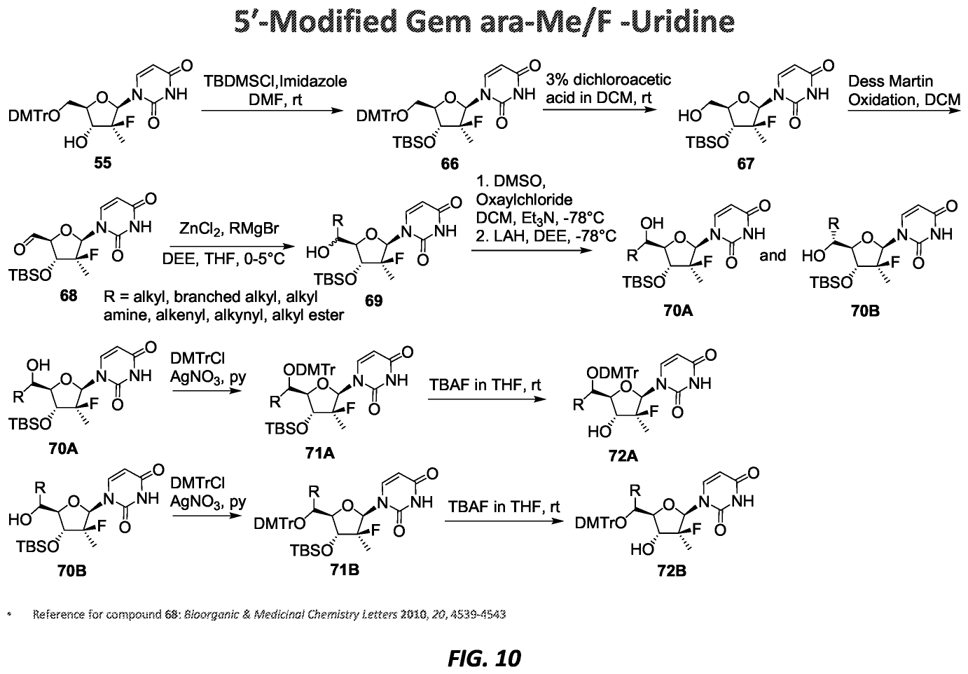

In some embodiments of any one of the aspects, a compound of formula (III) is Compound 69, 70A, 70B, 71A, 71B, 72A or 72B as shown in FIG. 10.

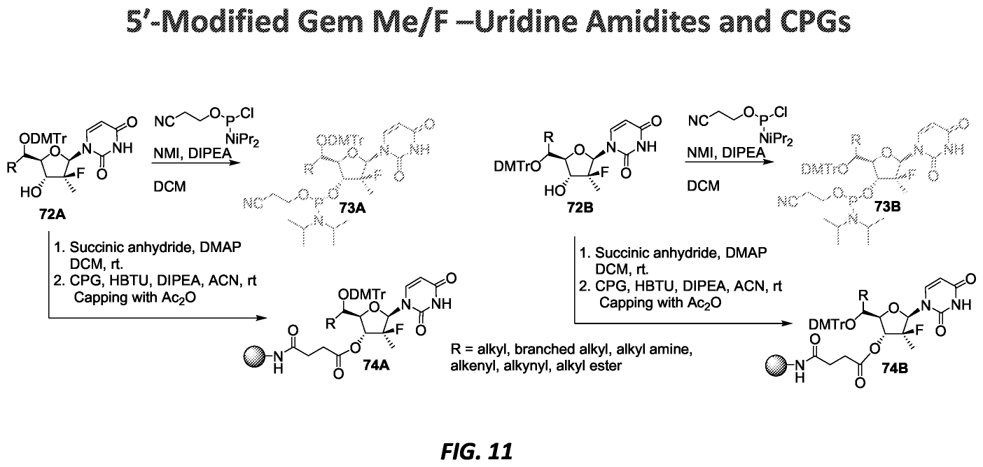

In some embodiments of any one of the aspects, a compound of formula (III) is Compound 73A, 73B, 74A or 74B as shown in FIG. 11.

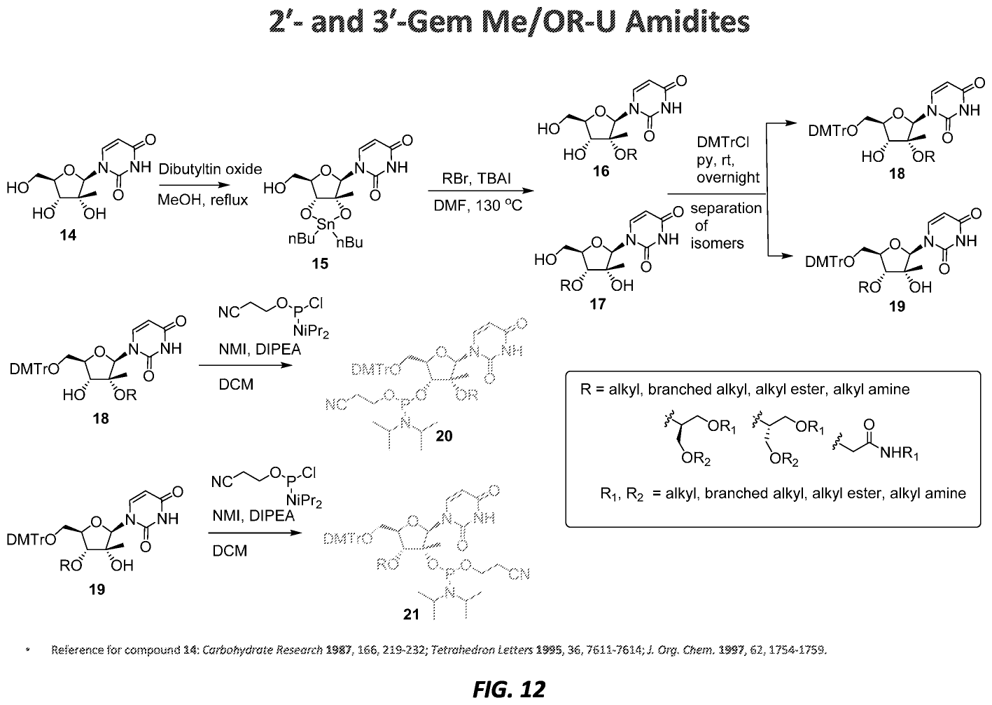

In some embodiments of any one of the aspects, a compound of formula (III) is Compound 15, 16, 17, 18, 19, 20 or 21 as shown in FIG. 12.

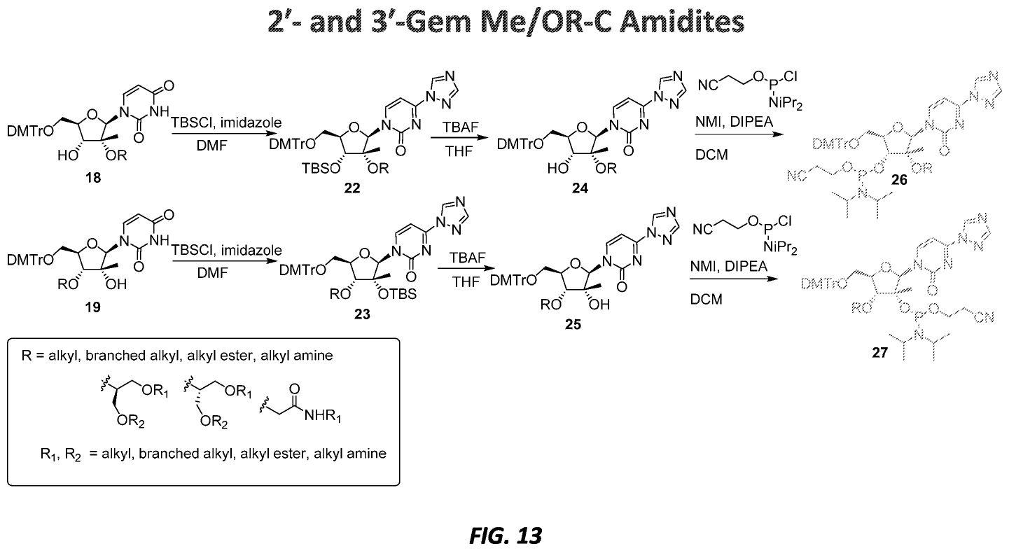

In some embodiments of any one of the aspects, a compound of formula (III) is Compound 22, 23, 24, 25, 26 or 27 as shown in FIG. 13.

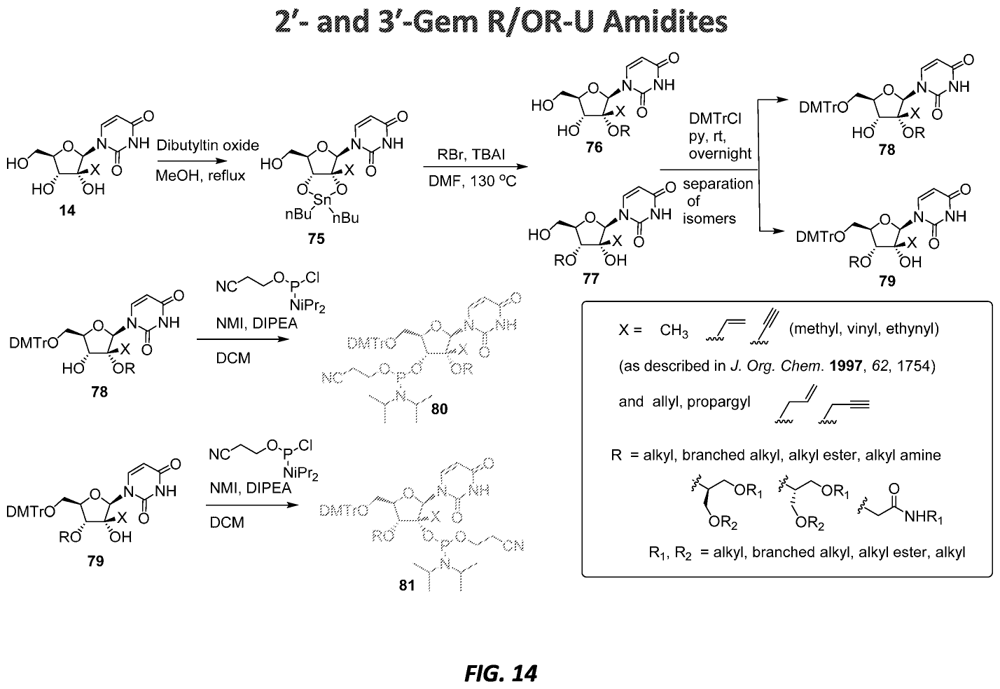

In some embodiments of any one of the aspects, a compound of formula (III) is Compound 75, 76, 77, 78, 79, 80 or 81 as shown in FIG. 14.

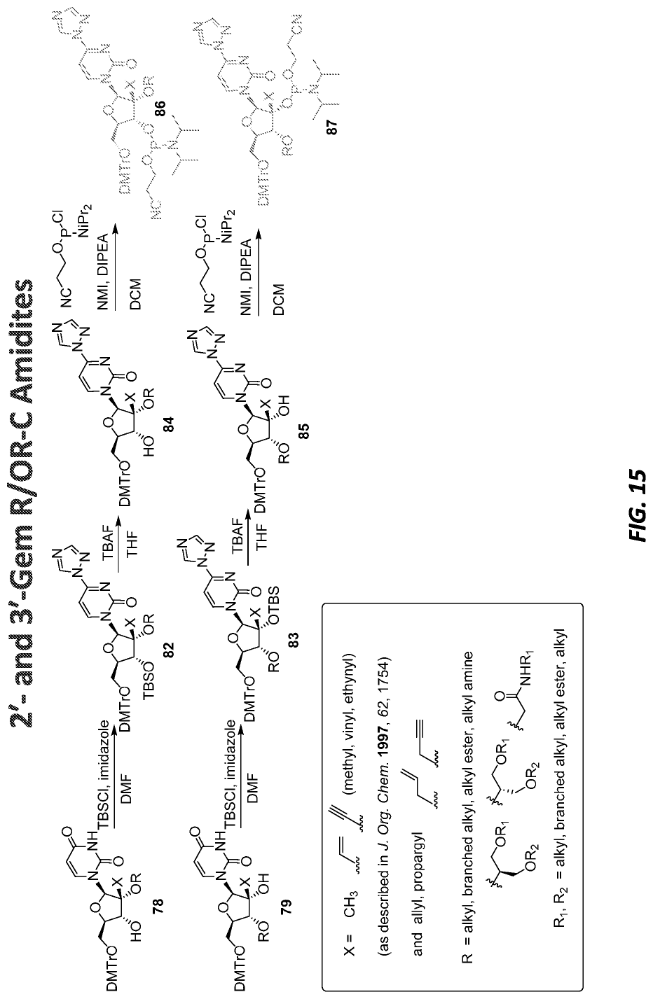

In some embodiments of any one of the aspects, a compound of formula (III) is Compound 82, 83, 84, 85, 86 or 87 as shown in FIG. 15.

In some embodiments of any one of the aspects, a compound of formula (III) is not where R^{a} is F; R^{b} is methyl; R³ is —OR^{c2}; reactive phosphorous group or linkage to a solid support; R^{c2} is hydrogen or hydroxyl protecting group R⁴ is H; R⁵ is —CH₂OR^{5b}, R^{5b} is H, hydroxyl protecting group or a phosphorus group; and B is adenine, cytosine, guanine or uracil, each of which can be unprotected, protected or modified.

In some embodiments of any one of the aspects, a compound of formula (III) is not where R^{a} is OH; R^{b} is methyl, vinyl or ethynyl; R³ is —OR^{c2}, reactive phosphorous group or linkage to a solid support; R^{c2} is hydrogen or hydroxyl protecting group R⁴ is H; R⁵ is —CH₂OR^{5b}, R^{5b} is H, hydroxyl protecting group or a phosphorus group; and B is adenine or guanine, each of can be unprotected, protected or modified.

In some embodiments of any one of the aspects, a compound of formula (III) is not the Compound 1-9, 14, 28-31, 34-36, 39-41, 44-55, 57-59, 66-68 as shown in FIGS. 1-12.

In some embodiments of any one of the aspects described herein, a compound of Formula (III) is not of structure

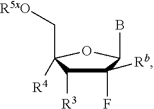

where:
R^{b} is hydrogen or a substituted or unsubstituted C₁-C₄ alkyl; R^{c} is-OR^{IX}, where:
   R^{Ix} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, a ligand carrying monomer, —F, —(CᵣC₆)alkyl, —(C₂-C₆)allyl, —(C(R³)₂)ₙOR³, —(C(R³)₂)ₙSR³, —(C(R³)₂)ₙN(R³)₂, —(C(R³)₂)ₙC(O)N(R³)₂, —(C(R³)₂)ₙO(CᵣC₆)alkyl, —(C(R³)₂)ₙS(CᵣC₆)alkyl, —(C(R³)₂)ₙO(C(R³)₂)ₙN((C₁-C₆)alkyl)₂, —(C(R³)₂)ₙON((C₁-C₆)alkyl)₂, —C(O)R³, —C(O)R³C(O)H, —C(O)R³C(O)OH, —C(O)R³C(O)R³, —C(O)R³C(O)NR³ —PO₂, —P(OR³)₂, —P(N(R³)₂)₂, —P(OR³)N(R³)₂, or a linker;
R⁴ is H;
R^{5x} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, or a ligand carrying monomer;
M represents independently for each occurrence an alkali metal or a transition metal with an overall charge of +1; and
n is an integer from 1-4.

In some embodiments of any one of the aspects described herein, the compound of Formula (III) is not

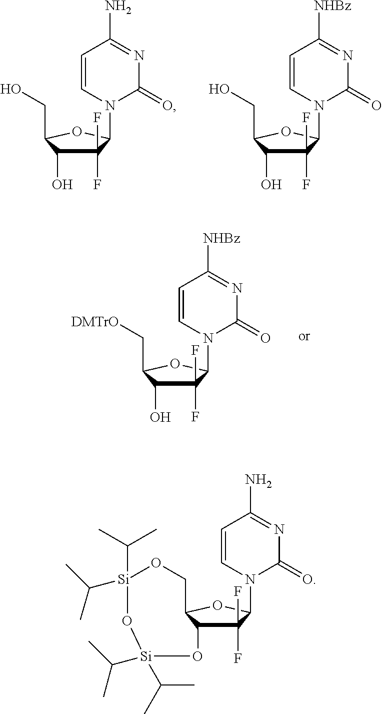

### Olignucleotides

In one aspect, provided herein is an oligonucleotide comprising: (i) at least one 2′-geminal-substituted nucleoside of formula (I) or (I′); and/or (ii) a 2′-geminal-substituted nucleoside of formula (II) or (II′) at the 5′-terminal nucleotide.

In some embodiments of any one of the aspects described herein, a 2′-geminal-substituted nucleoside of formula (II) at the 5′-terminal nucleotide is not of structure:

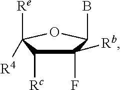

where:
R^{b} is hydrogen or a substituted or unsubstituted C₁-C₄ alkyl;
R^{c} is a bond to an internucleotide linkage to a subsequent nucleoside or —OR^{Ix}, where R^{Ix} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, a ligand carrying monomer, —F, —(CᵣC₆)alkyl, —(C₂-C₆)allyl, —(C(R³)₂)ₙOR³, —(C(R³)₂)ₙSR³, —(C(R³)₂)ₙN(R³)₂, —(C(R³)₂)ₙC(O)N(R³)₂, —(C(R³)₂)ₙO(CᵣC₆)alkyl, —(C(R³)₂)ₙS(CᵣC₆)alkyl, —(C(R³)₂)ₙO(C(R³)₂)ₙN((C₁-C₆)alkyl)₂, —(C(R³)₂)ₙON((C₁-C₆)alkyl)₂, —C(O)R³, —C(O)R³C(O)H, —C(O)R³C(O)OH, —C(O)R³C(O)R³, —C(O)R³C(O)NR³ —PO₂, —P(OR³)₂, —P(N(R³)₂)₂, —P(OR³)N(R³)₂, or a linker;
R⁴ is H;
R^{e} is —CH₂OR^{IIx}, where
R^{IIx} is —H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, or a ligand carrying monomer;
M represents independently for each occurrence an alkali metal or a transition metal with an overall charge of +1; and
n is an integer from 1-4.

In some embodiments of any one of the aspects described herein, the nucleoside of Formula (II) is not

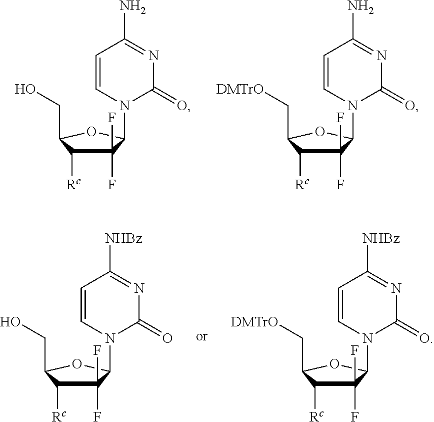

In some embodiments of any one of the aspects described herein, a 2′-geminal-substituted nucleoside of formula (I) is not of structure:

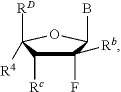

where:
R^{b} is hydrogen or a substituted or unsubstituted C₁-C₄ alkyl;
R^{c} is a bond to an internucleotide linkage to a subsequent nucleoside or —OR^{Ix}, where
R^{Ix} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, a ligand carrying monomer, —F, —(CᵣC₆)alkyl, —(C₂—C₆)allyl, —(C(R³)₂)ₙOR³, —(C(R³)₂)ₙSR³, —(C(R³)₂)ₙN(R³)₂, —(C(R³)₂)ₙC(O)N(R³)₂, —(C(R³)₂)ₙO(CᵣC₆)alkyl, —(C(R³)₂)ₙS(CᵣC₆)alkyl, —(C(R³)₂)ₙO(C(R³)₂)ₙN((C₁-C₆)alkyl)₂, —(C(R³)₂)ₙON((C₁-C₆)alkyl)₂, —C(O)R³, —C(O)R³C(O)H, —C(O)R³C(O)OH, —C(O)R³C(O)R³, —C(O)R³C(O)NR³ —PO₂, —P(OR³)₂, —P(N(R³)₂)₂, —P(OR³)N(R³)₂, or a linker;
R⁴ is H;
R^{d} is a bond to an internucleotide linkage to a preceding nucleoside;
M represents independently for each occurrence an alkali metal or a transition metal with an overall charge of +1; and
n is an integer from 1-4, and
1; and
n is an integer from 1-4.

In some embodiments of any one of the aspects described herein, the nucleoside of Formula (I) is not

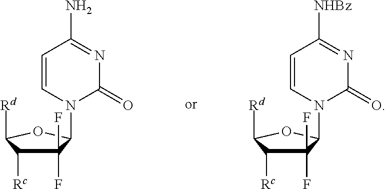

It is noted that the 2′-geminal-substituted nucleoside of formula (I) or (I′) can be located anywhere in the oligonucleotide. In some embodiments, the 2′-geminal-substituted nucleoside of formula (I) or (I′) is present at positions 2-10, counting from 5′-end, of the oligonucleotide. For example, the 2′-geminal-substituted nucleoside of formula (I) or (I′) is present at position 2, or at position 3, at position 4, at position 5, at position 6, at position 7, at position 8, at position 9, or at positon 10, counting from 5′-end, of the oligonucleotide. In some non-limiting examples, the 2′-geminal-substituted nucleoside of formula (I) or (I′) is present at position 2, counting from 5′-end, of the oligonucleotide. In some non-limiting examples, the 2′-geminal-substituted nucleoside of formula (I) or (I′) is present at position 3, counting from 5′-end, of the oligonucleotide. In some non-limiting examples, the 2′-geminal-substituted nucleoside of formula (I) or (I′) is present at position 4, counting from 5′-end, of the oligonucleotide. In some non-limiting examples, the 2′-geminal-substituted nucleoside of formula (I) or (I′) is present at position 5, counting from 5′-end, of the oligonucleotide. In some non-limiting examples, the 2′-geminal-substituted nucleoside of formula (I) or (I′) is present at position 6, counting from 5′-end, of the oligonucleotide. In some non-limiting examples, the 2′-geminal-substituted nucleoside of formula (I) or (I′) is present at position 7, counting from 5′-end, of the oligonucleotide. In some non-limiting examples, the 2′-geminal-substituted nucleoside of formula (I) or (I′) is present at position 8, counting from 5′-end, of the oligonucleotide. In some non-limiting examples, the 2′-geminal-substituted nucleoside of formula (I) or (I′) is present at position 9, counting from 5′-end, of the oligonucleotide. In some non-limiting examples, the 2′-geminal-substituted nucleoside of formula (I) or (I′) is present at position 10, counting from 5′-end, of the oligonucleotide.

In some embodiments, the oligonucleotide comprises at least one, e.g., 1, 2, 3, 4, 5, 6,7, 8, 9, 10 or more 2′-geminal-substituted nucleosides of formula (I) and/or (I′). For example, the oligonucleotide comprises, 1, 2, 3, 4, 5, 6, 7, 8, 9 or 10 2′-geminal-substituted nucleosides of formula (I) and/or (I′). In some embodiments, all the nucleosides in the oligonucleotide are 2′-geminal-substituted nucleosides described herein. In other words, the oligonucleotide solely comprises 2′-geminal-substituted nucleotides of formulae (I), (I′), (II) and/or (II′).

In some embodiments of any one of the aspects described herein, the oligonucleotide solely comprises 2′-geminal-substituted nucleotides of formulae (I) and (II).

In some embodiments of any one of the aspects described herein, the oligonucleotide solely comprises 2′-geminal-substituted nucleotides of formulae (I) and (II), and the oligonucleotide further comprises a ligand, e.g., a mono- or multi-valent N-acetylgalactosamine (GalNac) linked to the oligonucleotide. For example, the oligonucleotide solely comprises 2′-geminal-substituted nucleotides of formulae (I) and (II), and the oligonucleotide further comprises a ligand, e.g., a mono- or multi-valent N-acetylgalactosamine (GalNac) linked to its 3′-end.

In some embodiments, the 5′-terminal nucleotide of the oligonucleotide is a 2′-geminal-substituted nucleotide of formula (II) or (II′). In some other embodiments, the 5′-terminal nucleotide of the oligonucleotide is not a 2′-geminal-substituted nucleotide of formula (II) or (II′).

In some embodiments, the oligonucleotide further comprises a nucleoside with a modified sugar. By a “modified sugar” is meant a sugar or moiety other than 2′-deoxy (i.e, 2′-H), 2′-OH ribose sugar or a 2′-geminal-substituted nucleoside described herein. Some exemplary nucleotides comprising a modified sugar are 2′-F ribose, 2′-OMe ribose, 2′-0,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxypropyl (glycol nucleic acid, GNA). It is noted that the nucleoside with the modified sugar can be present at any position of the oligonucleotide.

In some embodiments of any one of the aspects described herein an oligonucleotide, e.g. antisense strand or sense strand of dsRNA described herein comprises at least one, e.g., 2, 3, 4, 5, 6, 7, 8, 9, 10 or more CeNA nucleotides or analogs thereof. In some embodiments, the CeNA nucleotide or analog thereof is

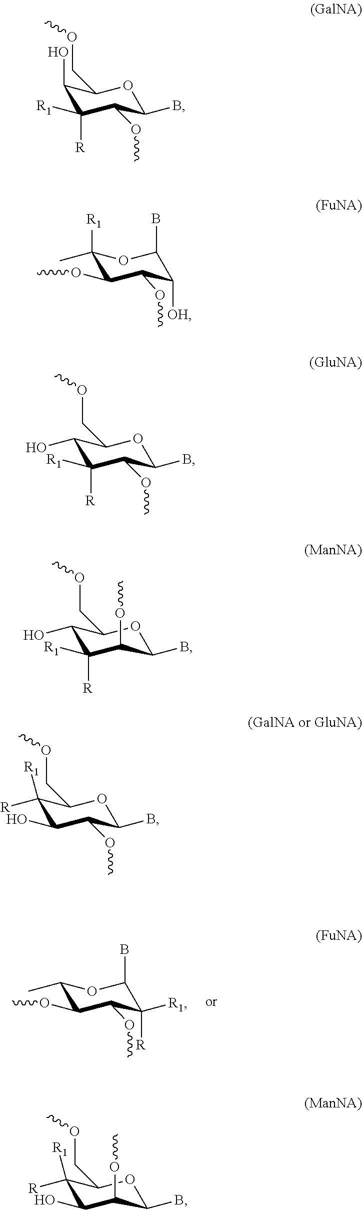

wherein:
R is F, Cl, Br, I, H, protected OH, OMe, F, O-MOE, O-alkyl, O-alkene, O-alkyne, O—C₁₆, branched lipids, or protected aminoalkyl;
R¹ is F, Cl, Br, I, H, protected OH, OMe, F, O-MOE, O-alkyl, O-alkene, O-alkyne, O—C₁₆, branched lipids, protected aminoalkyl; and
B is a nucleobase.

In some embodiments of any one of the aspects described herein the oligonucleotide, e.g. antisense strand or sense strand of dsRNA described comprising a CeNA nucleotide or analog thereof is prepared using a monomer selected from the group consisting of:

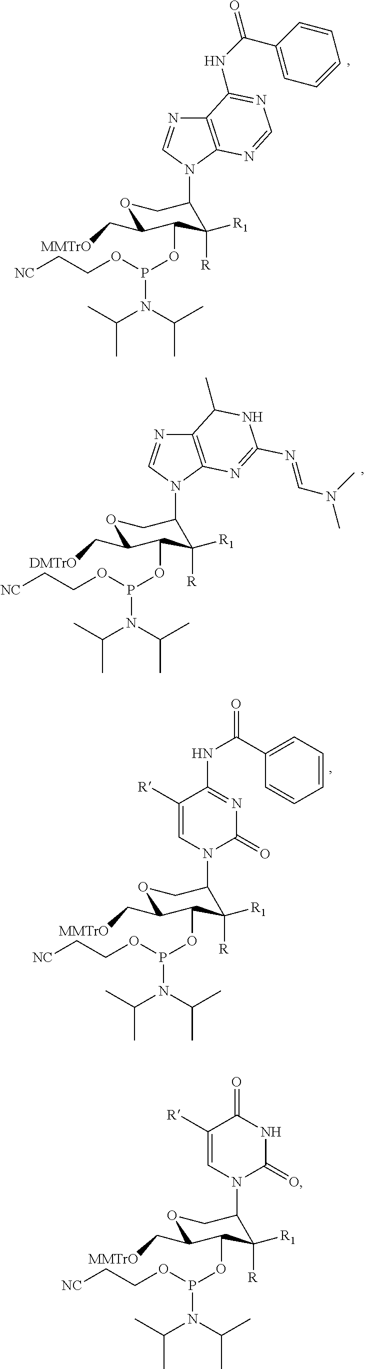

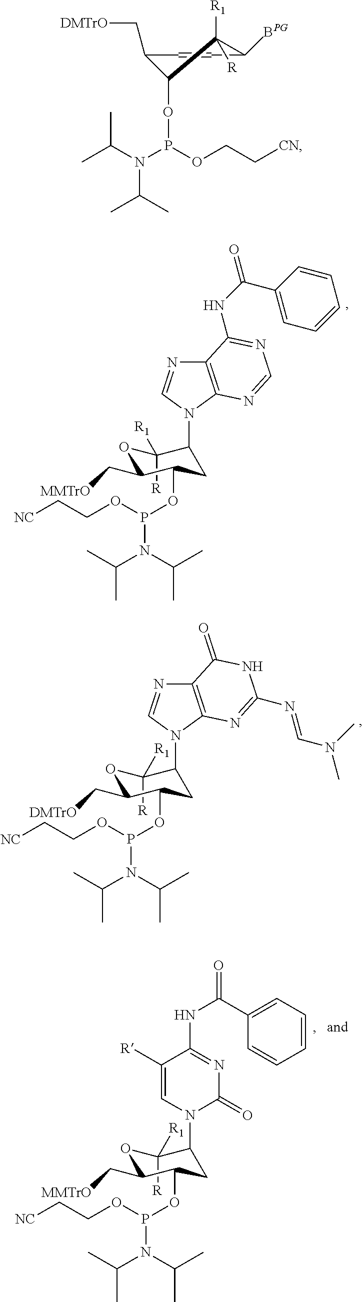

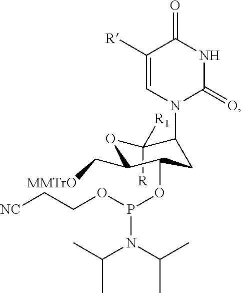

wherein:
R′ is H or CH₃;
R is F, Cl, Br, I, H, protected OH, OMe, F, O-MOE, O-alkyl, O-alkene, O-alkyne, O—C₁₆, branched lipids, or protected aminoalkyl;
R¹ is F, Cl, Br, I, H, protected OH, OMe, F, O-MOE, O-alkyl, O-alkene, O-alkyne, O—C₁₆, branched lipids, protected aminoalkyl;
PG is a protecting group; and
B is a nucleobase.

Some exemplary CeNA nucleosides and nucleotides, and analogs thereof are described in Kumar et al. Nucleic Acids Research, 2020, 48, 4028-4040; Declercq et al. J Am. Chem. Soc. 2002, 124, 928-933; Egli et al., J Am. Chem. Soc. 2011, 133, 16642-16649; Wang et al., J Am. Chem. Soc. 2000, 122, 8595-8602; Wan et al., J. Med. Chem. 2016, 59, 9645-9667; Ermolinsky et al., Russian Journal of Bioorganic Chemistry, 2002, 28, 50-57; Beheraet al., J. Am. Chem. Soc. 2020, 142, 456-467; Ghotekar et al., Org. Lett. 2020, 22, 537-541; Deshpandeet al., Tetrahedron Letters 2004, 45, 2255-2258; Deshpande et al., Tetrahedron 2007, 63, 602-608; Deshpandeet al., Carbohydrate Research 2008, 343, 1163-1170; Sanki et al., Tetrahedron 2008, 64, 10406-10416; and Rao et al., J Org. Chem. 2015, 80, 1499-1505, contents of all of which are incorporated herein by reference in their entireties.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′—F nucleotides. It is noted that the 2′-F nucleotides can be present at any position of the oligonucleotide.

In some embodiments, the oligonucleotide comprises, e.g., solely comprises 2′-geminal-substituted nucleosides and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′—OMe nucleotides. It is noted that the 2′-OMe nucleotides can be present at any position of the oligonucleotide.

In some embodiments, the oligonucleotide comprises, e.g., solely comprises solely comprises 2′-geminal-substituted nucleosides and 2′-OMe nucleosides. In some other embodiments, the oligonucleotide comprises, e.g., solely comprises 2′-geminal-substituted nucleosides, 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises 2′-geminal-substituted nucleosides and 2′-deoxy (2′-H) nucleotides. In some embodiments, the oligonucleotide comprises, e.g., solely comprises 2′-geminal-substituted nucleosides, 2′-OMe nucleosides, and 2′-deoxy (2′-H) nucleotides. In some embodiments, the oligonucleotide comprises, e.g., solely comprises 2′-geminal-substituted nucleosides, 2′-F nucleosides and 2′-deoxy (2′-H) nucleotides. In some embodiments, the oligonucleotide comprises, e.g., solely comprises 2′-geminal-substituted nucleosides, 2′-OMe nucleosides, 2′-F nucleosides and 2′-deoxy (2′-H) nucleotides.

In some embodiments, the oligonucleotide can comprise one or more, e.g., 1, 2, 3, 4, 5, 6, 7, 8, 9, 10 or more nucleotides comprising a non-natural nucleobase. A nucleotide comprising a non-natural nucleobase can be present anywhere in the oligonucleotide.

In some embodiments of any one of the aspects, the oligonucleotide can comprise one or more, e.g., 1, 2, 3, 4, 5, 6, 7, 8 or more modified internucleoside linkages. For example, the oligonucleotide can comprise 1, 2, 3, 4, 5 or 6 modified internucleoside linkages. For example, the oligonucleotide comprises 1, 2, 3 or 4 modified internucleoside linkages. In some embodiments, the oligonucleotide comprises at least two modified internucleoside linkages between the first five nucleotides counting from the 5′-end of the oligonucleotide and further comprises at least two modified internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects, the modified internucleoside linkage is a phosphorothioate. Accordingly, in some embodiments of any one of the aspects, the oligonucleotide comprises one or more, e.g., 1, 2, 3, 4, 5, 6, 7, 8 or more phosphorothioate internucleoside linkages. For example, the oligonucleotide comprises 1, 2, 3, 4, 5 or 6 phosphorothioate internucleoside linkages. For example, the oligonucleotide comprises 1, 2, 3 or 4 phosphorothioate internucleoside linkages. In some embodiments, the oligonucleotide comprises at least two phosphorothioate internucleoside linkages between the first five nucleotides counting from the 5′-end of the oligonucleotide and further comprises at least two phosphorothioate internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments, the oligonucleotide further comprises a ligand conjugated thereto.

In some embodiments, the oligonucleotide further comprises a solid support linked thereto.

The oligonucleotides described herein can range from few nucleotides (e.g., 2, 3, 4, 5, 6, 7, 8, 9 or 10 nucleotides) in length to hunderes of nucleotides in length. For example, the oligonucleotide can be from 5 nucleotides to 100 nucleotides in length. In some embodiments, the oligonucleotide is from 10 nucleotides to 50 nucleotides in length. For example, the oligonucleotide is between 15 and 35, more generally between 18 and 25, yet more generally between 19 and 24, and most generally between 19 and 21 base pairs in length. In some embodiments, longer oligonucleotides of between 25 and 30 nucleotides in length are preferred. In some embodiments, shorter oligonucleotides of between 10 and 15 nucleotides in length are preferred. In another embodiment, the oligonucleotide is at least 21 nucleotides in length.

### Double-Stranded RNAs

The skilled person is well aware that double-stranded RNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

Accordingly, in one aspect, provided herein is a double-stranded RNA (dsRNA) comprising a first strand (also referred to as an antisense strand or a guide strand) and a second strand (also referred to as a sense strand or passenger strand, wherein at least one of the first (i.e., the antisense strand) or the second strand (i.e., the sense strand) is an oligonucleotide described herein. In other words, at least one of the first (i.e., the antisense strand) or the second strand (i.e., the sense strand) comprises at least one 2′-geminal-substituted nucleotide of formulae (I) and/or (II).

In some preferred embodiments, the antisense strand is an oligonucleotide described herein. In other words, the antisense strand comprises at least one 2′-geminal-substituted nucleotide of formulae (I), (I′), (II) and/or (II′).

In some embodiments of the various aspects described herein, the antisense strand is substantially complementary to a target nucleic acid, e.g., a target gene or mRNA gene and the dsRNA is capable of inducing targeted cleavage of the target nucleic acid.

Each strand of the dsRNA molecule can range from 15-35 nucleotides in length. For example, each strand can be between, 17-35 nucleotides in length, 17-30 nucleotides in length, 25-35 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. For example, the sense strand and the antisense strand independently have a length of 18, 19, 20, 21, 22, 23, 24 or 25 nucleotides.

In some embodiments, the antisense strand is of length 15-35 nucleotides. In some embodiments, the antisense strand is 15-35, 17-35, 17-30, 25-35, 27-30, 17-23, 17-21, 17-19, 19-25, 19-23, 19-21, 21-25, 21-25, or 21-23 nucleotides in length. For example, the antisense strand can be 15, 16, 17, 18, 19, 20, 21, 22, 23, 24, 25, 26, 27, 28, 29, 30, 31, 32, 33, 34 or 35 nucleotides in length. In some embodiments, the antisense strand is 19, 20, 21, 22, 23, 24 or 25 nucleotides in length. For example, the antisense strand is 21, 22, 23, 24 or 25 nucleotides in length. In some particular embodiments, the antisense strand is 22, 23 or 24 nucleotides in length. For example, the antisense strand is 23 nucleotides in length.

Similar to the antisense strand, the sense strand can be, in some embodiments, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length. For example, the sense strand can be 15, 16, 17, 18, 19, 20, 21, 22, 23, 24, 25, 26, 27, 28, 29, 30, 31, 32, 33, 34 or 35 nucleotides in length. In some embodiments, the sense strand is 17, 18, 19, 20, 21, 22, 23, 24 or 25 nucleotides in length. For example, the sense strand is 19, 20, 21, 22 or 23 nucleotides in length. In some particular embodiments, the sense strand is 20, 21 or 22 nucleotides in length. For example, the sense strand is 21nucleotides in length

In some embodiments, the sense strand can be 15-35 nucleotides in length, and the antisense strand can be independent from the sense strand, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length, and the antisense strand is independently 15-35, 17-35, 17-30, 25-35, 27-30, 17-23, 17-21, 17-19, 19-25, 19-23, 19-21, 21-25, 21-25, or 21-23 nucleotides in length. For example, the sense and the antisense strand can be independently 15, 16, 17, 18, 19, 20, 21, 22, 23, 24, 25, 26, 27, 28, 29, 30, 31, 32, 33, 34 or 35 nucleotides in length. In some embodiments, the sense strand and the antisense strand are independently 17, 18, 19, 20, 21, 22, 23, 24 or 25 nucleotides in length. For example, the sense strand is 19, 20, 21, 22 or 23 nucleotides in length and the antisense strand is 21, 22, 23, 24 or 25 nucleotides in length. In some particular embodiments, the sense strand is 20, 21 or 22 nucleotides in length and the antisense strand is 22, 23 or 24 nucleotides in length. For example, the sense strand is 21 nucleotides in length and the antisense strand is 23 nucleotides in length.

The sense strand and antisense strand typically form a double-stranded or duplex region. Without limitations, the duplex region of a dsRNA agent described herein can be 12-35 nucleotide (or base) pairs in length. For example, the duplex region can be between 14-35 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length. In some embodiments, the duplex region is 18, 19, 20, 21, 22, 23, 24 or 25 nucleotide pairs in length. For example, the duplex region is 19, 20, 21, 22 or 23 nucleotide pairs in length. In some embodiments, the the duplex region is 20, 21 or 22 nucleotide pairs in length. For example, the dsRNA molecule has a duplex region of 21 base pairs.

As described herein, the dsRNA molecule described herein can comprise at least one, e.g., 1, 2, 3, 4, 5, 6, 7, 8, 9, 10 or more 2′-geminal-substituted nucleotide of formulae (I) and/or (II). Without limitations, the 2′-geminal-substituted nucleotides all can be present in one strand. The 2′-geminal-substituted nucleotide may occur on any nucleotide of the sense strand or antisense strand or both in any position of the strand.

In some embodiments, the antisense strand comprises 1, 2, 3, 4, 5, 6, 7, 8, 9, 10 or more 2′-geminal-substituted nucleotides described herein. The 2′-geminal-substituted nucleotide described herein can be present at any position of the antisense strand. For example, the 2′-geminal-substituted nucleotide described herein can be present at a terminal region of the antisense strand. For example, the 2′-geminal-substituted nucleotide described herein can be present at one or more of positions 1, 2, 3 and 4, counting from the 5′-end of the antisense strand. In another non-limiting example, the 2′-geminal-substituted nucleotide described herein nucleotide can be present at one or more of positions 1, 2, 3, 4, 5 and 6, counting from the 3′-end of the antisense strand. In some embodiments, the 2′-geminal-substituted nucleotide described herein nucleotide can be present at one or more of positions 18, 19, 20, 21, 22 and 23, counting from 5′-end of the antisense strand. The 2′-geminal-substituted nucleotide described herein nucleotide can also be located at a central region of the antisense strand. For example, the 2′-geminal-substituted nucleotide described herein nucleotide can be located at one or more of positions 6, 7, 8, 9, 10, 11, 12 and 13, counting from 5′-end of the antisense strand.

In some embodiments, the sense strand comprises 1, 2, 3, 4, 5, 6, 7, 8, 9, 10 or more 2′-geminal-substituted nucleotides described herein. The 2′-geminal-substituted nucleotide described herein can be present at any position of the sense strand. For example, the 2′-geminal-substituted nucleotide described herein can be present at a terminal region of the sense strand. For example, the 2′-geminal-substituted nucleotide described herein can be present at one or more of positions 1, 2, 3 and 4, counting from the 5′-end of the sense strand. In another non-limiting example, the 2′-geminal-substituted nucleotide described herein can be present at one or more of positions 1, 2, 3 and 4, counting from the 3′-end of the sense strand. In some embodiments, the 2′-geminal-substituted nucleotide can be present at one or more of positions 18, 19, 20 and 21, counting from 5′-end of the sense strand. The 2′-geminal-substituted nucleotide described herein can also be located at a central region of the 2′-geminal-substituted nucleotide sense strand. For example, the 2′-geminal-substituted nucleotide described herein can be located at one or more of positions 6, 7, 8, 9, 10, 11, 12 and 13, counting from 5′-end of the sense strand. In some embodiments, the sense strand does not comprise a 2′-geminal-substituted nucleotide.

As described herein, the dsRNA agent can comprise one or more, e.g., 1, 2, 3, 4, 5, 6, 7, 8, 9, 10 or more nucleotides comprising a modified sugar. Accordingly, in some embodiments, the dsRNA agent can comprise one or more, e.g., 1, 2, 3, 4, 5, 6, 7, 8, 9, 10 or more nucleotides independently selected from the group consisting of 2′-F, 2-OMe, acyclic nucleotides, locked nucleic acid (LNA), HNA, CeNA, 2′-methoxyethyl, 2′-O-allyl, 2′-C-allyl, 2′-O—N-methylacetamido (2′-O-NMA), a 2′-O-dimethylaminoethoxyethyl (2′-O-DMAEOE), 2′-O-aminopropyl (2′-O-AP), and 2′-ara-F. A nucleotide comprising modified sugar can be present anywhere in the dsRNA molecule. For example, a nucleotide comprising a modified sugar can be present in the sense strand or a nucleotide comprising a modified sugar can be present in the antisense strand. When two or more nucleotides comprising a modified sugar are present in the dsRNA molecule, they can all be in the sense strand, antisense strand or both in the sense and antisense strands.

As described herein, the dsRNA molecule described herein can comprise at least one, e.g., 1, 2, 3, 4, 5, 6, 7, 8, 9, 10 or more 2′-fluoro (2′-F) nucleotides. In some embodiments, the sense strand comprises 1, 2, 3, 4, 5, 6, 7, 8, 9, 10 or more 2′-fluoro nucleotides. The 2′-fluoro nucleotides can be located anywhere in the sense strand. For example, the sense strand comprises a 2′-fluoro nucleotide at position 10, counting from 5′-end of the sense strand. In some embodiments, the sense strand comprises a 2′-fluoro nucleotide at position 10, counting from 5′-end of the sense strand and the sense strand further comprises a 2′-fluoro nucleotide at position 8, 9, 11 or 12, counting from 5′-end of the sense strand. For example, the sense strand comprises a 2′-fluoro nucleotide at positions 9 10, counting from 5′-end of the sense strand. In another example, the sense strand comprises a 2′-fluoro nucleotide at positions 10 and 11, counting from 5′-end of the sense strand. In some embodiments, the sense strand comprises a 2′-fluoro nucleotide at positions 9, 10 and 11, counting from 5′-end of the sense strand. In some other embodiments, the sense strand comprises a 2′-fluoro nucleotide at positions 8, 9 and 10, counting from 5′-end of the sense strand. In yet some other embodiments, the sense strand comprises a 2′-fluoro nucleotide at positions 10, 11 and 12, counting from 5′-end of the sense strand.

In some embodiments, the antisense comprises 2′-fluoro nucleotides at positions 7, 10 and 11 from the 5′-end. In some other embodiments, the sense strand comprises 2′-fluoro nucleotides at positions 7, 9, 10 and 11 from the 5′-end. In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13 and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to a thermally destabilizing modification of the duplex in the antisense strand.

In some embodiments, the antisense strand comprises 1, 2, 3, 4, 5, 6, 7, 8, 9, 10 or more 2′-fluoro nucleotides. The 2′-fluoro nucleotides can be located anywhere in the antisense strand. For example, the antisense strand can comprise a 2′-fluoro nucleotide at position 14, counting from 5′-end of the antisense strand. In some embodiments, the antisense comprises 2′-fluoro nucleotides at positions 2, 14 and 16, counting from the 5′-end of the antisense strand. In some other embodiments, the antisense comprises 2′-fluoro nucleotides at positions 2, 6, 14 and 16 from the 5′-end. In still some embodiments, the antisense comprises 2′-fluoro nucleotides at positions 2, 6, 8, 9, 14 and 16 from the 5′-end.

In some embodiments, the antisense strand comprises at least one 2′-fluoro nucleotide adjacent to a destabilizing modification. For example, the 2′-fluoro nucleotide can be the nucleotide at the 5′-end or the 3′-end of a destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification. In some embodiments, the antisense strand comprises at least two 2′-fluoro nucleotides at the 3′-end of the destabilizing modification, i.e., at positions+1 and +2 from the position of the destabilizing modification.

In some embodiments, both the sense and the antisense strands comprise at least one 2′-fluoro nucleotide. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

As described herein, the dsRNA molecule described herein can comprise at least one, e.g., 1, 2, 3, 4, 5, 6, 7, 8, 9, 10 or more 2′-OMe nucleotides. Without limitations, the 2′-OMe nucleotides all can be present in one strand. The 2′-OMe nucleotide may occur on any nucleotide of the sense strand or antisense strand or both in any position of the strand.

In some embodiments, the sense strand comprises 1, 2, 3, 4, 5, 6, 7, 8, 9, 10 or more 2′-OMe nucleotides. The 2′-OMe nucleotides can be located anywhere in the sense strand. In some embodiments, the antisense strand comprises 1, 2, 3, 4, 5, 6, 7, 8, 9, 10 or more 2′-OMe nucleotides. The 2′-OMe nucleotides can be located anywhere in the antisense strand.

As described herein, the dsRNA molecule described herein can comprise at least one, e.g., 1, 2, 3, 4, 5, 6, 7, 8, 9, 10 or more 2′-deoxy, e.g., 2′-H ribose nucleotides. For example, the dsRNA can comprise 1, 2, 3, 4, 5, 6, 7, 8, 9 or 10 2′-deoxy, e.g., 2′-H nucleotides. The 2′-deoxy nucleotide may occur on any nucleotide of the sense strand or antisense strand or both in any position of the strand.

As described herein, the dsRNA can comprise at least one, e.g., at least two, at least three, at least four, at least five, at least six, at least seven or more, 2′-deoxy modifications in a central region of the sense strand and/or the antisense strand. For example, at least one of the sense stand and the antisense can comprise at least one, e.g., at least two, at least three, at least four, at least five, at least six, at least seven or more, 2′-deoxy modification in positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the sense strand or the antisense strand.

In some embodiments, the antisense strand comprises 1, 2, 3, 4, 5 or 6 of 2′-deoxy nucleotides. For example, antisense strand can comprise 2, 3, 4, 5 or 6 of 2′-deoxy nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from 5′-end of the antisense strand. In one non-limiting example, the antisense strand comprises a 2′-deoxy nucleotide at 1, 2, 3 or 4 of positions 2, 5, 7, and 12, counting from 5′-end of the antisense strand.

In some embodiments, the antisense comprises a 2′-deoxy nucleotide at positions 5 and 7, counting from 5′-end of the antisense strand. For example, the antisense strand comprises a 2′-deoxy nucleotide at positions 5, 7 and 12, counting from 5′-end of the antisense strand. In some embodiments, the antisense strand comprises a 2′-deoxy nucleotide at positions 2, 5 and 7, counting from 5′-end of the antisense strand. For example, the antisense strand comprises a 2′-deoxy nucleotide at positions 2, 5, 7 and 12, counting from 5′-end of the antisense strand. In some embodiments, the antisense strand comprises a 2′-deoxy nucleotide at positions 2, 5, 7, 12 and 14, counting, from 5′-end of the antisense strand. For example, the antisense strand comprises a 2′-deoxy nucleotide at positions 2, 5, 7, 12, 14 and 16, counting from 5′-end of the antisense strand

In some embodiments, the antisense comprises a 2′-deoxy nucleotide at position 2 or 12, counting from 5′-end of the antisense strand. For example, the antisense comprises a 2′-deoxy nucleotide at position 12, counting from 5′-end of the antisense strand.

In some embodiments, the dsRNA comprises at least three 2′-deoxy modifications, wherein the 2′-deoxy modifications are at positions 2 and 14 of the antisense strand, counting from 5′-end of the antisense strand, and at position 11 of the sense strand, counting from 5′-end of the sense strand.

In some embodiments, the dsRNA comprises at least five 2′-deoxy modifications, wherein the 2′-deoxy modifications are at positions 2, 12 and 14 of the antisense strand, counting from 5′-end of the antisense strand, and at positions 9 and 11 of the sense strand, counting from 5′-end of the sense strand.

In some embodiments, the dsRNA comprises at least seven 2′-deoxy modifications, wherein the 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In some embodiments, the antisense strand comprises at least five 2′-deoxy modifications at positions 2, 5, 7, 12 and 14, counting from 5′-end of the antisense strand.

In one non-limiting example, the sense strand does not comprise a 2′-deoxy nucleotide at position 11, counting from 5′-end of the sense strand.

In some embodiments, the dsRNA can comprise one or more, e.g., 1, 2, 3, 4, 5, 6, 7, 8, 9, 10 or more nucleotides comprising a non-natural nucleobase

A nucleotide comprising a non-natural nucleobase can be present anywhere in the dsRNA molecule. For example, a nucleotide comprising a non-natural nucleobase can be present in the sense strand or a nucleotide comprising a non-natural nucleobase can be present in the antisense strand. When two or more nucleotides comprising a non-natural nucleobase are present in the dsRNA molecule, they can all be in the sense strand, antisense strand or both in the sense and antisense strands.

The dsRNA molecule described herein can further comprise at least one phosphorothioate or methylphosphonate internucleoside linkage. The phosphorothioate or methylphosphonate internucleoside linkage modification may occur on any nucleotide of the sense strand or antisense strand or both in any position of the strand. For instance, the internucleoside linkage modification may occur on every nucleotide on the sense strand and/or antisense strand; each internucleoside linkage modification may occur in an alternating pattern on the sense strand or antisense strand; or the sense strand or antisense strand comprises both internucleoside linkage modifications in an alternating pattern. The alternating pattern of the internucleoside linkage modification on the sense strand may be the same or different from the antisense strand, and the alternating pattern of the internucleoside linkage modification on the sense strand may have a shift relative to the alternating pattern of the internucleoside linkage modification on the antisense strand.

In some embodiments, the dsRNA molecule comprises the phosphorothioate or methylphosphonate internucleoside linkage modification in the overhang region. For example, the overhang region comprises two nucleotides having a phosphorothioate or methylphosphonate internucleoside linkage between the two nucleotides. Internucleoside linkage modifications also may be made to link the overhang nucleotides with the terminal paired nucleotides within duplex region. For example, at least 2, 3, 4, or all the overhang nucleotides may be linked through phosphorothioate or methylphosphonate internucleoside linkage, and optionally, there may be additional phosphorothioate or methylphosphonate internucleoside linkages linking the overhang nucleotide with a paired nucleotide that is next to the overhang nucleotide. For instance, there may be at least two phosphorothioate internucleoside linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleoside linkages separated by 1, 2, 3, 4, 5, 6, 7, 8, 9, 10, 11, 12, 13, 14, 15 or 16 phosphate internucleoside linkages, wherein one of the phosphorothioate or methylphosphonate internucleoside linkages is placed at any position in the oligonucleotide sequence and the said sense strand is paired with an antisense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of two phosphorothioate or methylphosphonate internucleoside linkages separated by 1, 2, 3, 4, 5, 6, 7, 8, 9, 10, 11, 12, 13, 14, 15, 16, 17, or 18 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of three phosphorothioate or methylphosphonate internucleoside linkages separated by 1, 2, 3, 4, 5, 6, 7, 8, 9, 10, 11, 12, 13, 14, 15 or 16 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of four phosphorothioate or methylphosphonate internucleoside linkages separated by 1, 2, 3, 4, 5, 6, 7, 8, 9, 10, 11, 12, 13 or 14 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of five phosphorothioate or methylphosphonate internucleoside linkages separated by 1, 2, 3, 4, 5, 6, 7, 8, 9, 10, 11 or 12 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of six phosphorothioate or methylphosphonate internucleoside linkages separated by 1, 2, 3, 4, 5, 6, 7, 8, 9 or 10 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleoside linkages separated by 1, 2, 3, 4, 5, 6, 7 or 8 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleoside linkages separated by 1, 2, 3, 4, 5 or 6 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleoside linkages separated by 1, 2, 3 or 4 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the dsRNA molecule described herein further comprises one or more phosphorothioate or methylphosphonate internucleoside linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleoside linkage at one end or both ends of the sense and/or antisense strand.

In some embodiments, the dsRNA molecule described herein comprises one or more phosphorothioate or methylphosphonate internucleoside linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleoside linkage at position 8-16 of the duplex region counting from the 5′-end of the sense strand; the dsRNA molecule can optionally further comprise one or more phosphorothioate or methylphosphonate internucleoside linkage modification within 1-10 of the termini position(s).

In some embodiments, the dsRNA molecule described herein further comprises one to five phosphorothioate or methylphosphonate internucleoside linkage modification(s) within position 1-5 and one to five phosphorothioate or methylphosphonate internucleoside linkage modification(s) within the last 3 positions of the sense strand (counting from the 5′-end), and one to five phosphorothioate or methylphosphonate internucleoside linkage modification at positions 1 and 2 and one to five phosphorothioate or methylphosphonate internucleoside linkage modification within the last six positions of the antisense strand (counting from the 5′-end).

In some embodiments, the dsRNA molecule described herein further comprises one phosphorothioate internucleoside linkage modification within position 1-5 and one phosphorothioate or methylphosphonate internucleoside linkage modification within the last six positions of the sense strand (counting from the 5′-end), and one phosphorothioate internucleoside linkage modification at positions 1 and 2 and two phosphorothioate or methylphosphonate internucleoside linkage modifications within the last six the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and two phosphorothioate internucleoside linkage modifications within the last four positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and two phosphorothioate internucleoside linkage modifications within the last four positions of the sense strand (counting from the 5′-end), and one phosphorothioate internucleoside linkage modification at positions 1 and 2 and one phosphorothioate internucleoside linkage modification within the last six positions of the antisense strand (counting from the 5′-end).

In some embodiments, the dsRNA molecule described herein further comprises one phosphorothioate internucleoside linkage modification within position 1-5 and one phosphorothioate internucleoside linkage modification within the last four positions of the sense strand (counting from the 5′-end), and two phosphorothioate internucleoside linkage modifications at positions 1 and 2 and two phosphorothioate internucleoside linkage modifications within the last six positions of the antisense strand (counting from the 5′-end).

In some embodiments, the dsRNA molecule described herein further comprises one phosphorothioate internucleoside linkage modification within position 1-5 and one within the last six positions of the sense strand (counting from the 5′-end), and two phosphorothioate internucleoside linkage modification at positions 1 and 2 and one phosphorothioate internucleoside linkage modification within the last six positions of the antisense strand (counting from the 5′-end).

In some embodiments, the dsRNA molecule described herein further comprises one phosphorothioate internucleoside linkage modification within position 1-5 (counting from the 5′-end) of the sense strand, and two phosphorothioate internucleoside linkage modifications at positions 1 and 2 and one phosphorothioate internucleoside linkage modification within the last six positions of the antisense strand (counting from the 5′-end).

In some embodiments, the dsRNA molecule described herein further comprises two phosphorothioate internucleoside linkage modifications within position 1-5 (counting from the 5′-end) of the sense strand, and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and one within the last six positions of the sense strand (counting from the 5′-end), and two phosphorothioate internucleoside linkage modifications at positions 1 and 2 and one phosphorothioate internucleoside linkage modification within the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and two phosphorothioate internucleoside linkage modifications at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 20 and 21 of the sense strand (counting from the 5′-end), and one phosphorothioate internucleoside linkage modification at positions 1 and one at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 20 and 21 the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 21 and 22 of the sense strand (counting from the 5′-end), and one phosphorothioate internucleoside linkage modification at positions 1 and one phosphorothioate internucleoside linkage modification at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 21 and 22 the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 22 and 23 of the sense strand (counting from the 5′-end), and one phosphorothioate internucleoside linkage modification at positions 1 and one phosphorothioate internucleoside linkage modification at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 22 and 23 the antisense strand (counting from the 5′-end).

In some embodiments, the sense strand comprises at least two phosphorothioate internucleoside linkages between the first five nucleotides counting from the 5′ end of the sense strand. For example, the sense strand comprises phosphorothioate linkages between nucleotides 1 and 2, and between nucleotides 2 and 3, counting from 5′-end of the sense strand.

In some embodiments, the antisense strand comprises at least two phosphorothioate internucleoside linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand.

In some embodiments, the antisense strand comprises at least two phosphorothioate internucleoside linkages between the first five nucleotides counting from the 3′ end of the antisense strand. For example, the antisense strand comprises phosphorothioate linkages between nucleotides n and n−1, and between nucleotides n−1 and n−2, where n is length of the antisense strand, i.e, number of nucleotides in the antisense strand. In other words, the antisense strand comprises phosphorothioate linkages between nucleotides 1 and 2, and between nucleotides 2 and 3, counting from 3′-end of the antisense strand.

In some embodiments, the antisense strand comprises at least two phosphorothioate internucleoside linkages between the first five nucleotides counting from the 5′-end of the antisense strand and at least two phosphorothioate internucleoside linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand and between nucleotides 1 and 2, and between nucleotides 2 and 3, counting from 3′-end of the antisense strand.

In some embodiments, the sense strand comprises at least two phosphorothioate internucleoside linkages between the first five nucleotides counting from the 5′ end of the sense strand and the antisense strand comprises at least two phosphorothioate internucleoside linkages between the first five nucleotides counting from the 5′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 5′-end of the antisense strand.

In some embodiments, the sense strand comprises at least two phosphorothioate internucleoside linkages between the first five nucleotides counting from the 5′ end of the sense strand and the antisense strand comprises at least two phosphorothioate internucleoside linkages between the first five nucleotides counting from the 3′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 3′-end of the antisense strand.

In some embodiments, dsRNA molecule described herein comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 intemucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, dsRNA molecule described herein comprises a block is a stereochemistry block. In some embodiments, a block is an Rp block in that each intemucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, dsRNA molecule described herein comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleoside linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, dsRNA molecule described herein comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

Various publications describe multimeric siRNA which can all be used with the oligonucleotide and dsRNA of the invention. Such publications include WO2007/091269, U.S. Pat. No. 7,858,769, WO2010/141511, WO2007/117686, WO2009/014887 and WO2011/031520 which are hereby incorporated by their entirely.

In some embodiments, the dsRNA molecule described herein comprises one or more overhang regions and/or capping groups of dsRNA molecule at the 3′-end, or 5′-end or both ends of a strand. The overhang can be 1-10 nucleotides in length. For example, the overhang can be 1, 2, 3, 4, 5, 6, 7, 8, 9 or 10 nucleotides in length. In some embodiments, the overhang is 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target sequence or it can be complementary to the gene sequences being targeted or it can be the other sequence. The first and second strands can also be joined, e.g., by additional bases to form a hairpin, or by other non-base linkers.

In some embodiments, the nucleotides in the overhang region of the dsRNA molecule described herein can each independently be a modified or unmodified nucleotide including, but not limited to 2′-sugar modified, such as, 2′-Fluoro 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h'GNA, and any combinations thereof. For example, dTdT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA molecule described herein may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

The dsRNA molecule described herein may comprise only a single overhang, which can strengthen the interference activity of the dsRNA, without affecting its overall stability. For example, the single-stranded overhang is located at the 3-terminal end of the sense strand or, alternatively, at the 3-terminal end of the antisense strand. The dsRNA can also have a blunt end, located at the 5′-end of the antisense strand (or the 3′-end of the sense strand) or vice versa.

Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. For example, the single overhang is at least one, two, three, four, five, six, seven, eight, nine, or ten nucleotides in length. In some embodiments, the dsRNA has a 2 nucleotide overhang on the 3′-end of the antisense strand and a blunt end at the 5′-end of the antisense strand.

The dsRNA described herein can comprise one or more modified nucleotides. For example, every nucleotide in the sense strand and antisense strand of the dsRNA molecule can be modified. Each nucleotide can be modified with the same or different modification which can include one or more alteration of one or both of the non-linking phosphate oxygens and/or of one or more of the linking phosphate oxygens; alteration of a constituent of the ribose sugar; replacement of the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a central region, may only occur at a non-terminal tregion, or may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, the dsRNA molecule described herein comprises modifications of an alternating pattern, particular in the BT, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule described herein comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

In some embodiments of any one of the aspects described herein, the oligonucleotides described herein or at least one e.g., both strand of a dsRNA described herein are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g., RP(OH)(O)—O-5′-, R=alkyl, e.g., methyl, ethyl, isopropyl, propyl, etc.), 5′-alkenylphosphonates (i.e. vinyl, substituted vinyl, e.g., OH)₂(O)P-5′-CH= or (OH)₂(O)P-5′-CH₂—), 5′-alkyletherphosphonates (e.g., R(OH)(O)P—O-5′, R=alkylether, e.g., methoxymethyl (MeOCH₂—), ethoxymethyl, etc.) Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

In some embodiments of any one of the aspects described herein, the oligonucleotide or at least one (e.g., both) strand of a dsRNA described herein comprises a 5′-vinylphosphonate group. For example, the oligonucleotide or at least one (e.g., both) strand of a dsRNA described herein comprises a 5′-E-vinyl or at least one (e.g., both) strand of a dsRNA described herein phosphonate group. In some other non-limiting example, the oligonucleotide comprises a 5′-Z-vinylphosphonate group.

In one example, the 5′-modification can be placed in the antisense strand of a double-stranded nucleic acid, e.g., dsRNA molecule. For example, the antisense comprises a 5′-E-vinylphosphonate. In some other non-limiting example, the antisense strand comprises a 5′-Z-vinylphosphonate group.

In some embodiments, the sense strand comprises a 5′-morpholino, a 5′-dimethylamino, a 5′-deoxy, an inverted abasic, or an inverted abasic locked nucleic acid modification at the 5′-end.

The dsRNA agents of the invention can comprise thermally destabilizing modifications in the seed region of the antisense strand (i.e., at positions 2-9 of the 5′-end of the antisense strand) to reduce or inhibit off-target gene silencing. Without wishing to be bound by a theory, dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, thermally destabilizing modification of the duplex is located in positions 2-9, or preferably positions 4-8, from the 5′-end of the antisense strand. In some further embodiments, the thermally destabilizing modification of the duplex is located at position 5, 6, 7 or 8 from the 5′-end of the antisense strand.

In still some further embodiments, the thermally destabilizing modification of the duplex is located at position 7 from the 5′-end of the antisense strand.

The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s). In some embodiments, the thermally destabilizing modification of the duplex is located at position 2, 3, 4, 5, 6, 7, 8 or 9 from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA). For example, the thermally destabilizing modifications can include, but are not limited to, mUNA and GNA building blocks as follows:

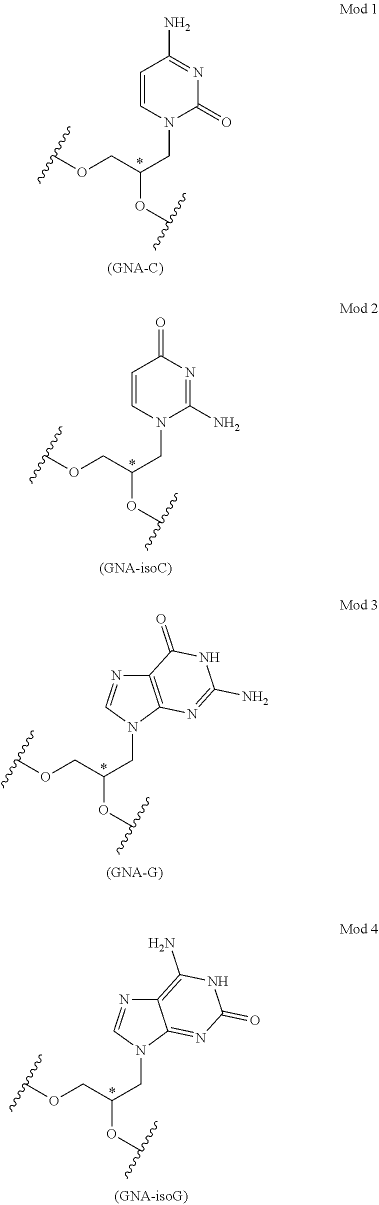 [SMILES: *OCC(Cn1ccc(N)nc1=O)O* ; *OCC(Cn1ccc(=O)nc1N)O* ; *OCC(Cn1cnc2c(=O)[nH]c(N)nc21)O* ; *OCC(Cn1cnc2c(N)[nH]c(=O)nc21)O*]

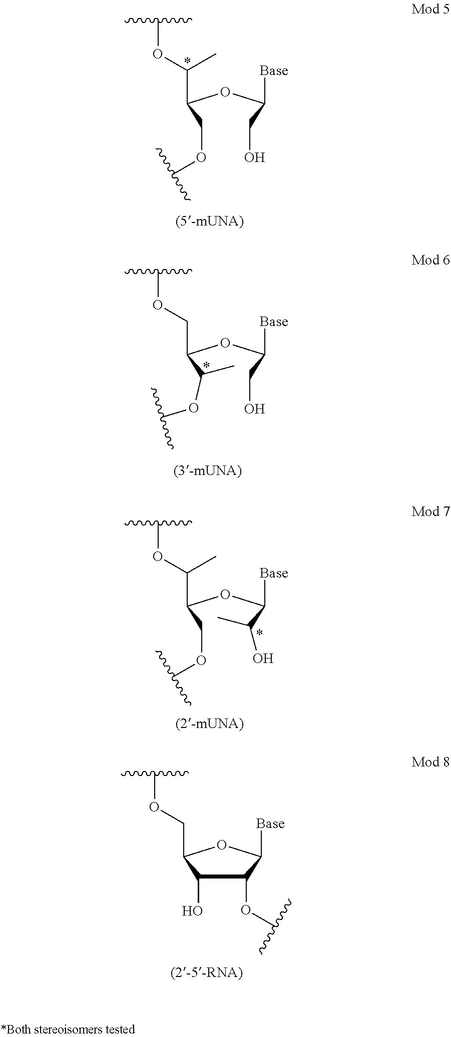

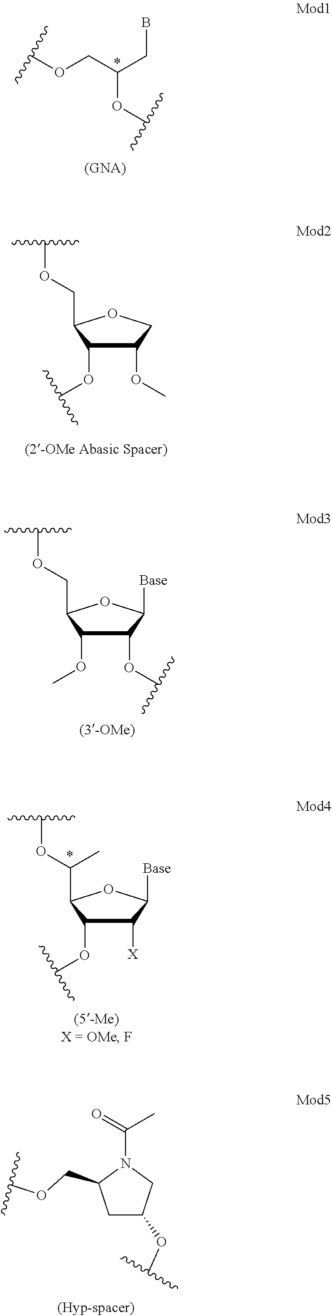

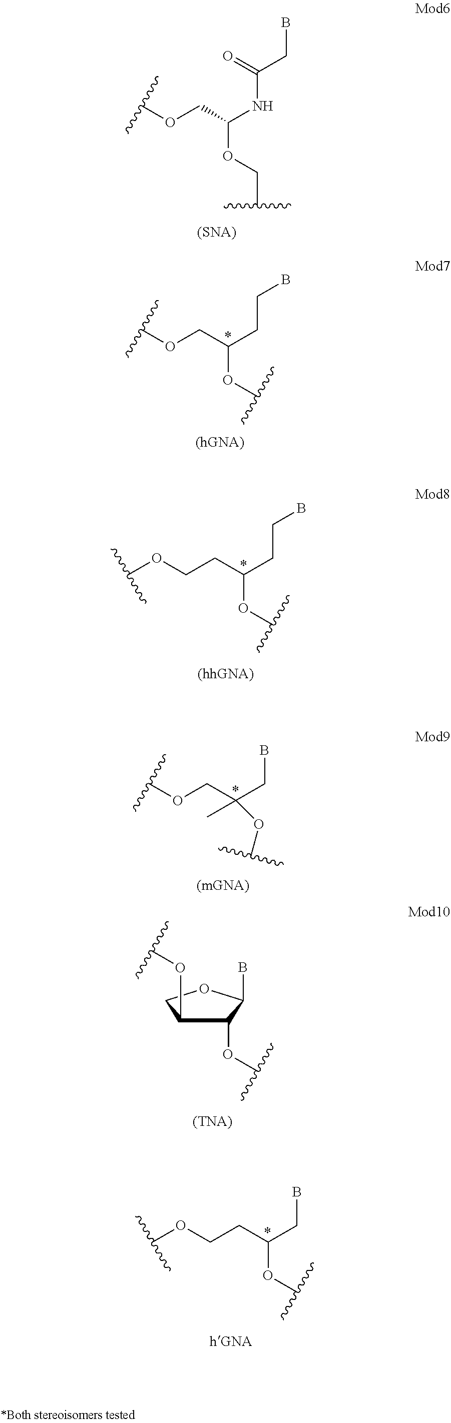

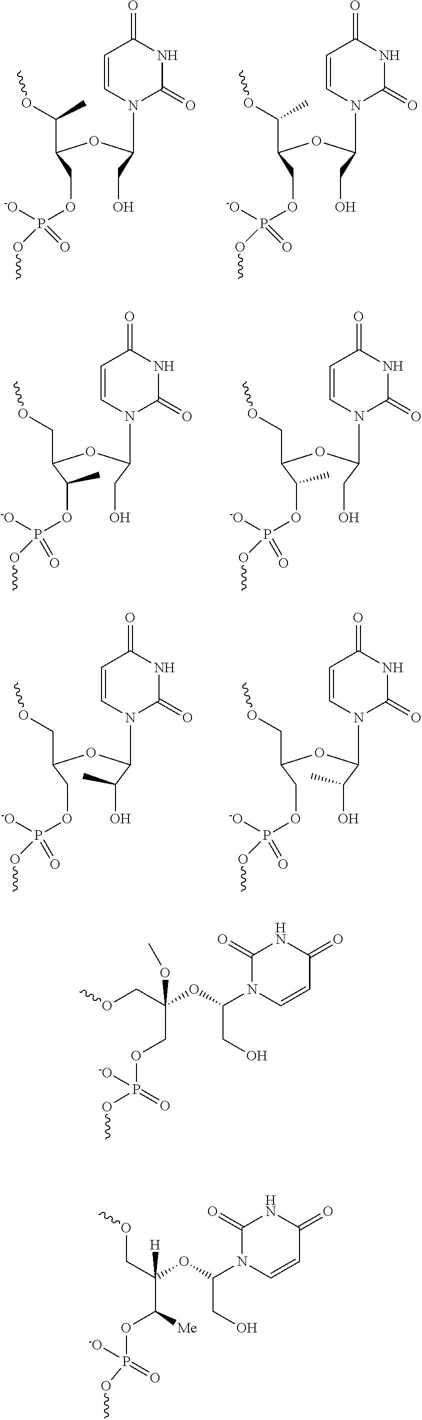

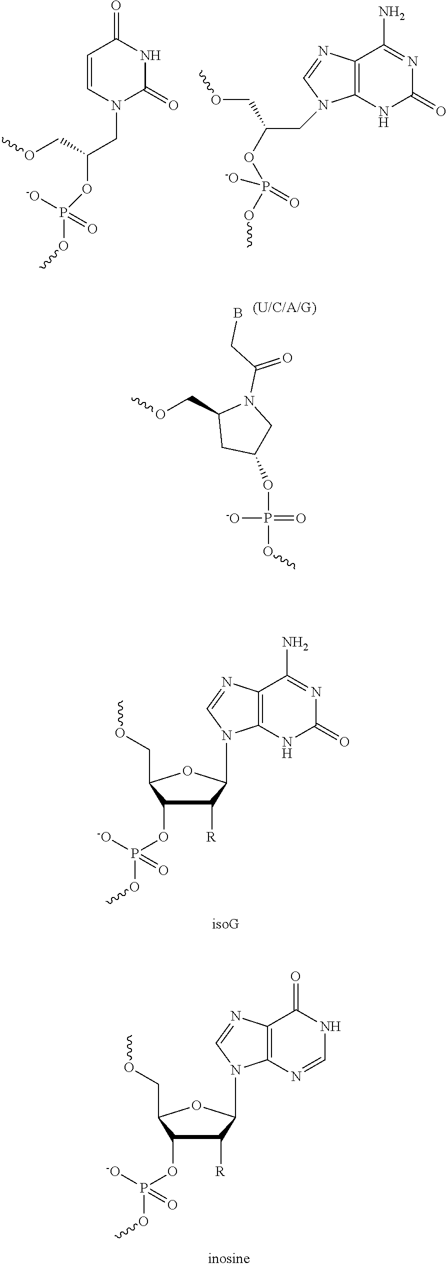

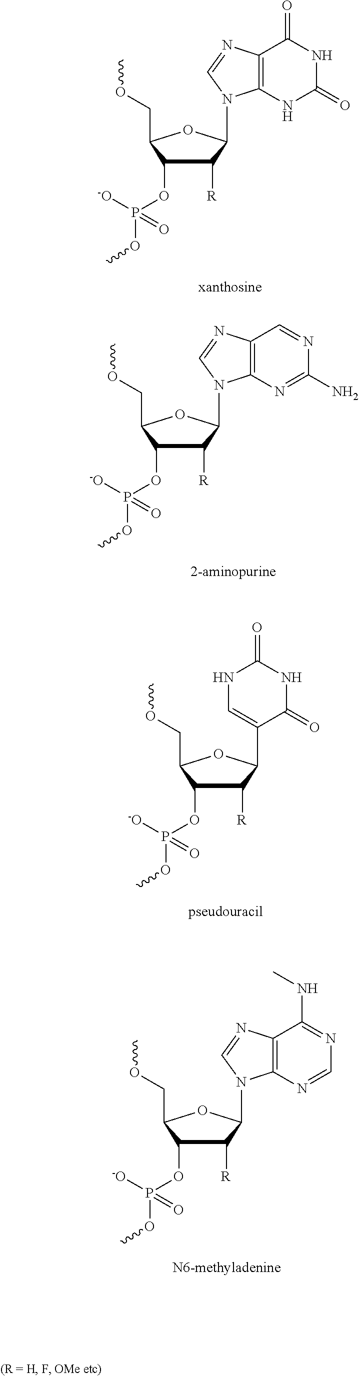

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

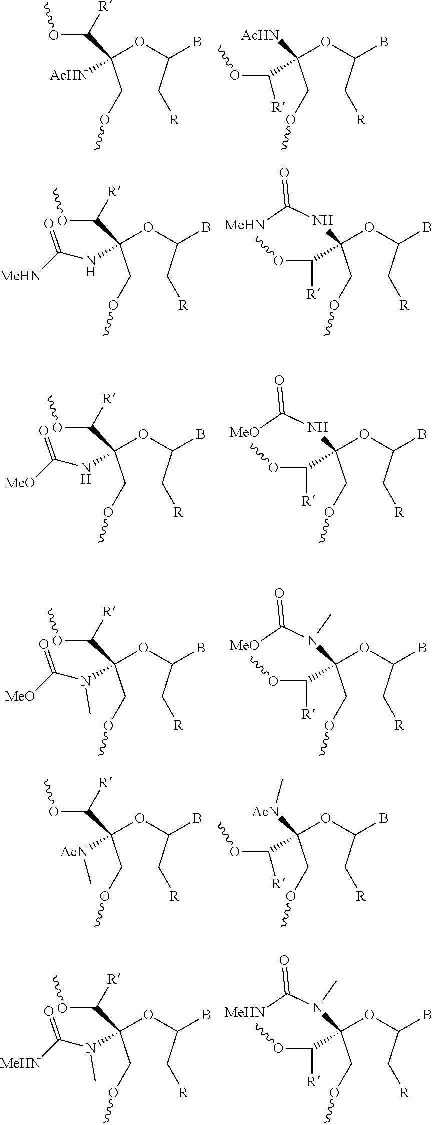

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; 06-alkyl-G; 2-thiouridine; 4-thiouridine; C5-modified pyrimidines; C2-modified purines; N8-modiifed purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

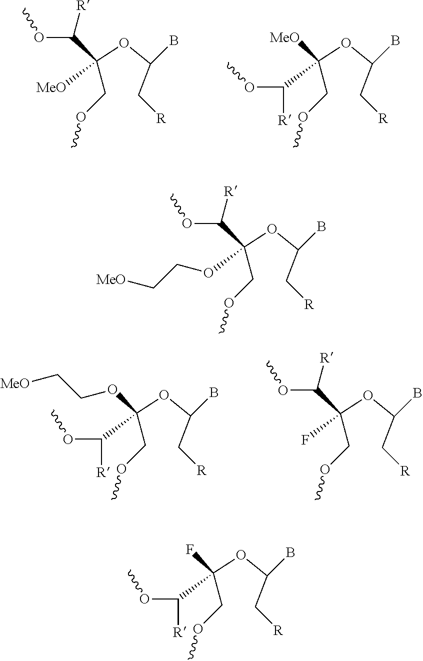

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

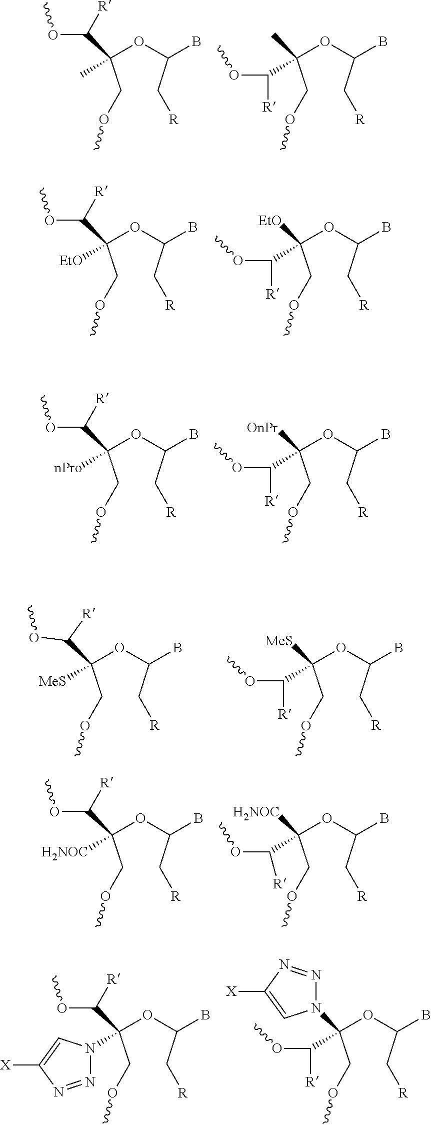

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurinOMexanthosine; N6-alkyl-A; O6-alkyl-G; 7-deazapurines; and
Stereochemistry is R or S and combination of R and S for the unspecified chiral centers.

In some embodiments, the destabilizing modification mUNA is selected from the group consisting of

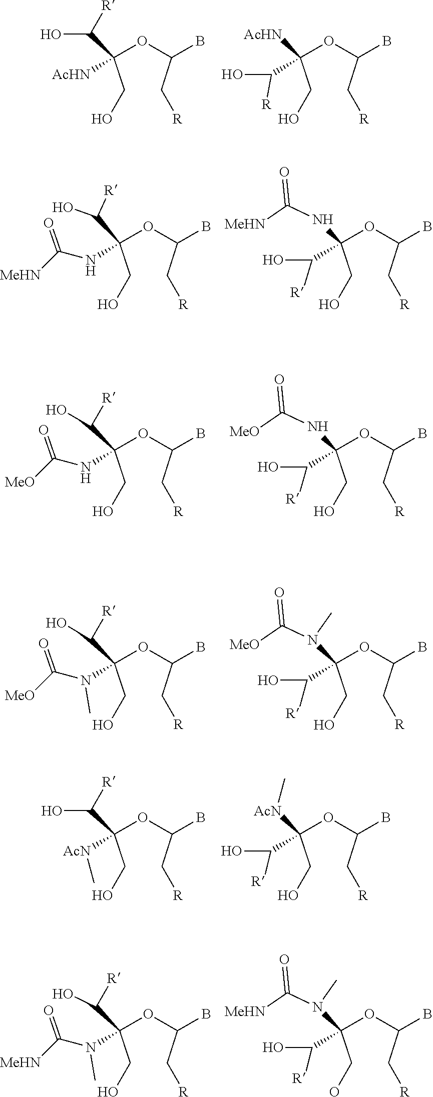

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the destabilizing modification mUNA is selected from the group consisting of

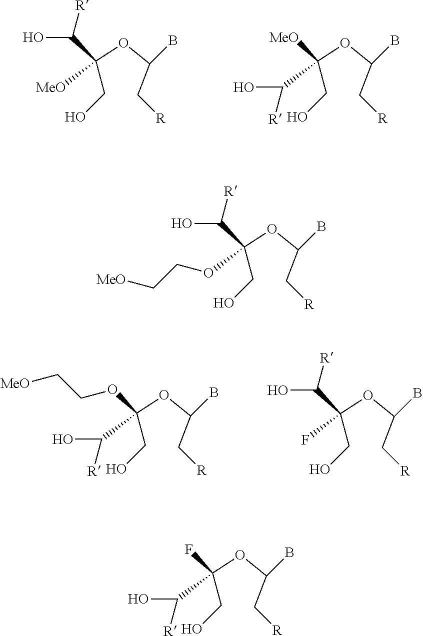

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

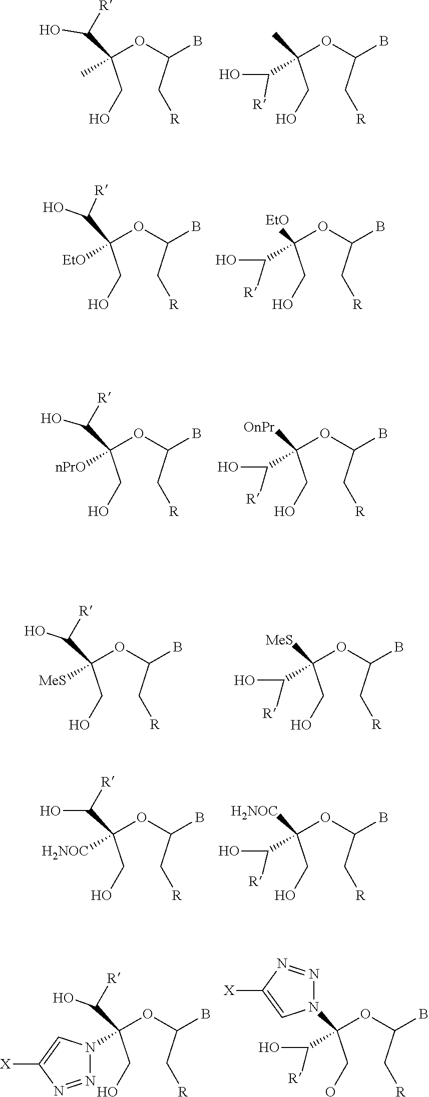

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 7-deazapurines; and
Stereochemistry is R or S and combination of R and S for the unspecified chiral centers

Exemplary abasic modifications include, but are not limited to the following:

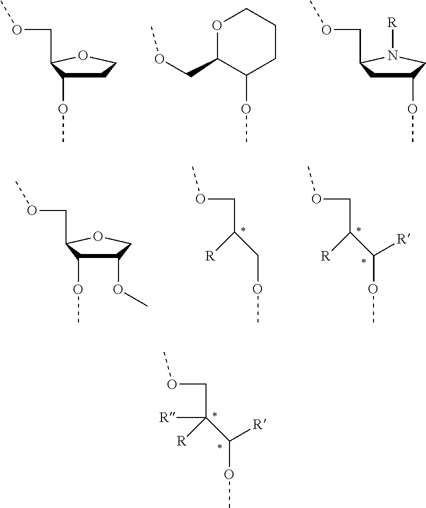

Wherein R=H, Me, Et or OMe; R′=H, Me, Et or OMe; R″=H, Me, Et or OMe

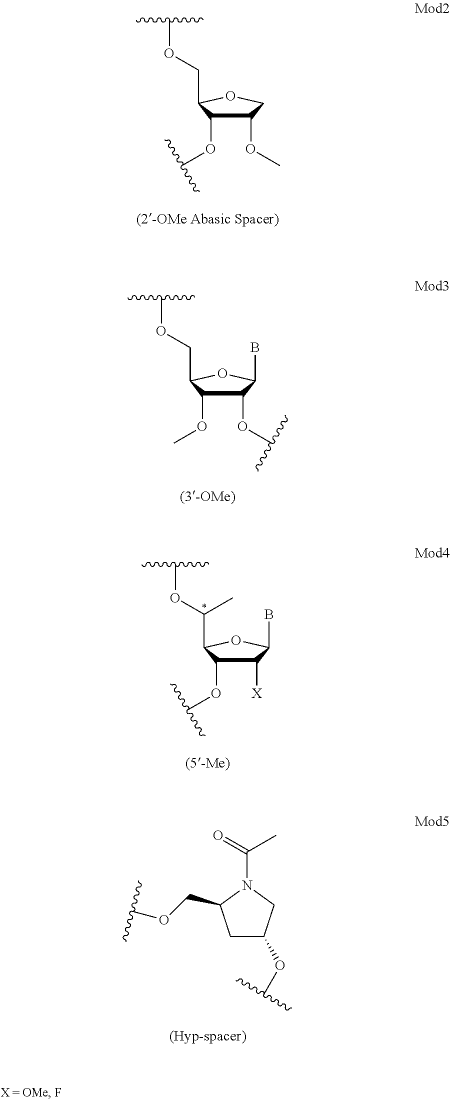

wherein B is a modified or unmodified nucleobase and the asterisk on each structure represents either R, S or racemic.

Exemplified sugar modifications include, but are not limited to the following:

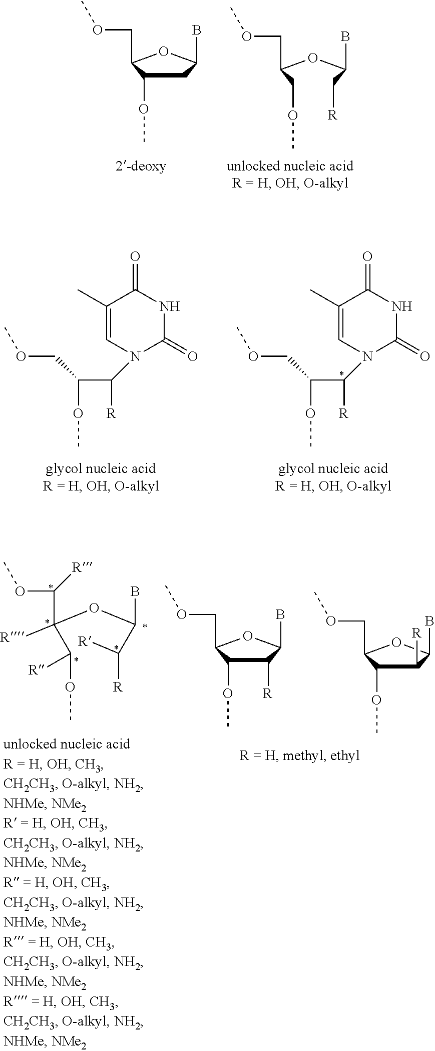

wherein B is a modified or unmodified nucleobase and the asterisk on each structure represents either R, S or racemic.

In some embodiments the thermally destabilizing modification of the duplex is selected from the mUNA and GNA building blocks described in Examples 1-3 herein. In some embodiments, the destabilizing modification is selected from the group consisting of GNA-isoC, GNA-isoG, 5′-mUNA, 4′-mUNA, 3′-mUNA, and 2′-mUNA. In some further embodiments of this, the dsRNA molecule further comprises at least one thermally destabilizing modification selected from the group consisting of GNA, 2′-OMe, 3′-OMe, 5′-Me, Hy p-spacer, SNA, hGNA, hhGNA, mGNA, TNA and h'GNA (Mod A-Mod K).

The term “acyclic nucleotide” refers to any nucleotide having an acyclic ribose sugar, for example, where any of bonds between the ribose carbons (e.g., C1′—C2′, C2′—C3′, C3′—C4′, C4′—O4′, or C1′—O4′) is absent and/or at least one of ribose carbons or oxygen (e.g., C1′, C2′, C3′, C4′ or O4′) are independently or in combination absent from the nucleotide. In some embodiments, acyclic nucleotide is

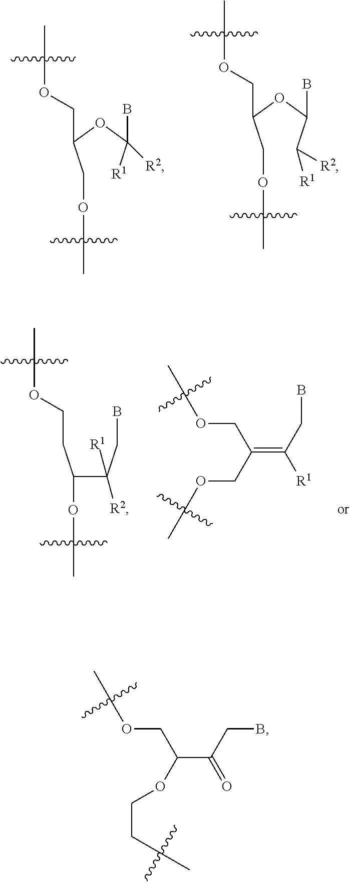

wherein B is a modified or unmodified nucleobase, R1 and R2 independently are H, halogen, OR3, or alkyl; and R3 is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′—C4′ being removed (i.e. the covalent carbon-oxygen-carbon bond between the C1′ and C4′ carbons). In another example, the C2′—C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

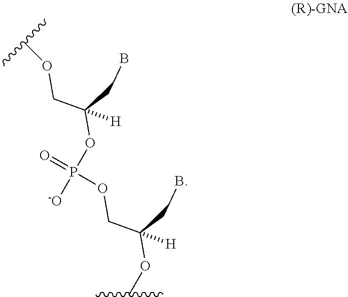

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA, such as:

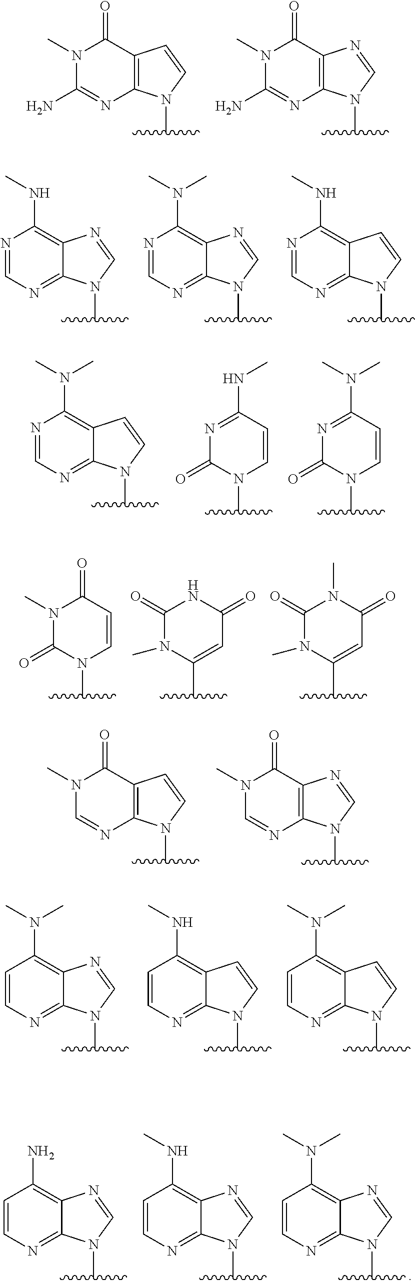

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

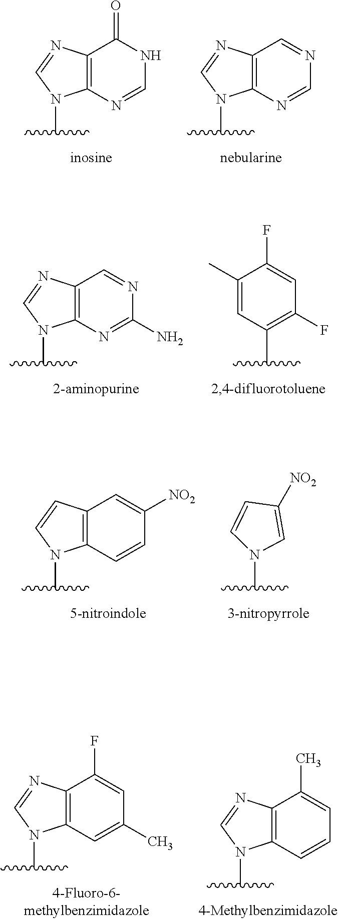

In some embodiments, the thermally destabilizing modification of the duplex in the seed region of the antisense strand includes one or more a-nucleotide complementary to the base on the target mRNA, such as:

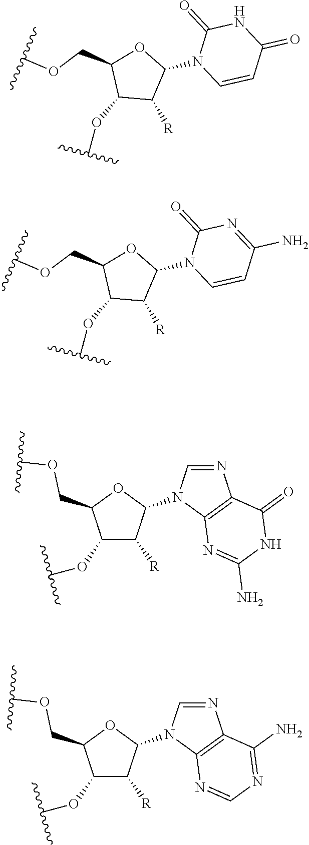

wherein R is H, OH, OCH₃, F, NH₂, NHMe, NMe₂ or O-alkyl

Exemplary phosphate modifications known to decrease the thermal stability of dsRNA duplexes compared to natural phosphodiester linkages are:

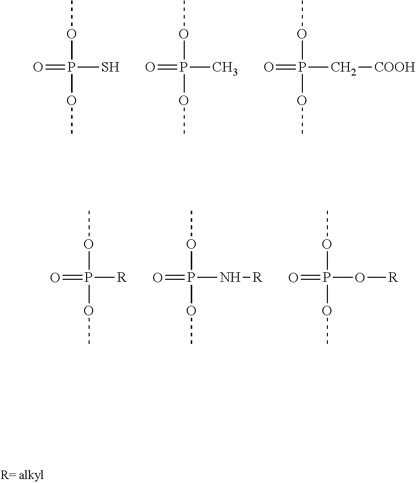

The alkyl for the R group can be a C₁-C₆alkyl. Specific alkyls for the R group include, but are not limited to methyl, ethyl, propyl, isopropyl, butyl, pentyl and hexyl.

It is noted a thermally destabilizing modification can replace a 2′-doexy nucleotide in the antisense strand. For example, a 2′-deoxy nucleotide at positions 2, 5, 7, 12, 14 and/or 16, counting from 5′-end, of the antisense strand can be replaced with a thermally destabilizing modification described herein. Thus, in some embodiments, the antisense strand comprises a thermally destabilizing modification at 1, 2, 3, 4, 5 and/or 6 of positions 2, 5, 7, 12, 14 and/or 16, counting from 5′-end of the antisense strand. For example, the antisense strand comprises a thermally destabilizing modification at positions 5 and 7, counting from 5′-end of the antisense strand.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand and/or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14 and 16 from the 5′-end. In some other embodiments, the antisense comprises stabilizing modifications at positions 2, 6, 14 and 16 from the 5′-end. In still some other embodiments, the antisense comprises stabilizing modifications at positions 2, 14 and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification. In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10 and 11 from the 5′-end. In some other embodiments, the sense strand comprises stabilizing modifications at positions 7, 9, 10 and 11 from the 5′-end. In some embodiments, the sense strand comprises stabilizing modifications at positions opposite or complimentary to positions 11, 12 and 15 of the antisense strand, counting from the 5′-end of the antisense strand. In some other embodiments, the sense strand comprises stabilizing modifications at positions opposite or complimentary to positions 11, 12, 13 and 15 of the antisense strand, counting from the 5′-end of the antisense strand. In some embodiments, the sense strand comprises a block of two, three or four stabilizing modifications.

In some embodiments, the sense strand does not comprise a stabilizing modification in position opposite or complimentary to the thermally destabilizing modification of the duplex in the antisense strand.

Exemplary thermally stabilizing modifications include, but are not limited to 2′-fluoro modifications. Other thermally stabilizing modifications include, but are not limited to LNA.

It is noted a thermally stabilizing modification can replace a 2′-fluoro nucleotide in the sense and/or antisense strand. For example, a 2′-fluoro nucleotide at positions 8, 9, 10, 11 and/or 12, counting from 5′-end, of the sense strand, can be replaced with a thermally stabilizing modification. Similarly, a 2′-fluoro nucleotide at position 14, counting from 5′-end, of the antisense strand, can be replaced with a thermally stabilizing modification.

For the dsRNA molecules to be more effective in vivo, the antisense strand must have some metabolic stability. In other words, for the dsRNA molecules to be more effective in vivo, some amount of the antisense stand may need to be present in vivo after a period time after administration. Accordingly, in some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 5 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 6 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 7 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 8 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 9 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 10 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 11 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 12 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 13 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 14 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 15 after in vivo administration.

### Ligands

Embodiments of the various aspects described herein include a ligand. Without wishing to be bound by a theory, ligands modify one or more properties of the attached molecule (e.g., the oligonucleotide described herein) including but not limited to pharmacodynamic, pharmacokinetic, binding, absorption, cellular distribution, cellular uptake, charge and clearance. Ligands are routinely used in the chemical arts and are linked directly or via an optional linking moiety or linking group to a parent compound. A preferred list of ligands includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred ligands amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxylpropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (SEQ ID NO:1) (GALA); AALAEALAEALAEALAEALAEALAAAAGGC (SEQ ID NO:2) (EALA); ALEALAEALEALAEA (SEQ ID NO:3); GLFEAIEGFIENGWEGMIWDYG (SEQ ID NO:4) (INF-7); GLFGAIAGFIENGWEGMIDGWYG (SEQ ID NO:5) (Inf HA-2); GLFEAIEGFIENGWEGMIDGWYGCGLFEAIEGFIENGWEGMID GWYGC (SEQ ID NO:6) (diINF-7); GLFEAIEGFIENGWEGMIDGGCGLFEAIEGFIENGWEGMIDGGC (SEQ ID NO:7) (diINF-3); GLFGALAEALAEALAEHLAEALAEALEALAAGGSC (SEQ ID NO:8) (GLF); GLFEAIEGFIENGWEGLAEALAEALEALAAGGSC (SEQ ID NO:9) (GALA-INF3); GLF EAI EGFI ENGW EGnI DG K GLF EAI EGFI ENGW EGnI DG (SEQ ID NO:10) (INF-5, n is norleucine); LFEALLELLESLWELLLEA (SEQ ID NO:11) (JTS-1); GLFKALLKLLKSLWKLLLKA (SEQ ID NO:12) (ppTG1); GLFRALLRLLRSLWRLLLRA (SEQ ID NO:13) (ppTG20); WEAKLAKALAKALAKHLAKALAKALKACEA (SEQ ID NO:14) (KALA); GLFFEAIAEFIEGGWEGLIEGC (SEQ ID NO:15) (HA); GIGAVLKVLTTGLPALISWIKRKRQQ (SEQ ID NO:16) (Melittin); HSWYG (SEQ ID NO:17); and CHK₆HC (SEQ ID NO:18).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (SEQ ID NO:19) (penetratin); GRKKRRQRRRPPQC (SEQ ID NO:20) (Tat fragment 48-60); GALFLGWLGAAGSTMGAWSQPKKKRKV (SEQ ID NO:21) (signal sequence based peptide); LLIILRRRIRKQAHAHSK(SEQ ID NO:22) (PVEC); GWTLNSAGYLLKINLKALAALAKKIL (SEQ ID NO:23) (transportan); KLALKLALKALKAALKLA (SEQ ID NO:24) (amphiphilic model peptide); RRRRRRRRR (SEQ ID NO:25) (Arg9); KFFKFFKFFK (SEQ ID NO:26) (Bacterial cell wall permeating peptide); LLGDFFRKSKEKIGKEFKRIVQRIKDFLRNLVPRTES (SEQ ID NO:27) (LL-37); SWLSKTAKKLENSAKKRISEGIAIAIQGGPR (SEQ ID NO:28) (cecropin P1); ACYCRIPACIAGERRYGTCIYQGRLWAFCC (SEQ ID NO:29) (α-defensin); DHYNCVSSGGQCLYSACPIFTKIQGTCYRGKAKCCK(SEQ ID NO:30) (β-defensin); RRRPRPPYLPRPRPPPFFPPRLPPRIPPGFPPRFPPRFPGKR-NH2 (SEQ ID NO:31) (PR-39); ILPWKWPWWPWRR-NH2 (SEQ ID NO:32) (indolicidin); AAVALLPAVLLALLAP (SEQ ID NO:33) (RFGF); AALLPVLLAAP (SEQ ID NO:34) (RFGF analogue); and RKCRIVVIRVCR (SEQ ID NO:35) (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc2 and GalNAc3; D-mannose, multivalent mannose, multivalent lactose, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of oligonucleotides described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleoside linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments of any one of the aspects, the ligand has a structure shown in any of Formula (IV)—(VII):

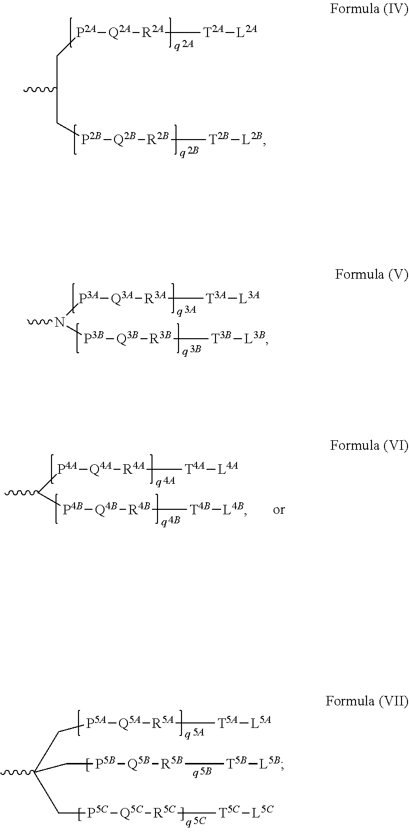

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different; P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)), C(O)NH, NHCH(R^{a})C(O), —C(O)—CH(R^{a})—NH—, CO, CH═N—O,

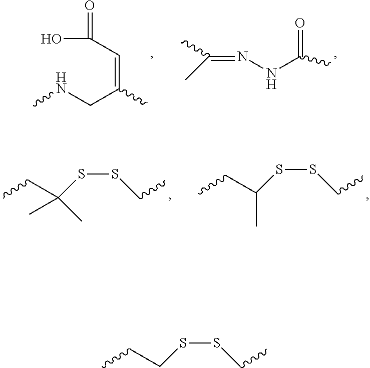

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

In some embodiments of any one of the aspects, the ligand is of Formula (VII):

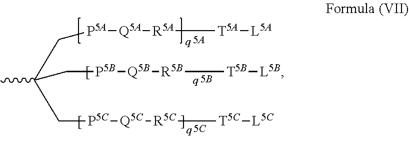

wherein L^{5A}, L^{5B} and L^{5C} represent a monosaccharide, such as GalNAc derivative.

Exemplary ligands include, but are not limited to, the following:

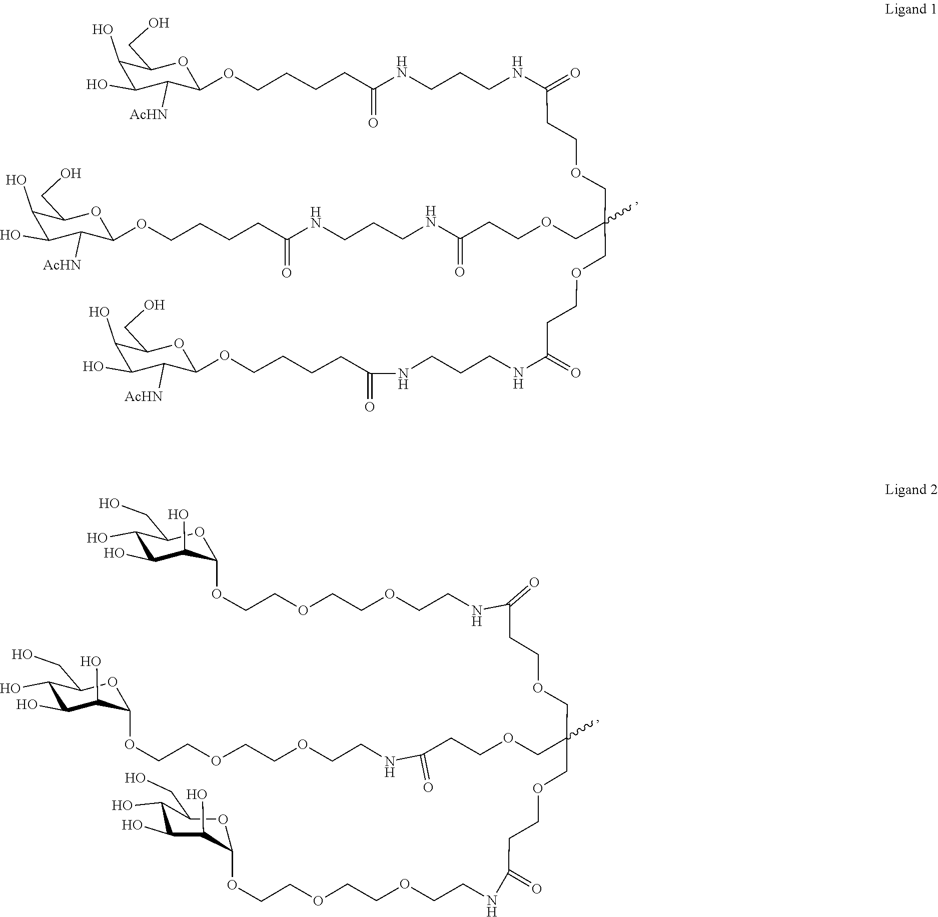

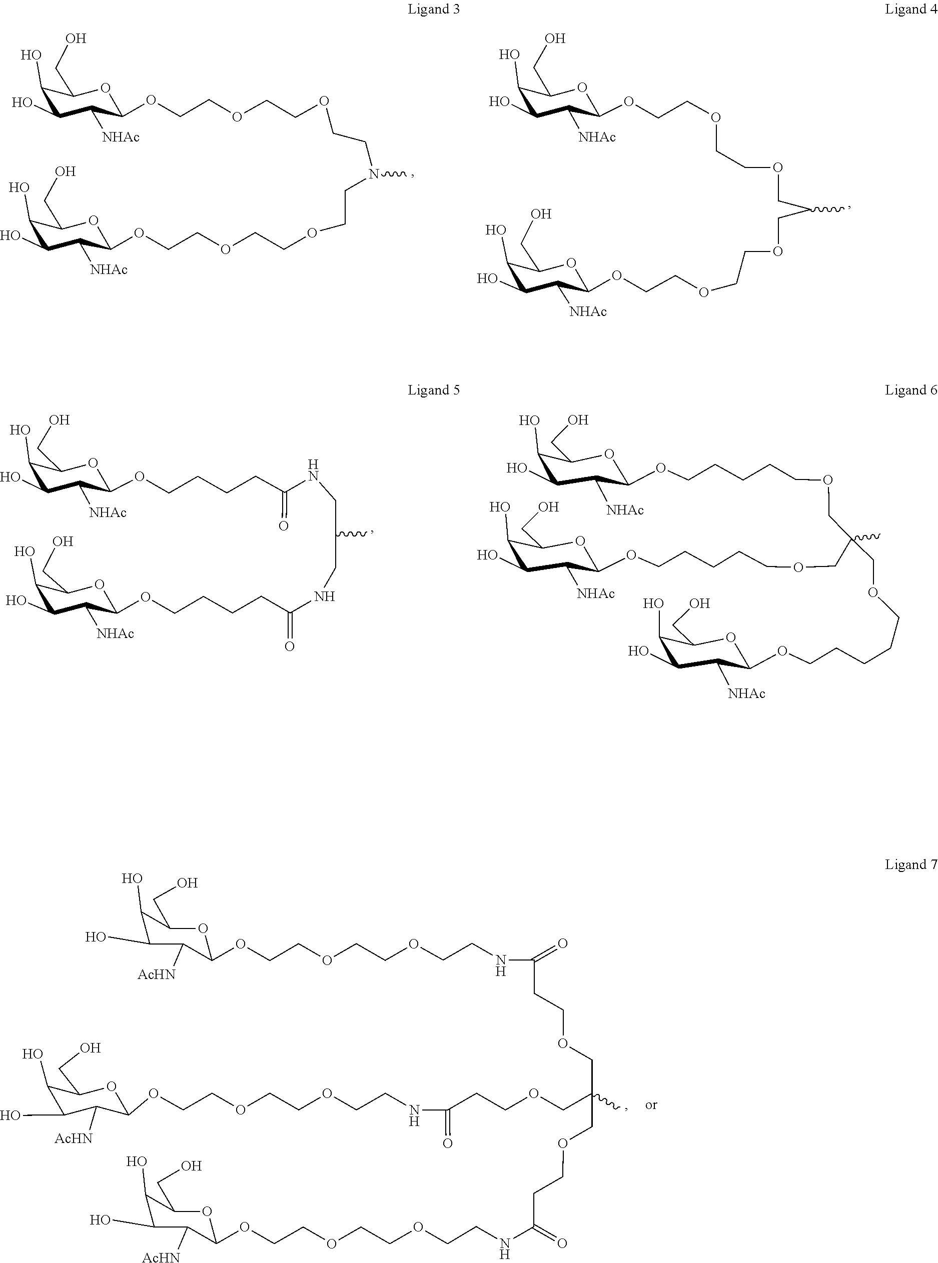

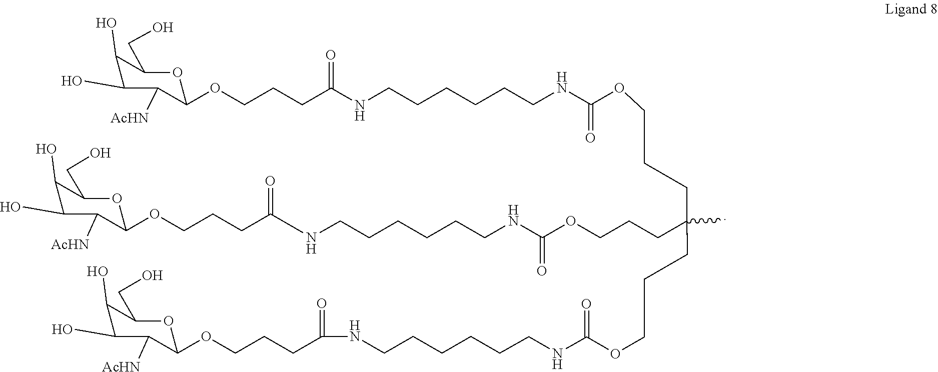 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In some embodiments of any one of the aspects described herein, the ligand is a ligand described in U.S. Pat. No. 5,994,517 or U.S. Pat. No. 6,906,182, content of each of which is incorporated herein by reference in its entirety.

In some embodiments, the ligand can be a tri-antennary ligand described in FIG. 3 of U.S. Pat. No. 6,906,182. For example, the ligand is selected from the following tri-antennary ligands:

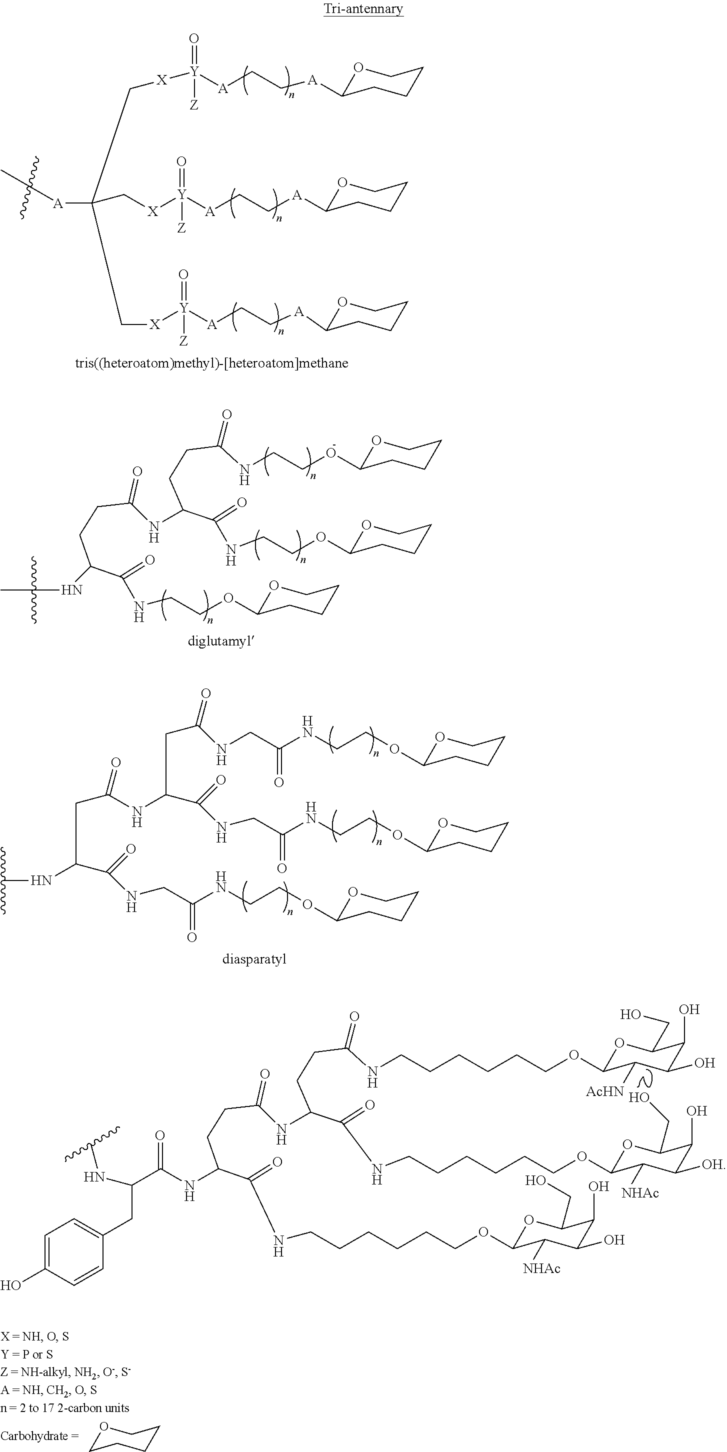

In some embodiments, the ligand can be a ligand described, e.g., in FIGS. 4A and 4B of US2021/0123048, contents of which are incorporated herein by reference in their entireties.

In some embodiments of any one of the aspects described herein, the ligand can be

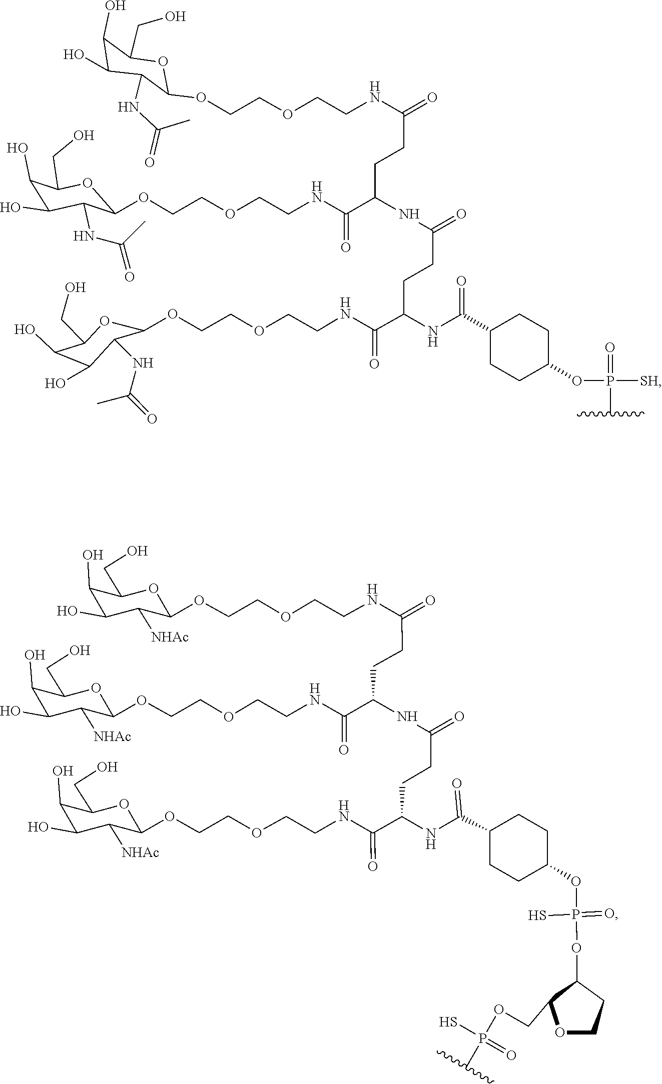

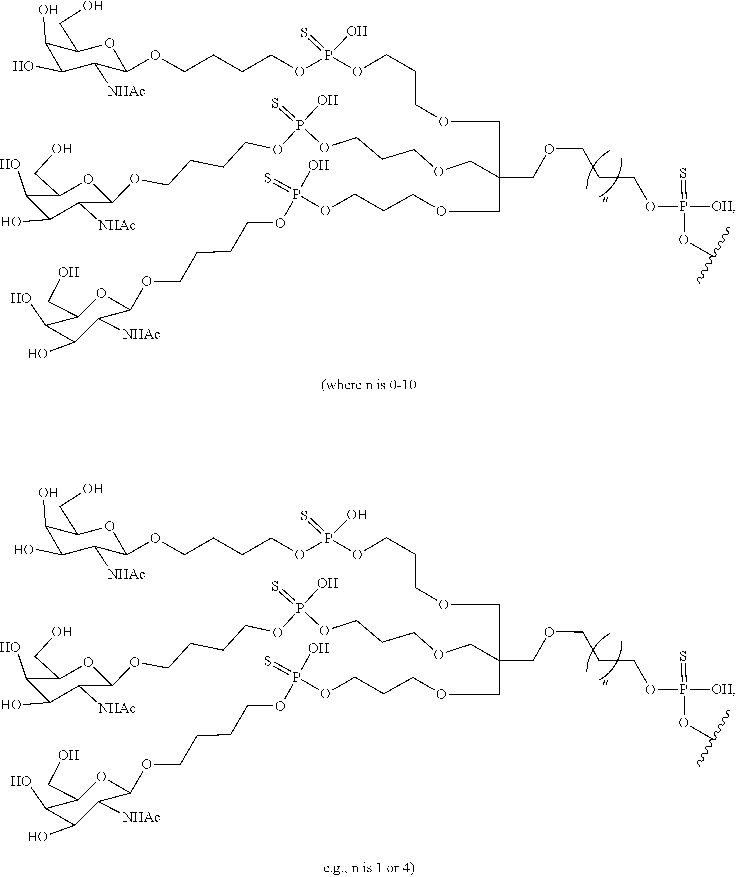

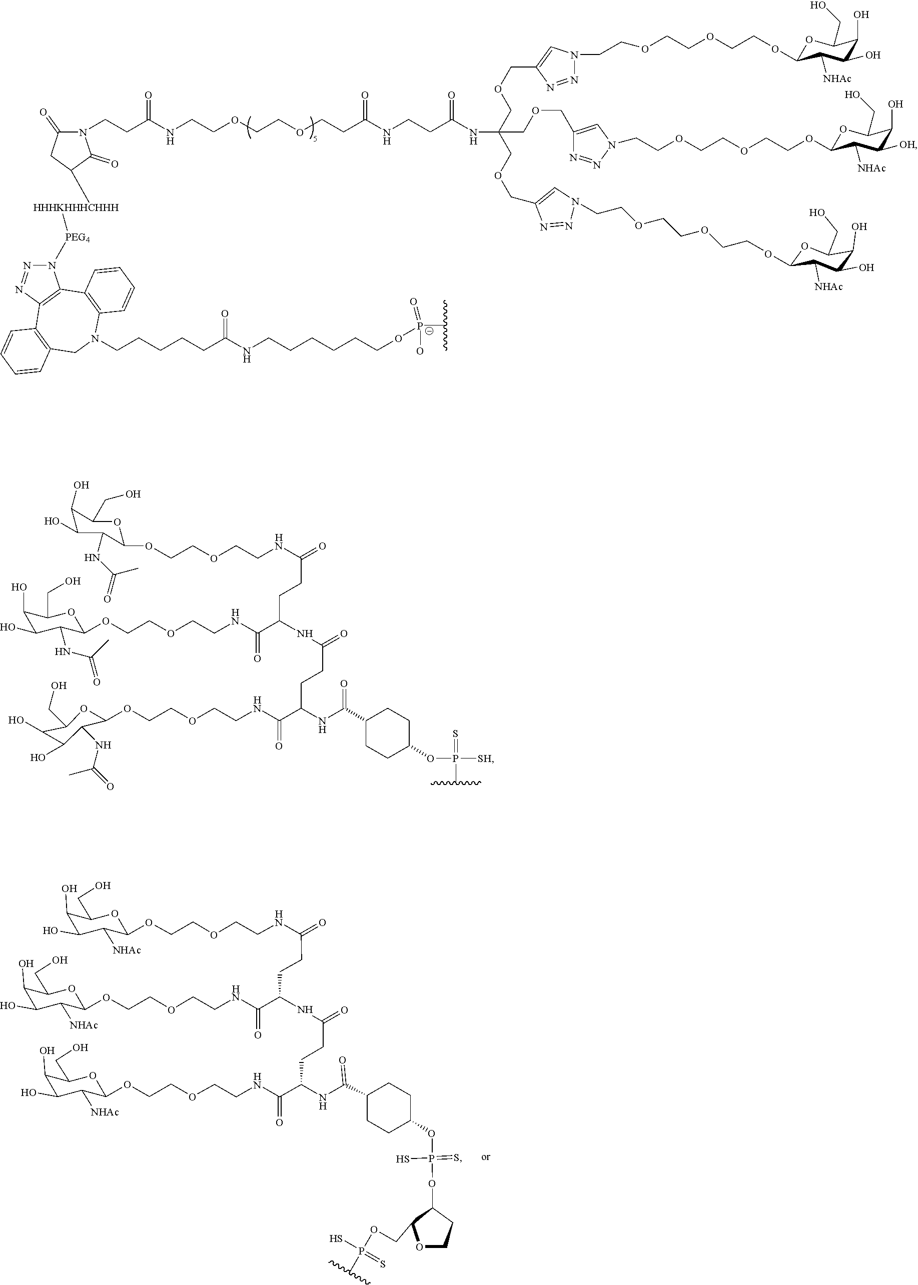

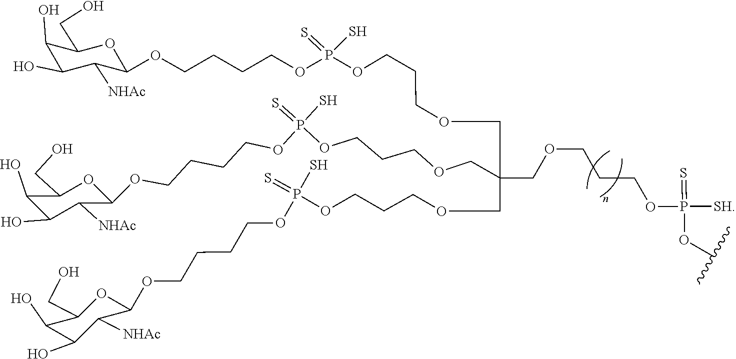

It is noted that when more than one ligands are present, they can be same or different. Accordingly, in some embodiments of any one of the aspects described herein, all ligands are same. In some other embodiments of any one of the aspects described herein, ligands are different.

Some exemplary ligands include, but are not limited to, peptides, centyrins, antibodies, antibody fragments, T-cell targeting ligands, B-cell targeting ligands, cancer cell targeting ligands (DUPA, folate, RGD), spleen targeting functionalities, lung targeting functionalitie, bone marrow targeting functionalities, antiCD-4 antobodies, antiCD-117 antibodies, phage Display peptides, cell permeation peptides (CPPs), itegrin ligands, multianionic ligands, multicationic ligands, carbohydrates (GalNAc, mannose, mannose-6 phosphate, fucose, glucose, monovalent and multivalent), kidney targeting ligands, blood-brain barrier (BBB) penetration ligands, lipids and amino acids (L-amino acids, D-amino acids, β-amino acids).

In some embodiments, the ligand comprises a lipophilic group. For example, the ligand can be a C₆₋₃₀aliphatic group or a C₁₀₋₃₀aliphatic group. In some embodiments, the ligand is a C₁₀₋₃₀alkyl, C₁₀₋₃₀alkenyl or C₁₀₋₃₀alkynyl group. For example, the ligand is a straight-chain or branched hexyl, octyl, decyl, dodecyl, tetradecyl, hexadecyl, octadecyl, icosyl, docosyl, or tetracosyl group. In some embodiments, the ligand is a straight-chain hexyl, octyl, decyl, dodecyl, tetradecyl, hexadecyl, octadecyl, icosyl, docosyl, or tetracosyl group. For example, the ligand is a straight-chain hexyl, octyl, decyl, dodecyl, hexadecyl, octadecyl, icosyl, or docosyl group. For example, the ligand is a straight-chain hexadecyl group. In another example, the ligand is a straight-chain docosyl group.

Uses of Oligonucleotides and dsRNAs

In some embodiments of any one of the aspects, the oligonucleotide described herein or the antisense strand of the dsRNA molecule described herein comprises a nucleotide sequence substantially complementary to a target nucleic acid, e.g., a target gene or mRNA.

Accordingly, in another aspect, the disclosure is directed to a use of an oligonucleotide and/or dsRNA molecule described herein for inhibiting expression of a target gene. In some embodiments, the present invention further relates to a use of an oligonucleotide and/or dsRNA molecule described herein for inhibiting expression of a target gene in vitro.

In another aspect, the disclosure is directed to a use of an oligonucleotide and/or dsRNA molecule described herein for use in inhibiting expression of a target gene in a subject. The subject may be any animal, such as a mammal, e.g., a mouse, a rat, a sheep, a cattle, a dog, a cat, or a human

In some embodiments, the oligonucleotide and/or dsRNA molecule described herein is administered in buffer.

In some embodiments, oligonucleotide and/or dsRNA molecule described herein described herein can be formulated for administration to a subject. A formulated oligonucleotide and/or dsRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide and/or dsRNA preparation can be formulated in combination with another agent, e.g., another therapeutic agent or an agent that stabilizes an oligonucleotide and/or dsRNA, e.g., a protein that complexes with oligonucleotide and/or dsRNA. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

In some embodiments, the oligonucleotide and/or dsRNA preparation includes another dsRNA compound, e.g., a second dsRNA that can mediate RNAi with respect to a second gene, or with respect to the same gene. Still other preparation can include at least 3, 5, ten, twenty, fifty, or a hundred or more different siRNA species. Such dsRNAs can mediate RNAi with respect to a similar number of different genes.

In some embodiments, the oligonucleotide and/or dsRNA preparation includes at least a second therapeutic agent (e.g., an agent other than a RNA or a DNA). For example, a oligonucleotide and/or dsRNA composition for the treatment of a viral disease, e.g., HIV, might include a known antiviral agent (e.g., a protease inhibitor or reverse transcriptase inhibitor). In another example, a dsRNA composition for the treatment of a cancer might further comprise a chemotherapeutic agent.

Exemplary formulations which can be used for administering the oligonucleotide and/or dsRNA according to the present invention are discussed below.

Liposomes. A oligonucleotide and/or dsRNA preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide and/or dsRNA composition. The lipophilic material isolates the aqueous interior from an aqueous exterior, which typically does not include the oligonucleotide and/or dsRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide and/or dsRNA are delivered into the cell where the dsRNA can specifically bind to a target RNA and can mediate RNAi. In some embodiments, the liposomes are also specifically targeted, e.g., to direct the oligonucleotide and/or dsRNA to particular cell types.

A liposome containing oligonucleotide and/or dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of oligonucleotide and/or dsRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging oligonucleotide and/or dsRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with dsRNA descreibed herein are useful for treating a dermatological disorder.

Liposomes that include oligonucleotide and/or dsRNA described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include oligonucleotide and/or dsRNA described herein can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. The oligonucleotide and/or dsRNA compositions can include a surfactant. In some embodiments, the dsRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the oligonucleotide and/or dsRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method, a first micellar composition is prepared which contains the oligonucleotide and/or dsRNA composition and at least the alkali metal alkyl sulphate. The first micellar composition is then mixed with at least three micelle forming compounds to form a mixed micellar composition. In another method, the micellar composition is prepared by mixing the dsRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, dsRNA preparations can be incorporated into a particle, e.g., a microparticle. Microparticles can be produced by spray-drying, but may also be produced by other methods including lyophilization, evaporation, fluid bed drying, vacuum drying, or a combination of these techniques.

### Pharmaceutical Compositions

The oligonucleotide and/or dsRNA described herein can be formulated for pharmaceutical use. The present invention further relates to a pharmaceutical composition comprising the oligonucleotide and/or dsRNA described herein. Pharmaceutically acceptable compositions comprise a therapeutically-effective amount of one or more of the dsRNA molecules in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a dsRNA molecule described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association an oligonucleotide and/or dsRNA with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The oligonucleotide and/or dsRNA described herein may be formulated for administration in any convenient way for use in human or veterinary medicine, by analogy with other pharmaceuticals.

The term “treatment” is intended to encompass therapy and cure. The patient receiving this treatment is any animal in need, including primates, in particular humans, and other mammals such as equines, cattle, swine and sheep; and poultry and pets in general.

The oligonucleotide and/or dsRNA described herein or a pharmaceutical composition comprising an oligonucleotide and/or dsRNA described herein can be administered to a subject using different routes of delivery. A composition that includes an oligonucleotide and/or dsRNA described herein described herein can be delivered to a subject by a variety of routes. Exemplary routes include: intravenous, subcutaneous, topical, rectal, anal, vaginal, nasal, pulmonary, ocular.

The oligonucleotide and/or dsRNA described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the oligonucleotide and/or dsRNA described herein in aerosol form. The vascular endothelial cells could be targeted by coating a balloon catheter with the oligonucleotide and/or dsRNA described herein and mechanically introducing the oligonucleotide and/or dsRNA described herein.

In one aspect, provided herein is a method of administering an oligonucleotide and/or dsRNA described herein, to a subject (e.g., a human subject). In another aspect, the present invention relates to an oligonucleotide and/or dsRNA described herein for use in inhibiting expression of a target gene in a subject. The method or the medical use includes administering a unit dose of the oligonucleotide and/or dsRNA described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of oligonucleotide and/or dsRNA described herein per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities.

In some embodiments, a subject is administered an initial dose and one or more maintenance doses. The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The oligonucleotide and/or dsRNA described herein can be administered to mammals, particularly large mammals such as nonhuman primates or humans in a number of ways.

In some embodiments, the administration of the oligonucleotide and/or dsRNA composition described herein is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of oligonucleotide and/or dsRNA composition described herein.

### Methods of Inhibiting Expression of a Target Gene

Aspects of the disclosure also relate to methods for inhibiting the expression of a target gene. The method comprises administering to the subject in an amount sufficient to inhibit expression of the target gene: (i) a double-stranded RNA described herein, where the wherein the first strand is complementary to a target gene; and/or (ii) an oligonucleotide described herein, wherein the oligonucleotide is complementary to a target gene.

The present disclosure further relates to a use of an oligonucleotide and/or dsRNA molecule described herein for inhibiting expression of a target gene in a target cell. The present disclosure further relates to a use of an oligonucleotide and/or dsRNA molecule described herein for inhibiting expression of a target gene in a target cell in vitro.

Another aspect the invention relates to a method of modulating the expression of a target gene in a cell, comprising administering to said cell an oligonucleotide and/or dsRNA molecule described herein. In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, INK gene, RAF gene, Erk1/2 gene, PCNA(p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

Exemplary Embodiments of the various aspects can be described by one or more of the numbered Embodiments:

Embodiment 1: An oligonucleotide comprising one or both of (a) and (b): at least one 2′-geminal-substituted nucleoside according to formula (I):

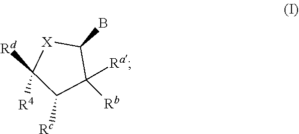

and (b) the 5′-terminal nucleoside is a 2′-geminal-substituted nucleoside of formula (II)

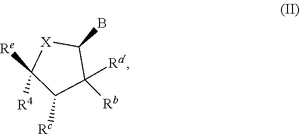

wherein in formulae (I) and (II):
X is O, S, C(R^{X})₂, or N(R^{XN});
each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{N}), or =CH₂;
R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
B is an optionally modified nucleobase;
R^{a′} is hydrogen, halogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a bond to an internucleoside linkage to a subsequent nucleoside;
R^{a2} is hydrogen or hydroxyl protecting group;
R^{a3} is hydrogen or sulfur protecting group;
R^{a4} is independently for each occurrence H, C₁-C₃₀alkyl, cyclyl, heterocyclyl, aryl, heteroaryl, aralkyl, sugar or R^{a5};
R^{a5} is independently for each occurrence amino (NH₂), alkylamino, dialkylamino, arylamino, diarylamino, heteroarylamino, or diheteroaryl amino;
m is 1-50;
n is 1-50;
R^{b} is optionally substituted C₁₋₃₀alkyl, optionally substituted C₂₋₃₀alkenyl, or optionally substituted C₂₋₃₀alkynyl;
R^{c} is a bond to an internucleoside linkage to a subsequent nucleoside, hydrogen, halogen, —OR^{c2}, —SR^{e3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, or a linker covalently attached to a solid support, and where, optionally, at least R^{c} or R^{b} is a bond to an internucleoside linkage to a subsequent nucleoside;
R^{c2} is hydrogen or hydroxyl protecting group;
R^{c3} is hydrogen or sulfur protecting group;
R^{c4} is independently for each occurrence H, C₁-C₃₀alkyl, cyclyl, heterocyclyl, aryl, heteroaryl, aralkyl, sugar or R^{c5};
R^{c5} is independently for each occurrence amino (NH₂), alkylamino, dialkylamino, arylamino, diarylamino, heteroarylamino, or diheteroaryl amino;
r is 1-50;
s is 1-50;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R^{a} taken together are 4′-C(R^{a11}R^{a12})ᵥY-2′ or 4′-Y—C(R^{a11}R^{a12})ᵥ-2′;
Y is —O—, —CH₂—, —CH(Me)-, —C(CH₃)₂—, —S—, —N(R^{a13})—, —C(O)—, —C(S)—, —S(O)—, —S(O)₂—, —OC(O)—, —C(O)O—, —N(R^{a13})C(O)—, or —C(O)N(R^{a13})—;
R^{a11} and R^{a12} independently are H, optionally substituted C₁-C₆alkyl, optionally substituted C₂-C₆alkenyl or optionally substituted C₂-C₆alkynyl;
R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
v is 1, 2 or 3;
or R⁴ and R^{c}taken together with the atoms to which they are attached form an optionally substituted C₃₋₈cycloalkyl, optionally substituted C₃₋₈cycloalkenyl, or optionally substituted 3-8 membered heterocyclyl;
R^{d} is —CH(R^{d1})—R^{d2} or —C(R^{d1})=CHR^{d2};
R^{d1} is hydrogen, optionally substituted C₁₋₃₀alkyl, optionally substituted —C₂₋₃₀alkenyl, or optionally substituted —C₂₋₃₀alkynyl;
R^{d2} is a bond to an internucleoside linkage to the preceding nucleoside;
R^{e} is optionally substituted C₁₋₆alkyl-R^{e1}, optionally substituted —C₂₋₆alkenyl-R^{e1}, or optionally substituted —C₂₋₆alkynyl-R^{e1};
R^{e1} is —OR^{e2}, —SR^{e3}, —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂;
R^{e2} is hydrogen or oxygen protecting group;
R^{e3} is hydrogen or sulfur protecting group;
each R^{e4} is independently hydrogen, optionally substituted C₁₋₃₀alkyl, optionally substituted C₂₋₃₀alkenyl, or optionally substituted C₂₋₃₀alkynyl, or an oxygen-protecting group;
and each R^{e5} is independently hydrogen, optionally substituted C₁₋₃₀alkyl, optionally substituted C₂₋₃₀alkenyl, or optionally substituted C₂₋₃₀alkynyl, or a sulfur-protecting group.

Embodiment 2: The oligonucleotide of Embodiment 1, wherein the 5′-terminal nucleoside is according to formula (IIA):

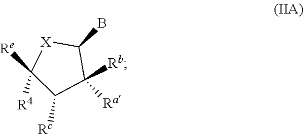

or according to formula (IIB):

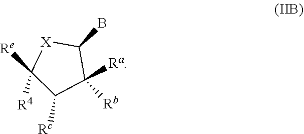

Embodiment 3: The oligonucleotide of Embodiment 1 or 2, wherein the 2′-geminal-substituted nucleoside is according to formula (IA):

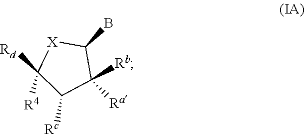

or according to formula (IB):

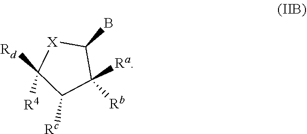

Embodiment 4: The oligonucleotide of anyone of Embodiments 1-3, wherein X is O.

Embodiment 5: The oligonucleotide of any one of Embodiments 1-4, wherein R^{a′} is hydrogen, halogen, —OR^{a2}, optionally substituted C₁-C₃₀alkyl, optionally substituted C₁-C₃₀alkoxy, —O(CH₂CH₂O)ₘCH₂CH₂OR^{a4}, or —NH(CH₂CH₂NH)ₙCH₂CH₂R^{a5}.

Embodiment 6: The oligonucleotide of any one of Embodiments 1-5, wherein R^{a′} is hydrogen, halogen, —OR^{a2}, or optionally substituted C₁-C₃₀alkoxy,

Embodiment 7: The oligonucleotide of any one of Embodiments 1-6, wherein R^{a′} is halogen, —OR^{a2}, or optionally substituted C₁-C₃₀alkoxy.

Embodiment 8: The oligonucleotide of any one of Embodiments 1-7, wherein R^{a′} is F, OH or optionally substituted C₁-C₃₀alkoxy.

Embodiment 9: The oligonucleotide of any one of Embodiments 1-8, wherein R^{a′} is C1-C₃₀alkoxy optionally substituted with an amino or C₁-C₆alkoxy.

Embodiment 10: The oligonucleotide of any one of Embodiments 1-9, wherein R^{b} is optionally substituted C₁₋₆alkyl, C₁₋₆haloalkyl, optionally substituted C₂₋₆alkenyl, or optionally substituted C₂₋₆alkynyl.

Embodiment 11: The oligonucleotide of any one of Embodiments 1-10, wherein R^{b} is methyl, ethyl, propyl, isopropyl, butyl, iso-butyl, t-butyl, vinyl, ethynyl, allyl or propargyl.

Embodiment 12: The oligonucleotide of any one of Embodiments 1-11, wherein R^{b} is methyl, vinyl, ethynyl, allyl or propargyl.

Embodiment 13: The oligonucleotide of any one of Embodiments 1-12, wherein R⁴ is hydrogen, optionally substituted C₁₋₆alkyl, optionally substituted C₂₋₆alkenyl, optionally substituted C₂₋₆alkynyl, or optionally substituted C₁₋₆alkoxy.

Embodiment 14: The oligonucleotide of any one of Embodiments 1-13, wherein R⁴ is hydrogen.

Embodiment 15: The oligonucleotide of any one of Embodiments 1-14, wherein R^{d} is —CH(R^{d1})—X^{d}—R^{d2}.

Embodiment 16: The oligonucleotide of any one of Embodiments 1-15, wherein X^{d} is O.

Embodiment 17: The oligonucleotide of any one of Embodiments 1-16, wherein R^{d1} is hydrogen or optionally substituted C₁-C₆alkyl.

Embodiment 18: The oligonucleotide of any one of Embodiments 1-17, wherein R^{d6} is hydrogen.

Embodiment 19: The oligonucleotide of any one of Embodiments 1-18, wherein R^{e} is C₁₋₆alkyl-R^{e1} or —C₂₋₆alkenyl-R^{e1}, C₁₋₆alkyl and C₂₋₆alkenyl are optionally substituted.

Embodiment 20: The oligonucleotide of any one of Embodiments 1-19, wherein R^{e} is —CH═CHR^{e1}.

Embodiment 21: The oligonucleotide of any one of Embodiments 1-20, wherein R^{e1} is —OR^{e2}, —P(O)(OR^{e4})₂, or —OP(O)(OR^{e4})₂.

Embodiment 22: The oligonucleotide of any one of Embodiments 1-21, wherein R^{e2} is hydrogen or optionally substituted C₁-C₆alkyl.

Embodiment 23: The oligonucleotide of any one of Embodiments 1-22, wherein the 2′-geminal-substituted nucleoside of formula (I) is at least at one of position 2, 3, 4, 5, 6, 7, 8, 9 or 10, counting from the 5′-end of the oligonucleotide.

Embodiment 24: The oligonucleotide of any one of Embodiments 1-23, wherein the 2′-geminal-substituted nucleoside of formula (I) is at position 7, counting from the 5′-end of the oligonucleotide.

Embodiment 25: The oligonucleotide of any one of Embodiments 1-24, wherein the oligonucleotide further comprises a ligand linked thereto.

Embodiment 26: The oligonucleotide of any one of Embodiments 1-25, wherein the oligonucleotide solely comprises 2′-geminal-substituted nucleosides of formulae (I) and (II).

Embodiment: The oligonucleotide of any of Embodiments 1-26, wherein the oligonucleotide further comprises at least one modified internucleoside 27linkage.

Embodiment 28: The oligonucleotide of any one of Embodiments 1-27, wherein the oligonucleotide further at least one modified nucleobase.

Embodiment 29: The oligonucleotide of Embodiment 1-28, wherein the wherein the oligonucleotide comprises at least one nucleoside modified at the 2′-position and wherein the nucleotide modified at the 2′-position is not a 2′-geminal nucleoside.

Embodiment 30: The oligonucleotide of any one of Embodiments 1-29, wherein the at least one nucleoside modified at the 2′-position is a 2′-F or 2′-OMe nucleoside.

Embodiment 31: The oligonucleotide of any one of Embodiments 1-30, wherein the oligonucleotide is from 10 to 50 nucleotides in length.

Embodiment: The oligonucleotide of any one of Embodiments 1-31, wherein the 5′-terminal nucleotide is a 2′-geminal-substituted 32nucleotide of formula (II).

Embodiment 33: The oligonucleotide of any one of Embodiments 1-32, wherein the oligonucleotide is linked to a solid support.

Embodiment 34: A double-stranded nucleic acid comprising a first oligonucleotide strand and a second oligonucleotide strand substantially complementary to the first strand, wherein the first strand and/or the second strand is an oligonucleotide of any one of Embodiments 1-33.

Embodiment 35: The double-stranded nucleic acid of Embodiment 34, wherein the first and second strand are independently 15 to 25 nucleotides in length.

Embodiment 36: The double-stranded nucleic acid of any one of Embodiments 34-35, wherein the first and/or the second strand has a 1-5 nucleotide overhang on its respective 5′-end or 3′-end.

Embodiment 37: The double-stranded nucleic acid of any one of Embodiments 34-36 wherein only one of the first or second strand has a 2 nucleotide single-stranded overhang on its 5′-end or 3′-end.

Embodiment 38: The double-stranded nucleic acid of any one of Embodiments 34-37, wherein only one strand has a 2 nucleotide single-stranded overhand on its 3′-end.

Embodiment 39: The double-stranded nucleic acid of any one of Embodiments 34-38, wherein the second strand comprises a ligand linked thereto.

Embodiment 40: The double-stranded nucleic acid of any one of Embodiments 34-39, wherein first strand is substantially complementary to a target nucleic acid and the double-stranded nucleic is capable of inducing RNA interference.

Embodiment 41: A method of reducing the expression of a target gene in a subject, comprising administering to the subject either: (i) a double-stranded RNA according to any one of Embodiments 34-40 wherein the first strand is complementary to a target gene; or (ii) an oligonucleotide according to any one of Embodiments 1-32, wherein the oligonucleotide is complementary to a target gene.

Embodiment 42: A compound of formula (III):

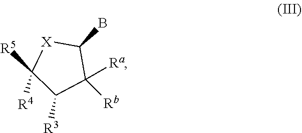

wherein: X is O, S, C(R^{X})₂, or N(R^{XN}); each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{N}), or =CH₂; R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group; B is an optionally modified nucleobase; R^{a} is hydrogen, halogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a reactive phosphorus group; R^{a2} is hydrogen or hydroxyl protecting group; R^{a3} is hydrogen or sulfur protecting group; R^{a4} is independently for each occurrence H, C₁-C₃₀alkyl, cyclyl, heterocyclyl, aryl, heteroaryl, aralkyl, sugar or R^{a}s; R^{a5} is independently for each occurrence amino (NH₂), alkylamino, dialkylamino, arylamino, diarylamino, heteroarylamino, or diheteroaryl amino; m is 1-50; n is 1-50; R^{b} is optionally substituted C₁₋₃₀alkyl, optionally substituted C₂₋₃₀alkenyl, or optionally substituted C₂₋₃₀alkynyl; R³ is hydrogen, halogen, —OR^{c2}, —SR^{c3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, a linker covalently attached to a solid support, or a reactive phosphorus group; R^{c2} is hydrogen or hydroxyl protecting group; R^{c3} is hydrogen or sulfur protecting group; R^{c4} is independently for each occurrence H, C₁-C₃₀alkyl, cyclyl, heterocyclyl, aryl, heteroaryl, aralkyl, sugar or R^{c5}; R^{c5} is independently for each occurrence amino (NH₂), alkylamino, dialkylamino, arylamino, diarylamino, heteroarylamino, or diheteroaryl amino; r is 1-50; s is 1-50; R⁴ is hydrogen, optionally substituted C₁₋₆alkyl, optionally substituted C₂₋₆alkenyl, optionally substituted C₂₋₆alkynyl, or optionally substituted C₁₋₆alkoxy; or R⁴ and R^{a} taken together are 4′-C(R^{a11}R^{a12})ᵥ—Y-2′ or 4′-Y—C(R^{a11}R^{a12})ᵥ-2′; Y is —O—, —CH₂—, —CH(Me)-, —C(CH₃)₂—, —S—, —N(R^{a13})—, —C(O)—, —C(S)—, —S(O)—, —S(O)₂—, —OC(O)—, —C(O)O—, —N(R^{a13})C(O)—, or —C(O)N(R^{a13})—; R^{a11} and R^{a12} independently are H, optionally substituted C₁-C₆alkyl, optionally substituted C₂-C₆alkenyl or optionally substituted C₂-C₆alkynyl; R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group; v is 1, 2 or 3; or R⁴ and R^{c}taken together with the atoms to which they are attached form an optionally substituted C₃₋₈cycloalkyl, optionally substituted C₃₋₈cycloalkenyl, or optionally substituted 3-8 membered heterocyclyl; R⁵ is optionally substituted C₁₋₆alkyl-R^{5a}optionally substituted —C₂₋₆alkenyl-R^{5a}, or optionally substituted —C₂₋₆alkynyl-R^{5a}; R^{5a} is —OR^{5b}, —SR^{5c}, hydrogen, a phosphorus group, a protected phosphorous group, a solid support or a linker to a solid support, provided that only one of R^{3a}, R³ and R⁵ is a linkage to a solid support; R^{5b} is H or hydroxyl protecting group; and R^{5c} is H or sulfur protecting group, and provided that only one of R^{3a}, R³, and R^{5a} is a solid support or linkage to a solid support; provided that only one of R^{3a}, R³ and R^{5a} is a reactive phosphorous, and provided that the compound is not where: R^{a} is F; R^{b} is methyl; R³ is —OR^{c2}; reactive phosphorous group or linkage to a solid support; R^{c2} is hydrogen or hydroxyl protecting group R⁴ is H; R⁵ is —CH₂OR^{5b}, R^{5b} is H, hydroxyl protecting group or a phosphorus group; and B is adenine, cytosine, guanine or uracil, each of which can be unprotected, protected or modified; or R^{a} is OH; R^{b} is methyl, vinyl or ethynyl; R³ is —OR^{c2}, reactive phosphorous group or linkage to a solid support; R^{c2} is hydrogen or hydroxyl protecting group R⁴ is H; R⁵ is —CH₂OR^{5b}, R^{5b} is H, hydroxyl protecting group or a phosphorus group; and B is adenine or guanine, each of can be unprotected, protected or modified.

Embodiment 43: The compound of Embodiment 42, wherein the compound is of formula (IIIA):

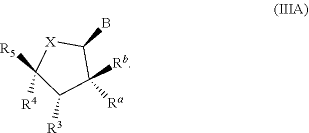

Embodiment 44: The compound of Embodiment 42, wherein the compound is of formula (IIIB):

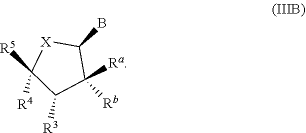

Embodiment 45: The compound of any one of Embodiments 42-44, wherein the reactive phosphorous group is phosphoramidite, H-phosphonate, alkyl-phosphonate, or phosphate triester.

Embodiment 46: The compound of any one of Embodiments 42-45, wherein the reactive phosphorous group is —OP(OR^{P})N(R^{P2})₂, —OP(SR^{P})N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}, wherein: R is an optionally substituted C₁₋₆alkyl; and each R^{P2} is independently optionally substituted C₁₋₆alkyl; or both R^{P2} taken together with the nitrogen atom to which they are attached form an optionally substituted 3-8 membered heterocyclyl; or R^{P} and one of R^{P2} taken together with the atoms to which they are attached form an optionally substituted 4-8 membered heterocyclyl; and R^{P3} is an optionally substituted C₁-C₃₀alkyl, optionally substituted C₂-C₃₀alkenyl, or optionally substituted C₂-C₃₀alkynyl.

Embodiment 47: The compound of Embodiment 46, wherein the reactive phosphorous group is —OP(OR^{P})N(R^{P2})₂.

Embodiment 48: The compound of Embodiment 46-47, wherein R^{P} is C₁₋₆alkyl substituted with cyano or —SC(O)Ph.

Embodiment 49: The compound of any one of Embodiments 46-48, wherein R^{P} is —CH₂CH₂CN.

Embodiment 50: The compound of any one of Embodiments 46-49, wherein each R^{P2} is independently methyl, ethyl, propyl, or isopropyl.

Embodiment 51: The compound of any one of Embodiments 46-50, wherein each R^{P2} is isopropyl.

Embodiment 52: The compound of any one of Embodiments 46-51, wherein R^{P3} is an optionally substituted C₁-C₆alkyl.

Embodiment 53: The compound of anyone of Embodiments 42-52, wherein X is O.

Embodiment 54: The compound of any one of Embodiments 42-53, wherein R^{a} is hydrogen, halogen, —OR^{a2}, or optionally substituted C₁-C₃₀alkoxy.

Embodiment 55: The compound of any one of Embodiments 42-54, wherein R^{a} is halogen, —OR^{a2}, or optionally substituted C₁-C₃₀alkoxy.

Embodiment 56: The compound of any one of Embodiments 42-55, wherein R^{a} is F, OH or optionally substituted C₁-C₃₀alkoxy.

Embodiment 57: The compound of any one of Embodiments 42-56, wherein R^{a} is C₁-C₃₀alkoxy optionally substituted with an amino or C₁-C₆alkoxy.

Embodiment 58: The compound of any one of Embodiments 42-57, wherein R^{b} is optionally substituted C₁₋₆alkyl, C₁₋₆haloalkyl, optionally substituted C₂₋₆alkenyl, or optionally substituted C₂₋₆alkynyl.

Embodiment 59: The compound of any one of Embodiments 42-58, wherein R^{b} is methyl, ethyl, propyl, isopropyl, butyl, iso-butyl, t-butyl, vinyl, ethynyl, allyl or propargyl.

Embodiment 60: The compound of any one of Embodiments 42-59, wherein R^{b} is methyl, vinyl, ethynyl, allyl or propargyl.

Embodiment 61: The compound of any one of Embodiments 42-60, wherein R³ is H, halogen, OR^{c2}, a reactive phosphorus group, or a linkage to a solid support.

Embodiment 62: The compound of any one of Embodiments 42-61, wherein R³ is a reactive phosphorus group, or a linkage to a solid support.

Embodiment 63: The compound of any one of Embodiments 42-62, wherein R³ is a reactive phosphorous group.

Embodiment 64: The compound of any one of Embodiments 42-63, wherein R⁴ is hydrogen, optionally substituted C₁₋₆alkyl, optionally substituted C₂₋₆alkenyl, optionally substituted C₂₋₆alkynyl, or optionally substituted C₁₋₆alkoxy.

Embodiment 65: The compound of any one of Embodiments 42-64, wherein R⁴ is hydrogen.

Embodiment 66: The compound of any one of Embodiments 42-65, wherein R⁵ is optionally substituted C₁₋₆alkyl-R^{5a} or optionally substituted —C₂₋₆alkenyl-R^{5a}

Embodiment 67: The compound of any one of Embodiments 42-66, wherein R^{5a} is —OR^{5b} or a phosphorous group.

Embodiment 68: The compound of any one of Embodiments 42-67, wherein R^{5a} is —OR^{5b}.

### Some Selected Definitions

For convenience, certain terms employed herein, in the specification, examples and appended claims are collected herein. Unless stated otherwise, or implicit from context, the following terms and phrases include the meanings provided below Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxy, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-l,l-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, NHCH₂CH₂CH₃, and NHCH(CH₃)₂. Exemplary dialkylamino includes, but is not limited to, N(C₁-C₁₀alkyl)₂, such as N(CH₃)₂, N(CH₂CH₃)₂, N(CH₂CH₂CH₃)₂, and N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

As used herein, the term “oxo” means double bonded oxygen, i.e., =O.

The term “carboxy” means the radical C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, —SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, /. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

The term “off-target” and the phrase “off-target effects” refer to any instance in which an effector molecule against a given target causes an unintended affect by interacting either directly or indirectly with another target sequence, a DNA sequence or a cellular protein or other moiety. For example, an “off-target effect” may occur when there is a simultaneous degradation of other transcripts due to partial homology or complementarity between that other transcript and the sense and/or antisense strand of an siRNA.

As used herein, the term “nucleoside” means a glycosylamine comprising a nucleobase and a sugar. Nucleosides includes, but are not limited to, naturally occurring nucleosides, abasic nucleosides, modified nucleosides, and nucleosides having mimetic bases and/or sugar groups.

As used herein, the term “nucleotide” refers to a glycosomine comprising a nucleobase and a sugar having a phosphate group covalently linked to the sugar. Nucleotides may be modified with any of a variety of substituents.

As used herein, the term “locked nucleic acid” or “LNA” or “locked nucleoside” or “locked nucleotide” refers to a nucleoside or nucleotide wherein the furanose portion of the nucleoside includes a bridge connecting two carbon atoms on the furanose ring, thereby forming a bicyclic ring system. Locked nucleic acids are also referred to as bicyclic nucleic acids (BNA).

As used herein, unless otherwise indicated, the term “methyleneoxy LNA” alone refers to β-D-methyleneoxy LNA.

As used herein, the term “MOE” refers to a 2′-O-methoxyethyl substituent.

As used herein, the term “gapmer” refers to a chimeric oligomeric compound comprising a central region (a “gap”) and a region on either side of the central region (the “wings”), wherein the gap comprises at least one modification that is different from that of each wing. Such modifications include nucleobase, monomeric linkage, and sugar modifications as well as the absence of modification (unmodified). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) in each wing are the same. In certain embodiments, the modification(s) in one wing are different from the modification(s) in the other wing. In certain embodiments, oligomeric compounds are gapmers having 2′-deoxynucleotides in the gap and nucleotides with high-affinity modifications in the wing.

The term ‘BNA’ refers to bridged nucleic acid, and is often referred as constrained or inaccessible RNA. BNA can contain a 5-, 6-membered, or even a 7-membered bridged structure with a “fixed” C₃′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

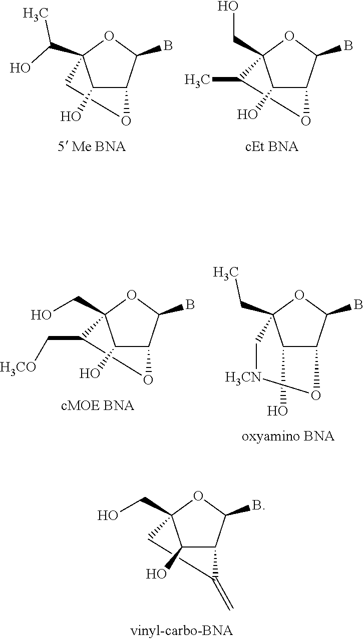

The term ‘LNA’ refers to locked nucleic acid, and is often referred as constrained or inaccessible RNA. LNA is a modified RNA nucleotide. The ribose moiety of an LNA nucleotide is modified with an extra bridge (e.g., a methylene bridge or an ethylene bridge) connecting the 2′ hydroxyl to the 4′ carbon of the same ribose sugar. For instance, the bridge can “lock” the ribose in the 3′-endo North) conformation:

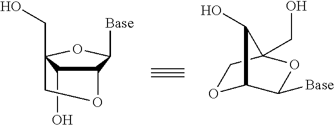

The term ‘ENA’ refers to ethylene-bridged nucleic acid, and is often referred as constrained or inaccessible RNA.

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, a “terminal region” of a strand refers to positions 1-4, e.g., positions 1, 2, 3, and 4, counting from the nearest end of the strand. For example, a 5′-terminal region refers to positions 1-4, e.g., positions 1, 2, 3 and 4 counting from the 5′-end of the strand. Similarly, a 3′-terminal region refers to positions 1-4, e.g., positions 1, 2, 3 and 4 counting from the 3′-end of the strand.

For example, a 5′-terminal region for the antisense strand is positions 1, 2, 3 and 4 counting from the 5′-end of the antisense strand. A preferred 5′-terminal region for the antisense strand is positions 1, 2 and 3 counting from the 5′-end of the antisense strand. A 3′-terminal region for the antisense strand can be positions 1, 2, 3, and 4 counting from the 3′-end of the strand. A preferred 3′-terminal region for the antisense strand is positions 1, 2 and 3 counting from the 3′-end of the antisense strand.

Similarly, a 5′-terminal region for the sense strand is positions 1, 2, 3 and 4 counting from the 5′-end of the sense strand. A preferred 5′-terminal region for the sense strand is positions 1, 2 and 3 counting from the 5′-end of the sense strand. A 3′-terminal region for the sense strand can be positions 1, 2, 3, and 4 counting from the 3′-end of the strand. A preferred 3′-terminal region for the sense strand is positions 1, 2 and 3 counting from the 3′-end of the sense strand.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As used herein, the term “in vitro” refers to events that occur in an artificial environment, e.g., in a test tube or reaction vessel, in cell culture, etc., rather than within an organism (e.g. animal or a plant). As used herein, the term “ex vivo” refers to cells which are removed from a living organism and cultured outside the organism (e.g., in a test tube). As used herein, the term “in vivo” refers to events that occur within an organism (e.g. animal, plant, and/or microbe).

As used herein, the term “subject” or “patient” refers to any organism to which a composition disclosed herein can be administered, e.g., for experimental, diagnostic, and/or therapeutic purposes. Typical subjects include animals (e.g., mammals such as mice, rats, rabbits, non-human primates, and humans) and/or plants. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to with domesticated animals and/or pets.

In some embodiments, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

As used herein, the term “dosage unit” refers to a form in which a pharmaceutical agent is provided. In certain embodiments, a dosage unit is a vial comprising lyophilized oligonucleotide or double-stranded oligonucleotide described herein. In certain embodiments, a dosage unit is a vial comprising reconstituted oligonucleotide or double-stranded oligonucleotide described herein.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: siRNAs with 2′-Deoxy-2′-α-F-2′-β-C-Methyl Pyrimidine Nucleotides: Modulation of Thermal and Metabolic Stabilities, Improved RNAi Activity Through Synergy with 5′-Phosphate Mimics and Seed-Region Mediated Off-Target Mitigation

Although 2′-deoxy-2′-α-F-2′-β-C-methyl (2′-F/Me) uridine nucleoside derivatives are a successful class of antiviral drugs, this modification had not been studied in oligonucleotides. Herein, we demonstrate the facile synthesis of 2′-F/Me-modified pyrimidine phosphoramidites and subsequent incorporation into oligonucleotides. In spite of the C3′-endo preorganization of the parent nucleoside, a single incorporation into RNA or DNA resulted in significant thermal destabilization of a duplex, due unfavorable enthalpy likely resulting from steric effects. When located at the terminus of an oligonucleotide, the 2′-F/Me modification imparted more resistance to degradation than the corresponding 2′-F nucleotides. Small interfering RNAs (siRNAs) modified at certain positions with 2′-F/Me had similar or better silencing activity than the optimized parent siRNA when delivered via a lipid nanoparticle or as a GalNAc conjugate. Modification in the seed region of the antisense strand at position 6 or 7 resulted in activity equivalent to the parent. Placement at position 7 of the antisense strand mitigated seed-based off-target effects. When combined with the 5′ vinyl phosphonate E isomer, the 2′-F/Me modification enhanced silencing compared to the parent; when combined with the Z isomer, it was equivalent. This is the first demonstration of equivalence of Z isomer activity; it is presumably driven by the steric and conformational features of the C-methyl-containing sugar ring. These data indicate that the 2′-F/Me nucleotides are promising tools for nucleic acid-based therapeutic applications to increase potency, duration, and safety.

RNA interference (RNAi) is a post-transcriptional pathway for gene regulation mediated by small interfering RNAs (siRNAs).¹⁻³ siRNAs, loaded onto Argonaute 2 (Ago2), the catalytic component of the RNA-induced silencing complex (RISC), target complementary mRNAs for degradation.⁴ This reduces the expression of the encoded protein. Synthetic siRNAs are powerful tools for fundamental research and are used clinically for treatment of multiple diseases, including hereditary transthyretin-mediated amyloidosis, acute hepatic porphyrias, primary hyperoxaluria type 1, and heterozygous familial hypercholesterolemia.⁵⁻¹⁹ For clinical use, siRNAs are formulated in lipid nanoparticles (LNPs)¹³ or conjugated with a tri-N-acetylgalactosamine (GalNAc), which results in hepatocyte-specific delivery via the asialoglycoprotein receptor.²⁰ Clinically used siRNAs are also chemically modified to improve potency, increase metabolic stability, avoid immune responses, and mitigate off-target effects.^{21-22,23} Chemical modifications currently used in clinically approved siRNAs are DNA, 2′-fluoro (2′-F), and 2′-O-methyl (2′-OMe). These modifications provide sufficient specificity and metabolic stability when combined with phosphorothioates at 5′ and 3′ termini to result in excellent safety and efficacy profiles in vivo.

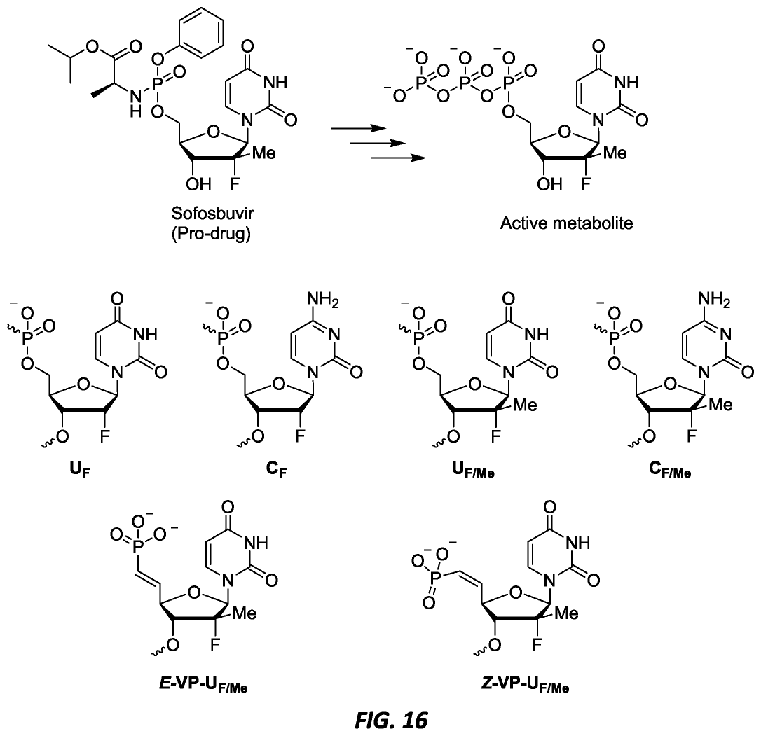

The chemical modifications 2′-F and 2′-OMe are pre-organized into the RNA-like C3′-endo conformation, resulting in enhanced binding to RNA, favorable binding to Ago2, and increased stability toward nuclease degradation relative to the parent ribonucleotide.²⁴⁻²⁵ Inventors reasoned that other nucleosides pre-organized into the C3′-endo conformation might be used to optimize siRNA activity. The C3′-endo conformation is favored upon alkylation of the sugar as in 2′-deoxy-2′-a-C-methyl thymidine.²⁶⁻²⁸ In 2′-C-methyluridine nucleoside, the 2′-C methyl substituent adopts the pseudo-equatorial conformation due to steric interactions, the C2′—OH group has a stabilizing O4′—O2′ gauche effect, the C-5′ side chain is pseudo-equatorial, and the base is pseudo-axial, satisfying the weak anomeric effect.^{12,29} Sugar-alkylated nucleosides have been developed for use as antiviral agents against the hepatitis C virus (HCV).^{25, 30-32} Sofosbuvir, the first ribonucleotide analog inhibitor to receive FDA approval for treatment of HCV,³³⁻³⁸ is a prodrug of 2′-deoxy-2′-α-C—F-2′-β-C-Me (2′-F/Me) uridine triphosphate (FIG. 16). The 2′-F/Me uridine triphosphate does not act as a substrate for human mitochondrial polymerases, likely due to the steric bulk around the 2′ position.³⁹

The enhanced population of C3′-endo sugar conformation of 2′-F/Me nucleotide, lack of mitochondrial toxicity, and potential for increased metabolic stability render it an interesting modification for RNAi applications. The lack of mitochondrial toxicity is particularly important as the 2′-F modification has some safety risk at very high concentrations.⁴⁰ In RNAi, a non-hydrolyzable, metabolically stable 5′ phosphate mimic such as (E) vinyl phosphonate (VP) at the 5′ terminus of the antisense strand enables selective recognition of this strand over the sense strand by the MID domain of Ago2, resulting in improved potency.^{22, 41-47} Hybridization-based off-target effects can be mitigated by mechanisms that ensure proper strand selection or thought the judicious incorporation of destabilizing modifications like glycol nucleic acids (GNA).⁴⁸ Herein we describe synthesis of 2′-F/Me pyrimidine phosphoramidites and the effects of this modification on duplex thermal stability, resistance towards exonuclease degradation, and RNAi on- and off-target activity of siRNAs. Our observations were rationalized using molecular modeling of appropriate nucleic acid-protein interactions.

### Results and Discussion

Synthesis of RNA oligonucleotides containing 2′-F/Me-pyrimidine. As shown in Scheme 1, thhe 2′-F/Me pyrimidine phosphoramidites (3 and 6) were synthesized from the corresponding commercially available nucleosides (1 and 4) using standard nucleoside protection with a 4,4′-dimethoxytrityl group at the 5′ position, a benzoyl group at the exocyclic amine of cytosine base, and phosphitylation (Scheme 1). The bis-pivaloyloxymethyl vinyl phosphonate 7 was synthesized using methods developed in our laboratories for other nucleosides.^{22, 41-47} The mixture of diastereomers (E/Z≈5/1) was separated into pure isomers 8 and 9 after the desilylation of 3′-hydroxy group. The 3′-hydroxy groups of these nucleoside monomers were converted to the phosphoroamidite forms (Scheme 1; see Supporting Information for details). The phosphoroamidite building blocks were site-specifically incorporated into oligonucleotides using an automated synthesizer. Cleavage from the solid support and subsequent deprotection of the synthesized oligonucleotides were performed under standard conditions using ammonium hydroxide solution. The crude oligonucleotides were purified by HPLC and characterized by LC-MS (see Supporting Information for details).

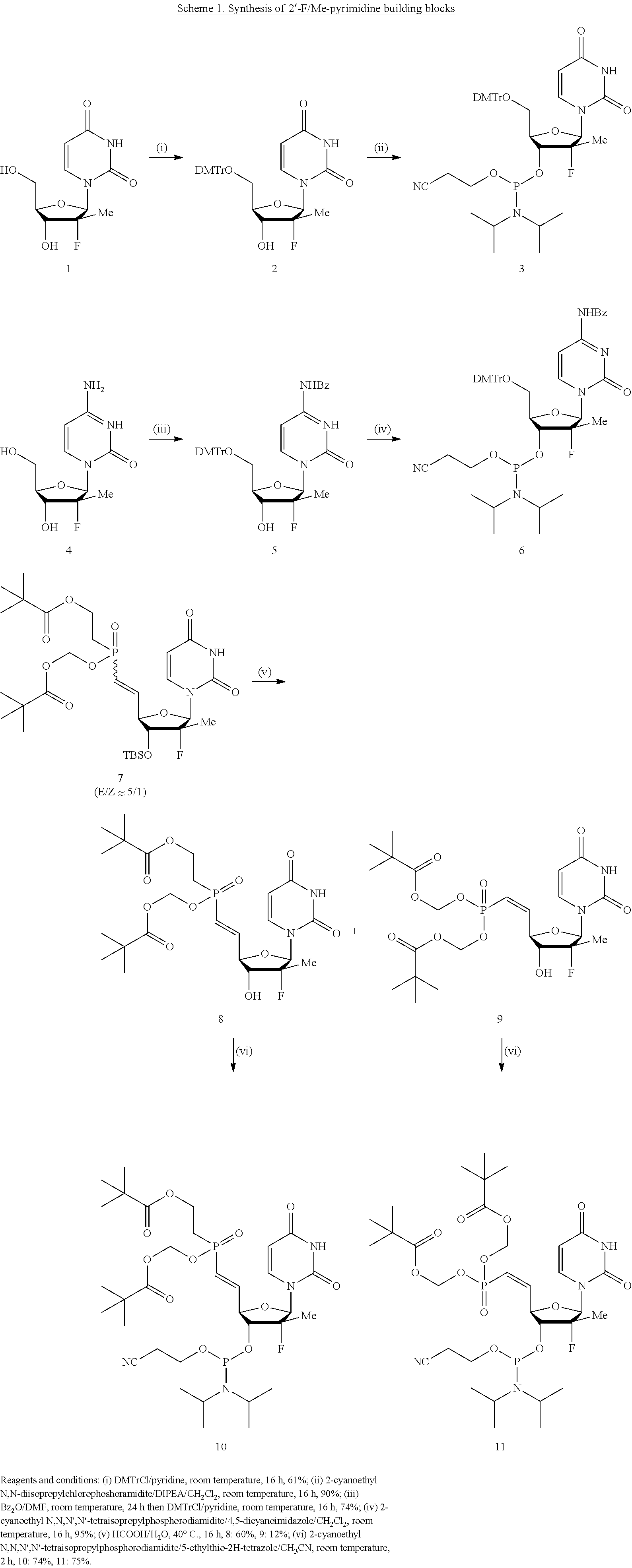

Thermodynamic stabilities of duplexes containing RNA strands modified with 2′-F/Me-pyrimidine nucleotides. Melting temperatures (Tm) and thermodynamic parameters of hybridization of 12-mer RNA duplexes containing a single, centrally located modification were evaluated (Table 4). Duplexes containing 2′-F-modified U or C (U_{F} and C_{F}, respectively) had similar or slightly increased Tₘs compared to the unmodified RNA duplex (ON3:2 and ON5:2 vs. ON1:2). The incorporation of 2′-F/Me-modified nucleotides (U_{F/Me} and C_{F/Me}), however, dramatically reduced the Tm by about 15° C. relative to the unmodified RNA duplex. There seemed to be little sequence dependence of this destabilization, as it was observed for both U_{F/Me}:A and C_{F/Me}:G base pairs, which were flanked by G:C or U:A base pairs in ON4:2 or ON6:2, respectively. This pattern of decreased thermal stability was also observed in the contexts of RNA:DNA and DNA:DNA duplexes, where even in high salt (1 M NaCl, PBS, pH 7.4) a clear transition was not apparent (Table 9). In particular, the shapes of melting curves for duplexes containing U_{F/Me} and the complementary DNA strand were quite broad.

TABLE 4
Thermal denaturation temperatures and thermodynamic parameters of duplexes
with an RNA strand containing 2′-F or 2′-F/Me^{a}

| | | Tₘ | ΔTₘ | ΔG₃₁₀ | ΔH | T₃₁₀ΔS |
|---|---|---|---|---|---|---|
| ON | Sequence | [° C.] | [° C.] | [kJ/mol] | [kJ/mol] | [kJ/mol] |
| 1 | 5′- | 54.1 | | −58 ± 0 | −453 ± 6 | −395 ± 6 |
| | r(UACAGUCUAUGU) | | | | | |
| | (SEQ ID NO: 36) | | | | | |
| 2 | 3′-r(AUGUCAGAUACA) | | | | | |
| | (SEQ ID NO: 37) | | | | | |
| 3 | 5′- | 53.9 | −0.2 | −60 ± 0 | −449 ± 4 | −388 ± 3 |
| | r(UACAGU_{F}CUAUGU) | | | | | |
| 2 | (SEQ ID NO: 38) | | | | | |
| | 3′-r(AUGUCAGAUACA) | | | | | |
| | (SEQ ID NO: 37) | | | | | |
| 4 | 5′- | 39.1 | −15.0 | −38 ± 0 | −343 ± 6 | −304 ± 6 |
| | r(UACAGU_{F/Me}CUAUGU) | | | | | |
| | (SEQ ID NO: 39) | | | | | |
| 2 | 3′-r(AUGUCAGAUACA) | | | | | |
| | (SEQ ID NO: 37) | | | | | |
| 5 | 5′-r(UACAGUC_{F}UAUGU) | 55.1 | +1.1 | −61 ± 0 | −444 ± 3 | −383 ± 3 |
| | (SEQ ID NO: 40) | | | | | |
| 2 | 3′-r(AUGUCAGAUACA) | | | | | |
| | (SEQ ID NO: 37) | | | | | |
| 6 | 5′-r(UACAGUC_{F/Me}UAUGU) | 38.3 | −15.8 | −38 ± 1 | −347 ± 7 | −309 ± 7 |
| | (SEQ ID NO: 41) | | | | | |
| 2 | 3′-r(AUGUCAGAUACA) | | | | | |
| | (SEQ ID NO: 37) | | | | | |

^{a}The absorbances of hybridized duplexes (2.5 M) at 260 nm were determined as a function of temperature in low-salt PBS (137 mM NaCl, 2.7 mM KCl, 10 mM Na₂HPO₄, 1.8 mM KH₂PO₄, pH 7.4). The Tₘ was determined as the maximum of the first derivative of the melting curve. Values are reported as the average of two independent experiments. ΔTₘ was calculated with respect to the unmodified RNA duplex. Thermodynamic parameters are an average of six determinations using the Varian Cary Bio-300 built-in software, with standard deviation reported.

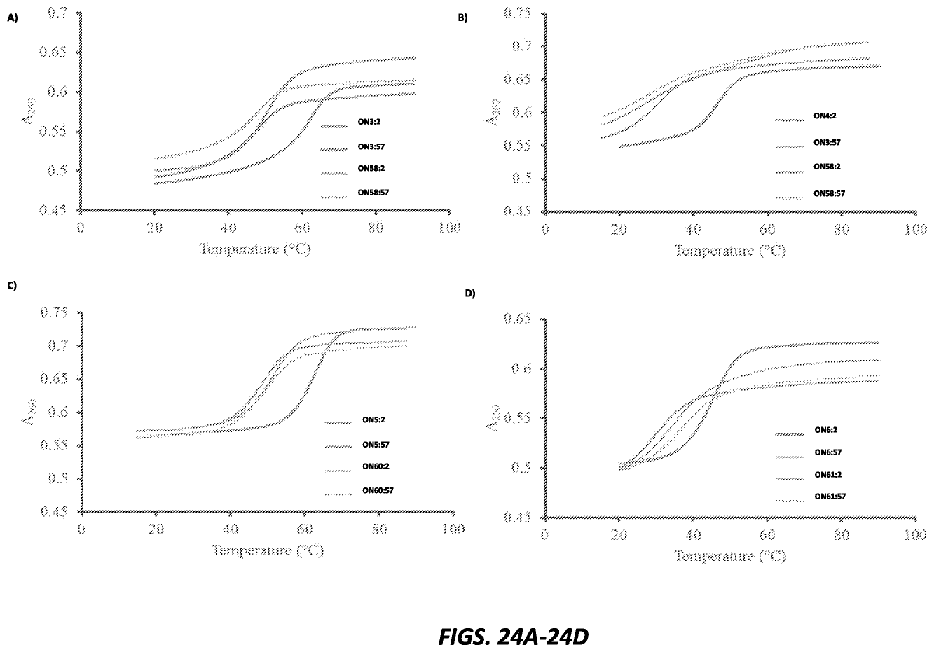

The thermodynamic parameters were obtained using the van′t Hoff method based on the hyperchromicity of the melting curves (Table 4). The duplexes formed from a modified RNA strand with a complementary RNA have relatively sharp transitions (FIGS. 24A-24D), thus supporting the assumption of a two-state model.⁴⁹ The changes in the Gibbs free energy of hybridization closely resemble the trends seen with Tₘ values that showed that 2′-F/Me modifications significantly reduced the thermal stability of the duplex. Both RNA and 2′-F-modified duplexes were thermodynamically favorable with ΔG₃₁₀ values of approximately −60 kJ/mol, whereas those formed from 2′-F/Me-modified strands were less favorable by about 20 kJ/mol compared to unmodified RNA (compare ON1:2 vs. ON4:2 and ON6:2). The reduced stability of the 2′-F/Me-modified duplex appears to be due to unfavorable enthalpic contributions, that are largely compensated by favorable entropic contributions. Even though U_{F/Me} and C_{F/Me} adopt a C3′-endo sugar pucker, which is pre-organized for binding to RNA, the destabilization suggests that other interactions (e.g., base pairing, stacking) that result in favorable enthalpic contributions are compromised.

Mismatch discrimination by RNA oligonucleotides modified with 2′-F/Me-pyrimidine nucleotides. Next, the ability to thermally discriminate a single-base mismatch in a duplex containing a modified nucleotide was evaluated (Table 5). Duplexes were formed between RNA strands containing either U_{F} (ON3) or U_{F/Me} (ON4) and a complementary RNA strand, with the exception of a single mismatch with the uridine derivative (ON7, ON8, or ON9) or a mismatch on the 3′ side of the modified nucleotide (ON10, ON11, ON12). We also evaluated duplexes formed between RNA strands containing either C_{F} (ON5) or C_{F/Me} (ON6) and ON10 and duplexes ON11 and ON12, resulting in mismatches to the modified nucleotide, and duplexes ON7, ON8, and ON9 resulting in mismatches to the 5′ side of the modified nucleotide. Due to the lower melting temperature caused by the mismatches, these duplexes were evaluated in high-salt buffer, where both canonical RNA duplexes and those containing a 2′-F nucleotide exhibited excellent and similar thermal discrimination, regardless of the proximity of the mismatch to the modification (Table 5).

TABLE 5
Thermal denaturation temperatures of modified duplexes containing a single
base pair mismatch^{a}

| | | | ΔTₘ (° C.) | | | | | |
|---|---|---|---|---|---|---|---|---|
| | | | 3′-AUGUCBGAUACA | | | 3′-AUGUCABAUACA | | |
| | | Tₘ | (SEQ ID NO: 107) | | | (SEQ ID NO: 108) | | |
| | | (° C.) | ON7 | ON8 | ON9 | ON10 | ON11 | ON12 |
| | Modified strand | ON2 | G | U | C | C | U | A |
| ON1 | 5′-UACAGUCUAUGU | 61.6 | −1.5 | −11.4 | −11.9 | −21.1 | −18.6 | −18.5 |
| | (SEQ ID NO: 36) | | | | | | | |
| ON3 | 5′- | 62.2 | −2.0 | −12.4 | −12.9 | −21.1 | −19.5 | −18.9 |
| | UACAGU_{F}CUAUGU | | | | | | | |
| | (SEQ ID NO: 38) | | | | | | | |
| ON4 | 5′- | 47.1 | −2.6 | −5.1 | −8.0 | −16.4 | −15.8 | −12.7 |
| | UACAGU_{F/Me}CUAUGU | | | | | | | |
| | (SEQ ID NO: 39) | | | | | | | |
| ON5 | 5′- | 63.1 | −2.6 | −12.1 | −12.0 | −21.9 | −19.8 | −19.7 |
| | UACAGUC_{F}UAUGU | | | | | | | |
| | (SEQ ID NO: 40) | | | | | | | |
| ON6 | 5′- | 45.2 | 0.0 | −9.9 | −13.9 | −15.7 | −13.3 | −13.1 |
| | UACAGUC_{F/Me}UAUGU | | | | | | | |
| | (SEQ ID NO: 41) | | | | | | | |

^{a}Tm was measured by monitoring the absorbance of hybridized duplexes (2.5 μM) with increasing temperature in high-salt PBS (932 mM NaCl, 18.4 mM KCl, 68 mM Na₂HPO₄, 12.2 mM KH₂PO₄, pH 7.4). ΔTₘ is calculated with respect to the corresponding complementary duplex (ON1:ON2).

When U_{F/Me} was located directly across from guanosine (ON4:ON7), the discrimination was increased by approximately 1° C. for the G:U wobble compared to unmodified RNA (ON4:ON7 vs. ON1:ON7), but the other mismatches were discriminated to a lesser degree than by unmodified RNA. Interestingly, the reduced discrimination was propagated to the 3′-adjacent base pair (e.g., ON4:ON10, ON4:ON11, ON4:ON12), suggesting that a local distortion caused by the modification increases the tolerance for mismatches nearby. C_{F/Me} reduced the thermal discrimination of a mismatched nucleobase located directly across from the modified nucleotide (e.g., ON6:ON10, ON6:ON11, ON6:ON12) by about 5° C. compared to unmodified RNA. This loss in thermal discrimination was largely recovered when the mismatch was on the 5′ side (e.g., ON6:ON7, ON6:ON8, ON6:ON9), suggesting that 2′-F/Me pyrimidines asymmetrically perturb the duplex in the 3′ direction. It is worth noting that even with reduced discrimination of mismatches, the remarkably low absolute melting temperatures would likely increase the overall specificity of hybridization at the biologically relevant temperature of 37° C.

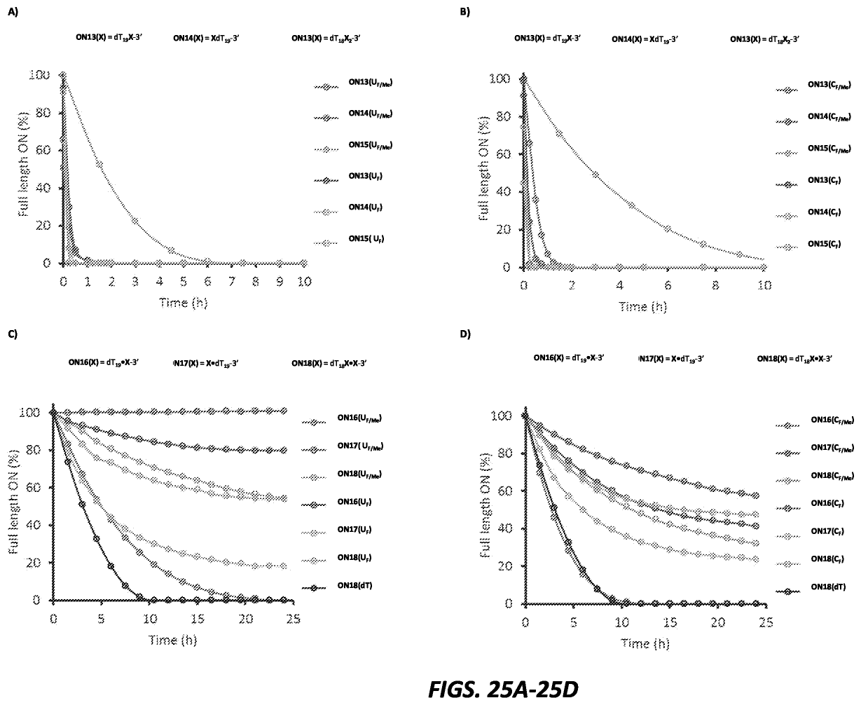

Exonuclease-mediated degradation of oligonucleotides with 2′-F/Me modifications. To assess the impact of 2′-F/Me modifications on metabolic stability, terminally modified poly-dT oligonucleotides were incubated in the presence of either a 3′ or 5′ exonuclease. Oligonucleotides with a full phosphodiester (PO) backbone containing a single 2′-F or 2′-F/Me residues at the terminus or the penultimate position (ON13, ON15, and ON15) were degraded within 1 h in the presence of the 3′ exonuclease snake venom phosphodiesterase (SVPD) (FIGS. 25A-25D). The doubly modified ON15(U_{F/Me}) and ON15(C_{F/Me}) had t_{1/2} values of approximately 1 and 2 h, respectively (Table 6). Oligonucleotides with a single phosphorothioate (PS) linkage and either U_{F/Me} or C_{F/Me} were clearly more resistant to SVPD-catalyzed degradation than were the corresponding 2′-F-modified oligonucleotides (ON14 vs. ON13 and ON17 vs. ON16), and two 2′-F/Me pyrimidine nucleotides connected via a PS linkage had an additional stabilizing effect (Table 6).

TABLE 6
Half-lives of oligonucleotides incubated with 3′ exonuclease SVPD ^{a}

| | t_{1/2} in presence of 3′-exonuclease (h) | | | | | |
|---|---|---|---|---|---|---|
| | ON13(X) | ON14(X) | ON15(X) | ON16(X) | ON17(X) | ON18(X) |
| X= | dT₁₉X | dT₁₈XdT | dT₁₈XX | dT₁₉•X | dT₁₈X•dT | dT₁₈X•X |
| U_{F} | <0.2 | <0.2 | <0.2 | 79 | 10 | 29 |
| U_{F/Me} | 0.2 | 0.2 | 0.9 | 3.2 | no deg. | 27 |
| C_{F} | <0.2 | <0.2 | <0.2 | 19 | 12 | 24 |
| C_{F/Me} | 0.6 | <0.2 | 2.0 | 1.6 | 31 | 15 |

^{a} The half-lives were determined by plotting the percent full-length oligonucleotide vs. time and fitting to the exponential decay function. For experimental conditions, see FIG. 2.
PS linkage, •;
no deg., no degradation observed within 24 h.

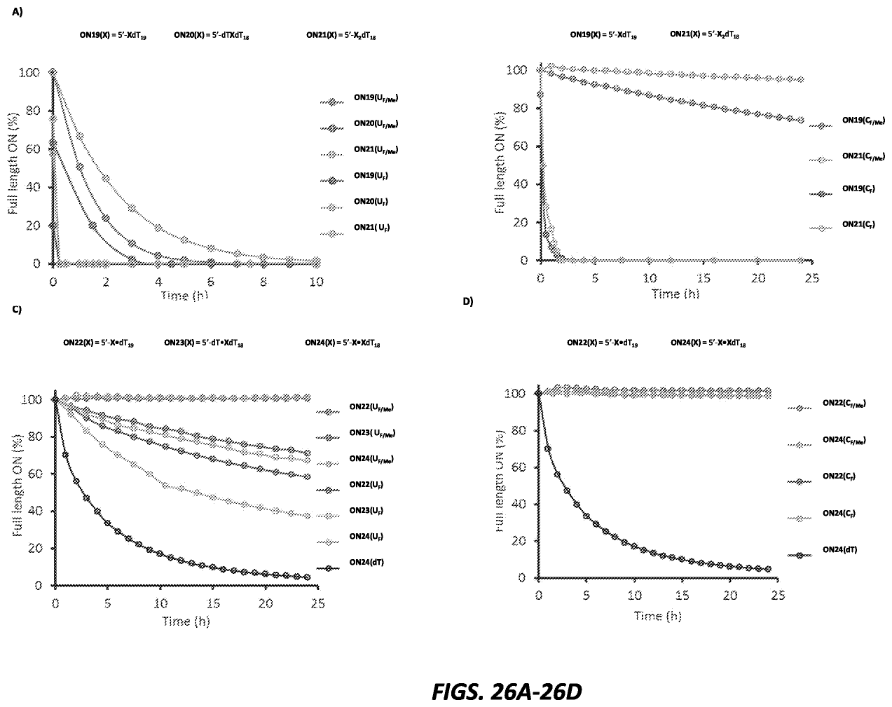

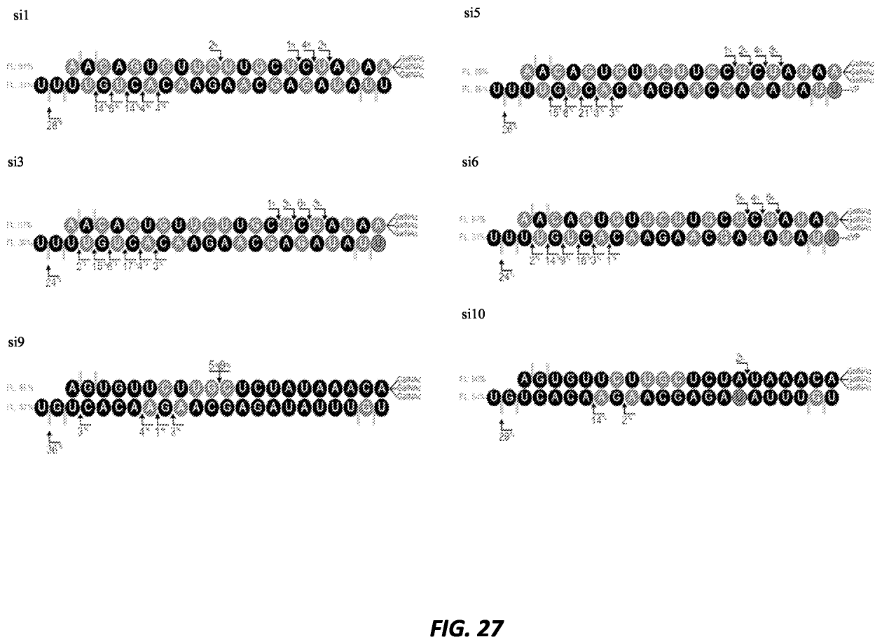

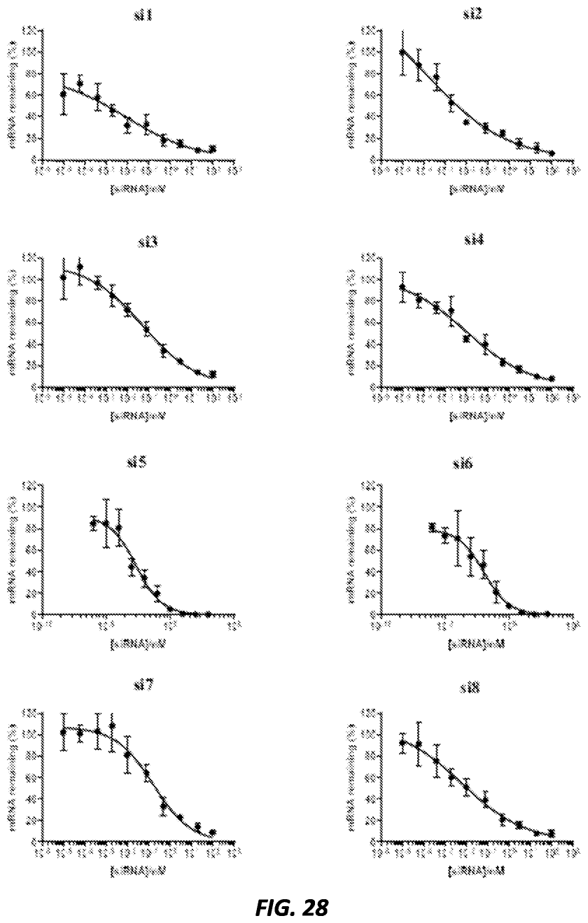

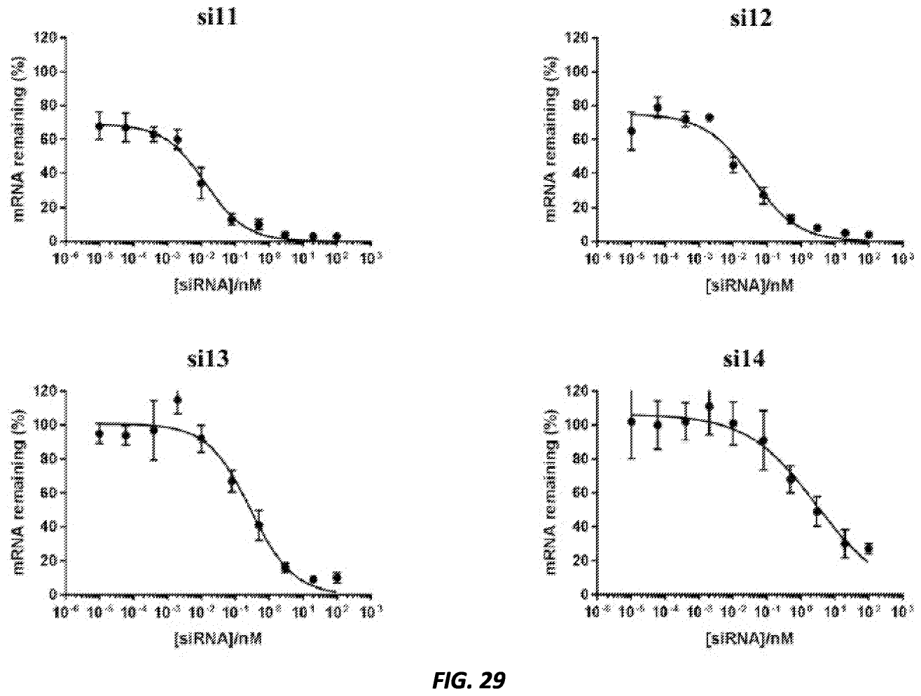

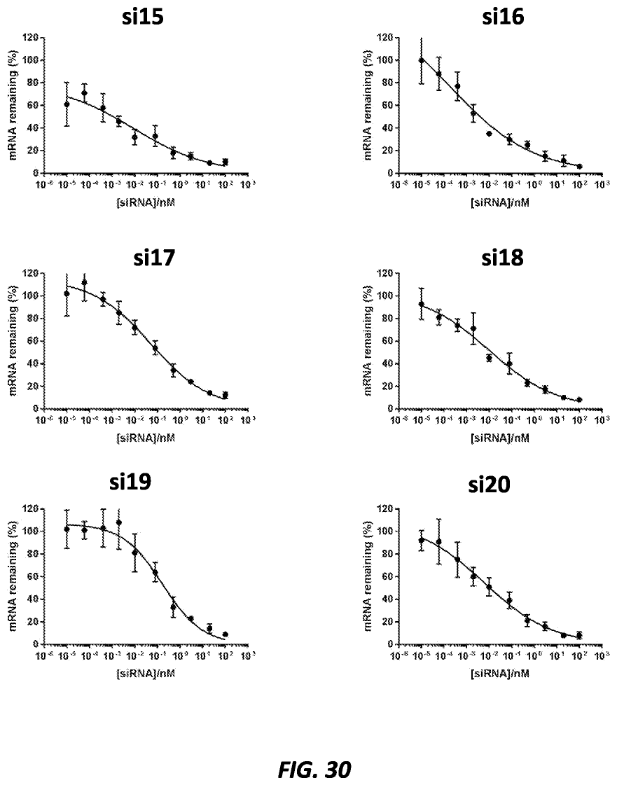

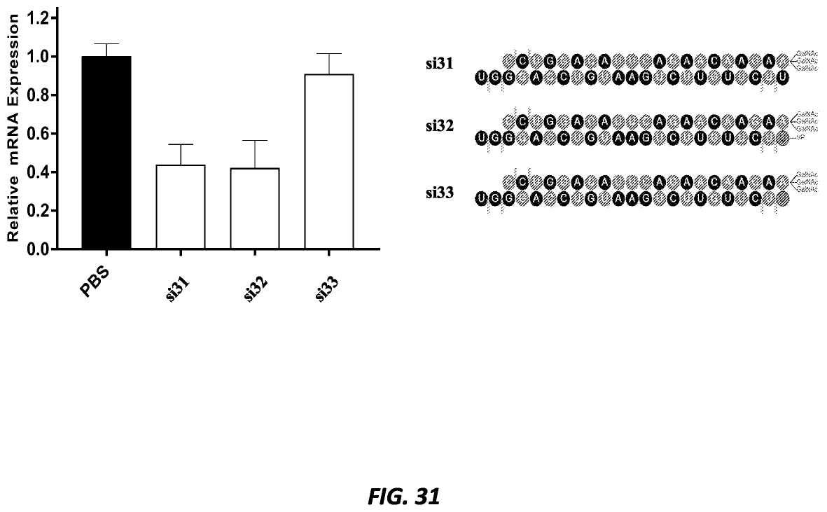

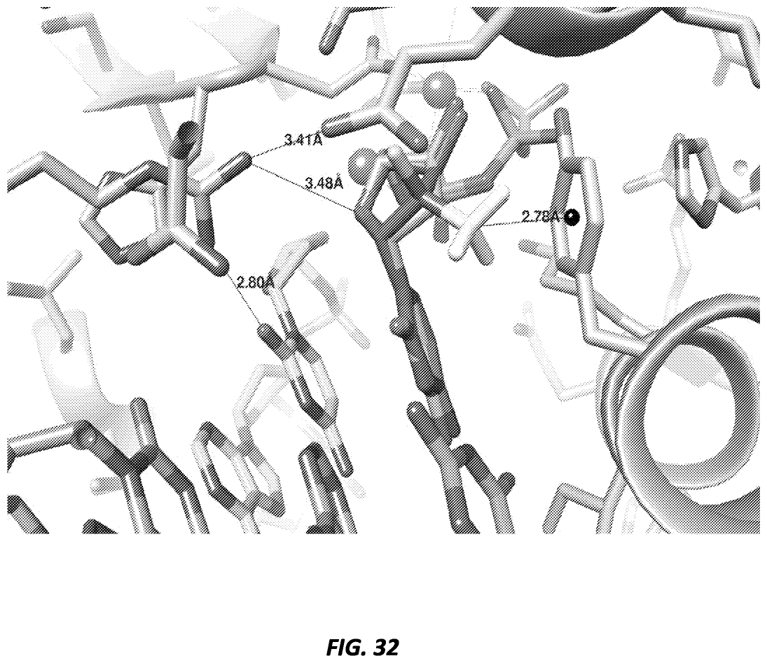

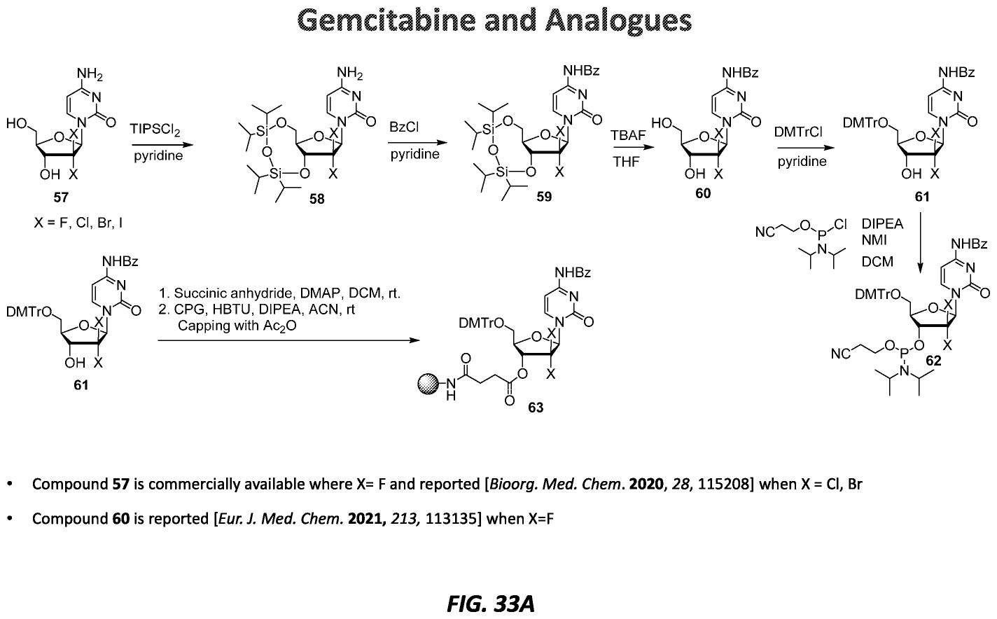

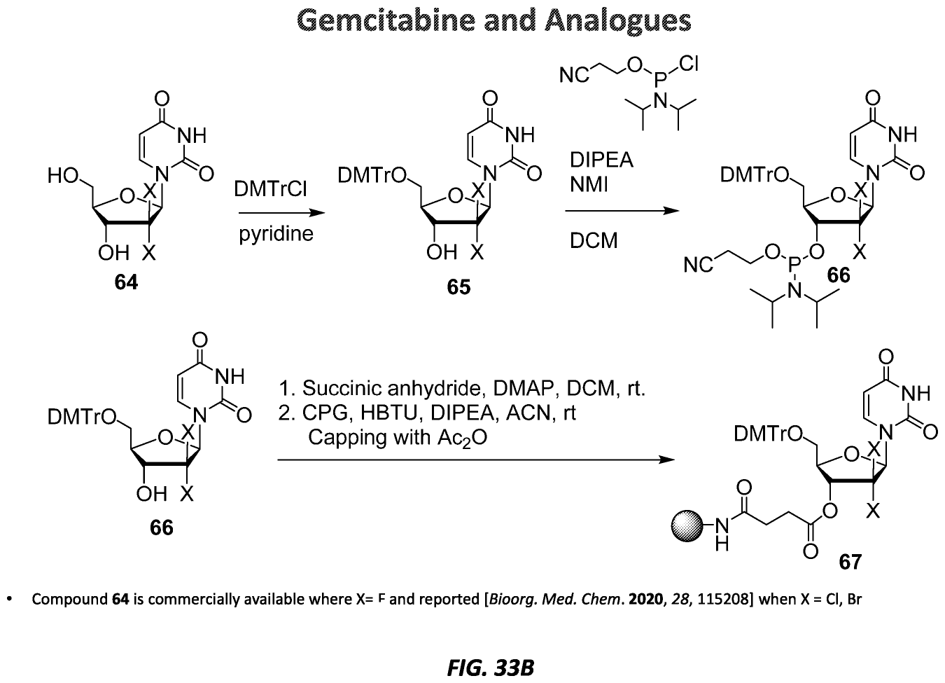

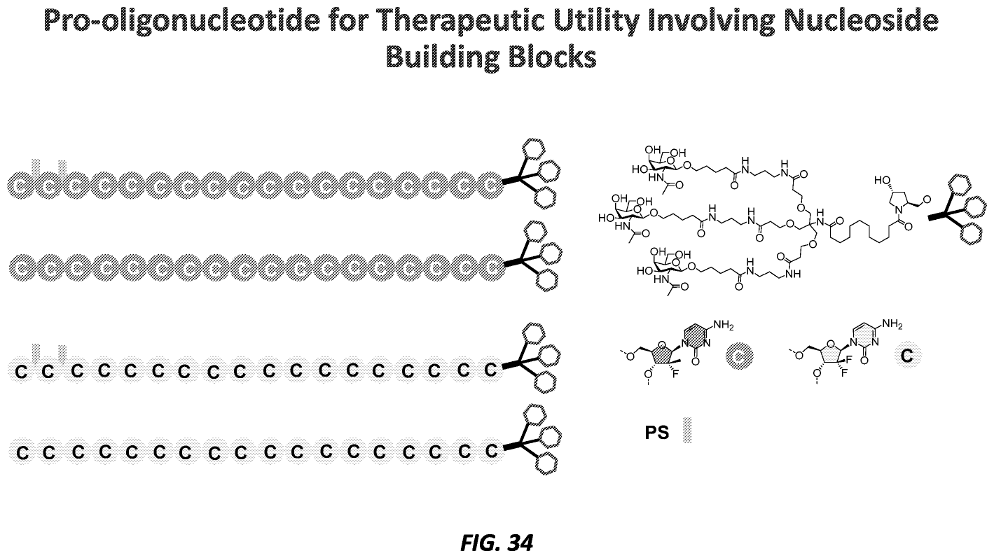

The stability of oligonucleotides 5′-modified towards 5′ exonuclease-mediated degradation with phosphodiesterase II (PDII) were also evaluated (Table 7). For oligonucleotides with full PO backbones (ON19), U_{F/Me} was slightly more stabilizing than U_{F}. For the cytidine derivatives the benefit of 2′-F/Me was striking: Only 25% degradation was observed after 24 h for the oligonucleotide with a single terminal C_{F/Me} modification, whereas the oligonucleotide with the terminal C_{F} was completely degraded within 1 h (ON19(C_{F/Me}) vS. ON19(C_{F}); FIG. 26A-26D). Addition of a terminal PS linkage (ON21), completely stabilized oligonucleotides modified with either C_{F/Me} or C_{F} against degradation, which could reflect a substrate preference of this particular enzyme, as previously noted.⁵⁰ In summary, U_{F/Me} and C_{F/Me} provide better 5′-exonuclease protection than U_{F} and C_{F}.

TABLE 7
Half-lives of modified oligonucleotides incubated with 5′ exonuclease PD II ^{a}

| | t_{1/2} in presence of 5′-exonuclease (h) | | | | | |
|---|---|---|---|---|---|---|
| | ON19(X) | ON20(X) | ON21(X) | (X)ON22 | (X)ON23 | (X)ON24 |
| X= | XdT₁₉ | dTXdT₁₈ | XXdT₁₈ | X•dT₁₉ | dT•XdT₁₈ | X•XdT₁₈ |
| U_{F} | <0.2 | <0.2 | <0.2 | 32 | 17 | 43 |
| U_{F/Me} | 0.9 | 0.6 | 1.8 | no deg. | 50 | no deg. |
| C_{F} | <0.2 | n.d. | 0.3 | no deg. | n.d. | 5.8 |
| C_{F/Me} | 55 | n.d. | 260 | no deg. | n.d. | no deg. |

^{a} Half-lives (t_{1/2}) were determined by plotting the percent full-length oligonucleotide vs. time and fitting to the exponential decay function. For experimental conditions, see FIG. 3.
n.d., not determined;
no deg., no degradation observed within 24 h.

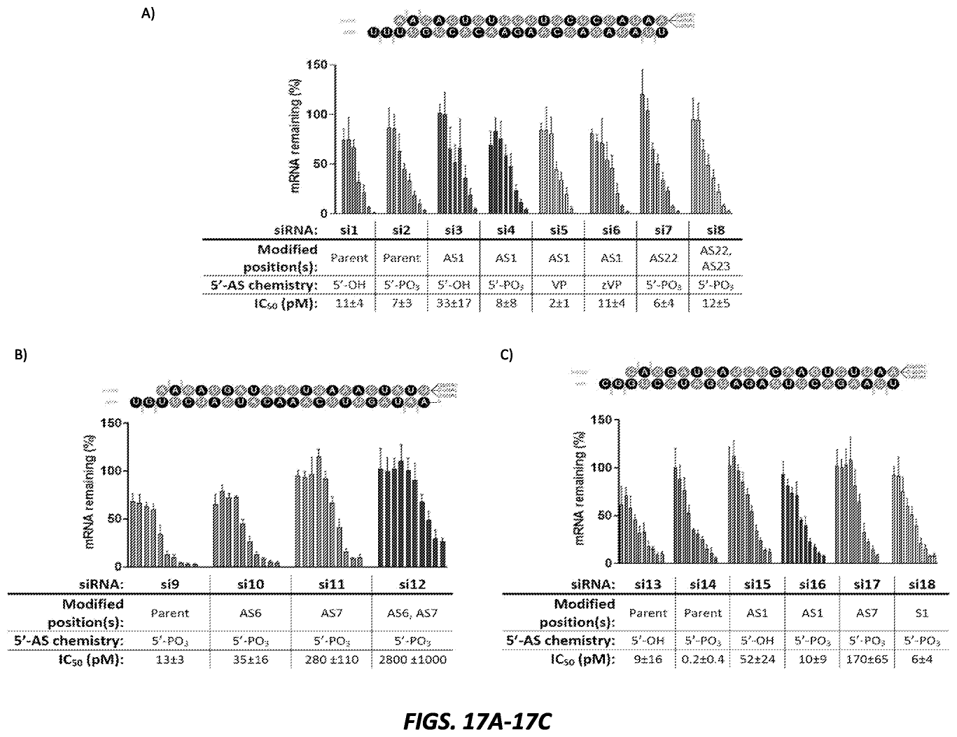

In vitro RNAi activity of siRNAs modified with 2-F/Me nucleotides. siRNAs with 2′-F/Me modifications were then evaluated for liver-specific gene silencing in cell culture by targeting four different mRNAs: Ttr, Pten, apo-B, and F7 (Table 8). Modified siRNAs targeting Ttr mRNA queried the effect of the 2′-F/Me modification at the 3′ and 5′ termini of the antisense strand (Table 8 and FIG. 17A). Evidence indicates that 5′ phosphorylation of the antisense strand is required f or efficient loading of siRNA into RISC and subsequent cleavage of target mRNA by Ago2.^{43, 51} Modification near the 5′ terminus can impede phosphorylation,⁵² so an siRNA with a 2′-F/Me modification at position 1 of the antisense strand was evaluated with either a pre-installed 5′ phosphate group or with a 5′-OH. The efficacy of the parent siRNA, which was modified with 2′-OMe, did not depend on the presence of a phosphate group (compare sit vs. si2). Modification at position 1 with U_{F/Me} in the absence of a pre-installed phosphate (si3) resulted in reduced activity, which was largely recovered when the strand was modified with a 5′ phosphate (si4) or with 5′-(E)-VP (si5). Surprisingly, 5′-(Z)—VP (si6) also enhanced RNAi activity. Single or double incorporation of U_{F/Me} was well tolerated at the 3′ terminus of the antisense strand, with similar activity as the parent (compare si7 and si8 vs. si2).

TABLE 8
In vitro efficacies of GalNAc-conjugated siRNAs with and without 2′-F/Me modifications

| siRNA | Target | Strand | Sequence (5′→3′) | IC₅₀ (pM) |
|---|---|---|---|---|
| si1 | Ttr | S | A_{F}•a•C_{F}aG_{F}uG_{F}uU_{F}C_{F}U_{F}uG_{F}CU_{F}cU_{F}aU_{F}aA_{F}* (SEQ ID | 11 ± 4 |
| | | | NO: 42) | |
| | | AS | u•U_{F}•aU_{F}aG_{F}aG_{F}cA_{F}agaA_{F}cA_{F}cU_{F}gU_{F}u•u•u (SEQ ID NO: | |
| | | | 47) | |
| si2 | Ttr | S | A_{F}•a•C_{F}aG_{F}uG_{F}uU_{F}C_{F}U_{F}uG_{F}cU_{F}cU_{F}aU_{F}aA_{F}* (SEQ ID | 7 ± 3 |
| | | | NO: 42) | |
| | | AS | Pu•U_{F}•aU_{F}aG_{F}aG_{F}cA_{F}agaA_{F}cA_{F}cU_{F}gU_{F}u•u•u (SEQ ID NO: | |
| | | | 47) | |
| si3 | Ttr | S | A_{F}•a•C_{F}aG_{F}uG_{F}uU_{F}C_{F}U_{F}uG_{F}cU_{F}cU_{F}aU_{F}aA_{F}* (SEQ ID | 33 ± 17 |
| | | | NO: 42) | |
| | | AS | U_{F/Me}•U_{F}•aU_{F}aG_{F}aG_{F}cA_{F}agaA_{F}cA_{F}cU_{F}gU_{F}u•u•u (SEQ ID | |
| | | | NO: 48) | |
| si4 | Ttr | S | A_{F}•a•C_{F}aG_{F}uG_{F}uU_{F}C_{F}U_{F}uG_{F}cU_{F}cU_{F}aU_{F}aA_{F}* (SEQ ID | 8 ± 8 |
| | | | NO: 42) | |
| | | AS | PU_{F/Me}•U_{F}•aU_{F}aG_{F}aG_{F}cA_{F}agaA_{F}cA_{F}cU_{F}gU_{F}u•u•u (SEQ ID | |
| | | | NO: 48) | |
| si5 | Ttr | S | A_{F}•a•C_{F}aG_{F}uG_{F}uU_{F}C_{F}U_{F}uG_{F}cU_{F}cU_{F}aU_{F}aA_{F}* (SEQ ID | 2 ± 1 |
| | | | NO: 42) | |
| | | AS | VP-U_{F}/Me•U_{F}•aU_{F}aG_{F}aG_{F}cA_{F}agaA_{F}cA_{F}cU_{F}gU_{F}u•u•u (SEQ | |
| | | | ID NO: 48) | |
| si6 | Ttr | S | A_{F}•a•C_{F}aG_{F}uG_{F}uU_{F}C_{F}U_{F}uG_{F}cU_{F}cU_{F}aU_{F}aA_{F}* (SEQ ID | 11 ± 4 |
| | | | NO: 42) | |
| | | AS | zVP-U_{F}/Me•U_{F}•aU_{F}aG_{F}aG_{F}cA_{F}agaA_{F}cA_{F}cU_{F}gU_{F}u•u•u (SEQ | |
| | | | ID NO: 48) | |
| si7 | Ttr | S | A_{F}•a•C_{F}aG_{F}uG_{F}uU_{F}C_{F}U_{F}uG_{F}cU_{F}cU_{F}aU_{F}aA_{F}* (SEQ ID | 6 ± 4 |
| | | | NO: 42) | |
| | | AS | Pu•U_{F}•aU_{F}aG_{F}aG_{F}cA_{F}agaA_{F}cA_{F}cU_{F}gU_{F}U•U_{F}/Me•u (SEQ ID | |
| | | | NO: 49) | |
| si8 | Ttr | S | A_{F}•a•C_{F}aG_{F}uG_{F}uU_{F}C_{F}U_{F}uG_{F}cU_{F}cU_{F}aU_{F}aA_{F}* (SEQ ID | 12 ± 5 |
| | | | NO: 42) | |
| | | AS | Pu•U_{F}•aU_{F}aG_{F}aG_{F}cA_{F}agaA_{F}cA_{F}cU_{F}gU_{F}u•U_{F}/Me•U_{F/Me} (SEQ | |
| | | | ID NO: 50) | |
| si9 | Pten | S | A_{F}•a•G_{F}aU_{F}gA_{F}uG_{F}U_{F}U_{F}uG_{F}aA_{F}aC_{F}uA_{F}uU_{F}* (SEQ ID | 13 ± 3 |
| | | | NO: 43) | |
| | | AS | Pa•A_{F}•uA_{F}gU_{F}uU_{F}cA_{F}aacA_{F}uC_{F}aU_{F}cU_{F}u•g•u (SEQ ID | |
| | | | NO: 51) | |
| si10 | Pten | S | A_{F}•a•G_{F}aU_{F}gA_{F}uG_{F}U_{F}U_{F}uG_{F}aA_{F}aC_{F}uA_{F}uU_{F}* (SEQ ID | 35 ± 16 |
| | | | NO: 43) | |
| | | AS | Pa•A_{F}•uA_{F}gU_{F/Me}uU_{F}cA_{F}aacA_{F}uC_{F}aU_{F}cU_{F}u•g•u (SEQ ID | |
| | | | NO: 52) | |
| si11 | Pten | S | A_{F}•a•G_{F}aU_{F}gA_{F}uG_{F}U_{F}U_{F}uG_{F}aA_{F}aC_{F}uA_{F}uU_{F}* (SEQ ID | 280 ± 110 |
| | | | NO: 43) | |
| | | AS | Pa•A_{F}•uA_{F}gU_{F}U_{F/Me}U_{F}cA_{F}aacA_{F}uC_{F}aU_{F}cU_{F}u•g•u (SEQ ID | |
| | | | NO: 53) | |
| si12 | Pten | S | A_{F}•a•G_{F}aU_{F}gA_{F}uG_{F}U_{F}U_{F}uG_{F}aA_{F}aC_{F}uA_{F}uU_{F}* (SEQ ID | 2800 ± 1000 |
| | | | NO: 43) | |
| | | AS | Pa•A_{F}•uA_{F}gU_{F/Me}U_{F/Me}U_{F}cA_{F}aacA_{F}uC_{F}aU_{F}cU_{F}u•g•u (SEQ | |
| | | | ID NO: 54) | |
| si13 | F7 | S | C_{F}•a•G_{F}gA_{F}uC_{F}aU_{F}C_{F}U_{F}cA_{F}aG_{F}uC_{F}uU_{F}aA_{F}* (SEQ ID | 9 ± 16 |
| | | | NO: 44) | |
| | | AS | u•U_{F}•aA_{F}gA_{F}cU_{F}uG_{F}agaU_{F}gA_{F}uC_{F}cU_{F}g•g•c (SEQ ID | |
| | | | NO: 55) | |
| si14 | F7 | S | C_{F}•a•G_{F}gA_{F}uC_{F}aU_{F}C_{F}U_{F}cA_{F}aG_{F}uC_{F}uU_{F}aA_{F}* (SEQ ID | 0.2 ± 0.4 |
| | | | NO: 44) | |
| | | AS | Pu•U_{F}•aA_{F}gA_{F}cU_{F}uG_{F}agaU_{F}gA_{F}uC_{F}cU_{F}g•g•c (SEQ ID | |
| | | | NO: 55) | |
| si15 | F7 | S | C_{F}•a•G_{F}gA_{F}uC_{F}aU_{F}C_{F}U_{F}cA_{F}aG_{F}uC_{F}uU_{F}aA_{F}* (SEQ ID | 52 ± 24 |
| | | | NO: 44) | |
| | | AS | U_{F/Me}•U_{F}•aA_{F}gA_{F}cU_{F}uG_{F}agaU_{F}gA_{F}uC_{F}cU_{F}g•g•c (SEQ ID | |
| | | | NO: 56) | |
| si16 | F7 | S | C_{F}•a•G_{F}gA_{F}uC_{F}aU_{F}C_{F}U_{F}cA_{F}aG_{F}uC_{F}uU_{F}aA_{F}* (SEQ ID | 10 ± 9 |
| | | | NO: 44) | |
| | | AS | PU_{F/Me}•U_{F}•aA_{F}gA_{F}cU_{F}uG_{F}agaU_{F}gA_{F}uC_{F}cU_{F}g•g•c (SEQ ID | |
| | | | NO: 56) | |
| si17 | F7 | S | C_{F}•a•G_{F}gA_{F}uC_{F}aU_{F}C_{F}U_{F}cA_{F}aG_{F}uC_{F}uU_{F}aA_{F}* (SEQ ID | 170 ± 65 |
| | | | NO: 44) | |
| | | AS | Pu•U_{F}•aA_{F}gA_{F}C_{F/Me}U_{F}uG_{F}agaU_{F}gA_{F}uC_{F}cU_{F}g•g•c (SEQ ID | |
| | | | NO: 57) | |
| si18 | F7 | S | C_{F/Me}•a•G_{F}gA_{F}uC_{F}aU_{F}C_{F}U_{F}cA_{F}aG_{F}uC_{F}uU_{F}aA_{F}* (SEQ ID | 6 ± 4 |
| | | | NO: 45) | |
| | | AS | Pu•U_{F}•aA_{F}gA_{Fc}U_{F}uG_{F}agaU_{F}gA_{F}uC_{F}cU_{F}g•g•c (SEQ ID | |
| | | | NO: 55) | |
| si19 | Ttr | S | 5′-a•g•uguuCFuUFGFCFucuauaaaca* (SEQ ID NO: 46) | 112 ± 6 |
| | | AS | 5′-u•G_{F}•uuuauagagcaA_{F}gAFacacu•g•u (SEQ ID NO: 58) | |
| si20 | Ttr | S | 5′-a•g•uguuCFuUFGFCFucuauaaaca* (SEQ ID NO: 46) | 60 ± 6 |
| | | AS | u•G_{F}•uuuaUF/MeagagcaAFgAFacacu•g•u (SEQ ID | |
| | | | NO: 59) | |

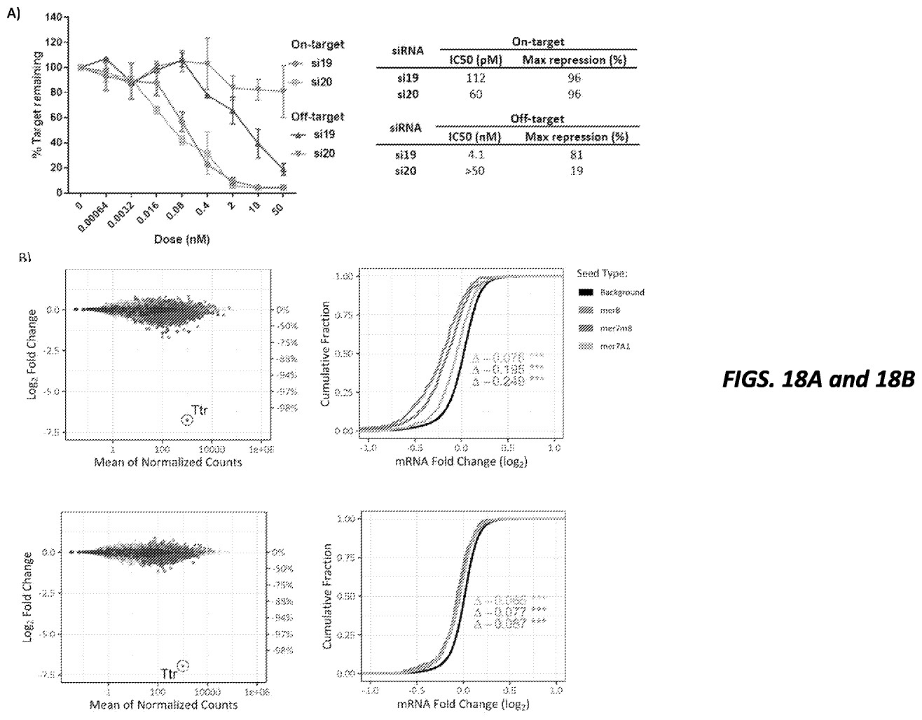

^{a}In vitro potency of fully 2′-modified siRNA targeting (A) Ttr, (B) Pten, and (C) F7. Primary mouse hepatocytes were transfected with 10 nM siRNA and 6-fold serial dilutions for Ttr of 100 nM siRNA and 5-fold serial dilutions for Pten and F7. Target mRNA was quantified using RT-qPCR after 24 h. For fitted dose response curves, see FIGS. S2-S4. 2′-F and 2′-OMe nucleotides are represented as U_{F} or u, respectively. PS linkage = · , VP = 5′-(E)-vinyl phosphonate, and zVP = 5′-(Z)-vinyl phosphonate. Error bars show standard deviations from mean.
^{b}Analyzed via dual-luciferase assay. For experimental conditions, see FIGS. 18 A and 18B.

Tolerance for modification in the seed region was evaluated for siRNA targeting Pten with single incorporations at positions 6 or 7 or incorporation at both positions (si10, si11, and si12, respectively; Table 8 and FIG. 17B). Modification at position 6 was well tolerated; si10 had activity similar to that of the parent siRNA (si9). Modification at position 7 reduced activity from an IC₅₀ of about 13 pm for the parent to 280 pM. This could be due to the unique structural requirements of Ago2 in this region that necessitates a kink in the siRNA structure.⁵³ When both positions were modified, there was considerable loss of potency (IC₅₀=2800 pM).

The siRNA targeting F7 has several positions that can be modified with 2′-F/Me pyrimidine nucleotides (Table 8 and FIG. 17C). The siRNA containing a pre-installed 5′ phosphate on the antisense strand had slightly improved activity compared to the parent construct (compare si14 to si13). Similarly, the siRNA with U_{F/Me} at position 1 of the antisense strand with a pre-installed 5′ phosphate had higher potency than the siRNA with this modification and a 5′-OH (compare si16 to si15). This suggests that 2′-F/Me modification at position 1 of the antisense strand is tolerated by RISC, but there is some impairment of endogenous phosphorylation. Modification at position 7 of the antisense strand had considerably lower activity than the parent (compare si17 to si13). In general, single of 2′-F/Me pyrimidine nucleotides in the seed region, modification of the 3′ overhang, and position 1 of the antisense in conjugation with VP were well tolerated, although there may be sequence- and target-dependent effects not revealed by this analysis.

Off-target effects of siRNAs modified with 2′-F/Me. Seed-mediated off-target activity contributes to hepatoxicity of siRNAs in rats, and one way to mitigate this off-target activity is to incorporate thermally destabilizing nucleotides such as GNA in the seed region.⁴⁸ To be effective, such thermally destabilizing modifications must maintain on-target activity while reducing off-target activity. As the 2′-F/Me nucleotide is well tolerated in the seed region, we measured the effect of 2′-F/Me on off-target activity using a luciferase reporter assay where four tandem seed matches to the siRNA are present in the luciferase 3′-untranslated region.⁵⁵⁻⁵⁷ The siRNAs used in this assay were designed to target Ttr. si19 is the parent and si20 has a 2′-F/Me at position 7 in the antisense strand. Consistent with in vitro activity, on-target activity of siRNA with 2′-F/Me in the seed region was similar to the activity of the parent; however, off-target activity was mitigated by incorporating a single 2′-F/Me nucleotide at position 7 in the antisense strand (FIG. 18A), consistent with its thermally destabilizing property.

To further evaluate the impact of 2′-F/Me on off-target activity, we used RNA sequencing to measure the level of transcriptional dysregulation upon siRNA treatment. Transfection of the parent siRNA targeting Ttr (si19) at 50 nM concentration into primary rat hepatocytes resulted in strong up- and down-regulation of hundreds of transcripts at 48 h, many of which contained a canonical seed-match (FIG. 18B). Consistent with the luciferase reporter assay, incorporation of 2′-F/Me at position 7 (si20) in the seed region of the antisense strand resulted in considerably less transcriptional dysregulation than observed with the parent siRNA. These results suggest that the 2′-F/Me modification mitigates off-target activity in a manner similar to GNA.

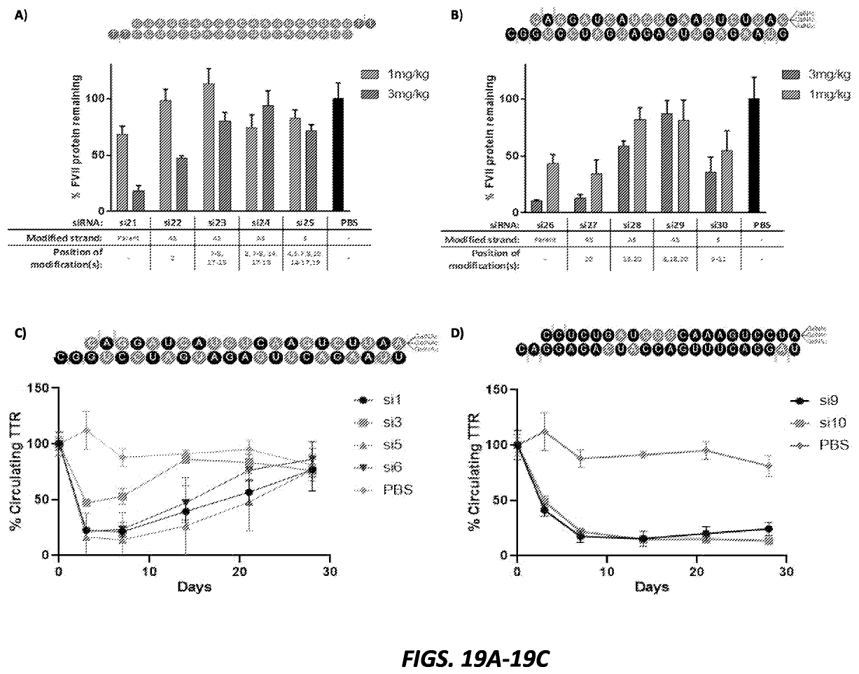

In vivo activity of 2′-F/Me-modified siRNA. Encouraged by the in vitro activity of 2′-F/Me-modified siRNAs, the inventors evaluated these siRNAs in mice using two different delivery platforms. First, siRNAs targeting F7 consisting of a 21-mer RNA duplex with two thymidine overhangs and terminal PS linkages were formulated in lipid nanoparticles optimized for hepatic delivery.⁵⁸ Mice (C57BL/6) were dosed with siRNA at either 1 or 3 mg/kg, and at 48 h post administration the serum F7 levels were quantified. The parent strand si21 had an ED₈₀ of 3 mg/kg as did the siRNA with a single 2′-F/Me modification at position 2 of the antisense strand (si22). The siRNAs with multiple 2′-F/Me modifications (si23, si24, and si25) had dramatically lower EDsos (FIG. 19A). These data suggest that there is either positional dependence or a limit on the percentage of 2′-F/Me modifications that are tolerated. As LNP formulations cannot be used for in vitro pharmacology evaluation, we did not carry out the in vitro assays with this formulation.

To assess the positional dependence, F7-targeted siRNAs conjugated to GalNAc, which can be administered subcutaneously were analyzed. The optimized template for GalNAc-conjugated siRNAs has fewer 2′-F/Me modifications than do those used with LNP formulations.⁵⁸ These siRNA are 21:23-mer asymmetric duplexes comprised of 2′-OMe and 2′-F nucleotides, terminal phosphorothioates, and a 3′-conjugated GalNAc ligand on the sense strand. Data for siRNAs with 2′-F/Me nucleotides at various positions are shown in FIGS. 19B-19D. Treatment with the parent siRNA (si26) at a dose of 1 mg/kg resulted in 60% reduction in circulating F7 protein, when assayed 10 days post administration. The siRNA with a 2′-F/Me at position 20 of the antisense strand (si27) had activity similar to that of the parent. However, when two 2′-F/Me nucleotides were incorporated at positions 18 and 20 of the antisense strand (si28), there was only 40% reduction in F7 when dosed at 3 mg/kg, and the siRNA with three 2′-F/Me modifications in the antisense strand was even less potent (si29). Multiple 2′-F/Me modifications were not well tolerated on the sense strand; si30 with three modifications at positions 9-11 lost activity compared to the parent. Optimal activity requires sense strand cleavage in this region,^{43,45,47} and this cleavage is likely inhibited by the consecutive placement of 2′-F/Me in these positions.

To further understand the impact of modification on position 1 of the antisense strand, which interacts with Ago2, in vivo potency of siRNAs modified at this position were assessed to day 28 after 1 mg/kg subcutaneous doses of Ttr-targeted siRNAs (FIG. 19C). Inventors have done extensive structure-activity relationship studies with this siRNA.^{43, 45, 47} As expected, the siRNA with 2′-F/Me at position 1 (si3) was less active than the parent, possibly because it does not serve as a kinase substrate. In support of this hypothesis, the siRNA with (E)-VP (si5) had slightly improved potency relative to the parent sit. (Z)—VP (si6) had slightly reduced activity. Thus, 5′-modification with (E)-VP and 2′-F/Me at position 1 of the antisense siRNA in combination with other optimized modifications results in an siRNA with excellent and improved in vivo efficacy compared to the parent (sit). Interestingly, the siRNA modified with both 2′-F/Me and (Z)—VP (si6) had silencing activity only slightly less than the parent (sit). This is the first observation of activity of a (Z)—VP-modified siRNA.⁴¹⁻⁴⁷ Steric impact of the 2′-F/Me may result in a more favorable conformation than when the (Z)—VP is used in conjunction with a 2′-OMe or a 2′-F substituent at position 1.

To assess the potential of the 2′-F/Me to mitigate off-target effects in the mouse model, we analyzed a GalNAc-conjugated siRNA with a 2′-F/Me at position 6 of the antisense strand (si10). This is an off-target prone sequence.^{48, 59} The 2′-F/Me-modified siRNA had potency equivalent to the parent siRNA (si9) (FIG. 19D). Thus, modification with 2′-F/Me is a promising approach for mitigating seed-mediated off-target effects without compromising on-target potency.

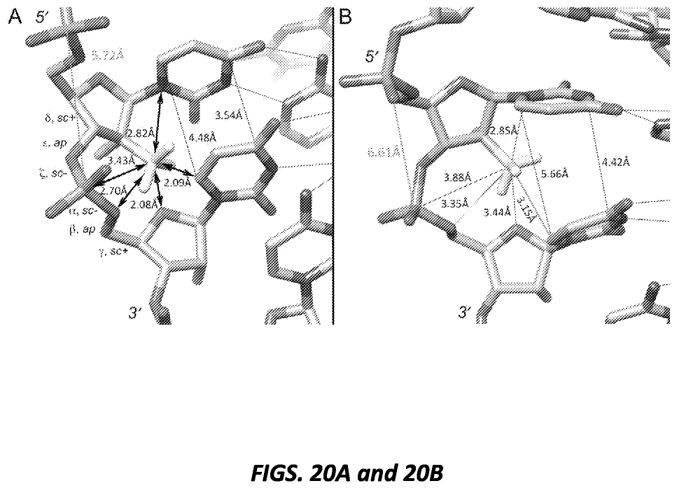

Structural consequences of 2′-F/Me in siRNA. Crystal structures of β-D-2′-deoxy-2′-α-fluoro-2′-β-C-methylcytidine⁶⁰ and the corresponding uridine nucleotide prodrug PSI-7977⁶¹ revealed that the modified sugar adopts a C3′-endo pucker. In an A-form RNA oligonucleotide with standard sc−/ap/sc+/sc+/ap/sc− backbone torsion angles (to) and riboses in the C3′-endo conformation, the pseudo-equatorial orientation of the 2′-β-C-methyl group results in short contacts to base, sugar, and phosphate atoms of the 3′-adjacent nucleotide (FIG. 20A). Some of these barely exceed the van der Waals radius of a methyl group (2 Å), which likely explain the observed destabilizations of RNA duplexes with 2′-F/Me U or C on one strand (Table 4). Avoiding these short contacts requires conformational changes that probably entail adjustments in the backbone and glycosidic torsion angles and likely result in local base unstacking. Indeed, energy minimization of the model duplex containing a 2′-F/Me residue using a standard molecular mechanics approach (Amber 14ff, UCSF Chimera)⁶² resulted in acceptable distances between methyl group and atoms from the 3′-adjacent nucleotide. Avoiding these inter-nucleotide steric conflicts resulted in loss of stacking between the uracil bases (FIG. 20B). Further, these changes were accompanied by a stretching of the sugar-phosphate backbone, manifested in an increased intrastrand phosphate-phosphate distance from 5.7 Å in the native duplex to 6.6 Å in the duplex with a modified residue (FIG. 20B). The altered spacing between phosphates and associated differences in the local electrostatic surface potential probably contribute to the improved resistance to nuclease degradation afforded by 2′-F/Me relative to 2′-F nucleotides (Tables 6 and 7).

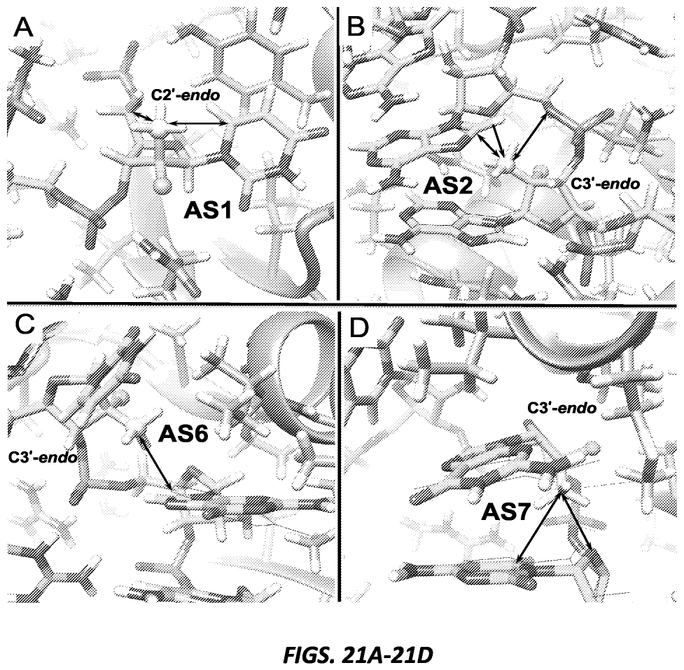

To gain a better understanding of the structural origins of the observed activities of siRNAs with 2′-F/Me nucleotides incorporated at positions 1, 2, or 6 in the antisense strand, we turned to the crystal structure of human Ago2 in complex with miR-20a (PDB ID 4F3T).⁵³ The modified nucleotide is tolerated quite well at positions 1 and 6 but results in a marked loss in activity at the position 2 (FIGS. 19A-19D). At the 5′-terminal position of the antisense strand, the phosphate group and base are held tightly in place by multiple interactions including salt bridges involving the phosphate group (FIG. 21A). The ribose of the nucleotide at position 1 of the antisense strand adopts a C2′-endo B-DNA like pucker, the antisense strand makes a sharp turn between positions 1 and 2, and the tight grip of the protein continues at positions 2 and 3. We used UCSF Chimera to add the methyl group in the 2′-β-C orientation at sugars of positions 1, 2, and 6 and replaced the native 2′-hydroxyl group with fluorine. The modified complex was then relaxed using molecular mechanics (Amber 14ff) as implemented in UCSF Chimera.⁶² Because of multiple interactions between protein and each nucleotide in the complex, the computational approach used does not result in significant movements of atoms triggered by short contacts arising from the additional methyl group.

At position 1, the pseudo-axial orientation of the 2′-β-C-methyl group leads to two relatively tight intra-nucleoside 1 . . . 5 contacts to C5′ and Ni (3.5 Å and 3.1 Å, respectively) in the refined model (FIG. 21A). Unless the sugar is flipped into a different pucker, these are difficult to avoid. However, the mold provided by Ago2 likely precludes substantial conformational changes. There are no other conflicts as a consequence of the additional methyl group, and we conclude that a 2′-F/Me-modified nucleotide at position 1 of the antisense strand is quite well accommodated by the Ago2 binding site, consistent with the high activity of siRNA with this modification (FIGS. 17A-17C). Conversely, incorporation of a modified nucleotide at position 2 results in clashes between the methyl group and atoms of the base, sugar, and phosphate moieties from the 3′-adjacent nucleotide (FIG. 21B). Expansion of the sugar-phosphate backbone between the positions 2 and 3 interferes with binding by Ago2, thus providing a structural rationalization for why the 2′-F/Me nucleotide is poorly tolerated at position 2 of the antisense strand. Finally, a strong kink between positions 6 and 7, as seen in the crystal structure of the Ago2 complex,⁵³ provides generous space for accommodation of a 2′-F/Me-modified nucleotide at position 6. A somewhat short contact in the initially built model between methyl group and the C8-H position of the guanidine at position 7 is mitigated by a slight rotation of the base around the glycosidic bond with a concomitant increase of the Me C8 distance to approximately 3.4 Å in the refined model (FIG. 21C). The computational model thus makes clear why the 2′-F/Me nucleotide at position 6 does not impair RNAi activity. The 2′-F/Me nucleotide incorporated at position 7 also resulted in a somewhat tight spacing between 2′-methyl group and C6 of the base of the 3′-adjacent nucleotide (FIG. 21D). In the refined model, the distance is 3.5 Å as the two bases are slightly pushed apart. However, a close contact between 2′-methyl and the 4′-oxygen of position 8 remains (2.8 Å). This clash is not seen between the corresponding atoms of positions 6 and 7 due to the kink between these residues (FIG. 21C). An interesting consequence of the modification at position 7 is a potentially favorable contact (3.6 Å) between the 2′-methyl moiety and the methyl group of the side chain of Met-364 of Ago2 (FIG. 21D).

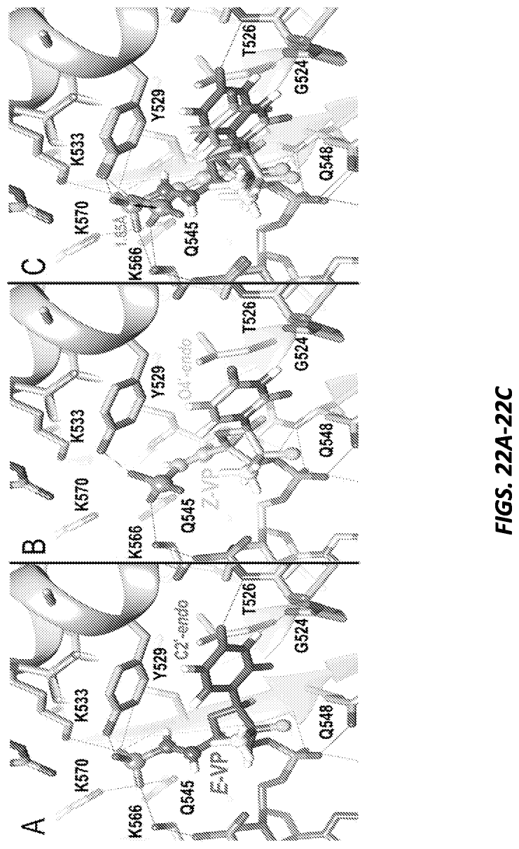

To model the interactions of E-VP and Z—VP 2′-F/Me uridines at position 1 of the antisense strand, we started from the crystal structure of human Ago2 in complex with miR-20a.²⁴ In this structure, the E-VP has an unusual C2′-endo (South) sugar pucker with a pseudo-axial 2′-—C-Me substituent (FIG. 22A). In the crystal structure with 5′-phosphorylated miRNA, the P—O5′—C5′—C4′ torsion angle is nearly antiperiplanar (ap) and the E-VP moiety is therefore accommodated with virtually no change in the orientation of the phosphate relative to the parent structure. The Z—VP model features an O4′-endo (East) sugar pucker and neither the 2′-F nor the 2′-Me substituent is in a pseudo-axial orientation (FIG. 22B). The phosphate engages in a salt bridge with Lys-566 and forms hydrogen bonds to Tyr-529 and Gln-545, and an overlay of the E-VP and Z—VP models shows that the phosphates are only 1.85 Å apart (FIG. 22C). The Z—VP phosphate does not reach quite as deep into the binding pocket as does the phosphate of the E-VP moiety; however, the Z—VP phosphate is able to establish favorable electrostatic interactions. A further slight change between the E-VP and Z—VP models concerns the orientation of the uracil base vis-á-vis the Tyr-529 side chains: In the Z—VP model the base is partially unstacked with a vertical shift of approximately 1 Å relative to the E-VP uracil ring (FIG. 22C). Overall, the model of the Z—VP 2′-F/Me uridine-modified strand is consistent with the observed in vivo potency of this modification (FIGS. 17A-17C), which is in contrast with the poor activity of the Z-isomer of VP in the context of 2′-OMe or 2′-F chemistries.⁴¹⁻⁴⁷

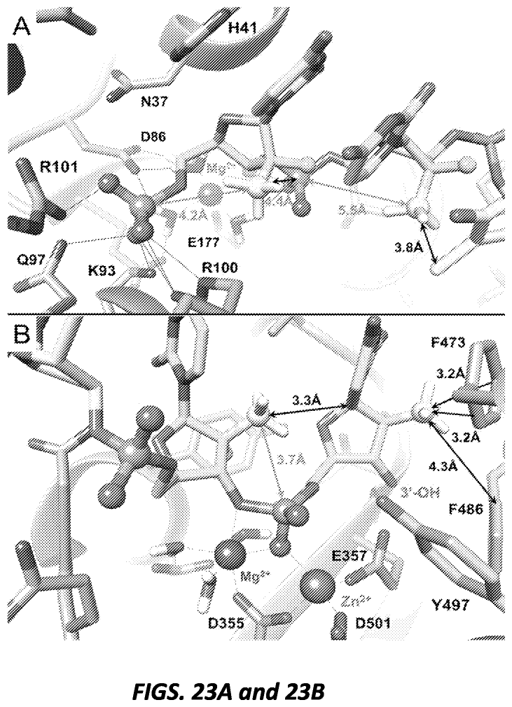

The molecular reasons for protection against the attack by exonucleases afforded by the 2′-F/Me-modification (FIGS. 25A-25D and 26A-26D) were assessed using models of complexes based on the crystal structures of Drosophila melanogaster 5′-3′ exoribonuclease Xrn1 bound to a 5′-phosphorylated trinucleotide P-d(TTT) (PDB ID 2Y35)⁶⁴ and Escherichia coli DNA polymerase I Klenow fragment 3′-5′ exonuclease bound to a DNA tetramer with a single Sp-PS moiety 3′-d(TPsTTT)-5′ (PDB ID 1KSP).⁶⁵ In both cases, terminal and penultimate dT were replaced by 2′-F/Me-U. The sugar puckers in the parent crystal structures were not altered: Sugars adopt a C2′-endo conformation at the Xrn1 active site (FIG. 23A) and a C3′-endo conformation at the active site of DNA polymerase I Klenow exonuclease (FIG. 23B). In the active site of Xrn1, the methyl group of the 5′-terminal 2′-F/Me-U sits quite close to the 5′ phosphate and the first bridging phosphate (4.2 Å and 4.4 Å, respectively, below the sum of the van der Waals radii, 4.8 Å). The methyl group of the 3′-terminal 2′-F/Me-U at the active site of Klenow 3′-exonuclease is a tight space limited by two Phe residues that form the floor of the active site (the closest distance is 3.2 Å; below the sum of van der Waals radii, 3.5 Å). These analyses rationalize the better protection of the 2′-F/Me modification relative to 2′-F alone.

A structural rationalization for poor mitochondrial polymerase gamma to incorporate 2′-F/Me nucleotides. We previously showed that native NTPs (rCTP, rUTP, dCTP, and dTTP) are efficiently incorporated by the mitochondrial DNA and RNA polymerases POLG and POLRMT, respectively.^{40, 66} Although 2′-F monomers are incorporated at high concentrations by POLRMT, the 2′-F/Me-modified nucleotide analogs are not substrates for either mitochondrial polymerase.¹⁵⁻¹⁷ The inability of POLG to incorporate 2′-F/Me-modified residues can be readily explained with an unfavorable interaction between the 2′-methyl moiety and the ‘gatekeeper’ residue Tyr-951 at the active site. Thus, when a 2′-F/Me CMP from a refined duplex structure (C3′-endo pucker) is superimposed on the incoming dCTP in the crystal structure of the ternary POLG complex⁶⁷ (PDB ID 4ZTZ, the gem methyl group points directly into the ring of that tyrosine thereby creating a clash (FIG. 32).

### Conclusions

Sofosbuvir is a ribonucleotide analog inhibitor that proved to be of immense value in treatment of HCV due to its highly effective 5′ phosphate prodrug to function as the inhibitor for the viral RNA polymerase and excellent safety profile. Inspired by the pharmacology of this drug, we evaluated the core nucleoside backbone in oligonucleotide-based RNAi therapeutics. For this purpose, we demonstrated facile synthesis of 2′-F/Me phosphoramidites and the corresponding uridine (E)-VP and (Z)—VP phosphoramidites. These compounds were incorporated into oligonucleotides and therapeutic siRNAs. These modifications are thermally destabilizing of both RNA:RNA and RNA:DNA duplexes, and likely perturb the duplex geometry on the 3′ side of the modification. We speculate that this perturbation results in the enhanced stabilization toward 5′- and 3′-exonuclease-mediated degradation provided by terminal 2′-F/Me modification. The steric clash due to 2′-β-C-methyl group appears to account for the destabilization of interactions with complementary oligonucleotides and with the nucleases.

In vivo gene-silencing activity analyses of siRNAs modified with 2′-F/Me revealed positional dependence of RNAi activity. When siRNAs were modified with either several 2′-F/Me residues or when only position 2 of the antisense strand was modified, potency was considerably lower than parent siRNAs when delivered into cells as either an LNP formulation or a GalNAc conjugate. The loss of activity due to substitution at position 2 is not surprising as only 2′-H, 2′-OH, and 2′-F are tolerated at this positions; even 2′-OMe impairs silencing.²¹ Each substitution of 2′-F/Me leads to a kinks in the antisense strand, so multiple 2′-F/Me substitutions lead to weak interactions Ago2 and less stable duplex formation with target mRNA, explaining the loss of potency due to multiple substitutions. Interestingly, the 2′-F/Me modification was well tolerated at position 1 of the antisense strand in conjugation with a (E)-5′-VP and in conjugation with the (Z)—VP isomer. This was interesting because the Z—VP is not tolerated when position 1 of the antisense strand is a natural ribonucleotide, 2′-F, or 2′-OMe. In conjunction with the 2′-F/Me, the Z—VP phosphate is able to establish favorable electrostatic interactions due to the distortion provided by the 2′-β-C-methyl group. The 2′-F/Me modification in the seed region, at positions 6 or 7, did not interfere with potency. Importantly, 2′-F/Me nucleotides at these positions mitigated off-target effects in a manner similar to other thermodynamically destabilizing modifications like GNA⁴⁸. A strong kink between positions 6 and 7, as seen in the crystal structure of the Ago2 complex,⁵³ provides generous space for accommodation of a 2′-F/Me-modified nucleotide at positions 6 and 7.

Finally, as 2′-F/Me nucleotides are poor substrate for polymerases,⁶⁸ this modification should not induce mitochondrial toxicity, and thus 2′-F/Me nucleotides represent a promising tool for RNAi applications to potentially improve site-specific potency, duration, and safety. Encouraged by our results, we plan to evaluate this modification systematically at each position of siRNAs targeting various mRNAs. This necessitates the synthesis of the purine 2′-F/Me nucleoside⁶⁹⁻⁷¹ phosphoramidites and solid supports, which is ongoing.

### REFERENCES

1. Elbashir, S. M.; Harborth, J.; Lendeckel, W.; Yalcin, A.; Weber, K.; Tuschl, T., Duplexes of 21-nucleotide RNAs mediate RNA interference in cultured mammalian cells. Nature 2001, 411 (6836), 494-498.
2. Wang, H. W.; Noland, C.; Siridechadilok, B.; Taylor, D. W.; Ma, E.; Felderer, K.; Doudna, J. A.; Nogales, E., Structural insights into RNA processing by the human RISC-loading complex. Nat Struct Mol Biol 2009, 16 (11), 1148-53.
3. Meister, G.; Landthaler, M.; Patkaniowska, A.; Dorsett, Y.; Teng, G.; Tuschl, T., Human Argonaute2 mediates RNA cleavage targeted by miRNAs and siRNAs. Mol Cell 2004, 15 (2), 185-97.
4. Liu, J.; Carmell, M. A.; Rivas, F. V.; Marsden, C. G.; Thomson, J. M.; Song, J. J.; Hammond, S. M.; Joshua-Tor, L.; Hannon, G. J., Argonaute2 is the catalytic engine of mammalian RNAi Science 2004, 305 (5689), 1437-41.
5. Hammond, S. M.; Aartsma-Rus, A.; Alves, S.; Borgos, S. E.; Buijsen, R. A. M.; Collin, R. W. J.; Covello, G.; Denti, M. A.; Desviat, L. R.; Echevarria, L.; Foged, C.; Gaina, G.; Garanto, A.; Goyenvalle, A. T.; Guzowska, M.; Holodnuka, I.; Jones, D. R.; Krause, S.; Lehto, T.; Montolio, M.; Van Roon-Mom, W.; Arechavala-Gomeza, V., Delivery of oligonucleotide-based therapeutics: challenges and opportunities. EMBO Mol. Med. 2021, 13(4), e13243.
6. Xiong, H.; Veedu, R. N.; Diermeier, S. D., Recent Advances in Oligonucleotide Therapeutics in Oncology. International Journal of Molecular Sciences 2021, 22 (7), 3295.
7. Smith, C. I. E.; Zain, R., Therapeutic Oligonucleotides: State of the Art. Annual Review of Pharmacology and Toxicology 2019, 59 (1), 605-630.
8. Shen, X.; Corey, D. R., Chemistry, mechanism and clinical status of antisense oligonucleotides and duplex RNAs. Nucleic acids research 2017, 46 (4), 1584-1600.
9. Stein, C. A.; Castanotto, D., FDA-Approved Oligonucleotide Therapies in 2017. Mol. Ther. 2017, 25 (5), 1069-1075.
10. Crooke, S. T.; Witztum, J. L.; Bennett, C. F.; Baker, B. F., RNA-Targeted Therapeutics. Cell Metabolism 2018, 27 (4), 714-739.
11. Setten, R. L.; Rossi, J. J.; Han, S.-p., The current state and future directions of RNAi-based therapeutics. Nat. Rev. Drug Discov. 2019, 18 (6), 421-446.
12. Roberts, T. C.; Langer, R.; Wood, M. J. A., Advances in oligonucleotide drug delivery. Nat. Rev. Drug Discov. 2020, 19 (10), 673-694.
13. Akinc, A.; Maier, M. A.; Manoharan, M.; Fitzgerald, K.; Jayaraman, M.; Barros, S.; Ansell, S.; Du, X.; Hope, M. J.; Madden, T. D.; Mui, B. L.; Semple, S. C.; Tam, Y. K.; Ciufolini, M.; Witzigmann, D.; Kulkarni, J. A.; van der Meel, R.; Cullis, P. R., The Onpattro story and the clinical translation of nanomedicines containing nucleic acid-based drugs. Nat Nanotechnol 2019, 14 (12), 1084-1087.
14. Balwani, M.; Sardh, E.; Ventura, P.; Peiró, P. A.; Rees, D. C.; Stolzel, U.; Bissell, D. M.; Bonkovsky, H. L.; Windyga, J.; Anderson, K. E.; Parker, C.; Silver, S. M.; Keel, S. B.; Wang, J.-D.; Stein, P. E.; Harper, P.; Vassiliou, D.; Wang, B.; Phillips, J.; Ivanova, A.; Langendonk, J. G.; Kauppinen, R.; Minder, E.; Horie, Y.; Penz, C.; Chen, J.; Liu, S.; Ko, J. J.; Sweetser, M. T.; Garg, P.; Vaishnaw, A.; Kim, J. B.; Simon, A. R.; Gouya, L., Phase 3 Trial of RNAi Therapeutic Givosiran for Acute Intermittent Porphyria. New England Journal of Medicine 2020, 382 (24), 2289-2301.
15. Chan, A.; Liebow, A.; Yasuda, M.; Gan, L.; Racie, T.; Maier, M.; Kuchimanchi, S.; Foster, D.; Milstein, S.; Charisse, K.; Sehgal, A.; Manoharan, M.; Meyers, R.; Fitzgerald, K.; Simon, A.; Desnick, R. J.; Querbes, W., Preclinical Development of a Subcutaneous ALAS1 RNAi Therapeutic for Treatment of Hepatic Porphyrias Using Circulating RNA Quantification. Mol. Ther. Nucleic Acids 2015, 4, e263.
16. Liebow, A.; Li, X.; Racie, T.; Hettinger, J.; Bettencourt, B. R.; Najafian, N.; Haslett, P.; Fitzgerald, K.; Holmes, R. P.; Erbe, D.; Querbes, W.; Knight, J., An Investigational RNAi Therapeutic Targeting Glycolate Oxidase Reduces Oxalate Production in Models of Primary Hyperoxaluria. J Am. Soc. Nephrol. 2017, 28 (2), 494-503.
17. Raal, F. J.; Kallend, D.; Ray, K. K.; Turner, T.; Koenig, W.; Wright, R. S.; Wijngaard, P. L. J.; Curcio, D.; Jaros, M. J.; Leiter, L. A.; Kastelein, J. J. P., Inclisiran for the Treatment of Heterozygous Familial Hypercholesterolemia. New England Journal of Medicine 2020, 382 (16), 1520-1530.
18. Ray, K. K.; Wright, R. S.; Kallend, D.; Koenig, W.; Leiter, L. A.; Raal, F. J.; Bisch, J. A.; Richardson, T.; Jaros, M.; Wijngaard, P. L. J.; Kastelein, J. J. P., Two Phase 3 Trials of Inclisiran in Patients with Elevated LDL Cholesterol. New England Journal of Medicine 2020, 382 (16), 1507-1519.
19. Fitzgerald, K.; White, S.; Borodovsky, A.; Bettencourt, B. R.; Strahs, A.; Clausen, V.; Wijngaard, P.; Horton, J. D.; Taubel, J.; Brooks, A.; Fernando, C.; Kauffman, R. S.; Kallend, D.; Vaishnaw, A.; Simon, A., A Highly Durable RNAi Therapeutic Inhibitor of PCSK9. N. Engl. J. Med. 2016, 376 (1), 41-51.
20. Nair, J. K.; Willoughby, J. L.; Chan, A.; Charisse, K.; Alam, M. R.; Wang, Q.; Hoekstra, M.; Kandasamy, P.; Kel'in, A. V.; Milstein, S.; Taneja, N.; O'Shea, J.; Shaikh, S.; Zhang, L.; van der Sluis, R. J.; Jung, M. E.; Akinc, A.; Hutabarat, R.; Kuchimanchi, S.; Fitzgerald, K.; Zimmermann, T.; van Berkel, T. J.; Maier, M. A.; Rajeev, K. G.; Manoharan, M., Multivalent N-acetylgalactosamine-conjugated siRNA localizes in hepatocytes and elicits robust RNAi-mediated gene silencing. J Am Chem Soc 2014, 136 (49), 16958-61.
21. Egli, M.; Manoharan, M., Re-Engineering RNA Molecules into Therapeutic Agents. Acc Chem Res 2019, 52 (4), 1036-1047.
22. Jonathan, O. S.; Christopher, S. T.; Rajat, D.; Babu, I. R.; Klaus, C.; Muthiah, M.; Martin, A. M.; Ivan, Z., An efficient deprotection method for 5′-O,O-bis(pivaloyloxymethyl)-(E)-vinylphosphonate containing oligonucleotides. Tetrahedron 2018, 74 (42), 6182-6186.
23. Khvorova, A.; Watts, J. K., The chemical evolution of oligonucleotide therapies of clinical utility. Nat Biotechnol 2017, 35 (3), 238-248.
24. Pallan, P. S.; Greene, E. M.; Jicman, P. A.; Pandey, R. K.; Manoharan, M.; Rozners, E.; Egli, M., Unexpected origins of the enhanced pairing affinity of 2′-fluoro-modified RNA. Nucleic Acids Res 2011, 39 (8), 3482-95.
25. Kawasaki, A. M.; Casper, M. D.; Freier, S. M.; Lesnik, E. A.; Zounes, M. C.; Cummins, L. L.; Gonzalez, C.; Cook, P. D., Uniformly modified 2′-deoxy-2′-fluoro phosphorothioate oligonucleotides as nuclease-resistant antisense compounds with high affinity and specificity for RNA targets. J Med Chem 1993, 36 (7), 831-41.
26. Chantal, S.; Marc-Olivier, B.; Alain; Karl-Heinz, A., The effects of 2′- and 3′-alkyl substituents on oligonucleotide hybridization and stability. Bioorganic & Medicinal Chemistry Letters 1994, 4 (16), 1969-1974.
27. Daniel, O. C.; Mariana, G.; Philippe, J. N.; Adolfo, M. I., Synthesis and properties of (2′S)- and (2′R)-2′-deoxy-2′-C-methyl oligonucleotides. Tetrahedron 2001, 57 (36), 7613-7621.
28. Robaldo, L.; Izzo, F.; Dellafiore, M.; Proietti, C.; Elizalde, P. V.; Montserrat, J. M.; Iribarren, A. M., Influence of conformationally restricted pyrimidines on the activity of 10-23 DNAzymes. Bioorganic & Medicinal Chemistry 2012, 20 (8), 2581-2586.
29. Koole, L. H.; Wu, J. C.; Neidle, S.; Chattopadhyaya, J., Structural properties of four isomeric C2′/C3′ modified uridines. Journal of the American Chemical Society 1992, 114 (7), 2687-2696.
30. Gamamik, A.; Arnold, J. J.; Sharma, S. D.; Feng, J. Y.; Ray, A. S.; Smidansky, E. D.; Kireeva, M. L.; Cho, A.; Perry, J.; Vela, J. E.; Park, Y.; Xu, Y.; Tian, Y.; Babusis, D.; Barauskus, O.; Peterson, B. R.; Gnatt, A.; Kashlev, M.; Zhong, W.; Cameron, C. E., Sensitivity of Mitochondrial Transcription and Resistance of RNA Polymerase II Dependent Nuclear Transcription to Antiviral Ribonucleosides. PLoS Pathogens 2012, 8 (11), e1003030.
31. Reddy, P. G.; Bao, D.; Chang, W.; Chun, B.-K.; Du, J.; Nagarathnam, D.; Rachakonda, S.; Ross, B. S.; Zhang, H.-R.; Bansal, S.; Espiritu, C. L.; Keilman, M.; Lam, A. M.; Niu, C.; Steuer, H. M.; Furman, P. A.; Otto, M. J.; Sofia, M. J., 2′-Deoxy-2′-α-fluoro-2′-β-C-methyl 3′,5′-cyclic phosphate nucleotide prodrug analogs as inhibitors of HCV NS5B polymerase: Discovery of PSI-352938. Bioorganic & Medicinal Chemistry Letters 2010, 20 (24), 7376-7380.
32. Zhou, S.; Mahmoud, S.; Liu, P.; Zhou, L.; Ehteshami, M.; Bassit, L.; Tao, S.; Domaoal, R. A.; Sari, O.; Schutter, C. D.; Amiralaei, S.; Khalil, A.; Ollinger Russell, O.; McBrayer, T.; Whitaker, T.; Abou-Taleb, N.; Amblard, F.; Coats, S. J.; Schinazi, R. F., 2′-Chloro,2′-fluoro Ribonucleotide Prodrugs with Potent Pan-genotypic Activity against Hepatitis C Virus Replication in Culture. J. Med. Chem. 2017, 60 (13), 5424-5437.
33. Wang, P.; Chun, B. K.; Rachakonda, S.; Du, J.; Khan, N.; Shi, J.; Stec, W.; Cleary, D.; Ross, B. S.; Sofia, M. J., An efficient and diastereoselective synthesis of PSI-6130: a clinically efficacious inhibitor of HCV NS5B polymerase. J Org Chem 2009, 74 (17), 6819-24.
34. Sofia, M. J.; Bao, D.; Chang, W.; Du, J.; Nagarathnam, D.; Rachakonda, S.; Reddy, P. G.; Ross, B. S.; Wang, P.; Zhang, H. R.; Bansal, S.; Espiritu, C.; Keilman, M.; Lam, A. M.; Steuer, H. M.; Niu, C.; Otto, M. J.; Furman, P. A., Discovery of a β-d-2′-deoxy-2′-α-fluoro-2′-β-C-methyluridine nucleotide prodrug (PSI-7977) for the treatment of hepatitis C virus. J Med Chem 2010, 53 (19), 7202-18.
35. Chun, B. K.; Du, J.; Zhang, H. R.; Chang, W.; Ross, B. S.; Jiang, Y.; Bao, D.; Espiritu, C. L.; Keilman, M.; Steuer, H. M.; Furman, P. A.; Sofia, M. J., Synthesis of stable isotope labeled analogs of the anti-hepatitis C virus nucleotide prodrugs PSI-7977 and PSI-352938. Nucleosides Nucleotides Nucleic Acids 2011, 30 (11), 886-96.
36. Ross, B. S.; Reddy, P. G.; Zhang, H. R.; Rachakonda, S.; Sofia, M. J., Synthesis of diastereomerically pure nucleotide phosphoramidates. J Org Chem 2011, 76 (20), 8311-9.
37. Du, J.; Bao, D.; Chun, B. K.; Jiang, Y.; Reddy, P. G.; Zhang, H. R.; Ross, B. S.; Bansal, S.; Bao, H.; Espiritu, C.; Lam, A. M.; Murakami, E.; Niu, C.; Micolochick Steuer, H. M.; Furman, P. A.; Otto, M. J.; Sofia, M. J., β-D-2′-α-F-2′-β-C-Methyl-6-O-substituted 3′,5′-cyclic phosphate nucleotide prodrugs as inhibitors of hepatitis C virus replication: a structure-activity relationship study. Bioorg Med Chem Lett 2012, 22 (18), 5924-9.
38. Keating, G. M.; Vaidya, A., Sofosbuvir: first global approval. Drugs 2014, 74 (2), 273-82.
39. Feng, J. Y.; Xu, Y.; Barauskas, O.; Perry, J. K.; Ahmadyar, S.; Stepan, G.; Yu, H.; Babusis, D.; Park, Y.; McCutcheon, K.; Perron, M.; Schultz, B. E.; Sakowicz, R.; Ray, A. S., Role of Mitochondrial RNA Polymerase in the Toxicity of Nucleotide Inhibitors of Hepatitis C Virus. Antimicrobial Agents and Chemotherapy 2016, 60 (2), 806-817.
40. Janas, M. M.; Zlatev, I.; Liu, J.; Jiang, Y.; Barros, S. A.; Sutherland, J. E.; Davis, W. P.; Liu, J.; Brown, C. R.; Liu, X.; Schlegel, M. K.; Blair, L.; Zhang, X.; Das, B.; Tran, C.; Aluri, K.; Li, J.; Agarwal, S.; Indrakanti, R.; Charisse, K.; Nair, J.; Matsuda, S.; Rajeev, K. G.; Zimmermann, T.; Sepp-Lorenzino, L.; Xu, Y.; Akinc, A.; Fitzgerald, K.; Vaishnaw, A. K.; Smith, P. F.; Manoharan, M.; Jadhav, V.; Wu, J. T.; Maier, M. A., Safety evaluation of 2′-deoxy-2′-fluoro nucleotides in GalNAc-siRNA conjugates. Nucleic Acids Res 2019, 47 (7), 3306-3320.
41. Lima, W. F.; Prakash, T. P.; Murray, H. M.; Kinberger, G. A.; Li, W.; Chappell, A. E.; Li, C. S.; Murray, S. F.; Gaus, H.; Seth, P. P.; Swayze, E. E.; Crooke, S. T., Single-stranded siRNAs activate RNAi in animals. Cell 2012, 150 (5), 883-94.
42. Prakash, T. P.; Lima, W. F.; Murray, H. M.; Li, W.; Kinberger, G. A.; Chappell, A. E.; Gaus, H.; Seth, P. P.; Bhat, B.; Crooke, S. T.; Swayze, E. E., Identification of metabolically stable 5′-phosphate analogs that support single-stranded siRNA activity. Nucleic Acids Res 2015, 43 (6), 2993-3011.
43. Parmar, R.; Willoughby, J. L.; Liu, J.; Foster, D. J.; Brigham, B.; Theile, C. S.; Charisse, K.; Akinc, A.; Guidry, E.; Pei, Y.; Strapps, W.; Cancilla, M.; Stanton, M. G.; Rajeev, K. G.; Sepp-Lorenzino, L.; Manoharan, M.; Meyers, R.; Maier, M. A.; Jadhav, V., 5′-(E)-Vinylphosphonate: A Stable Phosphate Mimic Can Improve the RNAi Activity of siRNA-GalNAc Conjugates. Chembiochem 2016, 17 (11), 985-9.
44. Prakash, T. P.; Kinberger, G. A.; Murray, H. M.; Chappell, A.; Riney, S.; Graham, M. J.; Lima, W. F.; Swayze, E. E.; Seth, P. P., Synergistic effect of phosphorothioate, 5′-vinylphosphonate and GalNAc modifications for enhancing activity of synthetic siRNA. Bioorg Med Chem Lett 2016, 26 (12), 2817-2820.
45. Elkayam, E.; Parmar, R.; Brown, C. R.; Willoughby, J. L.; Theile, C. S.; Manoharan, M.; Joshua-Tor, L., siRNA carrying an (E)-vinylphosphonate moiety at the 5′ end of the guide strand augments gene silencing by enhanced binding to human Argonaute-2. Nucleic Acids Res 2017, 45 (6), 3528-3536.
46. Haraszti, R. A.; Roux, L.; Coles, A. H.; Turanov, A. A.; Alterman, J. F.; Echeverria, D.; Godinho, B.; Aronin, N.; Khvorova, A., 5′-Vinylphosphonate improves tissue accumulation and efficacy of conjugated siRNAs in vivo. Nucleic Acids Res 2017, 45 (13), 7581-7592.
47. Parmar, R. G.; Brown, C. R.; Matsuda, S.; Willoughby, J. L. S.; Theile, C. S.; Charissé, K.; Foster, D. J.; Zlatev, I.; Jadhav, V.; Maier, M. A.; Egli, M.; Manoharan, M.; Rajeev, K. G., Facile Synthesis, Geometry, and 2′-Substituent-Dependent in Vivo Activity of 5′-(E)- and 5′-(Z)-Vinylphosphonate-Modified siRNA Conjugates. J Med Chem 2018, 61 (3), 734-744.
48. Janas, M. M.; Schlegel, M. K.; Harbison, C. E.; Yilmaz, V. O.; Jiang, Y.; Parmar, R.; Zlatev, I.; Castoreno, A.; Xu, H.; Shulga-Morskaya, S.; Rajeev, K. G.; Manoharan, M.; Keirstead, N. D.; Maier, M. A.; Jadhav, V., Selection of GalNAc-conjugated siRNAs with limited off-target-driven rat hepatotoxicity. Nat Commun 2018, 9 (1), 723.
49. Mergny, J. L.; Lacroix, L., Analysis of thermal melting curves. Oligonucleotides 2003, 13 (6), 515-37.
50. Bernardi, A.; Bernardi, G., Studies on acid hydrolases. IV. Isolation and characterization of spleen exonuclease. Biochim Biophys Acta 1968, 155 (2), 360-70.
51. Elkayam, E.; Parmar, R.; Brown, C. R.; Willoughby, J. L.; Theile, C. S.; Manoharan, M.; Joshua-Tor, L., siRNA carrying an (E)-vinylphosphonate moiety at the 5 end of the guide strand augments gene silencing by enhanced binding to human Argonaute-2. Nucleic Acids Res 2017, 45 (6), 3528-3536.
52. Kenski, D. M.; Cooper, A. J.; Li, J. J.; Willingham, A. T.; Haringsma, H. J.; Young, T. A.; Kuklin, N. A.; Jones, J. J.; Cancilla, M. T.; McMasters, D. R.; Mathur, M.; Sachs, A. B.; Flanagan, W. M., Analysis of acyclic nucleoside modifications in siRNAs finds sensitivity at position 1 that is restored by 5′-terminal phosphorylation both in vitro and in vivo. Nucleic Acids Res 2010, 38 (2), 660-71.
53. Elkayam, E.; Kuhn, C. D.; Tocilj, A.; Haase, A. D.; Greene, E. M.; Hannon, G. J.; Joshua-Tor, L., The structure of human argonaute-2 in complex with miR-20a. Cell 2012, 150 (1), 100-10.
54. Agarwal, V.; Bell, G. W.; Nam, J. W.; Bartel, D. P., Predicting effective microRNA target sites in mammalian mRNAs. Elife 2015, 4.
55. Doench, J. G.; Petersen, C. P.; Sharp, P. A., siRNAs can function as miRNAs. Genes Dev 2003, 17 (4), 438-42.
56. Vaish, N.; Chen, F.; Seth, S.; Fosnaugh, K.; Liu, Y.; Adami, R.; Brown, T.; Chen, Y.; Harvie, P.; Johns, R.; Severson, G.; Granger, B.; Charmley, P.; Houston, M.; Templin, M. V.; Polisky, B., Improved specificity of gene silencing by siRNAs containing unlocked nucleobase analogs. Nucleic Acids Res 2011, 39 (5), 1823-32.
57. Ui-Tei, K.; Naito, Y.; Nishi, K.; Juni, A.; Saigo, K., Thermodynamic stability and Watson-Crick base pairing in the seed duplex are major determinants of the efficiency of the siRNA-based off-target effect. Nucleic Acids Res 2008, 36 (22), 7100-9.
58. Jayaraman, M.; Ansell, S. M.; Mui, B. L.; Tam, Y. K.; Chen, J.; Du, X.; Butler, D.; Eltepu, L.; Matsuda, S.; Narayanannair, J. K.; Rajeev, K. G.; Hafez, I M.; Akinc, A.; Maier, M. A.; Tracy, M. A.; Cullis, P. R.; Madden, T. D.; Manoharan, M.; Hope, M. J., Maximizing the potency of siRNA lipid nanoparticles for hepatic gene silencing in vivo. Angew Chem Int Ed Engl 2012, 51 (34), 8529-33.
59. Schlegel, M. K.; Foster, D. J.; Kel'in, A. V.; Zlatev, I.; Bisbe, A.; Jayaraman, M.; Lackey, J. G.; Rajeev, K. G.; Charissé, K.; Harp, J.; Pallan, P. S.; Maier, M. A.; Egli, M.; Manoharan, M., Chirality Dependent Potency Enhancement and Structural Impact of Glycol Nucleic Acid Modification on siRNA. J Am Chem Soc 2017, 139 (25), 8537-8546.
60. Clark, J. L.; Hollecker, L.; Mason, J. C.; Stuyver, L. J.; Thamish, P. M.; Lostia, S.; McBrayer, T. R.; Schinazi, R. F.; Watanabe, K. A.; Otto, M. J.; Furman, P. A.; Stec, W. J.; Patterson, S. E.; Pankiewicz, K. W., Design, synthesis, and antiviral activity of 2′-deoxy-2′-fluoro-2′-C-methylcytidine, a potent inhibitor of hepatitis C virus replication. J Med Chem 2005, 48 (17), 5504-8.
61. Sofia, M. J.; Bao, D.; Chang, W.; Du, J.; Nagarathnam, D.; Rachakonda, S.; Reddy, P. G.; Ross, B. S.; Wang, P.; Zhang, H. R.; Bansal, S.; Espiritu, C.; Keilman, M.; Lam, A. M.; Steuer, H. M.; Niu, C.; Otto, M. J.; Furman, P. A., Discovery of a beta-d-2′-deoxy-2′-alpha-fluoro-2′-beta-C-methyluridine nucleotide prodrug (PSI-7977) for the treatment of hepatitis C virus. J Med Chem 2010, 53 (19), 7202-18.
62. Pettersen, E. F.; Goddard, T. D.; Huang, C. C.; Couch, G. S.; Greenblatt, D. M.; Meng, E. C.; Ferrin, T. E., UCSF Chimera—a visualization system for exploratory research and analysis. J Comput Chem 2004, 25 (13), 1605-12.
63. Pallan, P. S.; Greene, E. M.; Jicman, P. A.; Pandey, R. K.; Manoharan, M.; Rozners, E.; Egli, M., Unexpected origins of the enhanced pairing affinity of 2′-fluoro-modified RNA. Nucleic Acids Res. 2011, 39 (Copyright (C) 2017 American Chemical Society (ACS). All Rights Reserved.), 3482-3495.
64. Jinek, M.; Coyle, S. M.; Doudna, J. A., Coupled 5′ nucleotide recognition and processivity in Xrn1-mediated mRNA decay. Mol Cell 2011, 41 (5), 600-8.
65. Brautigam, C. A.; Steitz, T. A., Structural principles for the inhibition of the 3′-5′ exonuclease activity of Escherichia coli DNA polymerase I by phosphorothioates. J Mol Biol 1998, 277 (2), 363-77.
66. Akabane-Nakata, M.; Erande, N. D.; Kumar, P.; Degaonkar, R.; Gilbert, J. A.; Qin, J.; Mendez, M.; Woods, L. B.; Jiang, Y.; Janas, M. M.; O'Flaherty, D. K.; Zlatev, I.; Schlegel, M. K.; Matsuda, S.; Egli, M.; Manoharan, M., siRNAs containing 2′-fluorinated Northern-methanocarbacyclic (2′-F-NMC) nucleotides: in vitro and in vivo RNAi activity and inability of mitochondrial polymerases to incorporate 2′-F-NMC NTPs. Nucleic Acids Res 2021, 49 (5), 2435-2449.
67. Szymanski, M. R.; Kuznetsov, V. B.; Shumate, C.; Meng, Q.; Lee, Y. S.; Patel, G.; Patel, S.; Yin, Y. W., Structural basis for processivity and antiviral drug toxicity in human mitochondrial DNA replicase. Embo j 2015, 34 (14), 1959-70.
68. Arnold, J. J.; Smidansky, E. D.; Moustafa, I. M.; Cameron, C. E., Human mitochondrial RNA polymerase: structure-function, mechanism and inhibition. Biochim Biophys Acta 2012, 1819 (9-10), 948-60.
69. Reddy, P. G.; Bao, D.; Chang, W.; Chun, B. K.; Du, J.; Nagarathnam, D.; Rachakonda, S.; Ross, B. S.; Zhang, H. R.; Bansal, S.; Espiritu, C. L.; Keilman, M.; Lam, A. M.; Niu, C.; Steuer, H. M.; Furman, P. A.; Otto, M. J.; Sofia, M. J., 2′-deoxy-2′-α-fluoro-2′-β-C-methyl 3′,5′-cyclic phosphate nucleotide prodrug analogs as inhibitors of HCV NS5B polymerase: discovery of PSI-352938. Bioorg Med Chem Lett 2010, 20 (24), 7376-80.
70. Chang, W.; Bao, D.; Chun, B. K.; Naduthambi, D.; Nagarathnam, D.; Rachakonda, S.; Reddy, P. G.; Ross, B. S.; Zhang, H. R.; Bansal, S.; Espiritu, C. L.; Keilman, M.; Lam, A. M.; Niu, C.; Steuer, H. M.; Furman, P. A.; Otto, M. J.; Sofia, M. J., Discovery of PSI-353661, a Novel Purine Nucleotide Prodrug for the Treatment of HCV Infection. ACS Med Chem Lett 2011, 2 (2), 130-5.
71. Reddy, P. G.; Chun, B. K.; Zhang, H. R.; Rachakonda, S.; Ross, B. S.; Sofia, M. J., Stereoselective synthesis of PSI-352938: a β-D-2′-deoxy-2′-α-fluoro-2′-β-C-methyl-3′,5′-cyclic phosphate nucleotide prodrug for the treatment of HCV. J Org Chem 2011, 76 (10), 3782-90.

### Supporting Information

### Experimental

### Synthesis of Compound 2.

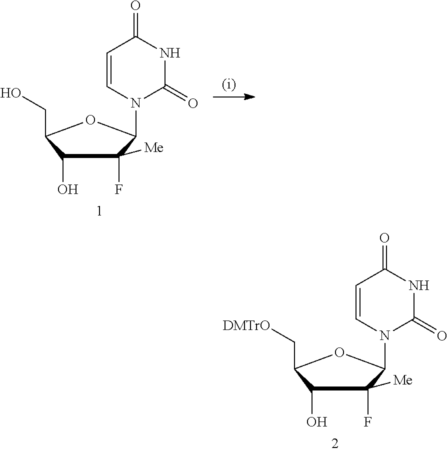

To a solution of compound 1¹ (10.1 g, 38.8 mmol) in anhydrous pyridine (100 mL) was added DMTrCl (14.5 g, 42.7 mmol) at 0° C. The reaction mixture was stirred for 16 h at ambient temperature then quenched by addition of MeOH. The solvent was removed under reduced pressure, and the residue was extracted with CH₂Cl₂ and saturated aqueous NaHCO₃ solution. The organic layer was separated, dried over anhydrous Na₂SO₄, filtered, and concentrated. The crude material was purified by flash silica gel column chromatography (0-4% MeOH in CH₂Cl₂) to give compound 2 (20.0 g, 35.6 mmol, 92%, R_{f}=0.36; developed with 4% MeOH in CH₂Cl₂). ¹H NMR (400 MHz, DMSO-d₆): δ 11.52 (s, 1H), 7.84 (d, J=8.0 Hz, 1H), 7.40-7.31 (m, 4H), 7.28-7.23 (m, 4H), 6.92-6.90 (m, 4H), 6.03 (d, J=19.2 Hz, 1H), 5.78 (d, J=8.0 Hz, 1H), 5.14 (d, J=8.0 Hz, 1H), 4.13-3.97 (m, 2H), 3.74 (s, 6H), 3.42 (dd, J=11.0, 3.8 Hz, 1H), 3.34-3.31 (m, 1H), 1.33 (s, 3H), 1.27 (s, 3H). ¹³C NMR (126 MHz, DMSO-d₆): δ 167.85, 162.70, 158.17, 158.06, 150.43, 144.54, 135.31, 134.92, 134.25, 131.18, 129.80, 128.18, 127.94, 127.74, 127.43, 126.85, 101.86, 101.34, 99.90, 86.02, 80.06, 71.25, 61.35, 55.06, 55.05, 16.52, 16.32. ¹⁹F NMR (376 MHz, DMSO-d₆): δ −162.62. HRMS calc. for C₃₁H₃₂FN₂O₇ [M+H]⁺ 563.2194, found 563.2200.

### Synthesis of Compound 3.

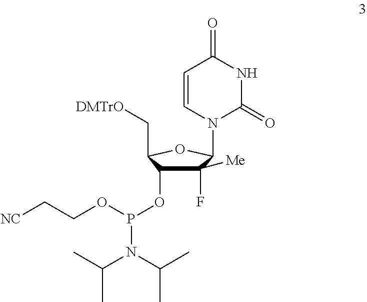

To a solution of compound 21 (8.00 g, 14.2 mmol) in anhydrous CH₂Cl₂ (80 mL) and N,N-diisopropylethylamine (9.89 mL, 56.8 mmol) was added 2-cyanoethyl N,N-diisopropylchlorophosphoramidite (5.00 g, 21.1 mmol). The reaction mixture was stirred at ambient temperature for 16 h under argon atmosphere. The reaction mixture was diluted with CH₂Cl₂ (300 mL) then washed with saturated aqueous NaHCO₃ (100 mL). The organic layer was separated, dried over anhydrous Na₂SO₄, filtered, and concentrated. The crude material was purified by flash silica gel column chromatography (0-50% EtOAc in hexanes) to give compound 3 (9.71 g, 12.7 mmol, 90%, R_{f}=0.15, 0.25 developed with 50% EtOAc in hexanes) as a white foam. ¹H NMR (400 MHz, DMSO-d₆): δ 11.55 (s, 1H), 7.91-7.88 (m, 1H), 7.40-7.23 (m, 9H), 6.92-6.88 (m, 4H), 6.08 (d, J=10.8 Hz, 1H), 5.08 (brs, 1H), 4.49-4.37 (m, 1H), 4.12-4.07 (m, 1H), 3.80-3.61 (m, 8H), 3.56-3.40 (m, 5H), 2.79 (t, J=5.8 Hz, 1H), 2.59-2.53 (m, 1H), 1.43-1.37 (m, 3H), 1.13-0.92 (m, 12H). ³¹P NMR (162 MHz, DMSO-d₆): δ 155.13, 154.08. ¹³C NMR (126 MHz, DMSO-d₆): δ 162.64, 158.21, 158.20, 150.38, 144.42, 144.39, 139.29, 135.09, 134.99, 134.83, 134.70, 129.90, 129.87, 129.85, 127.87, 127.86, 127.83, 127.76, 126.90, 126.85, 118.97, 118.62, 113.19, 113.17, 101.99, 100.91, 99.45, 88.41, 86.15, 79.71, 73.06, 58.23, 58.07, 58.00, 57.85, 55.03, 55.01, 55.00, 54.98, 42.69, 42.65, 42.59, 42.55, 24.31, 24.28, 24.25, 24.23, 24.21, 24.18, 24.15, 24.13, 19.76, 19.72, 19.70, 19.66, 17.22, 17.02, 16.85, 16.65. ¹⁹F NMR (376 MHz, DMSO-d₆): δ −160.84. HRMS calc. for C₄₀H₄₈FN₄NaO₈P [M+Na]⁺785.3092, found 785.3098.

### Synthesis of Compound 5.

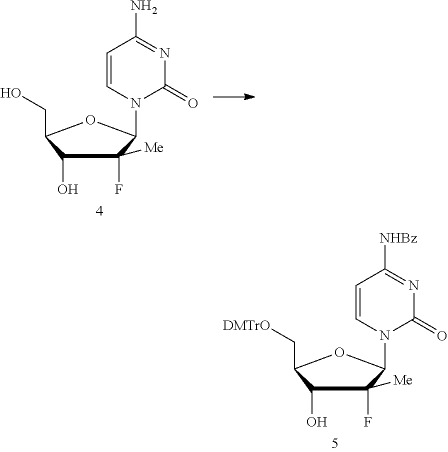

To a solution of compound 4 (5.00 g, 19.3 mmol) in anhydrous DMF (80 mL) was added benzoic anhydride (4.80 g, 21.2 mmol). The reaction mixture was stirred at ambient temperature for 24 h, and then the solvent was removed under reduced pressure. The residue was co-evaporated with pyridine twice and used for next step without further purification. To a solution of the crude material in anhydrous pyridine (80 mL) was added DMTrCl (7.19 g, 21.2 mmol) at 0° C. The reaction mixture was stirred for 16 h at ambient temperature then quenched by addition of MeOH. The solvent was removed under reduced pressure, and the residue was extracted with CH₂Cl₂ and saturated aqueous NaHCO₃ solution. The organic layer was separated, dried over anhydrous Na₂SO₄, filtered, and concentrated. The crude material was purified by flash silica gel column chromatography (0-80% EtOAc in hexanes) to give compound 5 (9.46 g, 14.2 mmol, 74%, R_{f}=0.33; developed with hexanes/EtOAc=1:3). ¹H NMR (400 MHz, DMSO-d₆): δ 11.33 (s, 1H), 8.40 (d, J=7.6 Hz, 1H), 8.00-7.98 (m, 2H), 7.65-7.61 (m, 1H), 7.54-7.50 (m, 2H), 7.42-7.34 (m, 4H), 7.30-7.25 (m, 5H), 7.16 (d, J=7.6 Hz, 1H), 6.95-6.91 (m, 4H), 6.21 (d, J=18.4 Hz, 1H), 5.80 (d, J=7.2 Hz, 1H), 4.21-4.04 (m, 2H), 3.77 (s, 3H), 3.76 (s, 3H), 3.47-3.37 (m, 2H), 1.31 (d, J=22.4 Hz, 3H). ¹³C NMR (126 MHz, DMSO-d₆): δ 167.37, 163.32, 158.21, 158.19, 154.53, 144.20, 135.47, 135.06, 133.05, 132.78, 129.82, 129.66, 128.45, 128.44, 127.99, 127.85, 126.94, 101.63, 100.18, 96.68, 86.15, 80.15, 70.99, 70.85, 61.08, 55.01, 16.45, 16.25. ¹⁹F NMR (376 MHz, DMSO-d₆): δ −163.72. HRMS calc. for C₃₈H₃₇FN₃O₇ [M+H]⁺ 666.2616, found 666.2620.

### Synthesis of Compound 6.

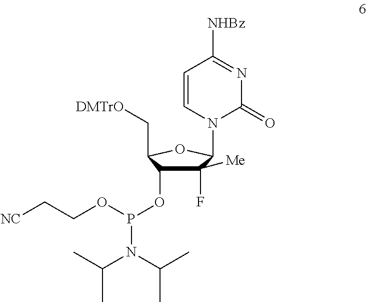

To a solution of compound 5 (9.40 g, 14.1 mmol) in anhydrous CH₂Cl₂ (70 mL) was added 2-cyanoethyl N,N,N′,N′-tetraisopropylphosphorodiamidite (5.37 mL, 16.9 mmol) and 4,5-dicyanoimidazole (1.67 g, 14.1 mmol). The reaction mixture was stirred at ambient temperature for 16 h under argon atmosphere. The reaction mixture was diluted with CH₂Cl₂ (300 mL) then washed with saturated aqueous NaHCO₃ (150 mL). The organic layer was separated, dried over anhydrous Na₂SO₄, filtered, and concentrated. The crude material was purified by flash silica gel column chromatography (30-50% EtOAc in hexanes) to give 6 (11.6 g, 13.4 mmol, 95%, R_{f}=0.18, 0.25 developed with 50% EtOAc in hexanes) as a pale yellow foam. ¹H NMR (400 MHz, DMSO-d₆): δ 11.35 (s, 1H), 8.43 (d, J=7.6 Hz, 1H), 8.00-7.97 (m, 2H), 7.65-7.61 (m, 1H), 7.53-7.50 (m, 2H), 7.44-7.26 (m, 9H), 7.11 (d, J=8.0 Hz, 1H), 6.94-6.90 (m, 4H), 6.29-6.24 (m, 1H), 4.54-4.37 (m, 1H), 4.20-4.14 (m, 1H), 3.82-3.75 (m, 7H), 3.65-3.43 (m, 6H), 2.78 (t, J=5.8 Hz, 1H), 2.61-2.54 (m, 1H), 1.45-1.38 (m, 3H), 1.17-0.93 (m, 12H). ³¹P NMR (162 MHz, DMSO-d₆): δ 155.21, 154.16. ¹³C NMR (126 MHz, DMSO-d₆): δ 167.30, 163.40, 158.24, 154.47, 144.11, 135.18, 135.01, 134.95, 134.85, 133.00, 132.76, 129.89, 129.87, 129.80, 129.77, 128.42, 127.94, 127.92, 127.90, 127.86, 126.99, 126.93, 118.98, 118.65, 113.26, 113.23, 113.22, 101.22, 101.02, 100.99, 99.76, 99.55, 99.52, 96.77, 89.39, 86.26, 79.92, 72.90, 60.60, 58.22, 58.06, 57.95, 57.80, 55.02, 55.00, 54.98, 54.96, 54.95, 54.93, 42.68, 42.58, 24.32, 24.31, 24.26, 24.22, 24.16, 24.12, 19.76, 19.74, 19.70, 19.68, 17.16, 16.96, 16.74, 16.54. ¹⁹F NMR (376 MHz, DMSO-d₆): δ −161.78. HRMS calc. for C₄₇H₅₄FN₅O₈P [M+H]⁺ 866.3694, found 866.3688.

### Synthesis of Compounds 8 and 9.

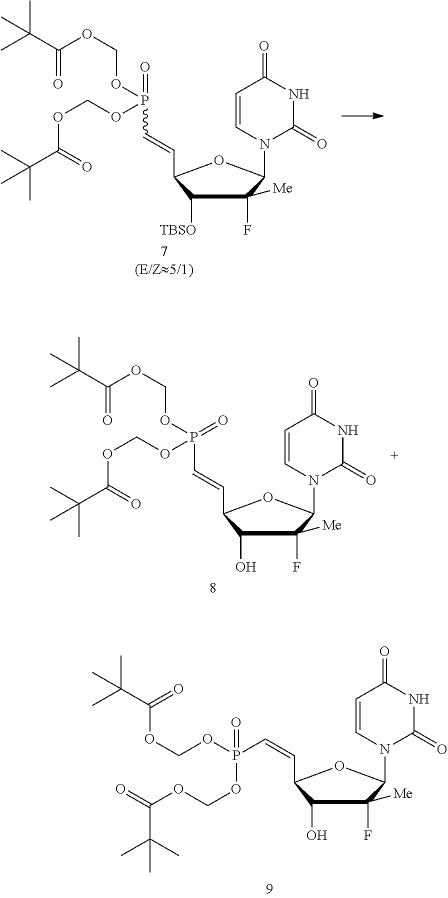

Compound 7 was synthesized following the reported procedure² and used as a mixture of diastereomers (E/Z≈5/1). A solution of compound 7 (15.0 g, 22.1 mmol) in HCOOH/H₂O (200 mL, 1:1, v:v) was stirred at 40° C. for 16 h, and then the solvent was removed under reduced pressure. The residue was purified by flash silica gel column chromatography (AcOEt) to give compound 8 (7.51 g, 13.31 mmol, 60%, R_{f}=0.36; developed with 80% EtOAc in hexanes) as a white foam and, separately, compound 9 (1.56 g, 2.77 mmol, 12%, R_{f}=0.32; developed with 80% EtOAc in hexanes) as a white foam. Compound 8: ¹H NMR (500 MHz, CD₃OD): δ 7.48 (d, J=8.5 Hz, 1H), 7.00 (ddd, J=24.0, 17.0, 4.5 Hz, 1H), 6.26-6.00 (m, 2H), 5.75 (d, J=8.0 Hz, 1H), 5.73-5.65 (m, 4H), 4.52-4.46 (m, 1H), 4.03-3.77 (m, 1H), (d, J=22.5, 3H), 1.23 (s, 9H), 1.22 (s, 9H). ³¹P NMR (202 MHz, CD₃OD): δ 18.30. ¹³C NMR (101 MHz, CD₃OD): δ 178.19, 173.10, 165.71, 152.06, 150.87, 142.29, 119.64, 117.74, 103.70, 102.56, 100.75, 83.30, 82.24, 82.00, 78.19, 61.66, 49.58, 39.90, 27.39, 21.01, 17.13, 16.88, 14.62. ¹⁹F NMR (470 MHz, CD₃OD): δ −161.65. HRMS calc. for C₂₃H₃₄FN₂NaO₇P [M+Na]⁺587.1782, found 587.1789. Compound 9: ¹H NMR (500 MHz, CD₃OD): δ 7.58 (d, J=8.0 Hz, 1H), 6.79 (ddd, J=55.0, 13.5, 9.0 Hz, 1H), 6.18-5.94 (m, 2H), 5.77-5.65 (m, 5H), 5.30-5.23 (m, 1H), 4.00-3.66 (m, 1H), 1.37 (d, J=22.5, 3H), 1.23 (s, 9H), 1.22 (s, 9H). ³¹P NMR (202 MHz, CD₃OD): δ 16.21. ¹³C NMR (101 MHz, CD₃OD): δ 178.19, 178.11, 165.76, 152.08, 150.93, 142.63, 122.64, 120.79, 103.49, 102.32, 100.52, 83.22, 83.17, 79.00, 78.98, 78.83, 78.82, 78.80, 39.89, 27.37, 17.30, 17.05. ¹⁹F NMR (470 MHz, CD₃OD): δ −161.69. HRMS calc. for C₂₃H₃₅FN₂O₇P [M+H]⁺ 565.1963, found 565.1965.

### Synthesis of Compound 10.

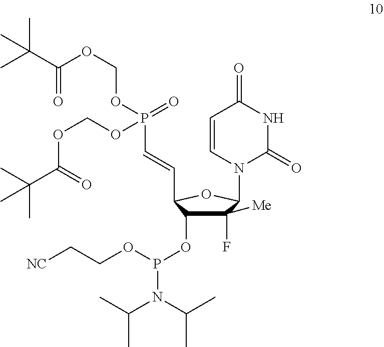

To a solution compound 8 (2.40 g, 4.26 mmol) and 5-(ethylthio)-1H-tetrazole (0.54 g, 4.26 mmol) in anhydrous acetonitrile (40 mL) was added 2-cyanoethyl N,N,N′,N′-tetraisopropylphosphordiamidite (1.54 g, 5.12 mmol). The reaction mixture was stirred at room temperature for 2 h. TLC analysis in ethyl acetate/hexanes (2:8, v/v) containing 0.15% triethylamine confirmed formation of the product. The reaction mixture was filtered, concentrated and loaded onto a silica gel column. The sample was eluted with 50% ethyl acetate in hexanes containing 0.15% triethylamine to afford a compound 10 (2.40 g, 3.14 mmol, 74%, R_{f}=0.12, 0.27 developed with 60% EtOAc in hexanes) as a white foam. ¹H NMR (500 MHz, CD₃CN): δ 9.31 (brs, 1H), 7.35-7.27 (m, 1H), 7.08-6.84 (m, 1H), 6.26-5.94 (m, 2H), 5.70-5.55 (m, 5H), 4.59-4.49 (m, 1H), 4.28-3.99 (m, 1H), 3.91-3.82 (m, 1H), 3.76-3.60 (m, 3H), 2.86-2.71 (m, 2H), 1.45-1.34 (m, 3H), 1.21-1.16 (m, 30H). ³¹P NMR (202 MHz, CD₃CN): δ 153.40, 151.44, 17.94, 17.24. ¹³C NMR (101 MHz, CD₃CN) 6 177.68, 177.64, 163.66, 163.65, 151.40, 149.06, 148.47, 120.62, 119.90, 118.74, 103.71, 82.87, 82.83, 82.82, 82.78, 80.85, 79.24, 59.52, 59.32, 44.31, 44.27, 44.19, 44.15, 39.48, 39.46, 27.20, 27.18, 25.16, 25.08, 25.05, 24.96, 24.89, 20.93, 20.85, 17.87, 17.84, 17.62, 17.59. ¹⁹F NMR (470 MHz, CD₃CN): δ −158.78. HRMS calc. for C₃₂H₃₅FN₂O₇P₂ [M+H]⁺ 765.3041, found 765.3050.

### Synthesis of Compound 11.

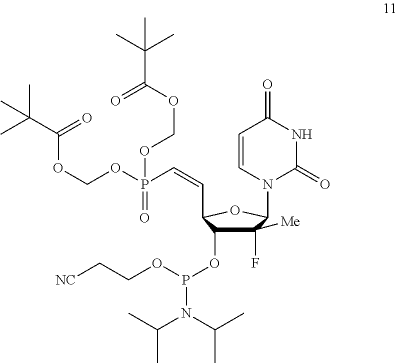

To a solution compound 9 (1.20 g, 2.13 mmol) and 5-(ethylthio)-1H-tetrazole (0.27 g, 2.13 mmol) in anhydrous acetonitrile (20 mL) was added 2-cyanoethyl N,N,N′,N′-tetraisopropylphosphordiamidite (0.77 g, 2.56 mmol). The reaction mixture was stirred at room temperature for 2 h. TLC analysis in ethyl acetate/hexanes (2:8, v/v) containing 0.15% triethylamine confirmed formation of the product. The reaction mixture was filtered, concentrated and loaded onto a silica gel column. The sample was eluted with 50% ethyl acetate in hexanes containing 0.15% triethylamine to afford a compound 11 (1.22 g, 1.59 mmol, 75%, R_{f}=0.18, 0.34 developed with 60% EtOAc in hexanes) as a white foam. ¹H NMR (500 MHz, CD₃CN): δ 9.30 (brs, 1H), 7.39-7.34 (m, 1H), 6.79-6.53 (m, 1H), 6.20-5.90 (m, 2H), 5.72-5.56 (m, 5H), 5.43-5.34 (m, 1H), 4.36-3.96 (m, 1H), 3.90-3.44 (m, 4H), 2.86-2.58 (m, 2H), 1.48-1.34 (m, 3H), 1.26-1.14 (m, 30H). ³¹P NMR (202 MHz, CD₃CN): δ 152.43, 150.46, 15.01, 14.84. ¹³C NMR (101 MHz, CD₃CN) δ 177.62, 177.58, 177.56, 177.55, 163.73, 163.72, 151.40, 148.47, 119.77, 103.46, 82.82, 82.77, 82.72, 82.66, 82.59, 82.54, 80.05, 79.89, 79.73, 79.58, 79.53, 79.41, 77.03, 59.71, 59.56, 59.53, 46.36, 46.30, 44.26, 44.14, 44.10, 43.97, 39.47, 39.46, 27.17, 27.14, 25.19, 25.11, 25.04, 25.02, 24.97, 24.94, 23.41, 22.62, 21.06, 21.01, 20.99, 20.93, 18.38, 18.33, 18.12, 18.08, 17.97, 17.95, 17.72, 17.69. ¹⁹F NMR (470 MHz, CD₃CN): 6-157.27. HRMS calc. for C₃₂H₃₅FN₂O₇P₂ [M+H]⁺ 765.3041, found 765.3039.

Solid-phase oligonucleotide synthesis. Oligonucleotides were synthesized on an ABI synthesizer using standard solid-phase synthesis and deprotection protocols.³ Synthesis was performed at 1-μmol scale using commercially available (5′-O-(4,4′-dimethoxytrityl)-2′-deoxy-2′-fluoro-, 5′-O-(4,4′-dimethoxytrityl)-2′-O-(tert-butyldimethylsilyl)-, or 5′-O-(4,4′-dimethoxytrityl)-2′-O-methyl-3′-O-(2-cyanoethyl-N,N-diisopropyl) phosphoramidite monomers of uridine, 4-N-acetylcytidine, 6-N-benzoyladenosine, and 2-N-isobutyrylguanosine. Either the succinate of the 3′ nucleoside or the GalNAc ligand were conjugated to the controlled pore glass via long chain alkylamine with ˜500 Å pore size. Upon completion of synthesis, oligonucleotides were treated with 0.5 M anhydrous piperidine in acetonitrile for 10 min, then washed thoroughly with anhydrous acetonitrile, and dried using nitrogen. Oligonucleotides were cleaved from solid support and deprotected using ammonium hydroxide at 30° C. for 17 h. After filtration through a nylon syringe filter (0.45 μm), oligonucleotides were either stored for later purification or, in the case of RNA, treated with Et₃N·HF at 60° C. for 10 min to deprotect the 2′-OH. Oligonucleotides were purified using IEX-HPLC using an appropriate gradient of mobile phase (0.15 M NaCl, 10% MeCN and 1.0 M NaBr, 10% MeCN) and desalted using size-exclusion chromatography with water as an eluent. Oligonucleotides were then quantified by measuring the absorbance at 260 nm using the following extinction coefficients: A, 13.86; T/U, 7.92; C, 6.57; and G,10.53. Purities were determined and identities were verified by analytical anion exchange chromatography mass spectrometry, respectively (Table 9).

Determination of thermal denaturation temperatures. Thermal denaturation temperatures were measured with equimolar concentrations of both strands (2.5 μM) in PBS (137 mM NaCl, 2.7 mM KCl, 10 mM Na₂HPO₄, 1.8 mM KH₂PO₄, pH 7.4) or high-salt PBS (931.6 mM NaCl, 18.4 mM KCl, 68 mM Na₂HPO₄, 12.2 mM KH₂PO₄, pH 7.4) by monitoring absorbance at 260 nm with increasing temperature (1° C./min). Values were reported as the maximum of the first derivative and are the average of at least two experiments.

Determination of thermodynamic parameters of hybridization. Thermodynamic parameters were determined using the van't Hoff method and base line fitting of the melting curve, which is available through the Cary WinUV Thermal software. Briefly, the user defines the baseline, which allows for the conversion to percent hybridized. User-input total concentration and molecularity are used to convert the equilibrium equation to the van't Hoff plot. By defining the upper and lower limits of the linear region, the slope and y-intercept can be calculated, which correspond to the enthalpy and entropy parameters, respectively. Each duplex was prepared and analyzed three times, with melting curves collected in duplicate, and results were averaged to yield the reported values.

Stability toward 3′- or 5′-specific exonucleases. Modified oligonucleotides were prepared in a final concentration of 0.1 mg/mL in either 50 mM Tris (pH 7.2), 10 mM MgCl₂ or 50 mM sodium acetate (pH 6.5), 10 mM MgCl₂ to assess the stability toward 3′- or 5′-specific exonucleases, respectively. The exonuclease (150 mU/mL SVPDE or 500 mU/mL phosphodiesterase II) and analysis was performed over time by IEX HPLC (Dionex DNAPac PA200,4×250 mm) using a gradient of 37-52% mobile phase (1 M NaBr, 20 mM sodium phosphate, pH 11, 15% MeCN; stationary phase: 20 mM sodium phosphate, 15% MeCN, pH 11) over 7.5 min with a flow of 1 mL/min. Samples were analyzed at given time points for up to 24 h. The quantity of full-length oligonucleotide was determined as the area under the absorbance curve at 260 nm. Percent full-length oligonucleotide was calculated by dividing by the area under the curve at the time of interest by the area under the curve at t=0 and multiplying by 100. Activity of enzyme was verified for each experiment by including a oligodeoxythymidylate with a terminal phosphorothioate linkage (5′-T₁₉•T or 5′-T•T₁₉ for 3′- or 5′-exonuclease activity, respectively, where • indicates the phosphorothioate linkage). Each aliquot of enzyme was thawed just prior to the experiment. The half-life was determined by fitting to first order kinetics.

mRNA quantification in vitro. Primary mouse hepatocytes were transfected with oligonucleotide using RNAiMAX reagent (ThermoFisher) according to manufacturer's recommendations. Briefly, cells were thawed just prior to transfection and plated onto 384-well plate with a seed density of ˜5000 cells/well in Williams Medium E supplemented with 10% fetal bovine serum. Pre-incubated lipid/siRNA complex (0.1 μL RNAiMax, siRNA, in 5 μL Opti-MEM for 15 min; both reagents from Thermo Fisher) was added to a 384-well collagen-coated plate (BioCoat; Corning). Cells were incubated for 20 h at 37° C. in an atmosphere of 5% CO₂. Media was then removed, and the cells were washed and lysed. RNA was extracted, using Dynabeads mRNA isolation kit (Invitrogen) according to manufacturer's protocol, then reverse transcribed using ABI high capacity cDNA reverse transcription kit. Quantification was accomplished by real-time quantitative PCR, where the cDNA (2 μL) was added to a master mix containing 0.5 μL mouse GAPDH TaqMan Probe, and 5 μL of the target TaqMan probe, and 0.5 μL Lightcycler 480 probe master mix. Amplification was done in an ABI 7900HT-RT-PCR system (Applied Biosystems) using the ΔΔCt (RQ) assay. Each data point was tested with at least four biological replicates. Each well was normalized to GAPDH control, and the mRNA remaining was calculated relative to cells treated with a non-targeting siRNA. IC₅₀ values were calculated from fitted curves using GraphPad Prism.

On- and off-target luciferase reporter assay. COS-7 cells were cultured at 37° C., 5% C02 in Dulbecco's Modified Eagle Medium supplemented with 10% fetal bovine serum. Cells were co-transfected in 96-well plates (15,000 cells/well) with 10 ng luciferase reporter plasmid and 0.64 pM to 50 nM siRNA in 5-fold dilutions using 2 μL Lipofectamine 2000 (Thermo Fisher Scientific) according to manufacturer's instructions. Cells were harvested at 48 hours after transfection for the dual luciferase assay (Promega) according to manufacturer's instructions. The on-target reporter plasmid contained a single site perfectly complementary to the antisense strand in the 3′ untranslated (3′ UTR) of Renilla luciferase. The off-target reporter plasmid contained four tandem seed-complementary sites separated by a 19-nucleotide spacer (TAATATTACATAAATAAAA (SEQ ID NO: 83)) in the 3′ UTR of Renilla luciferase. Both plasmids co-expressed firefly luciferase as a transfection control.

Off-target activity assessment using RNA-seq. Primary rat hepatocytes (BioreclamationIVT) were seeded in 96-well collagen I pre-coated plates (Gibco) at approximately 50,000 cells/well in 95 μL INVITROGRO CP Rodent Medium (BioreclamationIVT). Pre-incubated lipid/siRNA complex (0.25 μL RNAiMax (Thermo Fisher Scientific) and 1 μL siRNA in 3.75 μL Opti-MEM for 15 min) was added to transfect the cells and incubated for 48 h at 37° C. in an atmosphere of 5% CO₂. The final concentration of the siRNA was 50 nM, and each siRNA was tested in quadruplicate. The media was removed, RNA was extracted using the miRNeasy 96 kit (Qiagen), cDNA library was prepared with the TruSeq Stranded Total RNA Library Prep Kit (Illumina) and sequenced on the HiSeq or NextSeq500 sequencers (Illumina), all according to manufacturers' instructions. Raw RNAseq reads were filtered with minimal mean quality scores of 25 and minimal remaining length of 36, using fastq-mcf. Filtered reads were aligned to the Rattus norvegicus genome (Rnor_6.0) using STAR (ultrafast universal RNAseq aligner) with default parameters. Uniquely aligned reads were counted by feature Counts.⁴ Differential gene expression analysis was performed using the R package DESeq2

Analyses of siRNA potencies in mice. All procedures using mice were conducted by certified laboratory personnel using protocols consistent with local, state, and federal regulations. Experimental protocols were approved by the Institutional Animal Care and Use Committee, the Association for Assessment and Accreditation of Laboratory Animal Care International (accreditation number: 001345), and the Office of Laboratory Animal Welfare (accreditation number: #A4517-01). All control animals were randomly assigned to cages upon facility arrival. To calculate sample numbers necessary for animal studies, we determined the final number required to be one that would allow for confidence in the resulting data set utilizing the least number of animals, as required in accordance with IACUC guidelines.

Wild-type C57BL/6 female mice, approximately 6-8 weeks of age, were acquired from Charles River Laboratories. Animals (n=3 per group) were dosed with GalNAc-siRNA conjugates diluted into 1×PBS at either 0.3 mg/mL or 0.1 mg/mL via a single subcutaneous injection of 10 mg/kg or 1 mg/kg prepared in an injection volume of 10 μL per g body weight in PBS. For evaluation of mRNA in liver, mice were euthanized with carbon dioxide after collection of blood, the liver harvested, and snap-frozen in liquid nitrogen. The liver tissues were processed in a SPEX GenoGrinder, and a small amount powderized tissue transferred to tubes for lysis performed using the miRNeasy micro kit (Qiagen Cat 217084). cDNA synthesis was accomplished with the High-capacity cDNA Reverse Transcription Kit (applied Biosystems). A mixture of 1 μl of 10× buffer, 0.4 μl of 25× dNTPs, 1 μl of random primers, 0.5 μl of reverse transcriptase, 0.5 μl of RNase inhibitor and 6.6 μl of water per reactions were added per well. Levels of mRNA of interest were quantified by RT-qPCR and normalized to levels of GAPDH. Values are expressed as percent of relative to levels of targeted mRNA present in samples treated with a control siRNA. All data points are the average of two measurements.

The siRNA duplexes formulated with LNP as previously reported.⁵ The LNP (50% MC3, 38.5% cholesterol and 10% DSPC, 1.5% C₁₄ PEG 2000 lipid) formulated siRNAs were diluted to 0.003 mg/kg and 0.001 mg/kg for intravenous administration through the tail vein at a volume of 10 mL/kg. Serum was collected for pre-dose and at various time points post dose to assay circulating F7 protein levels using the Biophen assay as previously described⁶

Structural models and molecular mechanics minimizations. Crystal structures of the all-2′-F RNA duplex and the human Ago2-miR20a complex were retrieved from the Protein Data Bank (www.rcsb.org). 2′-F/Me nucleotides were incorporated using the structure editing option in UCSF Chimera (version 1.13 for Mac),⁷ and all RNA and RNA-protein complex models were energy-minimized using the Amber ff 14SB force field in combination with Gasteiger potentials as implemented in UCSF Chimera. Refinement included both steepest descent and conjugate gradient type minimization until convergence as judged by no further resulting geometric changes was reached. All drawings were generated using the program UCSF Chimera.

### Oligonucleotide Characterization

TABLE 9
MS (m/z) analysis of modified oligonucleotides^{a}

| Oligo- | | Mass (m/z) | |
|---|---|---|---|
| nucleotide | Sequence (5′-3′) | Calc. | Obs. |
| 3 | UACAGU_{F}CUAUGU (SEQ ID NO: 38) | 3759.3 | 3758.7 |
| 4 | UACAGU_{F/Me}CUAUGU (SEQ ID NO: 39) | 3773.3 | 3772.8 |
| 5 | UACAGUC_{F}UAUGU (SEQ ID NO: 40) | 3759.3 | 3758.3 |
| 6 | UACAGUC_{F/Me}UAUGU (SEQ ID NO: 41) | 3773.3 | 3772.0 |
| 13(U_{F/Me}) | dT₁₉U_{F/Me} | 6039.9 | 6039.1 |
| 13(U_{F}) | dT₁₉U_{F} | 6025.9 | 6024.8 |
| 13(C_{F/Me}) | dT₁₉C_{F/Me} | 6038.9 | 6038.1 |
| 13(C_{F}) | dT₁₉C_{F} | 6024.9 | 6025.1 |
| 14(U_{F/Me}) | dT₁₈U_{F/Me}dT | 6039.9 | 6039.2 |
| 14(U_{F}) | dT₁₈U_{F}dT | 6025.9 | 6025.7 |
| 14(C_{F/Me}) | dT₁₈C_{F/Me}dT | 6038.9 | 6038.0 |
| 14(C_{F}) | dT₁₈C_{F}dT | 6024.9 | 6024.2 |
| 15(U_{F/Me}) | dT₁₈U_{F/Me}U_{F/Me} | 6057.9 | 6057.3 |
| 15(U_{F}) | dT₁₈U_{F}U_{F} | 6029.8 | 6029.1 |
| 15(C_{F/Me}) | dT₁₈C_{F/Me}C_{F/Me} | 6055.9 | 6055.2 |
| 15(C_{F}) | dT₁₈C_{F}C_{F} | 6027.9 | 6027.1 |
| 16(U_{F/Me}) | dT₁₉•U_{F/Me} | 6056.0 | 6055.1 |
| 16(U_{F}) | dT₁₉•U_{F} | 6041.9 | 6041.3 |
| 16(C_{F/Me}) | dT₁₉•C_{F/Me} | 6055.0 | 6054.0 |
| 16(C_{F}) | dT₁₉•C_{F} | 6040.9 | 6040.8 |
| 17(U_{F/Me}) | dT₁₈U_{F/Me}•dT | 6055.9 | 6055.1 |
| 17(U_{F}) | dT₁₈U_{F}•dT | 6041.9 | 6041.6 |
| 17(C_{F/Me}) | dT₁₈C_{F/Me}•dT | 6054.9 | 6054.1 |
| 17(C_{F}) | dT₁₈C_{F}•dT | 6040.9 | 6041.0 |
| 18(U_{F/Me}) | dT₁₈U_{F/Me}•U_{F/Me} | 6073.9 | 6073.6 |
| 18(U_{F}) | dT₁₈U_{F}•U_{F} | 6045.9 | 6045.4 |
| 18(C_{F/Me}) | dT₁₈C_{F/Me}•C_{F/Me} | 6071.9 | 6071.2 |
| 18(C_{F}) | dT₁₈C_{F}•C_{F} | 6043.9 | 6043.1 |
| 19(U_{F/Me}) | U_{F/Me}dT₁₉ | 6039.9 | 6039.1 |
| 19(U_{F}) | U_{F}dT₁₉ | 6025.9 | 6025.3 |
| 19(C_{F/Me}) | C_{F/Me}dT₁₉ | 6038.9 | 6038.3 |
| 19(C_{F}) | C_{F}dT₁₉ | 6024.9 | 6023.9 |
| 20(U_{F/Me}) | dTU_{F/Me}dT₁₈ | 6039.9 | 6038.5 |
| 20(U_{F}) | dTU_{F}dT₁₈ | 6025.9 | 6025.6 |
| 21(U_{F/Me}) | U_{F/Me}B_{F/Me}dT₁₈ | 6362.1 | 6361.6 |
| 21(U_{F}) | U_{F}U_{F}dT₁₈ | 6029.8 | 6028.6 |
| 21(C_{F/Me}) | C_{F/Me}C_{F/Me}dT₁₈ | 6360.1 | 6359.4 |
| 21(C_{F}) | C_{F}C_{F}dT₁₈ | 6027.9 | 6027.1 |
| 22(U_{F/Me}) | U_{F/Me}•dT₁₉ | 6055.9 | 6054.9 |
| 22(U_{F}) | U_{F}•dT₁₉ | 6041.9 | 6040.8 |
| 22(C_{F/Me}) | C_{F/Me}•dT₁₉ | 6054.9 | 6054.1 |
| 22(C_{F}) | C_{F}•dT₁₉ | 6040.9 | 6039.9 |
| 23(U_{F/Me}) | dT•U_{F/Me}dT₁₈ | 6056.0 | 6054.6 |
| 23(U_{F}) | dT•U_{F}dT₁₈ | 6041.9 | 6041.3 |
| 24(U_{F/Me}) | U_{F/Me}•U_{F/Me}dT₁₈ | 6378.1 | 6377.6 |
| 24(U_{F}) | U_{F}•U_{F}dT₁₈ | 6045.9 | 6044.7 |
| 24(C_{F/Me}) | C_{F/Me}•C_{F/Me}dT₁₈ | 6376.1 | 6375.5 |
| 24(C_{F}) | C_{F}•C_{F}DT₁₈ | 6043.9 | 6043.1 |
| 25 | A_{F}•a•C_{F}aG_{F}uG_{F}uU_{F}C_{F}U_{F}uG_{F}cU_{F}cU_{F}aU_{F}aA_{F}* (SEQ ID NO: 42) | 8590.2 | 8590.0 |
| 26 | U_{F}•U_{F}•aU_{F}aG_{F}aG_{F}cA_{F}agaA_{F}cA_{F}cU_{F}gU_{F}u•u•u (SEQ ID NO: 60) | 7583.9 | 7582.4 |
| 27 | PU_{F}•U_{F}•aU_{F}aG_{F}aG_{F}cA_{F}agaA_{F}cA_{F}cU_{F}U_{F}U•u•u (SEQ ID NO: 60) | 7662.9 | 7663.3 |
| 28 | U_{F/Me}•U_{F}•aU_{F}aG_{F}aG_{F}cA_{F}agaA_{F}cA_{F}cU_{F}gU_{F}u•u•u (SEQ ID NO: 61) | 7597.9 | 7597.7 |
| 29 | PU_{F/Me}•U_{F}•aU_{F}aG_{F}aG_{F}cA_{F}agaA_{F}cA_{F}cU_{F}gU_{F}u•u•u (SEQ ID NO: 61) | 7676.9 | 7676.7 |
| 30 | VPU_{F/Me}•U_{F}•aU_{F}aG_{F}aG_{F}cA_{F}agaA_{F}cA_{F}cU_{F}gU_{F}u•u•u (SEQ ID NO: 61) | 7673.9 | 7672.4 |
| 31 | ZVPU_{F}Me•U_{F}•aU_{F}aG_{F}aG_{F}cA_{F}agaA_{F}cA_{F}cU_{F}gU_{F}u•u•u (SEQ ID NO: 61) | 7673.9 | 7672.3 |
| 32 | Pu•U_{F}•aU_{F}aG_{F}aG_{F}cA_{F}agaA_{F}cA_{F}cU_{F}U_{F}U•U_{F/Me}•u (SEQ ID NO: 49) | 7676.9 | 7676.7 |
| 33 | Pu•U_{F}•aU_{F}aG_{F}aG_{F}cA_{F}agaA_{F}cA_{F}cU_{F}U_{F}U•U_{F/Me}•U_{F/Me} (SEQ ID NO: 50) | 7678.8 | 7679.0 |
| 34 | A_{F}•a•G_{F}aU_{F}gA_{F}uG_{F}U_{F}U_{F}uG_{F}aA_{F}aC_{F}uA_{F}uU_{F}* (SEQ ID NO: 43) | 8678.2 | 8677.9 |
| 35 | Pa•A_{F}•uA_{F}gU_{F}uU_{F}cA_{F}aacA_{F}uC_{F}aU_{F}cU_{F}u•g•u (SEQ ID NO: 51) | 7595.9 | 7595.7 |
| 36 | Pa•A_{F}•uA_{F}gU_{F/Me}uU_{F}cA_{F}aacA_{F}uC_{F}aU_{F}cU_{F}u•g•u (SEQ ID NO: 52) | 7609.9 | 7610.7 |
| 37 | Pa•A_{F}•uA_{F}gU_{F}U_{F/Me}U_{F}cA_{F}aacA_{F}uC_{F}aU_{F}cU_{F}u•g•u (SEQ ID NO: 53) | 7597.9 | 7598.0 |
| 38 | Pa•A_{F}•uA_{F}gU_{F/Me}U_{F/Me}U_{c}A_{F}aacA_{F}uC_{F}aU_{F}cU_{F}u•g•u (SEQ ID NO: 54) | 7611.9 | 7612.5 |
| 39 | C_{F}•a•G_{F}gA_{F}uC_{F}aU_{F}C_{F}U_{F}cA_{F}aG_{F}uC_{F}uU_{F}aA_{F}* (SEQ ID NO: 44) | 8612.2 | 8611.7 |
| 40 | u•U_{F}•aA_{F}gA_{F}cU_{F}uG_{F}agaU_{F}gA_{F}uC_{F}cU_{F}g•g•c(SEQ ID NO: 55) | 7627.0 | 7626.1 |
| 41 | Pu•U_{F}•aA_{F}gA_{F}cU_{F}UG_{F}agaU_{F}gA_{F}uC_{F}cU_{F}g•g•c(SEQ ID NO: 55) | 7705.9 | 7706.4 |
| 42 | U_{F/Me}•U_{F}•aA_{F}gA_{F}cU_{F}uG_{F}agaU_{F}gA_{F}uC_{F}cU_{F}g•g•c (SEQ ID NO: 56) | 7628.9 | 7627.7 |
| 43 | PU_{F/Me}•U_{F}•aA_{F}gA_{F}cU_{F}uG_{F}agaU_{F}gA_{F}uC_{F}cU_{F}g•g•c (SEQ ID NO: 56) | 7707.9 | 7707.9 |
| 44 | Pu•U_{F}•aA_{F}gA_{F}C_{F/Me}U_{F}uG_{F}agaU_{F}gA_{F}uC_{F}cU_{F}g•g•c (SEQ ID NO: 57) | 7707.9 | 7708.2 |
| 45 | C_{F/Me}•a•G_{F}gA_{F}uC_{F}aU_{F}C_{F}U_{F}cA_{F}aG_{F}cC_{F}uU_{F}aA_{F}* (SEQ ID NO: 45) | 8626.2 | 8626.1 |
| 46 | a•g•uguuC_{F}uU_{F}G_{F}C_{F}ucuauaaaca* (SEQ ID NO: 46) | 8686.5 | 8685.8 |
| 47 | u•G_{F}•uuuauagagcaA_{F}gA_{F}acacu•g•u (SEQ ID NO: 58) | 7707.2 | 7706.6 |
| 48 | u•G_{F}•uuuaU_{F/Me}agagcaA_{F}gA_{F}acacu•g•u (SEQ ID NO: 59) | 7709.2 | 7708.6 |
| 49 | GGAU_{F}C_{F}AU_{F}C_{F}U_{F}C_{F}AAGU_{F}C_{F}U_{F}U_{F}AC_{F}dT•dT (SEQ ID NO: 62) | 6629.0 | 6627.8 |
| 50 | GU_{F}AAGAC_{F}U_{F}U_{F}GAGAU_{F}GAU_{F}C_{F}C_{F}dT•dT (SEQ ID NO: 63) | 6726.1 | 6724.9 |
| 51 | GU_{F/Me}AAGAC_{F}U_{F}U_{F}GAGAU_{F}GAU_{F}C_{F}C_{F}dT•dT (SEQ ID NO: 64) | 6740.1 | 6739.3 |
| 52 | GU_{F}AAGAC_{F/Me}U_{F/Me}U_{F/Me}GAGAU_{F}GAU_{F/Me}C_{F/Me}C_{F/Me}dT•dT (SEQ ID NO: 65) | 6810.2 | 6809.3 |
| 53 | GU_{F/Me}AAGAC_{F/Me}U_{F/Me}U_{F/Me}GAGAU_{F/Me}GAU_{F/Me}C_{F/Me}C_{F/Me}dT•dT (SEQ ID NO: 66) | 6838.3 | 6837.5 |
| 54 | GGAU_{F/Me}C_{F/Me}AU_{F/Me}C_{F/Me}U_{F/Me}C_{F/Me}AAGU_{F/Me}C_{F/Me}U_{F/Me}U_{F/Me}AC_{F/Me}dT•dT | 6783.3 | 6782.5 |
| | (SEQ ID NO: 67) | | |
| 55 | C_{F}•a•G_{F}gA_{F}uC_{F}aU_{F}C_{F}U_{F}cA_{F}aG_{F}uC_{F}uU_{F}aC_{F}* (SEQ ID NO: 68) | 8588.2 | 8557.2 |
| 58 | g•U_{F}•aA_{F}gA_{F}cU_{F}uG_{F}agaU_{F}gA_{F}uC_{F}cU_{F}g•g•c (SEQ ID NO: 69) | 7666.0 | 7664.9 |
| 59 | g•U_{F}•aA_{F}gA_{F}cU_{F}uG_{F}agaU_{F}gA_{F}uC_{F}cU_{F/Me}g•g•c (SEQ ID NO: 70) | 7680.0 | 7679.1 |
| 60 | g•U_{F}•aA_{F}gA_{F}CU_{F}UG_{F}agaU_{F}gA_{F}uC_{F/Me}CU_{F/Me}g•g•c (SEQ ID NO: 71) | 7694.0 | 7693.2 |
| 61 | g•U_{F}•aA_{F}gA_{F}cU_{F/Me}uG_{F}agaU_{F}gA_{F}uCF_{/Me}CU_{F/Me}g•g•c (SEQ ID NO: 72) | 7708.1 | 7707.0 |
| 62 | C_{F}•a•G_{F}gA_{F}uC_{F}AU_{F/Me}C_{F/Me}U_{F/Me}CA_{F}aG_{F}uC_{F}uU_{F}aC_{F}* (SEQ ID NO: 73) | 8630.3 | 8629.6 |
| 63 | dTdAdCdAdGU_{F}dCdTdAdTdGdT (SEQ ID NO: 74) | 3639.4 | 3638.9 |
| 64 | dTdAdCdAdGU_{F/Me}dCdTdAdTdGdT (SEQ ID NO: 75) | 3653.4 | 3652.9 |
| 65 | dTdAdCdAdGdUC_{F}dTdAdTdGdT (SEQ ID NO: 76) | 3653.4 | 3652.4 |
| 66 | dTdAdCdAdGC_{F/Me}dCdTdAdTdGdT (SEQ ID NO: 77) | 3667.4 | 3666.2 |

^{a}Uppercase letters (B) = ribonucleotides, prefix d (dB) = deoxyribonucleotides, subscript F (BF) = 2′-fluoro nucleotides, lowercase letters (b) = 2′-OMe nucleotides, subscript F/Me (B_{F/M}e) = 2′-F/Me modified nucleotides, • = phosphorothiate linkage, P = 5′-monophosphate, VP = 5′-(E)-vinyl phosphonate, zVP = 5′-(Z)-vinyl phosphonate * = triantennary GalNAc ligand.

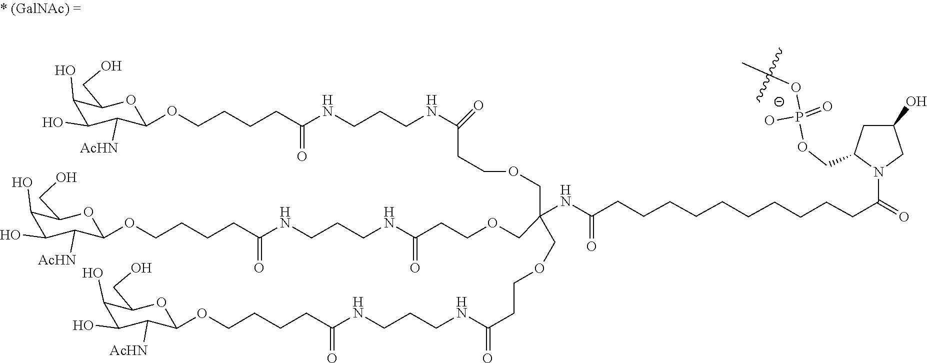 [SMILES: *OP(=O)([O-])OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

TABLE 10
Thermal denaturation temperatures of singly modified duplexes in high-salt PBSª

| | | Tₘ [ΔTₘ] (° C.) | Tₘ [ΔTₘ] (° C.) |
|---|---|---|---|
| | | ON2: | ON57: |
| | | 3′-r(AUGUCUGAUACA) | 3′-d(ATGTCACATACA) |
| | Modified strand | (SEQ ID NO: 84) | (SEQ ID NO: 85) |
| ON1 | 5′-r(UACAGUCUAUGU) (SEQ ID NO: 36) | 61.6 | 47.1 |
| ON3 | 5′-r(UACAGU_{F}CUAUGU)(SEQ ID NO: 38) | 62.2 [+0.6] | 47.8 [+0.8] |
| ON4 | 5′-r(UACAGU_{F/Me}CUAUGU) (SEQ ID | 47.1 [-14.5] | 30.1 [-16.9] |
| | NO: 39) | | |
| ON5 | 5′-r(UACAGUC_{F}UAUGU) (SEQ ID NO: 40) | 63.1 [+1.5] | 47.6 [+0.6] |
| ON6 | 5′-r(UACAGUC_{F/Me}UAUGU) (SEQ ID | 45.2 [-16.4] | 29.6 [-17.5] |
| | NO: 41) | | |
| ON56 | 5′-d(TACAGTCTATGT) (SEQ ID NO: 78) | 49.7 | 51.1 |
| ON58 | 5′-d(TACAGU_{F}CTATGT) (SEQ ID NO: 79) | 50.5 [+0.8] | 48.2 [-2.9] |
| ON59 | 5′-d(TACAGU_{F/Me}CTATGT) (SEQ ID | 27.9 [-21.8] | 28.2 [-22.9] |
| | NO: 80) | | |
| ON60 | 5′-d(TACAGTC_{F}TATGT) (SEQ ID NO: 81) | 51.6 [+1.9] | 50.2 [-0.9] |
| ON61 | 5′-d(TACAGTC_{F/ME}TATGT) (SEQ ID | 34.5 [-15.2] | 36.6 [-14.5] |
| | NO: 82) | | |

^{a}Tₘ was measured by monitoring the absorbance of hybridized duplexes (2.5 μM) with increasing temperature (A₂₆₀ vs T) in high-salt PBS (932 mM NaCl, 18.4 mM KCl, 68 mM Na₂HPO₄, 12.2 mM KH₂PO₄, pH 7•4). Tₘ was determined as the maximum of the first derivative of the melting curve (A₂₆₀ vs T) and reported as the average of two independent experiments• ΔTₘ is calculated with respect to the corresponding complementary duplex.

### REFERENCES

1. Clark, J. L.; Hollecker, L.; Mason, J. C.; Stuyver, L. J.; Tharnish, P. M.; Lostia, S.; McBrayer, T. R.; Schinazi, R. F.; Watanabe, K. A.; Otto, M. J.; Furman, P. A.; Stec, W. J.; Patterson, S. E.; Pankiewicz, K. W., Design, synthesis, and antiviral activity of 2′-deoxy-2′-fluoro-2′-C-methylcytidine, a potent inhibitor of hepatitis C virus replication. J Med Chem 2005, 48 (17), 5504-8.
2. Pradere, U.; Amblard, F.; Coats, S. J.; Schinazi, R. F., Synthesis of 5′-methylene-phosphonate furanonucleoside prodrugs: application to D-2′-deoxy-2′-α-fluoro-2′-β-C-methyl nucleosides. Org Lett 2012, 14 (17), 4426-9.
3. Damha, M. J.; Ogilvie, K. K., Oligoribonucleotide synthesis. The silyl-phosphoramidite method. Methods Mol Biol 1993, 20, 81-114.
4. Liao, Y.; Smyth, G. K.; Shi, W., featureCounts: an efficient general purpose program for assigning sequence reads to genomic features. Bioinformatics 2014, 30 (7), 923-30.
5. Akinc, A.; Goldberg, M.; Qin, J.; Dorkin, J. R.; Gamba-Vitalo, C.; Maier, M.; Jayaprakash, K. N.; Jayaraman, M.; Rajeev, K. G.; Manoharan, M.; Koteliansky, V.; Rohl, I.; Leshchiner, E. S.; Langer, R.; Anderson, D. G., Development of Lipidoid-siRNA Formulations for Systemic Delivery to the Liver. Molecular Therapy 2009, 17 (5), 872-879.
6. Akinc, A.; Querbes, W.; De, S.; Qin, J.; Frank-Kamenetsky, M.; Jayaprakash, K. N.; Jayaraman, M.; Rajeev, K. G.; Cantley, W. L.; Dorkin, J. R.; Butler, J. S.; Qin, L.; Racie, T.; Sprague, A.; Fava, E.; Zeigerer, A.; Hope, M. J.; Zerial, M.; Sah, D. W. Y.; Fitzgerald, K.; Tracy, M. A.; Manoharan, M.; Koteliansky, V.; Fougerolles, A. d.; Maier, M. A., Targeted Delivery of RNAi Therapeutics With Endogenous and Exogenous Ligand-Based Mechanisms. Molecular Therapy 2010, 18 (7), 1357-1364.
7. Pettersen, E. F.; Goddard, T. D.; Huang, C. C.; Couch, G. S.; Greenblatt, D. M.; Meng, E. C.; Ferrin, T. E., UCSF Chimera—a visualization system for exploratory research and analysis. J Comput Chem 2004, 25 (13), 1605-12.

### Synthesis of Additional Exemplary Compounds

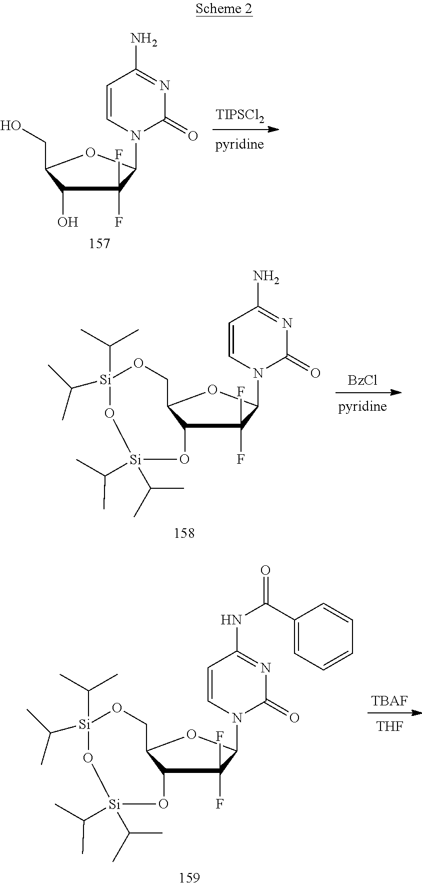

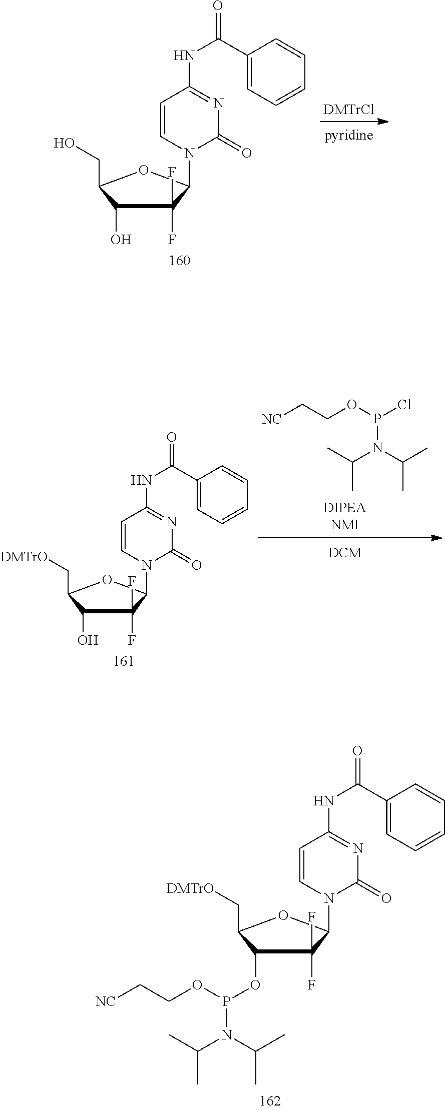

### Experimental Details:

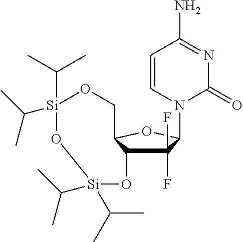

1-[(6aR,8R)-9,9-difluoro-2,2,4,4-tetraisopropyl-6,6a,8,9a-tetrahydrofuro[3,2-f][1,3,5,2,4]trioxadisilocin-8-yl]-4-amino-pyrimidin-2-one 158: To a clear solution of 157 (1.7 g, 6.46 mmol) in pyridine (30 mL) was added dichloro-1,1,3,3-tetraisopropyldisiloxane (2.04 g, 6.46 mmol) in single portion. Reaction mixture was stirred at 22° C. for 16 hr. All volatile matters were removed under high vacuum pump and residue was dissolved in DCM (100 mL). Organic layer was washed with 10% NaHCO₃ solution (50 mL) and brine (2×40 mL). DCM layer was separated, dried over anhydrous Na₂SO₄, filtered and filtrate was evaporated to dryness. crude compound was purified by flash column chromatography (gradient: 0-5% MeOH in DCM) to afford 158 (2.4 g, 73% yield) as white solid. ¹H NMR (600 MHz, DMSO-d₆) δ 7.49 (d, J=7.5 Hz, 1H), 7.43 (s, 2H), 6.35-6.04 (m, 1H), 4.40-4.28 (m, 1H), 4.17 (dd, J=13.0, 3.4 Hz, 1H), 3.96 (ddd, J=14.4, 11.2, 3.0 Hz, 2H), 1.15-0.97 (m, 28H) ppm. ¹⁹F NMR (565 MHz, DMSO-d₆) δ −116.76 ppm. ¹³C NMR (151 MHz, DMSO-d₆) δ 165.64, 154.41, 139.29, 123.98, 122.29, 120.57, 94.85, 83.46, 77.85, 70.33, 60.34, 54.91, 17.18, 17.06, 17.04, 17.02, 16.72, 16.70, 16.51, 12.58, 12.29, 11.96, 11.95 ppm.

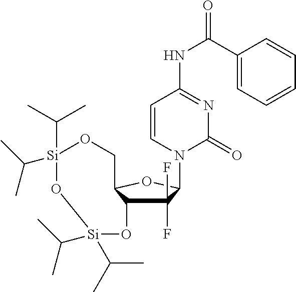

N-[1-[(6aR,8R)-9,9-difluoro-2,2,4,4-tetraisopropyl-6,6a,8,9a-tetrahydrofuro[3,2-f][,3,5,2,4]trioxadisilocin-8-yl]-2-oxo-pyrimidin-4-yl]benzamide 159: To a clear solution of 158 (2.4 g, 4.75 mmol) in pyridine (30 mL) at 0° C. was added benzoyl chloride (667.13 mg, 4.75 mmol) in three portions. Reaction mixture was stirred for 8 hr at 22° C. and then quenched with 10% NaHCO₃ solution (25 mL). DCM (30 mL) was added, and organic layer was separated, dried over anhydrous Na₂SO₄, filtered and filtrate was evaporated to dryness. Crude compound thus obtained, was purified by flash column chromatography (gradient: 30-50% EtOAc in hexane) to afford 159 (2.75 g, 95% yield) as white foam. ¹H NMR (600 MHz, DMSO-d₆) δ 11.44 (s, 1H), 8.05-7.99 (m, 3H), 7.66-7.60 (m, 1H), 7.55-7.49 (m, 2H), 7.46-7.41 (m, 1H), 6.21 (s, 1H), 4.46 (s, 1H), 4.24 (dd, J=13.0, 3.6 Hz, 1H), 4.09-3.99 (m, 2H), 1.16-0.99 (m, 28H) ppm. ¹⁹F NMR (565 MHz, DMSO-d₆) δ −116.79 ppm. ¹³C NMR (151 MHz, DMSO-d₆) δ 167.47, 167.35, 163.85, 153.92, 143.56, 132.90, 129.24, 128.55, 128.46, 123.89, 122.18, 120.45, 96.92, 95.40, 84.32, 78.43, 70.35, 60.40, 17.18, 17.06, 17.03, 16.72, 16.70, 16.51, 12.55, 12.29, 11.98, 11.96 ppm.

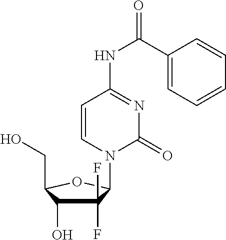

N-[1-[(2R,5R)-3,3-difluoro-4-hydroxy-5-(hydroxymethyl)tetrahydrofuran-2-yl]-2-oxo-pyrimidin-4-yl]benzamide 160: To a clear solution of 159 (2.75 g, 4.51 mmol) in THF (30 mL) was added TBAF (2.83 g, 10.82 mmol) in single portion and stirred for 3 hr at 22° C. All the volatile matters were evaporated under high vacuum pump and the crude residue thus obtained, was purified by flash column chromatography (gradient: 0-5% MeOH in DCM) to afford 160 (1.22 g, 74% yield) as white foam. ¹H NMR (600 MHz, DMSO-d₆) δ 11.39 (s, 1H), 8.32 (d, J=7.6 Hz, 1H), 8.03-7.97 (m, 2H), 7.66-7.61 (m, 1H), 7.52 (td, J=8.1, 2.1 Hz, 2H), 7.43-7.39 (m, 1H), 6.34 (dd, J=6.6, 2.1 Hz, 1H), 6.21 (t, J=7.3 Hz, 1H), 5.33 (td, J=5.5, 2.0 Hz, 1H), 4.27-4.17 (m, 1H), 3.92 (dq, J=8.3, 2.6 Hz, 1H), 3.86-3.80 (m, 1H), 3.71-3.64 (m, 1H) ppm. ¹⁹F NMR (565 MHz, DMSO-d₆) δ −116.86 ppm. ¹³C NMR (151 MHz, DMSO-d₆) δ 167.45, 163.73, 154.20, 144.71, 132.99, 132.87, 128.55, 128.47, 124.70, 122.99, 121.27, 96.67, 95.41, 84.41, 84.21, 83.99, 81.07, 81.05, 81.02, 68.50, 68.35, 68.21, 58.78, 48.61 ppm.

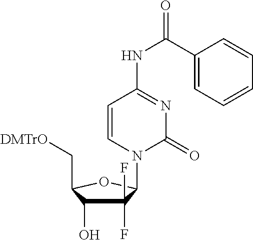

N-[1-[(2R,5R)-5-[[bis(4-methoxyphenyl)-phenyl-methoxy]methyl]-3,3-difluoro-4-hydroxy-tetrahydrofuran-2-yl]-2-oxo-pyrimidin-4-yl]benzamide 161: To a clear solution of 160 (1.22 g, 3.32 mmol) in pyridine (30 mL) was added 4,4′-dimethoxytrityl chloride (1.35 g, 3.99 mmol) in two portions. Reaction mixture was stirred at for 16 hr, diluted with DCM (30 mL) and then quenched with 10% NaHCO₃ (30 mL). Organic layer was washed with brine (2×20 mL), separated, dried over anhydrous Na₂SO₄, and filtered. Filtrate was evaporated under high vacuum pump and crude mass obtained, was purified by flash column chromatography (10-50% EtOAc in hexane) to afford 161 (1.95 g, 88% yield) as yellowish white foam. ¹H NMR (600 MHz, DMSO-d₆) δ 11.41 (s, 1H), 8.23 (d, J=7.6 Hz, 1H), 8.03-7.98 (m, 2H), 7.67-7.61 (m, 1H), 7.55-7.49 (m, 2H), 7.43-7.38 (m, 2H), 7.38-7.32 (m, 2H), 7.32-7.24 (m, 6H), 6.96-6.90 (m, 4H), 6.43 (d, J=6.7 Hz, 1H), 6.26 (t, J=7.1 Hz, 1H), 4.47-4.36 (m, 1H), 4.09 (ddd, J=9.1, 4.5, 2.3 Hz, 1H), 3.76 (d, J=0.8 Hz, 6H), 3.46 (dd, J=11.2, 4.7 Hz, 1H), 3.36 (dd, J=11.1, 2.3 Hz, 1H) ppm. ¹⁹F NMR (565 MHz, DMSO-d₆) δ −117.26 ppm. ¹³C NMR (151 MHz, DMSO-d₆) δ 167.44, 163.74, 158.25, 158.23, 154.11, 144.32, 135.33, 135.04, 132.97, 132.88, 129.79, 129.69, 128.53, 128.47, 128.00, 127.76, 126.94, 124.43, 122.71, 121.00, 96.78, 95.40, 86.03, 84.38, 79.16, 79.13, 79.09, 69.11, 68.96, 68.81, 61.58, 59.76, 55.04 ppm.

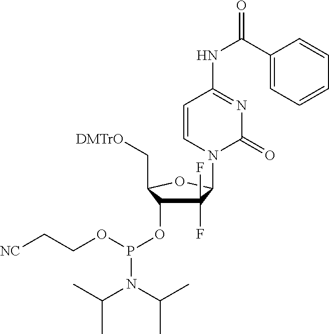

N-[1-[(2R,5R)-5-[[bis(4-methoxyphenyl)-phenyl-methoxy]methyl]-4-[2-cyanoethoxy-(diisopropylamino)phosphanyl]oxy-3,3-difluoro-tetrahydrofuran-2-yl]-2-oxo-pyrimidin-4-yl]benzamide 162: To a clear solution of 161 (1.1 g, 1.64 mmol) in DCM (30 mL) was added N-methylimidazole (269.72 mg, 3.29 mmol, 261.86 L) and diisopropylethylamine (1.06 g, 8.21 mmol, 1.43 mL) in single portions. After stirring the reaction mixture for 5 minutes at 22° C., 2-cyanoethyl-N,N-diisopropylchlorophosphoramidite (777.54 mg, 3.29 mmol, 733.53 μL) was added and continued stirring for 1 hr and TLC was checked. Starting material was consumed and reaction mixture was diluted with DCM (20 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-50% EtOAc in hexane) to afford 162 (1.08 g, 76% yield) as white foam. ¹H NMR (600 MHz, CD₃CN) δ 9.24 (s, 1H), 8.26-8.11 (m, 1H), 7.97-7.92 (m, 2H), 7.67-7.61 (m, 1H), 7.55-7.50 (m, 2H), 7.50-7.43 (m, 2H), 7.40-7.22 (m, 7H), 6.94-6.86 (m, 4H), 6.28 (t, J=7.3 Hz, 1H), 4.83-4.53 (m, 1H), 4.19 (ddt, J=9.1, 6.1, 2.3 Hz, 1H), 3.79 (d, J=5.7 Hz, 6H), 3.64-3.44 (m, 4H), 2.63 (t, J=5.9 Hz, 1H), 2.49 (t, J=6.0 Hz, 1H), 1.27-1.06 (m, 12H), 1.00 (d, J=6.8 Hz, 2H) ppm. ³¹P NMR (243 MHz, CD₃CN) 6 152.74, 151.77 ppm. ¹⁹F NMR (565 MHz, CD₃CN) δ −115.92, −116.03 ppm. ¹³C NMR (151 MHz, CD₃CN) δ 171.66, 168.05, 164.32, 159.87, 155.53, 145.44, 145.36, 145.22, 136.41, 136.35, 136.31, 136.30, 134.24, 134.00, 131.15, 131.12, 129.65, 129.19, 129.12, 129.02, 129.00, 128.18, 128.13, 125.19, 125.08, 123.47, 123.45, 123.36, 121.74, 121.63, 119.36, 119.19, 114.20, 97.76, 87.72, 86.23, 86.02, 85.82, 80.42, 72.17, 72.02, 71.92, 71.87, 71.77, 71.49, 71.35, 71.24, 71.09, 62.84, 62.80, 62.06, 61.54, 60.96, 60.03, 59.90, 59.76, 55.94, 55.91, 55.32, 48.36, 48.33, 44.29, 44.25, 44.20, 44.17, 24.92, 24.88, 24.87, 24.76, 24.74, 24.71, 24.69, 22.78, 22.76, 21.35, 21.33, 21.14, 20.96, 20.92, 20.90, 20.85, 20.12, 20.05 ppm.

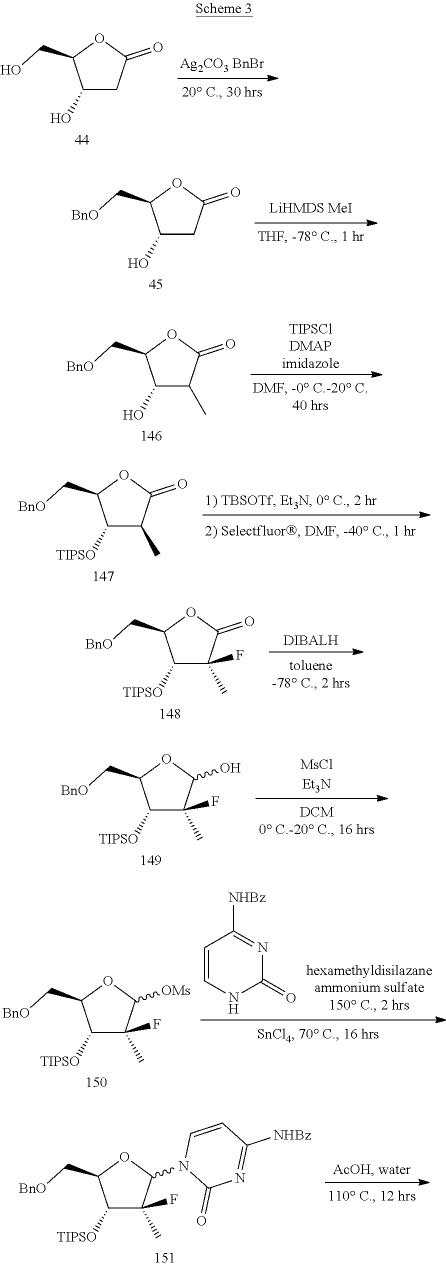

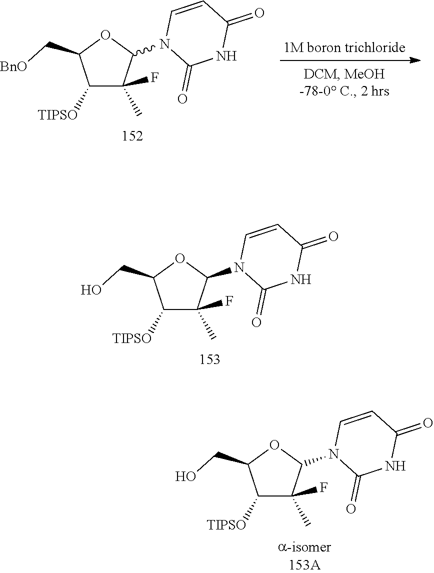

### Compound 45

To a solution of 44 (40 g, 302.77 mmol) in toluene (1600 mL) was added BnBr (54.37 g, 317.91 mmol) and Ag₂CO₃ (250.46 g, 908.30 mmol). The reaction mixture was stirred at 20° C. for 30 hours. For additional vials in 40 g scale were set up as described above. The combined reaction mixture was diluted with tetrahydrofuran (1500 mL) and filtered, and the filter cake was rinsed with ethyl acetate (2×1000 mL). The combined organic layers were concentrated under reduced pressure. The residue was purified by silica gel chromatography (eluting with petroleum ether/ethyl acetate=10/1 to 3/1) to give 45 (120 g, 539.97 mmol, yield 35.67%) as a yellow oil. ¹H NMR: (400 MHz, METHANOL-d₄) δ ppm 2.25-2.63 (m, 1H) 2.90 (dd, J=18.01, 6.63 Hz, 1H) 3.66-3.75 (m, 2H) 4.27-4.45 (m, 1H) 4.51-4.59 (m, 2H) 7.19-7.41 (m, 5H).

### Compound 146

To a solution of 45 (20 g, 89.99 mmol) and Mel (26.82 g, 188.99 mmol) in THF (400 mL) was added LiHMDS (1 M, 206.99 mL, 2.3 eq) dropwise at −78° C. The reaction mixture was stirred at −78° C. for 60 minutes. The mixture was poured into a saturated aqueous NH₄Cl solution (800 mL). Three additional vials in 20 g scale were set up as described above. And the mixture was extracted with ethyl acetate (3×1000 mL). The combined organic layers were dried over Na₂SO₄, filtered and concentrated, the residue was purified by silica gel chromatography eluted with petroleum ether/ethyl acetate=10:1 to 3:1 to get 146 (16 g, 67.72 mmol, yield 18.81%) as a colorless oil. ¹H NMR: (400 MHz, METHANOL-d₄) δ ppm 1.11-1.28 (m, 4H) 2.51-2.98 (m, 1H) 3.62-3.71 (m, 1H) 3.81 (dd, J=11.40, 2.19 Hz, 1H) 3.96 (dd, J=8.44, 7.34 Hz, 1H) 4.25 (ddd, J=7.34, 4.93, 2.19 Hz, 1H) 4.42 (t, J=3.51 Hz, 1H) 4.50-4.62 (m, 1H) 4.50-4.60 (m, 1H) 7.24-7.38 (m, 5H)

### Compound 147

To a solution of 146 (15 g, 63.49 mmol) in DMF (300 mL) was added IMIDAZOLE (21.61 g, 317.44 mmol), DMAP (38.78 g, 317.44 mmol) and TIPSCl (48.96 g, 253.95 mmol) at 0° C. The reaction mixture was stirred at 20° C. for 40 hours. The reaction mixture was diluted with water (300 mL), and the mixture was extracted with ethyl acetate (2×200 mL). The combined organic layers were washed with water (2×200 mL), brine (2×200 mL) and concentrated under reduced pressure. One additional vial in 20 g scale was set up as described above. The residue was purified by silica gel chromatography (eluting with petroleum ether/ethyl acetate=100/1 to 30/1) to give 147 (25 g, 63.68 mmol, yield 50.15%) as a colorless oil. ¹H NMR: (400 MHz, CHLOROFORM-d) δ ppm 1.05-1.07 (m, 65H) 1.30-1.36 (m, 3H) 2.62 (dd, J=7.45, 5.70 Hz, 1H) 3.58-3.68 (m, 1H) 3.70-3.77 (m, 1H) 4.20-4.36 (m, 2H) 4.49-4.63 (m, 2H) 7.28-7.39 (m, 4H).

### Compound 148

To a solution of 147 (25 g, 63.68 mmol) in DCM (400 mL) was added TEA (20.62 g, 203.77 mmol, 28.36 mL, 3.2 eq) and TBSOTf (26.93 g, 101.88 mmol) at 0° C. The reaction was stirred for 2 hrs at 0° C., and then quenched with water (100 mL). The aqueous was extracted with dichloromethane (3×100 mL). The combined organic layers were concentrated under reduced pressure. The residue was dissolved in DMF (400 mL) and cooled to −40° C., and then Selectfluor F (45.12 g, 127.36 mmol) was added and the reaction was stirred at −40° C. for 1 hour. The reaction mixture was quenched by addition of water (400 mL), and the aqueous layer was extracted with ethyl acetate (3×200 mL). The combined organic layer was dried over Na₂SO₄ and concentrated under reduced pressure. The residue was purified by column chromatography on silica gel (eluted with petroleum ether/ethyl acetate=50/1) to give 148 (18 g, 43.84 mmol, yield 68.85%) as a colorless oil. ¹H NMR: (400 MHz, CHLOROFORM-d) δ ppm 1.02-1.14 (m, 57H) 1.54-1.64 (m, 3H) 3.67-3.73 (m, 1H) 3.78-3.85 (m, 1H) 4.07-4.15 (m, 1H) 4.57-4.60 (m, 2H) 4.73-4.86 (m, 1H) 7.29-7.43 (m, 5H)

### Compound 149

To a solution of 148 (18 g, 43.84 mmol) in toluene (360 mL) was added DIBAL-H (1 M, 175.36 mL) dropwise at −78° C. The reaction mixture was stirred at −78° C. for 2 hrs. The reaction was quenched by addition of methanol (20 mL). The mixture was allowed to warm to room temperature and aqueous HCl (300 mL, 0.1 mol/L) was added to the mixture. The mixture was stirred for 10 mins and the organic layer was separated. The aqueous layer was extracted with methyl t-butyl ether (4×200 mL). The combined organic layers were dried over Na₂SO₄ and concentrated under reduced pressure. The residue was purified by column chromatography on silica gel (eluted with petroleum ether/ethyl acetate=20/1 to 4/1) to give 149 (13.5 g, 32.72 mmol, yield 74.63%) as a colorless oil. ¹H NMR: (400 MHz, CHLOROFORM-d) δ ppm 0.98-1.21 (m, 21H) 1.39-1.69 (m, 3H) 3.23 (br d, J=10.76 Hz, 1H) 3.46-3.71 (m, 2H) 3.86-3.95 (m, 1H) 4.19 (br d, J=13.38 Hz, 1H) 4.37 (br t, J=6.50 Hz, 1H) 4.47-4.73 (m, 2H) 4.95 (br d, J=6.50 Hz, 1H) 5.17(br t, J=9.13 Hz, 1H) 7.28-7.44 (m, 4H)

### Compound 150

To a solution of 149 (13.5 g, 32.72 mmol) in DCM (150 mL) at 0° C. was added TEA (6.62 g, 65.44 mmol) and MsCl (6.76 g, 59.01 mmol). The resulting suspension was stirred for 10 minutes and then warmed to 20° C. and stirred for 16 hrs. The reaction was quenched with aqueous NaHCO₃ (1 mol/L, 150 mL) and diluted with ethyl acetate (400 mL). The layers were separated. The organic layer was washed with aqueous HCl (100 mL, 1 mol/L), water (100 mL) and brine (100 mL), dried over Na₂SO₄ and concentrated under reduced pressure to give a yellow oil, which was diluted with heptane (120 mL) and filtered through Celite®. The filtrate was concentrated under reduced pressure to give 150 (15 g, 30.57 mmol, yield 93.43%) as a yellow oil. ¹H NMR (400 MHz, CHLOROFORM-d) 0.96-1.20 (m, 25H) 1.47-1.71 (m, 4H) 3.68 (d, J=5.00 Hz, 1H) 3.71-3.77 (m, 1H) 4.02 (td, J=6.63, 3.75 Hz, 1H) 4.20 (br dd, J=3.88, 0.75 Hz, 1H) 4.26 (br dd, J=3.88, 0.88 Hz, 1H) 4.42 (q, J=4.88 Hz, 1H) 4.56-4.62 (m, 2H) 4.63-4.76 (m, 1H) 5.91 (s, 1H) 6.11 (d, J=10.76 Hz, 1H) 7.28-7.42 (m, 5H) ppm.

### Compound 151

A mixture of N-(2-oxo-1H-pyrimidin-4-yl)benzamide (28.07 g, 130.43 mmol), AMMONIUM SULFATE (6.46 g, 48.91 mmol), and HEXAMETHYLDISILAZANE (288.00 mL) was heated to 140° C., becoming a solution within 10 minutes. After 2 hours, the solution was cooled to room temperature and concentrated under reduced pressure to give a thick oil, which was placed under high vacuum. The solution of 150 (16 g, 32.61 mmol) in DCE (200 mL) was added to the residue and followed by dropwise addition of SnCl₄ (17.84 g, 68.47 mmol) at 20° C., and the resulting solution was stirred at 20° C. for 30 minutes then at 70° C. for 16 hrs. After cooling to room temperature then to 0° C., the reaction mixture was quenched by adding to an ice-cold aqueous solution of Na₂CO₃ (1 mol/L, 200 mL) and stirred for 5 minutes before warming to 20° C. and stirring for 20 minutes. Celite® (50 g) was added, and the mixture was stirred for 15 minutes, then filtered through a pad of Celite®, and the solid was rinsed with ethyl acetate (5×200 mL). The filtrate was washed with water (200 mL) and brine (200 mL). The combined aqueous layers were back extracted with ethyl acetate (2×250 mL). One additional vial in 9 g scale was set up as described above. The combined organic layers were dried over MgSO₄ and concentrated under reduced pressure, and the residue was purified by silica gel chromatography eluted with petroleum ether/ethyl acetate=10:1 to 1:1 to get 151(27 g, 44.28 mmol, yield 87.05%) as a colorless oil. ¹H NMR: (400 MHz, CHLOROFORM-d) δ ppm 0.97-1.19 (m, 21H) 1.45-1.67 (m, 3H) 3.56-3.66 (m, 1H) 3.67-3.74 (m, 1H) 3.74-3.81 (m, 1H) 4.08 (br d, J=4.50 Hz, 1H) 4.27-4.37 (m, 1H) 4.37-4.44 (m, 1H) 4.53-4.68 (m, 3H) 6.22-6.62 (m, 1H) 7.29-7.45 (m, 5H) 7.49-7.58 (m, 2H) 7.59-7.66 (m, 1H) 7.83-8.13 (m, 3H) 8.67 (br s, 1H) ppm.

### Compound 152

A mixture of 151 (27 g, 44.28 mmol) in the mixture of AcOH (300 mL) and H₂O (75 mL) was heated to 110° C. and stirred for 12 hrs. The combined mixture was concentrated under reduced pressure, and the residue was purified by silica gel chromatography eluted with petroleum ether/ethyl acetate=10:1 to 5:1 to give 152 (18 g, 35.53 mmol, yield 80%) as a colorless oil. ¹H NMR: (400 MHz, CHLOROFORM-d) δ ppm 0.98-1.18 (m, 21H) 1.40-1.60 (m, 3H) 3.51-3.61 (m, 1H) 3.62-3.70 (m, 1H) 3.70-3.81 (m, 1H) 4.01 (q, J=4.48 Hz, 1H) 4.32 (d, J=13.82 Hz, 1H) 4.40 (dd, J=17.73, 4.65 Hz, 1H) 4.51-4.65 (m, 2H) 5.52 (dd, J=8.25, 2.14 Hz, 1H) 5.68 (dd, J=8.19, 2.08 Hz, 1H) 6.07 (d, J=13.82 Hz, 1H) 6.35 (d, J=16.75 Hz, 1H) 7.29-7.40 (m, 4H) 7.44-7.51 (m, 1H) 7.57 (d, J=8.19 Hz, 1H) 7.60-7.64 (m, 1H) 8.07-8.16 (m, 1H) 8.60-8.82 (m, 1H)

### Compound 153 and Isomer −153A

To a solution of 152 (9 g, 17.76 mmol) in DCM (180 mL) at −78° C. was added BCl₃ (1 mol/L, 88.81 mL) at −70° C. And the solution was stirred at −78 to −70° C. for 1 hr and then allowed to warm to 0° C. in the cooling bath for 1 hr. The mixture was quenched with methanol (150 mL) and the solution was warmed to 20° C. One additional vial in 9 g scale was set up as described above. The mixture was concentrated under reduced pressure, and the residue was purified by Preparative HPLC (neutral system), and the desired eluants were lyophilized respectively to give 153 (1 g, 2.40 mmol, yield 6.76%) as a white solid and isomer (4 g, 9.60 mmol, yield 27.03%) as a white solid. ¹H NMR for 153: ET48980-107-P1H (400 MHz, DMSO-d₆) δ ppm 0.94-1.19 (m, 21H) 1.36-1.54 (m, 3H) 3.55-3.65 (m, 1H) 3.67-3.76 (m, 1H) 3.80 (q, J=4.42 Hz, 1H) 4.41 (dd, J=19.01, 5.13 Hz, 1H) 5.22 (t, J=5.25 Hz, 1H) 5.67 (d, J=8.13 Hz, 1H) 5.93 (d, J=13.76 Hz, 1H) 7.72 (dd, J=8.25, 1.88 Hz, 1H) 11.44 (br s, 1H)

### Preparative HPLC Method:

a. Instrument: Shimadzu LC-8A preparative HPLC
b. Column: Welch Xtimate C18 250*100 mm #10 um
c. Mobile phase: A for H₂O (10 mM NH₄HCO₃) and B for ACN
d. Gradient: B from 40% to 70% in 20 min
e. Flow rate: 260 mL/min
f. Wavelength: 220 &254 nm

LCMS for 153 (ESI+): m/z 417 (M+1), RT: 3.275 min.

LCMS method: LC/MS(The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH₄HCO₃, mobile phase B was Acetonitrile. The column used for chromatography was a Xbridge-C18 2.1*50 mm 5 um. Detection methods are diode array (DAD), and evaporative light scattering detection (ELSD). MS mode was positive electrospray ionization. MS range was 100-1000.

¹H NMR for 153A: ET48980-107-P2C (400 MHz, DMSO-d₆) δ ppm 1.01-1.08 (m, 18H) 1.09-1.18 (m, 3H) 1.30-1.42 (m, 3H) 3.47-3.57 (m, 2H) 4.31 (td, J=6.16, 2.19 Hz, 1H) 4.42 (dd, J=14.95, 2.44 Hz, 1H) 5.14 (t, J=5.63 Hz, 1H) 5.63 (d, J=8.13 Hz, 1H) 6.14 (d, J=15.88 Hz, 1H) 7.53 (d, J=8.13 Hz, 1H) 11.48 (s, 1H)

LCMS for 153A (ESI+): m/z 417 (M+1), RT: 3.139 min

LCMS method: LC/MS(The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH4HCO3, mobile phase B was Acetonitrile. The column used for chromatography was a Xbridge-C18 2.1*50 mm 5 um. Detection methods are diode array (DAD), and evaporative light scattering detection (ELSD). MS mode was positive electrospray ionization. MS range was 100-1000.

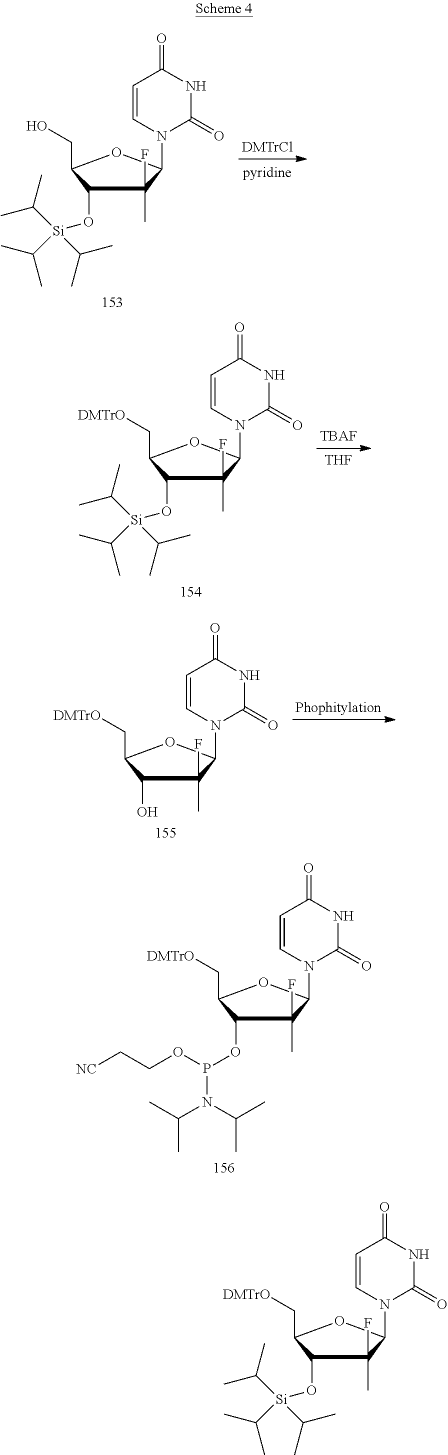

1-[(2S,5R)-5-[[bis(4-methoxyphenyl)-phenyl-methoxy]methyl]-3-fluoro-3-methyl-4-triisopropyl silyloxy-tetrahydrofuran-2-yl]pyrimidine-2,4-dione 154: To a clear solution of 153 (0.22 g, 528.14 μmol) in pyridine (20 mL) was added 4,4′-dimethoxytrityl chloride (214.74 mg, 633.76 μmol) in two portions. Reaction mixture was stirred at for 16 hr, diluted with DCM (30 mL) and then quenched with 10% NaHCO₃ (30 mL). Organic layer was washed with brine (2×20 mL), separated, dried over anhydrous Na₂SO₄, and filtered. Filtrate was evaporated under high vacuum pump and crude mass obtained, was purified by flash column chromatography (10-50% EtOAc in hexane) to afford 154 (0.35 g, 92% yield) as white foam. ¹H NMR (600 MHz, DMSO-d₆) δ 11.52 (s, 1H), 7.66 (dd, J=8.2, 1.9 Hz, 1H), 7.40-7.35 (m, 2H), 7.35-7.30 (m, 2H), 7.28-7.21 (m, 5H), 6.93-6.87 (m, 4H), 5.99 (d, J=12.3 Hz, 1H), 5.49 (d, J=8.1 Hz, 1H), 4.51-4.37 (m, 1H), 3.94 (td, J=6.3, 2.6 Hz, 1H), 3.74 (d, J=1.0 Hz, 6H), 3.43 (dd, J=10.7, 2.7 Hz, 1H), 3.26 (dd, J=10.8, 5.3 Hz, 1H), 1.51-1.38 (m, 3H), 0.94 (d, J=6.7 Hz, 9H), 0.91-0.82 (m, 12H) ppm. ¹³C NMR (151 MHz, DMSO-d₆) δ 162.80, 158.26, 150.48, 144.39, 140.65, 135.04, 134.97, 129.76, 129.74, 127.88, 127.69, 126.89, 113.21, 113.18, 101.86, 101.46, 100.60, 85.80, 81.27, 81.23, 76.12, 75.92, 61.98, 59.76, 55.08, 17.75, 17.64, 17.62, 17.45, 11.88 ppm. ¹⁹F NMR (565 MHz, DMSO-d₆) δ −155.53 ppm.

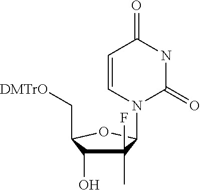

1-[(2R,5R)-5-[[bis(4-methoxyphenyl)-phenyl-methoxy]methyl]-3-fluoro-4-hydroxy-3-methyl-tetrahydrofuran-2-yl]pyrimidine-2,4-dione 155: To a clear solution of 154 (0.33 g, 459.02 μmol) in THF (20 mL) was added TBAF (156.02 mg, 596.73 μmol) in single portion and stirred for 5 hr at 22° C. All the volatile matters were evaporated under high vacuum pump and the crude residue thus obtained, was purified by flash column chromatography (gradient: 0-5% MeOH in DCM) to afford 155 (0.22 g, 85% yield) as white foam. ¹H NMR (600 MHz, DMSO-d₆) δ 11.49 (d, J=2.2 Hz, 1H), 7.48 (dd, J=8.2, 2.2 Hz, 1H), 7.42-7.37 (m, 2H), 7.33 (t, J=7.8 Hz, 2H), 7.29-7.21 (m, 5H), 6.93-6.88 (m, 4H), 5.99-5.91 (m, 2H), 5.52 (dd, J=8.2, 2.2 Hz, 1H), 4.09-4.03 (m, 1H), 3.91 (td, J=5.6, 3.6 Hz, 1H), 3.74 (d, J=1.0 Hz, 6H), 3.31-3.23 (m, 2H), 1.45-1.27 (m, 3H) ppm. ¹³C NMR (151 MHz, DMSO-d₆) δ 162.82, 158.14, 150.49, 144.68, 140.80, 135.43, 135.27, 129.74, 129.70, 127.92, 127.68, 126.81, 113.26, 101.79, 101.32, 100.56, 81.66, 81.63, 74.96, 74.76, 62.79, 59.76, 55.06, 16.74, 16.58 ppm. ¹⁹F NMR (565 MHz, DMSO-d₆) δ−157.12 ppm.

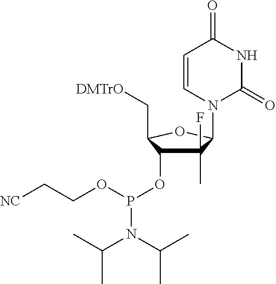

3-[[(2R,5R)-2-[[bis(4-methoxyphenyl)-phenyl-methoxy]methyl]-5-(2,4-dioxopyrimidin-1-yl)-4-fluoro-4-methyl-tetrahydrofuran-3-yl]oxy-(diisopropylamino)phosphanyl]oxypropane nitrile 156: To a clear solution of 155 (0.22 g, 391.05 μmol) in DCM (10 mL) was added N-methylimidazole (64.21 mg, 782.11 μmol, 62.34 L) and DIPEA (252.70 mg, 1.96 mmol, 340.56 μL) in single portions. After stirring the reaction mixture for 5 minutes at 22° C., 2-cyanoethyl N,N-diisopropylchlorophosphoramidite (185.11 mg, 782.11 μmol, 174.63 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-60% EtOAc in hexane) to afford 156 (0.28 g, 94% yield) as white foam. ¹H NMR (600 MHz, CD₃CN) δ 9.04 (s, 1H), 7.57-7.43 (m, 3H), 7.37-7.29 (m, 6H), 7.28-7.21 (m, 1H), 6.91-6.84 (m, 4H), 5.99 (dd, J=15.5, 4.4 Hz, 1H), 5.48 (dd, J=8.2, 3.0 Hz, 1H), 4.43-4.30 (m, 1H), 4.14-4.06 (m, 1H), 3.85-3.68 (m, 7H), 3.67-3.53 (m, 2H), 3.47-3.37 (m, 1H), 3.37-3.30 (m, 1H), 2.61 (t, J=5.9 Hz, 1H), 2.42 (t, J=6.1 Hz, 1H), 1.63-1.35 (m, 3H), 1.26 (dd, J=11.2, 6.7 Hz, 1H), 1.16 (dd, J=8.6, 6.8 Hz, 9H), 1.04 (d, J=6.8 Hz, 3H) ppm. ¹³C NMR (151 MHz, CD₃CN) δ 163.57, 159.75, 151.51, 145.88, 142.03, 142.00, 141.96, 141.93, 136.72, 136.66, 136.65, 136.58, 131.08, 131.06, 131.04, 129.07, 128.99, 128.91, 127.97, 127.95, 119.37, 119.19, 114.11, 114.09, 102.69, 102.61, 102.36, 101.45, 101.33, 87.56, 87.44, 87.37, 87.25, 87.24, 87.22, 82.94, 82.92, 82.91, 82.71, 82.68, 82.65, 78.59, 78.49, 78.39, 78.28, 78.12, 78.02, 77.91, 77.81, 63.36, 62.83, 62.80, 60.96, 59.68, 59.54, 59.49, 59.36, 55.93, 55.91, 48.37, 48.34, 44.22, 44.14, 44.09, 44.01, 24.96, 24.91, 24.84, 24.82, 24.79, 24.76, 24.72, 22.78, 22.76, 21.34, 21.14, 20.98, 20.93, 20.85, 20.80, 20.12, 20.06, 18.09, 17.92, 17.84, 17.68 ppm. ¹⁹F NMR (565 MHz, CD₃CN) δ −158.30, −158.31, −158.74, −158.74 ppm. ³¹P NMR (243 MHz, CD₃CN) δ 150.80, 150.53 ppm.

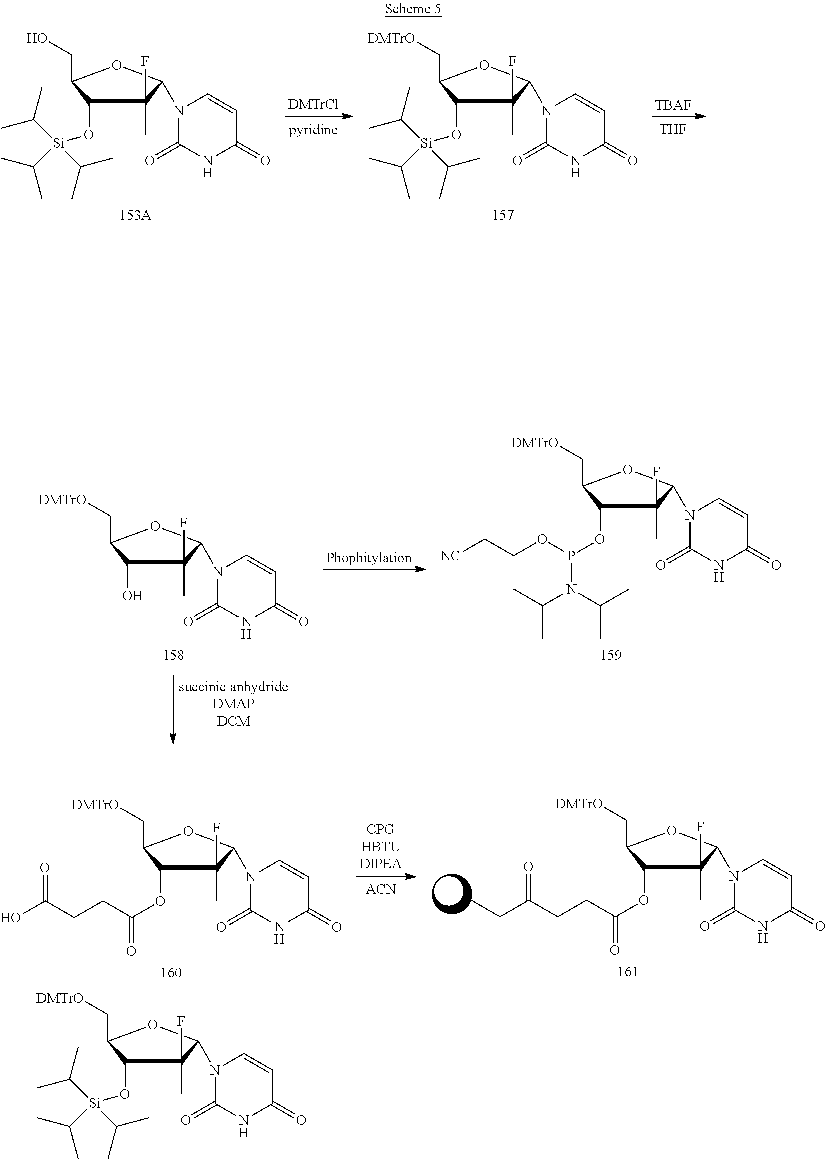

1-[(2S,5R)-5-[[bis(4-methoxyphenyl)-phenyl-methoxy]methyl]-3-fluoro-3-methyl-4-triisopropyl silyloxy-tetrahydrofuran-2-yl]pyrimidine-2,4-dione 157: To a clear solution of 153A (1.0 g, 2.40 mmol) in pyridine (20 mL) was added 4,4′-dimethoxytrityl chloride (976.08 mg, 2.88 mmol) in two portions. Reaction mixture was stirred at for 16 hr, diluted with DCM (30 mL) and then quenched with 10% NaHCO₃ (30 mL). Organic layer was washed with brine (2×20 mL), separated, dried over anhydrous Na₂SO₄, and filtered. Filtrate was evaporated under high vacuum pump and crude mass obtained, was purified by flash column chromatography (10-50% EtOAc in hexane) to afford 157 (1.61 g, 93% yield) as white foam. ¹H NMR (600 MHz, DMSO-d₆) δ 11.50 (d, J=2.2 Hz, 1H), 7.61 (d, J=8.2 Hz, 1H), 7.42-7.37 (m, 2H), 7.31 (dd, J=8.5, 7.1 Hz, 2H), 7.28-7.20 (m, 5H), 6.93-6.87 (m, 4H), 6.18 (d, J=15.2 Hz, 1H), 5.65 (dd, J=8.2, 2.2 Hz, 1H), 4.51-4.42 (m, 2H), 3.73 (d, J=0.9 Hz, 6H), 3.25-3.13 (m, 2H), 1.38-1.25 (m, 3H), 1.04-0.92 (m, 22H) ppm. ¹³C NMR (151 MHz, DMSO-d₆) δ 162.89, 158.17, 150.61, 144.60, 140.45, 135.18, 129.67, 129.65, 127.84, 127.59, 126.78, 113.20, 104.65, 103.46, 101.59, 85.90, 85.89, 85.80, 76.78, 76.58, 62.87, 59.76, 55.05, 17.75, 17.72, 15.28, 15.12, 11.68 ppm. ¹⁹F NMR (565 MHz, DMSO-d₆) δ −145.32 ppm.

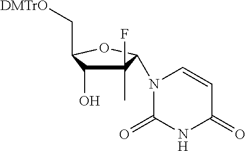

1-[(2S,5R)-5-[[bis(4-methoxyphenyl)-phenyl-methoxy]methyl]-3-fluoro-4-hydroxy-3-methyl-tetrahydrofuran-2-yl]pyrimidine-2,4-dione 158: To a clear solution of 157 (1.1 g, 1.53 mmol) in THF (20 mL) was added TBAF (520.07 mg, 1.99 mmol, 575.93 L) in single portion and stirred for 4 hr at 22° C. All the volatile matters were evaporated under high vacuum pump and the crude residue thus obtained, was purified by flash column chromatography (gradient: 0-5% MeOH in DCM) to afford 158 (0.8 g, 93% yield) as white foam. ¹H NMR (600 MHz, DMSO-d₆) δ 11.45 (d, J=2.3 Hz, 1H), 7.63 (d, J=8.2 Hz, 1H), 7.44-7.39 (m, 2H), 7.35-7.20 (m, 8H), 6.94-6.87 (m, 4H), 6.22 (d, J=4.5 Hz, 1H), 6.12 (d, J=16.6 Hz, 1H), 5.69 (dd, J=8.2, 2.2 Hz, 1H), 4.52 (ddd, J=7.5, 5.2, 2.5 Hz, 1H), 4.09 (ddd, J=15.7, 4.6, 2.6 Hz, 1H), 3.74 (s, 6H), 3.19 (ddd, J=9.6, 7.1, 2.1 Hz, 1H), 3.03 (dd, J=9.9, 5.2 Hz, 1H), 1.36-1.09 (m, 3H) ppm. ¹³C NMR (151 MHz, DMSO-d₆) δ 162.98, 158.11, 150.74, 144.80, 140.82, 135.53, 135.40, 129.66, 127.88, 127.64, 126.74, 113.24, 113.23, 105.05, 103.89, 101.61, 90.32, 90.04, 86.88, 85.53, 75.40, 75.20, 67.03, 63.46, 63.43, 55.04, 54.92, 25.14, 15.19, 15.03 ppm. ¹⁹F NMR (565 MHz, DMSO-d₆) δ −143.86 ppm.

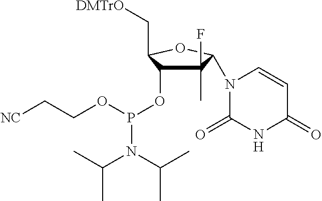

3-[[(2R,5S)-2-[[bis(4-methoxyphenyl)-phenyl-methoxy]methyl]-5-(2,4-dioxopyrimidin-1-yl)-4-fluoro-4-methyl-tetrahydrofuran-3-yl]oxy-(diisopropylamino)phosphanyl]oxypropane nitrile 159: To a clear solution of 158 (0.94 g, 1.67 mmol) in DCM (20 mL) was added N-methylimidazole (274.36 mg, 3.34 mmol, 266.36 L) and DIPEA (1.08 g, 8.35 mmol, 1.46 mL) in single portions. After stirring the reaction mixture for 5 minutes at 22° C., 2-cyanoethyl-N,N-diisopropylchlorophosphoramidite (790.92 mg, 3.34 mmol, 746.15 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-60% EtOAc in hexane) to afford 159 (0.97 g, 76% yield) as white foam. ¹H NMR (600 MHz, CD₃CN) δ 9.10 (s, 1H), 7.52 (d, J=8.2 Hz, 1H), 7.50-7.44 (m, 3H), 7.38-7.28 (m, 7H), 7.27-7.21 (m, 1H), 6.88 (ddd, J=9.0, 5.1, 2.0 Hz, 4H), 6.17 (dd, J=15.8, 2.6 Hz, 1H), 5.64 (dd, J=10.8, 8.2 Hz, 1H), 4.80-4.58 (m, 1H), 4.40-4.30 (m, 1H), 3.85-3.66 (m, 8H), 3.63-3.54 (m, 1H), 3.28 (ddt, J=9.6, 7.0, 2.5 Hz, 1H), 3.22-3.13 (m, 1H), 2.60 (t, J=5.9 Hz, 1H), 2.51 (t, J=6.0 Hz, 1H), 1.43-1.32 (m, 3H), 1.18 (d, J=6.8 Hz, 4H), 1.11 (dd, J=6.8, 2.3 Hz, 6H) ppm. ¹³C NMR (151 MHz, CD₃CN) δ 163.75, 163.70, 159.72, 151.67, 151.65, 145.97, 141.39, 141.33, 136.86, 136.76, 130.99, 130.98, 129.00, 128.95, 128.87, 127.89, 119.31, 119.28, 114.09, 114.07, 102.66, 102.59, 92.00, 91.97, 91.72, 87.87, 87.85, 87.77, 87.75, 87.23, 87.21, 79.66, 79.55, 79.44, 79.33, 79.15, 79.05, 78.93, 78.83, 64.48, 64.45, 64.42, 60.96, 59.77, 59.64, 59.44, 59.31, 55.90, 44.19, 44.11, 44.03, 25.00, 24.95, 24.89, 24.84, 24.78, 21.15, 20.95, 20.92, 20.90, 20.87, 16.49, 16.36, 16.21, 14.51 ppm. ¹⁹F NMR (565 MHz, CD₃CN) δ −144.63, −145.20 ppm. ³¹P NMR (243 MHz, CD₃CN) δ 152.37, 149.99 ppm.

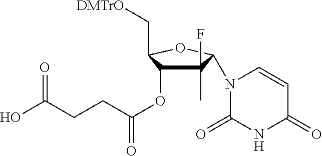

4-[(2R,5S)-2-[[bis(4-methoxyphenyl)-phenyl-methoxy]methyl]-5-(2,4-dioxopyrimidin-1-yl)-4-fluoro-4-methyl-tetrahydrofuran-3-yl]oxy-4-oxo-butanoic acid 160: To a clear solution of 158 (0.4 g, 711.00 μmol) in DCM (10.00 mL) was added DMAP (347.45 mg, 2.84 mmol) and succinic anhydride (142.30 mg, 1.42 mmol) in single portion. Reaction mixture was stirred at 22° C. for 4 hr. To this reaction mixture was added DCM (10 mL) and washed with 10% NH₄Cl solution (2×10 mL). Organic layer was separated, dried over anhydrous Na₂SO₄, filtered and the filtrate was evaporated to dryness. Crude compound was purified by flash column chromatography (gradient: 0-5% MeOH in DCM) to afford 160 (0.4 g, 85% yield) as white foam.

¹H NMR (600 MHz, DMSO-d₆) δ 12.35 (s, 1H), 11.51 (d, J=2.2 Hz, 1H), 7.61 (d, J=8.2 Hz, 1H), 7.44-7.39 (m, 2H), 7.35-7.20 (m, 8H), 6.94-6.86 (m, 4H), 6.18 (d, J=15.5 Hz, 1H), 5.65 (dd, J=8.2, 2.1 Hz, 1H), 5.11 (dd, J=14.9, 1.7 Hz, 1H), 4.81 (t, J=6.4 Hz, 1H), 3.74 (s, 6H), 3.23 (ddd, J=9.8, 7.4, 2.2 Hz, 1H), 3.09 (dd, J=9.9, 5.1 Hz, 1H), 2.57 (dd, J=8.0, 5.9 Hz, 2H), 2.53 (dd, J=5.9, 1.7 Hz, 2H), 1.40-1.22 (m, 3H) ppm. ¹⁹F NMR (565 MHz, DMSO-d₆) δ −144.76 ppm. ¹³C NMR (151 MHz, DMSO-d₆) δ 173.47, 170.88, 162.95, 162.31, 158.13, 150.71, 144.65, 140.24, 140.20, 135.39, 135.26, 129.65, 128.92, 127.89, 127.65, 127.62, 127.42, 126.76, 113.26, 113.24, 112.77, 104.39, 103.23, 101.60, 90.65, 90.38, 85.70, 85.10, 76.94, 76.70, 63.27, 63.24, 55.04, 35.79, 30.77, 28.91, 28.63, 14.95, 14.80 ppm.

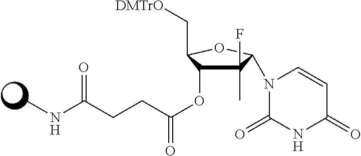

Added 160 (0.4 g, 603.63 μmol) and DIPEA (387.72 mg, 3.00 mmol, 522.53 μL) into rb flask. Then added dry DCM (10.00 mL). Stirred well to dissolve and then HBTU (228.68 mg, 603.00 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 mL total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG 161 in high vacuum overnight.

Checking the Loading: Weighted out 40 mg and loaded into 250 mL volumetric flask. Then added 0.1 M toluene-p-sulfonic acid in ACN up to measure line. Sonicated and settled for 1 hour. Checked loading by spectrophotometer and Beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 4.0 g, Loading: 86 μmol/g.

### Compound 227

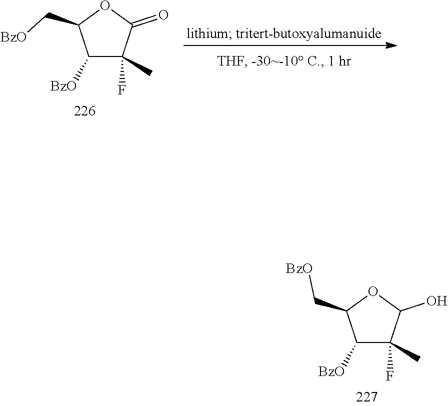

Lithium;tritert-butoxyalumanuide (1.00 M in THF, 375 mL, 1.12 eq) was added dropwise to a solution of Compound 226 (125.0 g, 336 mmol, 1.00 eq) in THF (575 mL) at −30° C. ˜−25° C., then the reaction was stirred at −10° C. for 1 hr. TLC (Petroleum ether/EtOAc=3/1) showed the reaction was completely. The reaction mixture was quenched by addition of NH₄Cl (1000 mL) at 0° C. under N₂, then the four reactions were combined and then extracted with EtOAc (1000 mL×3). The combined organic layers were washed with brine (1000 mL), dried over Na₂SO₄, filtered, and concentrated under reduced pressure to give a residue. The residue was purified by column chromatography (SiO₂, Petroleum ether/Ethyl acetate=100/1 to 1/1) to give compound 227 (366 g, 72.8% yield) as white solid. ¹H NMR: DMSO-d₆, 400 MHz, δ 7.98-8.00 (m, 2H), 7.90-7.92 (m, 2H), 7.68-7.72 (m, 1H), 7.59-7.63 (m, 1H), 7.53-7.57 (m, 2H), 7.40-7.44 (m, 2H), 7.32 (d, J=4.4 Hz, 1H), 5.50-5.58 (m, 1H), 5.18-5.22 (m, 1H), 4.41-4.54 (m, 3H), 1.43 (d, J=22.8 Hz, 3H).

### Compound 228

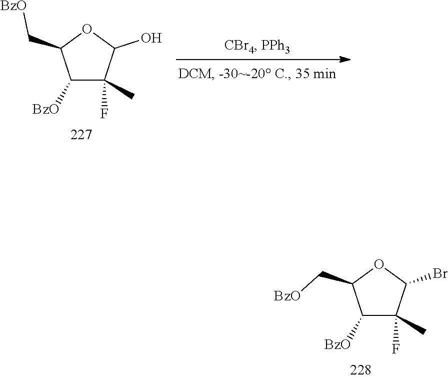

The reactions were carried out following the literature procedure¹. A solution of PPh₃ (73.5 g, 280 mmol, 1.40 eq) in DCM (1950 mL) was added to a three-necked flask, then the reaction was cooled to −20° C., 227 (75.0 g, 200 mmol, 1.00 eq) was added to above reaction, stirred at −20˜−30° C. for 15 min, then CBr₄ (99.6 g, 300 mmol, 1.50 eq) was added to above reaction at −30 −20° C., then the reaction was stirred at −25 ˜−30° C. for 20 min. TLC (Petroleum ether/EtOAc=3/1) showed the reaction was completely. Silica gel (165.0 g) was added to above reaction at −20 ˜−25° C., then the reaction was filtered through a pad of silica gel (488 g) and concentrated under reduced pressure to give a residue below 30° C. The residue was purified by column chromatography (SiO₂, Petroleum ether/Ethyl acetate=0/1 to 4/1) to give compound 228 (133 g, 75.9% yield) as yellow oil. ¹H NMR CDCl₃, 400 MHz, δ 8.12-8.15 (m, 2H), 8.02-8.04 (m, 2H), 7.56-7.64 (m, 2H), 7.42-7.50 (m, 5H), 6.35 (s, 1H), 5.28-5.30 (m, 1H), 4.86-4.90 (m, 1H), 4.75-4.80 (m 1H), 4.61-4.66 (m, 1H), 1.72 (d, J=22.4 Hz, 3H).

### Compound 234

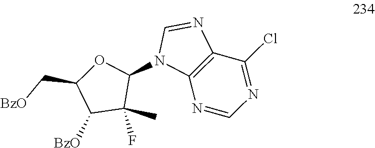

t-BuOK (37.2 g, 332 mmol, 2.90 eq) was added to a solution of Compound 228 (53.0 g, 343 mmol, 3.00 eq) in t-BuOH (1150 mL), then the reaction was stirred at 30° C. for 1 hr, then a solution of Compound 3 (50.0 g, 114 mmol, 1.00 eq) in ACN (250 mL) was added to above reaction, then the reaction was stirred at 65° C. for 12 hrs. The reaction mixture was poured to NH₄Cl (1500 mL), and extracted with EtOAc (500 mL×2), the combined organic layers were washed with brine (500 mL), then dried over Na₂SO₄, filtered, and concentrated under reduced pressure to give a residue. The residue was purified by column chromatography (SiO₂, Petroleum ether/Ethyl acetate=100/1 tol/1) to give compound 234 (69.0 g, 39.3% yield) as white solid. ¹H NMR ( ): CDCl₃, 400 MHz, δ 7.99-8.03 (m, 2H), 7.62-7.66 (m, 1H), 7.55-7.58 (m, 1H), 7.47-7.51 (m, 2H), 7.39-7.44 (m, 2H), 6.37 (d, J=18 Hz, 1H), 6.22-6.30 (m, 1H), 4.87-4.90 (m, 1H), 4.79-4.83 (m, 1H), 4.67-4.72 (m, 1H), 1.33 (d, J=22.4 Hz, 3H).

### Compound 235

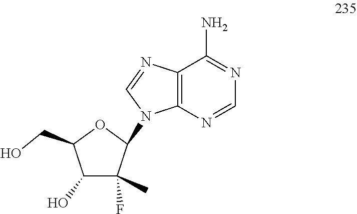 [SMILES: C[C@@]1(F)[C@H](O)[C@@H](CO)O[C@H]1n1cnc2c(N)ncnc21]

NH₃·H₂O (120 mL) was added to a solution of compound 234 (12.0 g, 23.4 mmol, 1.00 eq) in dioxane (50 mL) then the reaction was stirred at 95° C. for 12 hrs. TLC (DCM/MeOH=10/1) showed the reaction was completely. The reaction was concentrated under reduced pressure to give a residue and the residue was purified by prep-HPLC (column: Phenomenex Titank C18 Bulk 250×100 mm 10u; mobile phase: [water (10 mM NH₄HCO₃)-ACN]; B %: 1%-25%, 20 min) to give compound 235 (32.0 g) as white solid. ¹H NMR: DMSO-d₆, 400 MHz, δ 8.43 (s, 1H), 8.15 (s, 1H), 7.37 (s, 2H), 6.18 (d, J=18 Hz, 1H), 5.68 (d, J=7.2 Hz, 1H), 5.24-5.26 (m, 1H), 4.24-4.35 (m, 1H), 3.84-3.95 (m, 2H), 3.67-3.73 (m, 1H), 1.05 (d, J=22.8 Hz, 3H).

### Compound 236

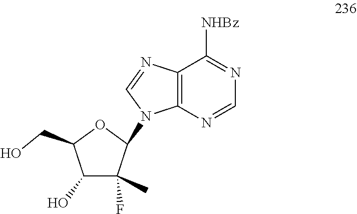

TMSCl (35.6 g, 328 mmol, 6.00 eq) was added to a solution of Compound 235 (15.5 g, 54.7 mmol, 1.00 eq) in Py (329 mL) then the reaction was stirred at 15° C. for 3 hrs. TLC (DCM/MeOH=10/1) showed the reaction was completely. BzCl (15.3 g, 109 mmol, 2.00 eq) was added to above solution at 0° C., then the reaction was stirred at 15° C. for 2.5 hrs. TLC (DCM/MeOH =10/1) showed the reaction was completely. H₂O (51.8 g) was added to the reaction solution and stirred at 0° C. for 15 min. Then NH₃·H₂O (155 mL) was added at 0˜5° C. and stirred at 0° C. for another 15 min. TLC (DCM/MeOH=10/1) showed the reaction was completely. The reaction mixture was quenched by addition NH₄Cl (200 mL), diluted with EtOAc (200 mL) and extracted with EtOAc (200 mL×3). The combined organic layers were washed with brine (200 mL), dried over Na₂SO₄, filtered, and concentrated under reduced pressure to give a residue. The residue was purified by column chromatography (SiO₂,DCM/MeOH=100/1 to 10/1) and further purification by prep-HPLC (column: Phenomenex Titank C18 Bulk 250×100 mm 10u; mobile phase: [water (10 mM NH₄HCO₃)-ACN]; B %: 1%-30%, 20 min) to give 236 (10.6 g, 24.8% yield, 98.7% purity) as white solid. ¹H NMR (ET43664-53-P1B): DMSO-d₆, 400 MHz, δ 11.26 (s, 1H), 8.79 (d, J=8.4 Hz, 2H), 8.04 (d, J=7.2 Hz, 2H), 7.53-7.66 (m, 3H), 6.35 (d, J=17.6 Hz, 1H), 5.77 (d, J=7.2 Hz, 1H), 5.28-5.31 (m, 1H), 4.29-4.39 (m, 1H), 3.99 (d, J=9.2 Hz, 1H), 3.87-3.91 (m, 1H), 3.70-3.75 (m, 1H), 1.12 (d, J=22.4 Hz, 3H). ¹⁹F NMR (ET43664-53-P1B): DMSO-d₆, 376 MHz, δ: −161.38. LCMS [M+H]⁺=388.1

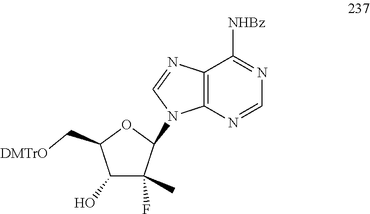

N-[(9S)-9-[(2R,4R,5R)—S-[[bis(4-methoxyphenyl)-phenyl-methoxy]methyl]-3-fluoro-4-hydroxy-3-methyl-tetrahydrofuran-2-yl]purin-6-yl]benzamide 237: To a clear solution of 236 (1.4 g, 3.61 mmol) in pyridine (30 mL) was added 4,4′-dimethoxytrityl chloride (1.47 g, 4.34 mmol) in two portions. Reaction mixture was stirred at for 16 hr, diluted with DCM (30 mL) and then quenched with 10% NaHCO₃ (30 mL). Organic layer was washed with brine (2×20 mL), separated, dried over anhydrous Na₂SO₄ and filtered. Filtrate was evaporated under high vacuum pump and crude mass obtained, was purified by flash column chromatography (10-50% EtOAc in hexane) to afford 237 (1.5 g, 60% yield) as white solid. ¹H NMR (600 MHz, DMSO-d₆) δ 11.27 (s, 1H), 8.64 (s, 1H), 8.59 (s, 1H), 8.08-8.02 (m, 2H), 7.68-7.62 (m, 1H), 7.56 (t, J=7.7 Hz, 2H), 7.41-7.36 (m, 2H), 7.30-7.16 (m, 7H), 6.86-6.77 (m, 4H), 6.48-6.33 (m, 1H), 5.76 (d, J=7.7 Hz, 1H), 4.60-4.46 (m, 1H), 4.22-4.16 (m, 1H), 3.71 (d, J=6.4 Hz, 6H), 3.54 (dd, J=10.6, 6.9 Hz, 1H), 3.29 (dd, J=10.6, 2.0 Hz, 1H), 1.22-1.14 (m, 3H) ppm. ¹⁹F NMR (565 MHz, DMSO-d₆) δ −159.52 ppm. ¹³C NMR (151 MHz, DMSO-d₆) δ 165.64, 158.06, 158.04, 151.71, 151.41, 150.64, 149.62, 144.71, 135.58, 135.33, 133.32, 132.51, 129.74, 129.70, 128.53, 128.48, 127.75, 127.73, 126.68, 125.75, 123.91, 113.13, 113.08, 101.39, 100.20, 89.22, 88.95, 85.57, 80.67, 72.51, 72.39, 63.41, 59.76, 55.00, 54.98, 54.92, 16.64, 16.47 ppm.

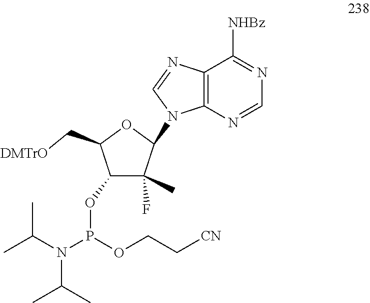

N-[(9S)-9-[(2R,4R,5R)-5-[[bis(4-methoxyphenyl)-phenyl-methoxy]methyl]-4-[[bis(1-methylethyl)amino]-(2-cyanoethoxy)phosphanyl]oxy-3-fluoro-3-methyl-tetrahydrofuran-2-yl]purin-6-yl]benzamide 238: To a clear solution of 237 (1.0 g, 1.45 mmol) in DCM (29.35 mL) was added N-methylimidazole (238.06 mg, 2.90 mmol, 230.91 L) and DIPEA (936.89 mg, 7.25 mmol, 1.26 mL) in single portions. After stirring the reaction mixture for 5 minutes at 22° C., 2-cyanoethyl-N,N-diisopropylchlorophosphoramidite (686.30 mg, 2.90 mmol, 647.45 μL) was added and continued stirring for 0.5 hr and TLC was checked. Starting material was consumed and reaction mixture was diluted with DCM (20 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (30-70% EtOAc in hexane) to afford 238 (1.0 g, 77% yield) as white foam. ¹H NMR (600 MHz, CD₃CN) δ 9.33 (s, 1H), 8.53 (d, J=4.5 Hz, 1H), 8.34 (d, J=13.6 Hz, 1H), 7.99 (d, J=7.6 Hz, 2H), 7.67-7.61 (m, 1H), 7.57-7.51 (m, 2H), 7.46-7.41 (m, 2H), 7.33-7.17 (m, 6H), 6.83-6.74 (m, 4H), 6.38-6.31 (m, 1H), 4.98-4.87 (m, 1H), 4.36-4.29 (m, 1H), 3.86-3.67 (m, 7H), 3.63-3.51 (m, 4H), 2.62 (ddd, J=6.5, 5.4, 1.9 Hz, 1H), 2.48-2.40 (m, 1H), 1.34-1.26 (m, 3H), 1.17-1.09 (m, 9H), 0.99 (d, J=6.8 Hz, 3H) ppm. ³¹P NMR (243 MHz, CD₃CN) δ 150.63, 149.67 ppm. ¹⁹F NMR (565 MHz, CD₃CN) δ−159.13, −159.32 ppm. ¹³C NMR (151 MHz, CD₃CN) δ 166.11, 159.69, 159.66, 159.64, 159.61, 152.90, 152.42, 151.09, 145.95, 145.93, 143.68, 143.54, 136.72, 136.57, 136.56, 134.79, 133.61, 131.15, 131.13, 131.10, 131.08, 129.66, 129.15, 129.11, 129.03, 128.77, 128.74, 127.85, 125.83, 125.76, 119.54, 119.34, 114.00, 113.94, 113.90, 102.34, 102.17, 102.14, 101.13, 100.93, 91.15, 91.06, 90.88, 90.80, 87.23, 87.20, 82.15, 81.91, 81.88, 75.57, 75.47, 75.35, 75.24, 75.14, 64.07, 63.56, 60.96, 59.42, 59.32, 59.29, 59.19, 55.88, 55.86, 55.84, 44.07, 43.99, 24.97, 24.92, 24.87, 24.82, 24.80, 24.75, 21.15, 20.94, 20.92, 20.89, 20.87, 17.92, 17.89, 17.81, 17.80, 17.75, 17.73, 17.65, 17.63 ppm.

### Compound 239

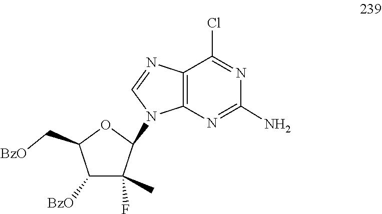

To a solution of compound 228 (58.8 g, 343 mmol, 3.00 eq) in t-BuOH (1150 mL) was added t-BuOK (37.2 g, 331 mmol, 2.90 eq), and stirred for 1 h, then a solution of compound 3 (50.0 g, 114 mmol, 1.00 eq) in ACN (250 mL) was added to above solution under N₂ at 15° C. Then the mixture was stirred at 65° C. for 16 h. LCMS (ET44598-18-P1A1) indicated the reaction was completed. To the reaction mixture was added solid NH₄Cl (3000 mL), then EtOAc (1500 mL) was added, filtered through celite. The combined organic layers were washed with brine (500 mL), dried over Na₂SO₄, filtered, and concentrated under reduced pressure to give a residue. The residue was purified by column chromatography (SiO₂, Petroleum ether/Ethyl acetate=10/1 to 0/1). Compound 239 (30 g, 57.0 mmol, 49.8% yield) was obtained as a white solid.

### Compound 240

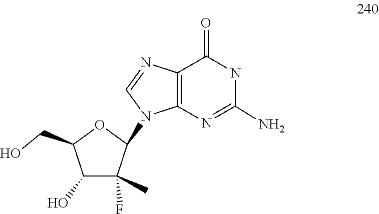 [SMILES: C[C@@]1(F)[C@H](O)[C@@H](CO)O[C@H]1n1cnc2c(=O)[nH]c(N)nc21]

HCl (2 M, 142 mL, 10 eq) was added to a solution of compound 239 (15 g, 28.5 mmol, 1.00 eq) in dioxane (150 mL), then stirred at 90° C. for 16 h. LCMS (ET44598-26-P1A) indicated the reaction was completed. The reaction mixture was concentrated under reduced pressure to give a residue. Compound 240 (15 g, 50.1 mmol, 87.8% yield) was obtained as a yellow oil.

### Compound 241

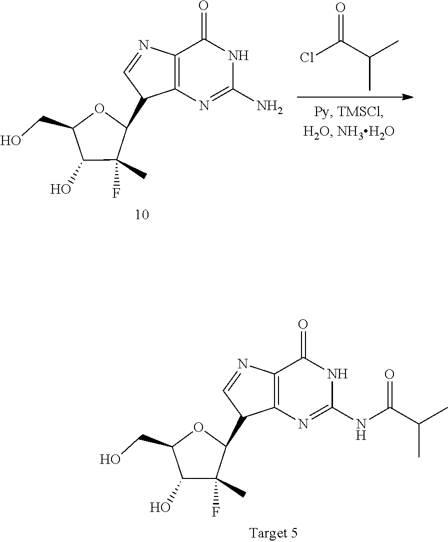

TMSCl (28.2 g, 260 mmol, 33 mL, 6.0 eq) was added to a solution of compound 240 (6.5 g, 21.7 mmol, 1.00 eq) in Py (65 mL) at 20° C., then stirred for 2 h. Then 2-methylpropanoyl chloride (9.26 g, 86.8 mmol, 9.08 mL, 2 eq) was added at 0° C., then stirred for 2 h at 20° C. Then H₂O (56 mL) was added to above solution at 0° C. and stirred at 0° C. for 20 min, then NH₃·H₂O (106 g, 762 mmol, 117 mL, 25% purity, 17.5 eq) was added and stirred for 30 min at 20° C. TLC (Dichloromethane:Methanol=4:1, R_{f} of R1=0.20, R_{f} of P1=0.50) indicated the reaction was completed. TLC (Dichloromethane:Methanol=10:1, R_{f} of R1=0.20, R_{f} of P1=0.70) indicated the reaction was completed. TLC (Dichloromethane:Methanol=10:1, R_{f} of R1=0.70, R_{f} of P1 =0.20) indicated the reaction was completed. The reaction mixture was concentrated under reduced pressure to give a residue. The residue was purified by column chromatography (SiO₂, DCM:MeOH=100/1, 30/1, 10/1). 241 (6.5 g, crude) was obtained as a yellow solid.

### Compound 242

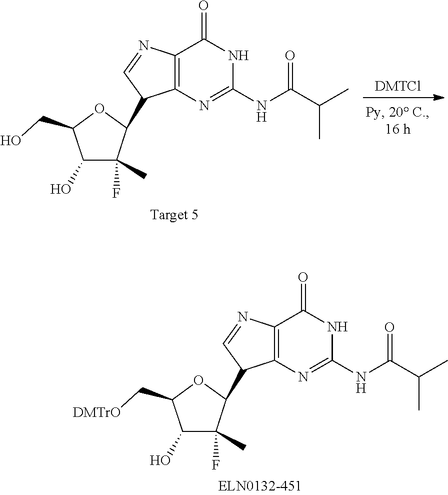

DMTCl (4.13 g, 12.1 mmol, 1.00 eq) was added to a solution of 241 (4.5 g, 12.2 mmol, 1.00 eq) in Py (45 mL) at 20° C., then stirred at 20° C. for 16 h. TLC (Dichloromethane:Methanol =10:1, R_{f} of R1=0.20, R_{f} of P1=0.70) indicated the reaction was completed. The reaction mixture was quenched by addition NaHCO₃ (100 mL), and extracted with DCM (50 mL×3). The combined organic layers were washed with brine (50 mL), dried over, filtered and concentrated under reduced pressure to give a residue. The residue was purified by prep-HPLC (column: Phenomenex Titank C18 Bulk 250*100 mm 10u; mobile phase: [water (10 mM NH₄HCO₃)-ACN]; B %: 50%-70%, 20 min). Compound 242 (3.8 g, 5.66 mmol, 46.4% yield) was obtained as a yellow solid. LCMS: ET44598-40-P1C1. ¹H NMR: ET44598-40-P1A1, 400 MHz, MeOH-d₄ δ 8.19 (s, 1H), 7.39-7.49 (m, 2H), 7.14-7.37 (m, 7H), 6.73-6.90 (m, 4H), 6.19 (d, J=18.0 Hz, 1H), 4.37-4.54 (m, 1H), 4.19 (m, 1H), 3.70-3.80 (m, 6H), 3.45-3.63 (m, 2H), 2.58-2.81 (m, 1H), 1.17-1.24 (m, 8H).

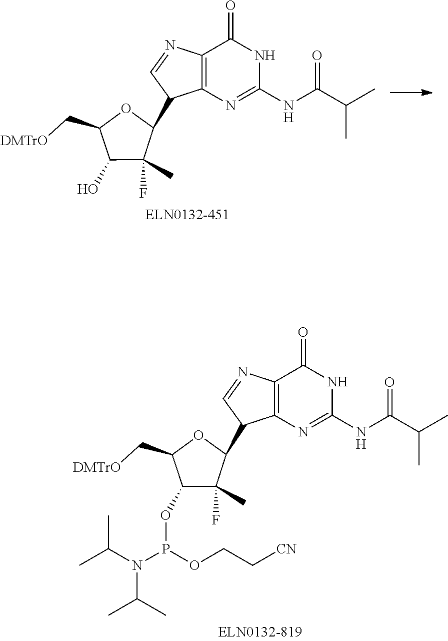

N-[9-[(2R,3R,4R,5R)-5-[[bis(4-methoxyphenyl)-phenyl-methoxy]methyl]-4-[2-cyanoethoxy-(diisopropylamino)phosphanyl]oxy-3-fluoro-3-methyl-tetrahydrofuran-2-yl]-6-oxo-1H-purin-2-yl]-2-methyl-propanamide 243: To a clear solution of 242 (1.0 g, 1.49 mmol) in DCM (30 mL) was added N-methylimidazole (244.45 mg, 2.98 mmol, 237.33 μL) and diisopropylethylamine (962.02 mg, 7.44 mmol, 1.30 mL) in single portions. After stirring the reaction mixture for 5 minutes at 22° C., 2-cyanoethyl N,N-diisopropylchlorophosphoramidite (704.71 mg, 2.98 mmol, 664.82 μL) was added and continued stirring for 0.5 hr and TLC was checked. Starting material was consumed and reaction mixture was diluted with DCM (20 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (60-90% EtOAc in hexane) to afford 243 (1.04 g, 80% yield) as white foam. ¹H NMR (600 MHz, CD₃CN) δ 12.07 (s, 1H), 9.40 (s, 1H), 8.07-7.76 (m, 1H), 7.45 (ddt, J=9.8, 8.1, 1.3 Hz, 2H), 7.36-7.18 (m, 7H), 6.20-5.99 (m, 1H), 4.63-4.48 (m, 1H), 4.25 (tdd, J=11.1, 5.0, 3.1 Hz, 1H), 3.81-3.70 (m, 7H), 3.67-3.42 (m, 3H), 2.77-2.56 (m, 2H), 1.32-1.09 (m, 22H), 1.00 (d, J=6.8 Hz, 3H) ppm. ³¹P NMR (243 MHz, CD₃CN) δ 150.24, 149.11 ppm. ¹⁹F NMR (565 MHz, CD₃CN) δ −172.62, −172.85 ppm. ¹³C NMR (151 MHz, CD₃CN) δ 181.12, 180.96, 171.66, 159.74, 159.70, 159.67, 159.60, 156.32, 156.27, 149.28, 149.23, 149.15, 148.93, 145.95, 145.84, 138.10, 136.74, 136.65, 136.46, 131.27, 131.23, 131.20, 131.13, 131.10, 131.06, 129.21, 129.11, 128.99, 128.85, 128.73, 127.92, 127.86, 119.57, 114.03, 114.00, 113.90, 113.86, 102.30, 102.29, 101.89, 101.87, 101.09, 101.08, 100.68, 100.65, 91.08, 90.60, 90.33, 87.36, 87.22, 86.94, 82.09, 81.64, 81.61, 76.24, 74.94, 64.31, 63.06, 60.96, 58.92, 58.78, 58.27, 58.14, 55.90, 55.88, 55.86, 55.85, 55.32, 44.06, 43.98, 36.61, 36.42, 24.95, 24.89, 24.83, 24.80, 24.75, 24.71, 21.17, 21.15, 21.13, 20.92, 20.87, 19.38, 19.25, 19.22, 19.19, 19.16, 19.13, 18.18, 18.15, 18.01, 17.98, 17.74, 17.58, 14.51 ppm.

### Compound 229

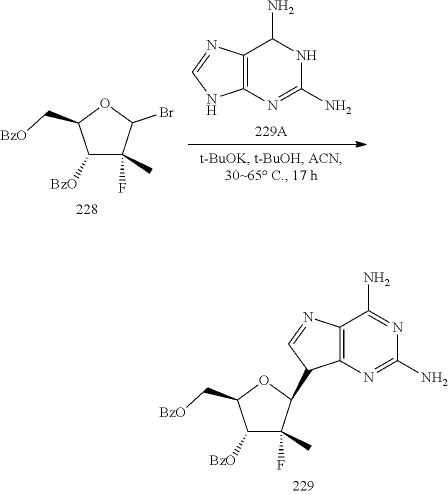

The reaction was carried out following the literature procedure¹. t-BuOK (40.9 g, 365 mmol, 2.90 eq) was added to a white mixture of compound 229A (56.7 g, 377 mmol, 3.00 eq) (compound 229A was evaporation with ACN (300 mL)×3) in t-BuOH (1265 mL) at 30° C., the yellow resulting mixture was stirred for 1 h at 30° C. (oil bath) under N₂. Then a solution of compound 228 (55.0 g, 126 mmol, 1.00 eq) in ACN (275 mL) was added at 30° C., then the light-yellow mixture was stirred at 65° C. (oil bath) (inner temperature: 50˜55° C.) for 16 h. TLC indicated no 228, one major new spot with larger polarity was detected and LCMS showed ˜25% desired MS. The reaction mixture was cooled to 30° C., added to a stirred saturated NH₄Cl (10 L) and EtOAc (6 L), filtered through a pad of kieselguhr (aqueous layer: pH˜7), the organic layer was separated and washed with brine (1 L), dried over Na₂SO₄, filtered, and concentrated under reduced pressure to give a residue. The residue was purified by column chromatography (SiO₂, Petroleum ether/Ethyl acetate=3/1, 1/1, 1/2, 0/1). Compound 229 (44.9 g, 88.7 mmol, 23.5% yield) was obtained as a yellow solid. ¹H NMR (ET44179-41-P1A): DMSO-d6, 400 MHz, 8.08-7.99 (m, 2H), 7.95 (s, 1H), 7.93-7.87 (m, 2H), 7.75-7.68 (m, 1H), 7.64-7.52 (m, 3H), 7.46-7.37 (m, 2H), 6.89 (s, 2H), 6.26 (d, J=19.6 Hz, 1H), 5.92 (s, 2H), 4.92-4.81 (m, 1H), 4.73-4.59 (m, 2H), 1.28 (d, J=23.2 Hz, 3H). ¹⁹F NMR (ET44179-41-P1A): CDCl₃, 376 MHz, −155.890, −163.981, −169.424. LCMS calculated for C₂₅H₂₃FN₆O₅ [M+H]⁺m/z=507.17, found 507.10/548.10.

### Compound 230

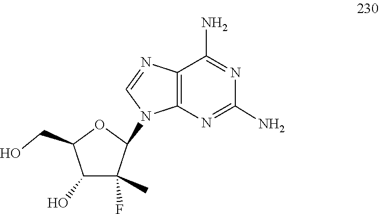 [SMILES: C[C@@]1(F)[C@H](O)[C@@H](CO)O[C@H]1n1cnc2c(N)nc(N)nc21]

Compound 229 (44.9 g, 88.6 mmol, 1.00 eq) was dissolved in MeOH (449 mL), NaOMe (6.39 g, 35.5 mmol, 30% purity, 0.40 eq) in MeOH (10 mL) was added dropwise at 16 20° C., stirred at 16° C. for 1 h under N₂, white solid was detected. TLC (DCM/MeOH=20/1) indicated compound 229 was consumed completely and LCMS indicated desired MS. AcOH (˜2.13 g) was added dropwise to the reaction, adjust pH=6˜7, then filtered, the solid was washed with ACN and collected. Compound 230 (18.5 g, 62.0 mmol, 70.0% yield) was obtained as a white solid. ¹H NMR: DMSO-d6, 400 MHz, 7.99 (s, 1H), 6.97 (s, 2H), 6.00 (d, J=18.4 Hz, 1H), 5.89 (s, 2H), 5.62 (d, J=6.8 Hz, 1H), 5.25 (t, J=5.2 Hz, 1H), 4.30-4.13 (m, 1H), 3.94-3.80 (m, 2H), 3.74-3.64 (m, 1H), 1.07 (dd, J=22.4 Hz, 3H). ¹⁹F NMR: DMSO-d6, 376 MHz, −161.215, −174.032. LCMS calculated for C₁₁H₁₅FN₆O₃ [M+H]⁺m/z=298.12, found 299.1.

### Compound 231

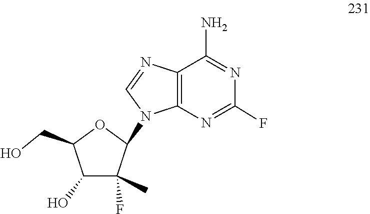 [SMILES: C[C@@]1(F)[C@H](O)[C@@H](CO)O[C@H]1n1cnc2c(N)nc(F)nc21]

Py (122 mL) was added to a three neck polyethylene bottle, cooled to −20° C. under N₂, HF-Py (134 g, 949 mmol, 122 mL, 70% purity, 15.3 eq)(70% HF, 30% Py) was added to the bottle below 0° C., compound 230 (18.5 g, 62.0 mmol, 1.00 eq) was added, then tert-butyl nitrite (22.4 g, 217 mmol, 25.8 mL, 3.50 eq) was added in one portion, and the yellow solution (pH˜4) was stirred at 0˜10° C. for 2 h under N₂. TLC (DCM/MeOH=5/1, R_{f} of 230=0.24, R_{f} of 231=0.50) indicated compound 230 was consumed completely. The reaction was poured slowly into stirred water (1000 mL) and NaHCO₃solid (700 g), the resulting solution (pH=7˜8) was extracted with EtOAc (1000 mL×4), the combined organic layers were washed with brine (500 mL), dried over Na₂SO₄, filtered, and concentrated under reduced pressure to give a residue at 45° C. The crude product was triturated with Petroleum ether/Ethyl acetate=1/1 (80 mL) at 20° C. for 2 h, then triturated with MeOH (60 mL) at 20° C. for 2 h. 231 (15.0 g, 49.8 mmol, 80.3% yield) was obtained as a white solid. ¹H NMR: DMSO-d6, 400 MHz, 8.41 (s, 1H), 7.93 (br s, 2H), 6.07 (d, J=17.6 Hz, 1H), 5.71 (d, J=7.2 Hz, 1H), 5.22 (t, J=4.8 Hz, 1H), 4.32-4.15 (m, 1H), 3.94 (d, J=9.2 Hz, 1H), 3.90-3.82 (m, 1H), 3.74-3.65 (m, 1H), 1.08 (d, J=22.4 Hz, 3H). ¹⁹F NMR: DMSO−d6, 376 MHz, −51.643, −161.322. LCMS calculated for C₁₁H₁₃F₂N₅O₃ [M+H]⁺m/z=302.10, found 302.1/324.1.

### Compound 232

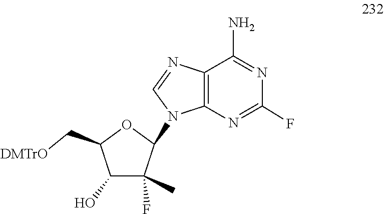

Compound 231 (14.8 g, 49.1 mmol, 1.00 eq) was evaporation with anhydrous Py (160 mL×4) at 45° C., then dissolved in Py (148 mL), then added DMTrCl (18.3 g, 54.0 mmol, 1.10 eq) at 20° C., and the orange solution was stirred at 20° C. for 4 h under N₂. TLC (DCM/MeOH=10/1, R_{f} of 231=0.24, R_{f} of 232=0.90) indicated 231 was consumed completely. Saturated NaHCO₃ (300 mL) was added to the reaction, stirred, the resulting solution (pH=8) was extracted with EtOAc (300 mL, 200 mL, 100 mL), the combined organic layers were washed with brine (150 mL), dried over Na₂SO₄, filtered, and concentrated under reduced pressure to give a residue at 45° C. The residue was purified by column chromatography (SiO₂, Petroleum ether/Ethyl acetate (0.5% TEA, DCM)=5/1, 3/1, 2/1, 1/1, 0/1). 232 (15.3 g, 25.4 mmol, 51.6% yield) was obtained as a yellow solid. ¹H NMR: DMSO-d6, 400 MHz, 8.27 (s, 1H), 8.09-7.80 (m, 2H), 7.40 (d, J=7.2 Hz, 2H), 7.33-7.15 (m, 7H), 6.81 (dd, J=8.4, 16.4 Hz, 4H), 6.20 (d, J=18.8 Hz, 1H), 5.74 (d, J=7.6 Hz, 1H), 4.54-4.34 (m, 1H), 4.14 (t, J=7.6 Hz, 1H), 3.72 (d, J=3.6 Hz, 6H), 3.65-3.52 (m, 1H), 3.25 (d, J=10.0 Hz, 1H), 1.15 (d, J=22.4 Hz, 3H). ¹⁹F NMR: DMSO-d6, 376 MHz, −51.601, −160.025. LCMS calculated for C₃₂H₃₁F₂N₅O₅ [M+H]⁺m/z=603.23, found 604.2/303.1.

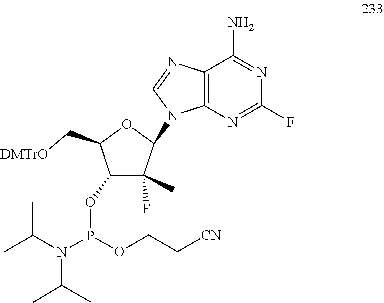

3-[[(2R,3R,5R)-5-[(9S)-6-amino-2-fluoro-purin-9-yl]-2-[[bis(4-methoxyphenyl)-phenyl-methoxy]methyl]-4-fluoro-4-methyl-tetrahydrofuran-3-yl]oxy-[bis(1-methylethyl)amino]phosphanyl]oxypropanenitrile 233: To a clear solution of 232 (1.0 g, 1.66 mmol) in DCM (30 mL) was added N-methylimidazole (272.03 mg, 3.31 mmol, 263.85 L) and diisopropylethylamine (1.07 g, 8.28 mmol, 1.44 mL) in single portions. After stirring the reaction mixture for 5 minutes at 22° C., 2-cyanoethyl-N,N-diisopropylchlorophosphoramidite (784.21 mg, 3.31 mmol, 739.82 L) was added and continued stirring for 0.5 hr and TLC was checked. Starting material was consumed and reaction mixture was diluted with DCM (20 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-70% EtOAc in hexane) to afford 233 (1.08 g, 81% yield) as white foam. ¹H NMR (600 MHz, CD₃CN) δ 8.07 (d, J=13.1 Hz, 1H), 7.46-7.40 (m, 2H), 7.34-7.16 (m, 7H), 6.84-6.73 (m, 4H), 6.38 (s, 2H), 6.22-6.03 (m, 1H), 4.80-4.70 (m, 1H), 4.32-4.23 (m, 1H), 3.84-3.65 (m, 7H), 3.61-3.48 (m, 5H), 2.65-2.58 (m, 1H), 2.42 (t, J=6.1 Hz, 1H), 1.31-1.23 (m, 4H), 1.14-1.08 (m, 8H), 0.97 (d, J=6.8 Hz, 3H). ¹⁹F NMR (565 MHz, CD₃CN) δ −52.43, −52.54, −159.60, −159.75 ppm. ³¹P NMR (243 MHz, CD₃CN) δ 150.86, 149.65 ppm. ¹³C NMR (151 MHz, CD₃CN) δ 160.44, 159.55, 159.54, 159.49, 159.07, 158.61, 158.47, 151.42, 151.29, 151.27, 145.80, 140.74, 136.57, 136.51, 136.43, 136.37, 131.07, 131.06, 130.97, 130.96, 130.90, 129.01, 128.97, 128.93, 128.65, 128.63, 127.73, 127.70, 119.31, 119.21, 119.18, 119.16, 119.13, 119.09, 113.86, 113.83, 113.80, 113.78, 102.10, 102.08, 101.90, 101.87, 100.89, 100.69, 100.66, 90.79, 90.63, 90.53, 90.37, 87.13, 87.11, 81.86, 81.71, 81.68, 75.28, 75.19, 75.17, 75.08, 75.04, 74.94, 74.84, 63.80, 63.35, 60.86, 59.34, 59.21, 59.17, 59.04, 55.82, 55.80, 55.77, 55.75, 43.96, 43.93, 43.87, 43.85, 24.88, 24.84, 24.82, 24.80, 24.77, 24.73, 21.14, 20.83, 20.78, 20.73, 17.84, 17.81, 17.70, 17.68, 17.65, 17.54, 17.52 ppm.

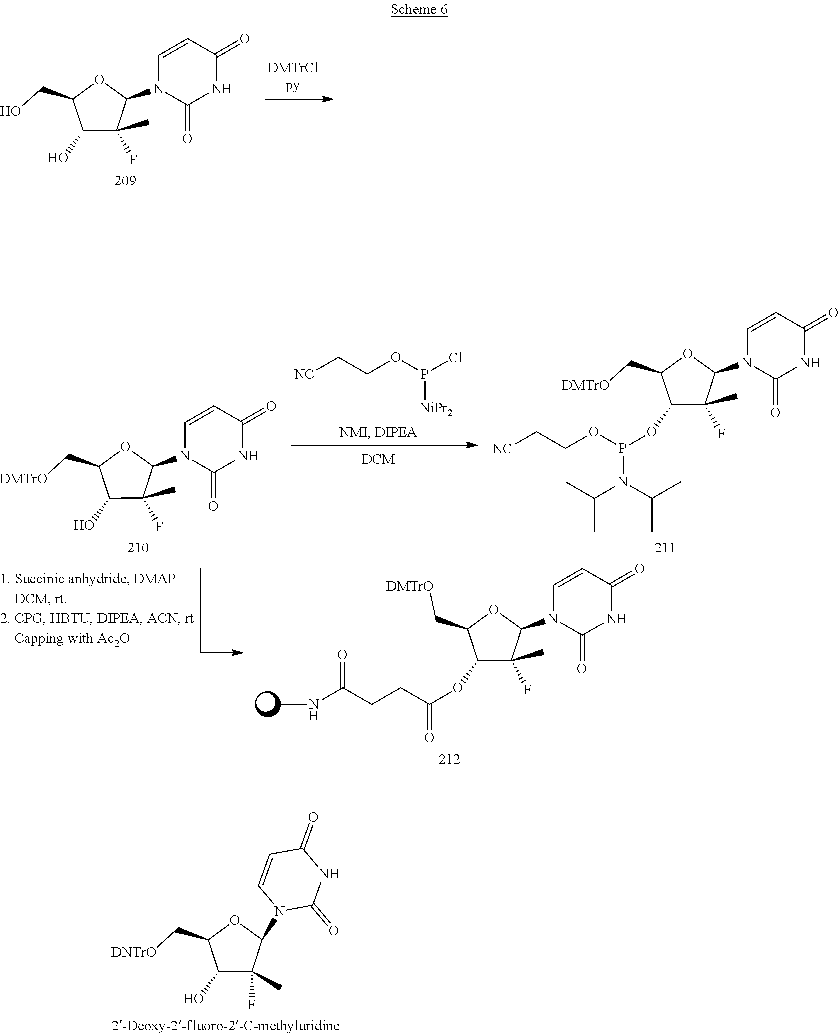

2′-Deoxy-2′-fluoro-2′-C-methyluridine (209) was prepared from the literature procedure.^{2,3}

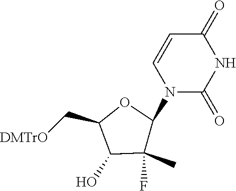

1-[(2R,5R)-5-[[bis(4-methoxyphenyl)-phenyl-methoxy]methyl]-3-fluoro-4-hydroxy-3-methyl-tetrahydrofuran-2-yl]pyrimidine-2,4-dione 210: To a clear solution of 209 (1.0 g, 3.84 mmol) in pyridine (30 mL) was added 4,4′-dimethoxytrityl chloride (1.56 g, 4.61 mmol) in two portions. Reaction mixture was stirred at for 16 hr, diluted with DCM (30 mL) and then quenched with 10% NaHCO₃ (30 mL). Organic layer was washed with brine (2×20 mL), separated, dried over anhydrous Na₂SO₄, and filtered. Filtrate was evaporated under high vacuum pump and crude mass obtained, was purified by flash column chromatography (0-5% MeOH in DCM) to afford 210 (1.95 g, 90% yield) as white foam. ¹H NMR (600 MHz, DMSO-d₆) δ 11.53-11.50 (m, 1H), 7.84 (d, J=8.1 Hz, 1H), 7.40-7.31 (m, 4H), 7.29-7.22 (m, 5H), 6.94-6.88 (m, 4H), 6.07-5.97 (m, 1H), 5.78 (s, 1H), 5.14 (s, 1H), 4.12-4.04 (m, 1H), 4.00-3.97 (m, 1H), 3.74 (s, 6H), 3.41 (dd, J=11.1, 4.1 Hz, 1H), 1.37-1.25 (m, 3H) ppm. ¹⁹F NMR (565 MHz, DMSO-d₆) δ −159.88 ppm. ¹³C NMR (151 MHz, DMSO-d₆) δ 162.72, 158.19, 158.17, 150.45, 144.55, 135.32, 134.93, 129.81, 127.95, 127.75, 126.86, 113.29, 101.88, 101.22, 100.03, 86.02, 80.06, 71.31, 61.32, 59.75, 55.06, 16.50, 16.33 ppm.

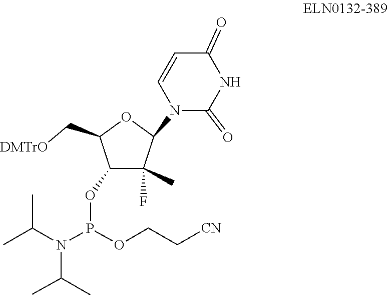

3-[[(2R,5R)-2-[[bis(4-methoxyphenyl)-phenyl-methoxy]methyl]-5-(2,4-dioxopyrimidin-1-yl)-4-fluoro-4-methyl-tetrahydrofuran-3-yl]oxy-(diisopropylamino)phosphanyl]oxy propanenitrile 211: To a clear solution of 210 (1.9 g, 3.38 mmol) in dichloromethane (30 mL) at 22° C. was added N-methyl imidazole (415.91 mg, 5.07 mmol, 403.80 μL) and diisopropylethylamine (2.18 g, 16.89 mmol, 2.94 mL) The reaction mixture was stirred for 5 minutes at rt and 2-cyanoethyl-N,N-diisopropylchlorophosphoramidite (1.60 g, 6.75 mmol, 1.51 mL) was added slowly into it. Reaction was kept for stirring at 22° C. and TLC was checked after 1 hr. Reaction mixture was diluted with dichloromethane (20 mL) and washed with 10% NaHCO₃ solution (20×2 mL). Organic layer separated, dried over anhydrous Na₂SO₄, filtered and the filtrate was evaporated to dryness. The crude mass obtained was purified by flash column chromatography (gradient: 40-80% EtOAc in hexane) to afford 211 (2.0 g, 78% yield) as white foam. ¹H NMR (600 MHz, CD₃CN) δ 9.15 (s, 1H), 7.93-7.86 (m, 1H), 7.44 (dt, J=8.3, 1.3 Hz, 2H), 7.37-7.24 (m, 6H), 6.92-6.85 (m, 4H), 6.16-5.99 (m, 1H), 5.07 (dd, J=17.6, 8.1 Hz, 1H), 4.49-4.32 (m, 1H), 4.17-4.08 (m, 1H), 3.91-3.64 (m, 7H), 3.63-3.42 (m, 2H), 2.69-2.64 (m, 1H), 2.48-2.41 (m, 1H), 1.57-1.35 (m, 3H), 1.21-1.12 (m, 10H), 1.01 (d, J=6.8 Hz, 3H) ppm. ³¹P NMR (243 MHz, CD₃CN) δ 150.69, 149.37 ppm. 1 ¹⁹F NMR (565 MHz, CD₃CN) δ −159.70 ppm. ¹³C NMR (151 MHz, CD₃CN) δ 163.57, 159.86, 159.82, 151.53, 151.51, 145.71, 140.85, 136.39, 136.31, 136.27, 136.18, 131.35, 131.29, 131.25, 129.25, 129.18, 128.99, 128.96, 128.12, 128.07, 119.62, 119.38, 114.15, 114.12, 103.10, 103.07, 102.23, 102.21, 101.95, 101.92, 101.02, 101.00, 100.73, 100.71, 90.16, 87.80, 87.75, 81.33, 81.14, 81.12, 74.67, 74.56, 74.47, 74.29, 74.19, 74.09, 61.84, 61.37, 60.96, 59.33, 59.20, 59.05, 58.92, 55.95, 55.91, 55.32, 44.09, 44.00, 24.98, 24.93, 24.90, 24.88, 24.86, 24.75, 24.71, 21.15, 20.99, 20.93, 20.88, 18.11, 18.07, 17.94, 17.91, 17.53, 17.36 ppm.

### REFERENCES

(1) Reddy, P. G.; Chun, B.-K.; Zhang, H.-R.; Rachakonda, S.; Ross, B. S.; Sofia, M. J. J Org. Chem. 2011, 76, 3782.
(2) Sofia, M. J.; Bao, D.; Chang, W.; Du, J.; Nagarathnam, D.; Rachakonda, S.; Reddy, P. G.; Ross, B. S.; Wang, P.; Zhang, H.-R.; Bansal, S.; Espiritu, C.; Keilman, M.; Lam, A. M.; Steuer, H. M. M.; Niu, C.; Otto, M. J.; Furman, P. A. J. Med. Chem. 2010, 53, 7202.
(3) Maiti, M.; Maiti, M.; Rozenski, J.; De Jonghe, S.; Herdewijn, P. Org. Biomol. Chem. 2015, 13, 5158.

TABLE 11
Abbreviations used in sequences

| | |
|---|---|
| Af or A_{F} | 2′-deoxy-2′-fluoroadenosine-3′-phosphate |
| Afs | 2′-deoxy-2′-fluoroadenosine-3′-phosphorothioate |
| Cf or C_{F} | 2′-deoxy-2′-fluorocytidine-3′-phosphate |
| Cfs | 2′-deoxy-2′-fluorocytidine-3′-phosphorothioate |
| Gf or G_{F} | 2′-deoxy-2′-fluoroguanosine-3′-phosphate |
| Gfs | 2′-deoxy-2′-fluoroguanosine-3′-phosphorothioate |
| Uf or U_{F} | 2′-deoxy-2′-fluorouridine-3′-phosphate |
| Ufs | 2′-deoxy-2′-fluorouridine -3′-phosphorothioate |
| a | 2′-O-methyladenosine-3′-phosphate |
| as | 2′-O-methyladenosine-3′ - phosphorothioate |
| c | 2′-O-methylcytidine-3′-phosphate |
| cs | 2′-O-methylcytidine-3′- phosphorothioate |
| g | 2′-O-methylguanosine-3′-phosphate |
| gs | 2′-O-methylguanosine-3′ - phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| dA | 2′-deoxyadenosine-3′-phosphate (Deoxy-Adenosine) |
| dAs | 2′-deoxyadenosine-3′-phosphorothioate |
| dC | 2′-deoxycytidine-3′-phosphate (Deoxy-Cytidine) |
| dCs | 2′-deoxycytidine-3′-phosphorothioate |
| dG | 2′-deoxyguanosine-3′-phosphate (Deoxy-Guanosine) |
| dG | 2′-deoxyguanosine-3′-phosphorothioate |
| dT | 2′ -deoxythymidine-3′-phosphate (Deoxy-Thymidine) |
| dTs | 2′-deoxythymidine-3′-phosphorothioate |
| VP | 5′-E-vinylphosphonate |
| zVP | 5′-(Z)-vinylphosphonate |
| U_{F/M} | 2′-deoxy-2′-fluoro-2′-C-methyl-uridine-3′-phosphate |
| C_{F/M} | 2′-deoxy-2′-fluoro-2′-C- methyl-cytidine-3′-phosphate |
| U_{F/MS} | 2′-deoxy-2′-fluoro- 2′-C-methyl-uridine-3′-phosphorothioate |
| C_{F/MS} | 2′-deoxy-2′-fluoro-2′-C-methyl-cytidine-3′-phosphorothioate |
| s | Phosphorothioate linkage - indicates modification of 3′-phosphate for 3′- |
| | phosphorothioate on the preceding nucleotide |
| “•” symbol | Phosphorothioate linkages-indicates modification of 3′-phosphate for 3′- |
| | phosphorothioate on the preceding nucleotide |
| L96 | (2S,4R)-1-[29-[[2-(acetylamino)-2-deoxy-β-D-galactopyranosyl]oxy]-14,14-bis[[3-[[3-[[5-[[2-(acetylamino)-2-deoxy-B-D- |
| | galactopyranosyl]oxy]-1-oxopentyl]amino]propyl]amino]-3-oxopropoxy]methyl]-1,12,19,25-tetraoxo-16-oxa-13,20,24- |
| | triazanonacos-1-y1]-4-hydroxy-2-hydroxymethylpyrrolidine |
| | 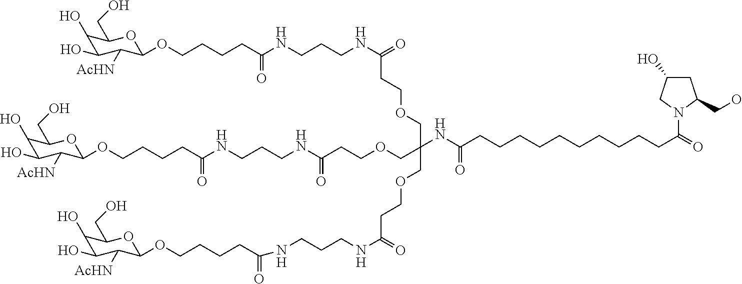 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| uL96 | 2′-O-methyluridine-3′-phosphate ((2S,4R)-1-[29-[2-(acetylamino)-2-deoxy-β-D-galactopyranosyl]oxy]-14,14-bis[[3-[[3-[5-[[2- |
| | (acetylamino)-2-deoxy-β-D-galactopyranosyl]oxy]-1-oxopentyl]amino]propyl]amino]-3-oxopropoxy]methyl]-1,12,19,25-tetraoxo- |
| | 16-oxa-13,20,24-triazanonacos-1-yl]-4-hydroxy-2-pyrrolidinyl)methyl ester |
| | 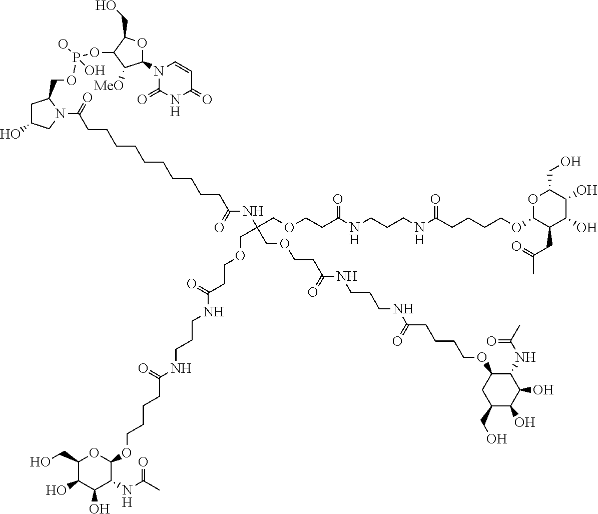 |

*3′-terminal nucleotides are 3′—OH unless conjugated to (L) or otherwise indicated

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A double-stranded RNA (dsRNA) comprising a sense strand and an antisense strand complementary to the sense strand, wherein the dsRNA has a double-stranded region of at least about 15 base-pairs, and wherein the antisense strand comprises one or both of (a) and (b):
(a) a 5′-terminal nucleoside that is a 2′-geminal-substituted nucleoside of formula (II) or (II′): 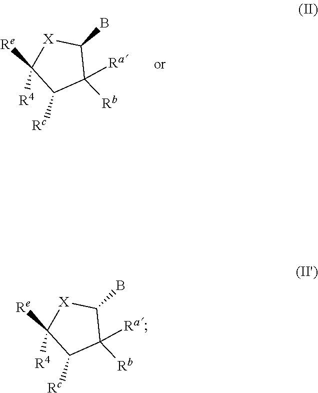
and
(b) a 2′-geminal-substituted nucleoside according to formula (I) or (I′) at least at one of positions 2-9 (e.g., at position 2, 3, 4, 5, 6, 7, 8 and/or 9), counting from the 5′-end of the antisense strand: 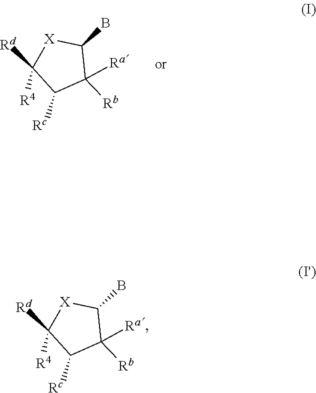
wherein in formulae (I), (I′), (II) and (II′):
X is O, S, C(R^{X})₂, or N(R^{XN});
each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{N}), or =CH₂;
R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
B is an optionally modified nucleobase;
R^{a′} is halogen, hydrogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a bond to an internucleoside linkage to a subsequent nucleoside;
R^{a2} is hydrogen or hydroxyl protecting group;
R^{a3} is hydrogen or sulfur protecting group;
R^{a4} is independently for each occurrence H, C₁-C₃₀alkyl, cyclyl, heterocyclyl, aryl, heteroaryl, aralkyl, sugar or R^{a5};
R^{a5} is independently for each occurrence amino (NH₂), alkylamino, dialkylamino, arylamino, diarylamino, heteroarylamino, or diheteroaryl amino;
m is 1-50;
n is 1-50;
R^{b} is optionally substituted C₁₋₃₀alkyl, optionally substituted C₂₋₃₀alkenyl, optionally substituted C₂₋₃₀alkynyl, or halogen;
R^{c} is a bond to an internucleoside linkage to a subsequent nucleoside, hydrogen, halogen, —OR^{c2}, —SR^{c3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, or a linker covalently attached to a solid support, and where, optionally, at least R^{c} or R^{a} is a bond to an internucleoside linkage to a subsequent nucleoside;
R^{c2} is hydrogen or hydroxyl protecting group;
R^{c3} is hydrogen or sulfur protecting group;
R^{c4} is independently for each occurrence H, C₁-C₃₀alkyl, cyclyl, heterocyclyl, aryl, heteroaryl, aralkyl, sugar or R^{c5};
R^{c5} is independently for each occurrence amino (NH₂), alkylamino, dialkylamino, arylamino, diarylamino, heteroarylamino, or diheteroaryl amino;
r is 1-50;
s is 1-50;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R^{a} taken together are 4′-C(R^{a11}R^{a12})ᵥ—Y-2′ or 4′-Y—C(R^{a11}R^{a12})ᵥ-2′;
Y is —O—, —CH₂—, —CH(Me)-, —C(CH₃)₂—, —S—, —N(R^{a13})—, —C(O)—, —C(S)—, —S(O)—, —S(O)₂—, —OC(O)—, —C(O)O—, —N(R^{a13})C(O)—, or —C(O)N(R^{a13})—;
R^{a11} and R^{a12} independently are H, optionally substituted C₁-C₆alkyl, optionally substituted C₂-C₆alkenyl or optionally substituted C₂-C₆alkynyl;
R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
v is 1, 2 or 3;
or R⁴ and R^{c}taken together with the atoms to which they are attached form an optionally substituted C₃₋₈cycloalkyl, optionally substituted C₃₋₈cycloalkenyl, or optionally substituted 3-8 membered heterocyclyl;
R^{d} is —CH(R^{d1})—R^{d2} or —C(R^{d1})=CHR^{d2};
R^{d1} is hydrogen, optionally substituted C₁₋₃₀alkyl, optionally substituted —C₂₋₃₀alkenyl, or optionally substituted —C₂₋₃₀alkynyl;
R^{d2} is a bond to an internucleoside linkage to the preceding nucleoside;
R^{e} is optionally substituted —C₂₋₆alkenyl-R^{e1}, optionally substituted C₁₋₆alkyl-R^{e1}, or optionally substituted —C₂₋₆alkynyl-R^{e1};
R^{e1} is —P(O)(OR^{e4})₂, —OR^{e2}, —SR^{e3}, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂;
R^{e2} is hydrogen or oxygen protecting group;
R^{e3} is hydrogen or sulfur protecting group;
each R^{e4} is independently hydrogen, optionally substituted C₁₋₃₀alkyl, optionally substituted C₂₋₃₀alkenyl, or optionally substituted C₂₋₃₀alkynyl, or an oxygen-protecting group;
and each R^{e5} is independently hydrogen, optionally substituted C₁₋₃₀alkyl, optionally substituted C₂₋₃₀alkenyl, or optionally substituted C₂₋₃₀alkynyl, or a sulfur-protecting group.

2. The dsRNA of any one of the preceding claims, wherein the 5′-terminal nucleotide of the antisense strand is a 2′-geminal-substituted nucleotide of formula (II).

3. The dsRNA of any one of the preceding claims, wherein the antisense strand comprises a vinylphosphonate (e.g., E-vinylphosphonate) group at its 5′-end.

4. The dsRNA of any one of the preceding claims, wherein the 5′-terminal nucleotide of the antisense strand is a 2′-geminal-substituted nucleotide of formula (II), and wherein Re is vinyl phosphonate (e.g., R is R is —CH═CHR^{e1} and R¹ is —P(O)(OR^{e4})₂).

5. The dsRNA of any one of the preceding claims, wherein the antisense strand comprises a nucleoside of Formula (I) at least at position 3, counting from the 5′-end of the antisense strand.

6. The dsRNA of any one of the preceding claims, wherein the antisense strand comprises a nucleoside of Formula (I) at least at position 4, counting from the 5′-end of the antisense strand.

7. The dsRNA of any one of the preceding claims, wherein the antisense strand comprises a nucleoside of Formula (I) at least at least at position 5, counting from the 5′-end of the antisense strand.

8. The dsRNA of any one of the preceding claims, wherein the antisense strand comprises a nucleoside of Formula (I) at least at least at position 6, counting from the 5′-end of the antisense strand.

9. The dsRNA of any one of the preceding claims, wherein the antisense strand comprises a nucleoside of Formula (I) at least at least at position 7, counting from the 5′-end of the antisense strand.

10. The dsRNA of any one of the preceding claims, wherein the antisense strand comprises a nucleoside of Formula (I) at least at least at position 8, counting from the 5′-end of the antisense strand.

11. The dsRNA of any one of the preceding claims, wherein the antisense strand comprises a nucleoside of Formula (I) at least at least at position 9, counting from the 5′-end of the antisense strand.

12. The dsRNA of any one of the preceding claims, wherein:
(i) the 2′-geminal-substituted nucleoside of formula (II) is according to formula (IIA): 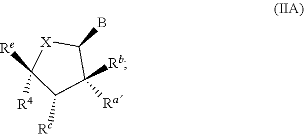
or according to formula (IIB): 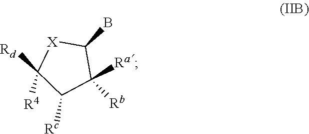
or
(ii) the 2′-geminal-substituted nucleoside of formula (II′) is according to formula (IIA′): 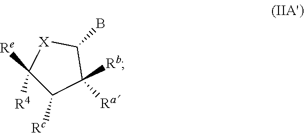
or according to formula (IIB′):

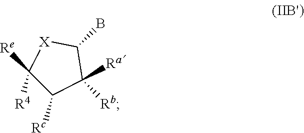

13. The dsRNA of any one of the preceding claims, wherein:
(i) the 2′-geminal-substituted nucleoside of formula (I) is according to formula (IA): 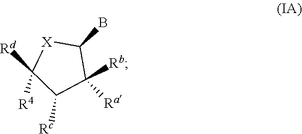
or according to formula (IB): 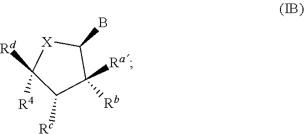
or
(ii) the 2′-geminal-substituted nucleoside of formula (I′) is according to formula (IA′): 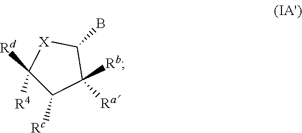
or according to formula (IB′):

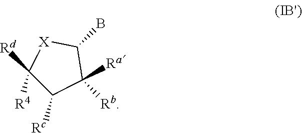

14. The dsRNA of any one of the preceding claims, wherein X is O.

15. The dsRNA of any one of the preceding claims, wherein R^{a′} is hydrogen, halogen, —OR^{a2}, optionally substituted C₁-C₃₀alkyl, optionally substituted C₁-C₃₀alkoxy, —O(CH₂CH₂O)ₘCH₂CH₂OR^{a4}, or —NH(CH₂CH₂NH)ₙCH₂CH₂—R^{a5}.

16. The dsRNA of any one of the preceding claims, wherein R^{a′} is halogen, hydrogen, —OR^{a2}, or optionally substituted C₁-C₃₀alkoxy,

17. The dsRNA of any one of the preceding claims, wherein R^{a′} is halogen, —OR^{a2}, or optionally substituted C₁-C₃₀alkoxy.

18. The dsRNA of any one of the preceding claims, wherein R^{a′} is F, Cl, OH or optionally substituted C₁-C₃₀alkoxy (e.g., R^{a′} is F, Cl)

19. The dsRNA of any one of the preceding claims, wherein R^{a′} is C₁-C₃₀alkoxy optionally substituted with an amino or C₁-C₆alkoxy.

20. The dsRNA of any one of the preceding claims, wherein R^{b} is optionally substituted C₁₋₆alkyl, C₁₋₆haloalkyl, optionally substituted C₂₋₆alkenyl, optionally substituted C₂₋₆alkynyl or halogen.

21. The dsRNA of any one of the preceding claims, wherein R^{b} is methyl, ethyl, propyl, isopropyl, butyl, iso-butyl, t-butyl, vinyl, ethynyl, allyl or propargyl.

22. The dsRNA of any one of the preceding claims, wherein R^{b} is methyl, vinyl, ethynyl, allyl or propargyl.

23. The dsRNA of any one of the preceding claims, wherein R⁴ is hydrogen, optionally substituted C₁₋₆alkyl, optionally substituted C₂₋₆alkenyl, optionally substituted C₂₋₆alkynyl, or optionally substituted C₁₋₆alkoxy.

24. The dsRNA of any one of the preceding claims, wherein R⁴ is hydrogen.

25. The dsRNA of any one of the preceding claims, wherein R^{d} is —CH(R^{d1})-X^{d}-R^{d2}.

26. The dsRNA of any one of the preceding claims, wherein X^{d} is O.

27. The dsRNA of any one of the preceding claims, wherein R^{d1} is hydrogen or optionally substituted C₁-C₆alkyl.

28. The dsRNA of any one of the preceding claims, wherein R^{d6} is hydrogen.

29. The dsRNA of any one of the preceding claims, wherein R^{e} is —C₂₋₆alkenyl-R^{e1} or C₁₋₆alkyl-R^{e1}, C₁₋₆alkyl and C₂₋₆alkenyl is optionally substituted.

30. dsRNA of any one of the preceding claims, wherein R^{e} is —CH═CHR^{e1}.

31. The dsRNA of any one of the preceding claims, wherein R^{e1} is —P(O)(OR⁴)₂, —OR^{e2}, or —OP(O)(OR^{e4})₂.

32. The dsRNA of any one of the preceding claims, wherein R^{e2} is hydrogen or optionally substituted C₁-C₆alkyl.

33. The dsRNA of any one of the preceding claims, wherein the 2′-geminal-substituted nucleoside of formula (I) or (I′) is at least at one of position 2, 3, 4, 5, 6, 7, 8, 9 or 10, counting from the 5′-end of the antisense strand.

34. The dsRNA of any one of the preceding claims, wherein the 2′-geminal-substituted nucleoside of formula (I) is at position 7, counting from the 5′-end of the antisense strand.

35. The dsRNA of any one of the preceding claims, wherein the antisense strand further comprises at least one modified internucleoside linkage.

36. The dsRNA of any one of the preceding claims, wherein the antisense strand further comprises at least one modified nucleobase.

37. The dsRNA of any one of the preceding claims, wherein the wherein the antisense strand further comprises at least one nucleoside modified at the 2′-position and wherein the nucleotide modified at the 2′-position is not a 2′-geminal nucleoside.

38. The dsRNA of any one of the preceding claims, wherein the 2′-geminal-substituted nucleoside of formula (II) is of formula (IIA): 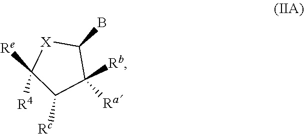
wherein:
X is O;
R^{a′} is halogen (e.g., F, Cl or Br), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl or Br);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{e} is —CH═CHR, where R^{e1} is —P(O)(OR^{e4})₂.

39. The dsRNA of any one of claims 1-38, wherein the 2′-geminal-substituted nucleoside of formula (II) is of formula (IIB): 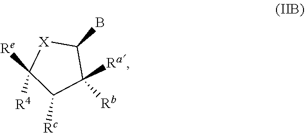
wherein:
X is O;
R^{a′} is halogen (e.g., F, Cl or Br), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl or Br);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R is —CH═CHR^{e1}, where R^{e1} is —P(O)(OR^{e4})₂.

40. The dsRNA of any one of the preceding claims, wherein the 2′-geminal-substituted nucleoside of formula (I) is of formula (IA): 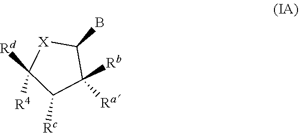
wherein:
X is O;
R^{a′} is halogen (e.g., F, Cl or Br), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl or Br);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{d} is a bond to an internucleoside linkage to a preceding nucleoside, and
provided that only one of a R^{a′} and R^{c} is a bond to an internucleoside linkage to a subsequent nucleoside.

41. The dsRNA of any one of claims 1-39, wherein the 2′-geminal-substituted nucleoside of formula (I) is of formula (IB): 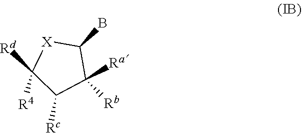
wherein:
X is O;
R^{a′} is halogen (e.g., F, Cl or Br), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl or Br);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{d} is a bond to an internucleoside linkage to a preceding nucleoside, and
provided that only one of a R^{a′} and R^{c} is a bond to an internucleoside linkage to a subsequent nucleoside.

42. The dsRNA of any one of the preceding claims, wherein the antisense strand is at least about 17, e.g., about 18, about 19, about 20, about 21, about 22, about 23, about 24, about 25, about 26, about 27, about 28, about 29, about 30 or more (e.g., about 17-42), nucleotides in length.

43. The dsRNA of any one of the preceding claims, wherein the antisense strand is about 19, about 20, about 21, about 22, about 23, about 24, about 25 or about 26 nucleotides in length.

44. The dsRNA of any one of the preceding claims, wherein the antisense strand is about 22, about 23, about 24, or about 25 nucleotides in length.

45. The dsRNA of any one of the preceding claims, wherein the sense strand is at least about 15, about 16, e.g., about 17, about 18, about 19, about 20, about 21, about 22, about 23, about 24, about 25, about 26, or more (e.g., about 15-40), nucleotides in length.

46. The dsRNA of any one of the preceding claims, wherein the sense strand is about 19, about 20, about 21, about 22, about 23, about 24 or about 25 nucleotides in length.

47. The dsRNA of any one of the preceding claims, wherein the sense strand is about 21 nucleotides in length.

48. The dsRNA of any one of the preceding claims, wherein:
(a) the sense strand is 15 nucleotides in length and the antisense strand is 18, 19, 20, 21, or 22 (e.g., 20) nucleotides in length;
(b) the sense strand is 19 nucleotides in length and the antisense strand is 19, 20, or 21 nucleotides in length;
(c) the sense strand is 20 nucleotides in length and the antisense strand is 20, 21, or 22 nucleotides in length;
(d) the sense strand is 21 nucleotides in length and the antisense strand is 21, 22, or 23 nucleotides in length; or
(e) the sense strand is 20-24 (e.g., 22) nucleotides in length and the antisense strand is 34-38 (e.g. 36) nucleotides in length.

49. The dsRNA of any one of the preceding claims, wherein the dsRNA has a double-stranded region of at least about 15, e.g., about 16, about 17, about 18, about 19, about 20, about 21, about 22, about 23, about 24, about 25 or more base-pairs.

50. The dsRNA of any one of the preceding claims, wherein the dsRNA has a double-stranded region of about 21 base-pairs.

51. The dsRNA of any one of the preceding claims, wherein the sense strand is about 21 nucleotides in length and the antisense strand is about 21, about 22, about 23, about 24 or about 25 nucleotides in length, and wherein the dsRNA comprises a double-stranded region of at least 18, e.g., 19, 20 or 21 base-pairs.

52. The dsRNA of any one of the preceding claims, wherein the dsRNA comprises at least one single-stranded overhang comprising 1-5 nucleotides (e.g., 1 or 2 nucleotides).

53. The dsRNA of any one of the preceding claims, wherein the antisense strand comprises a single-stranded overhang at its 3′-end.

54. The dsRNA of any one of the preceding claims, wherein the dsRNA comprises at least one blunt-end.

55. The dsRNA of any one of the preceding claims, wherein the antisense strand comprises a blunt end at its 5′-end.

56. The dsRNA of any one of the preceding claims, wherein the antisense strand comprises a single-stranded overhang at its 3′-end and a blunt end at its 5′-end.

57. The dsRNA of any one of the preceding claims, wherein the dsRNA comprises at least 4 phosphorothioate internucleoside linkages, e.g., at least 6 phosphorothioate internucleoside linkages or at least 8 phosphorothioate at least 10 phosphorothioate internucleoside linkages.

58. The dsRNA of any one of the preceding claims, wherein the antisense strand comprises at least two, e.g., three, four, six or more phosphorothioate internucleoside linkages

59. The dsRNA of any one of the preceding claims, wherein the antisense strand comprises a phosphorothioate internucleoside linkage between positions 1 and 2, counting from the 3′-end of the strand, and a phosphorothioate internucleoside linkage between positions 1 and 2, counting from the 5′-end of the strand.

60. The dsRNA of any one of the preceding claims, wherein the antisense strand comprises a phosphorothioate internucleoside linkage between positions 1 and 2, and between positions 2 and 3, counting from the 3′-end of the strand, and a phosphorothioate internucleoside linkage between positions 1 and 2, counting from the 5′-end of the strand.

61. The dsRNA of any one of the preceding claims, wherein the antisense strand comprises a phosphorothioate internucleoside linkage between positions 1 and 2, and between positions 2 and 3, counting from the 3′-end of the strand, and a phosphorothioate internucleoside linkage between positions 1 and 2, and between positions 2 and 3, counting from the 5′-end of the strand.

62. The dsRNA of any one of the preceding claims, wherein the antisense strand comprises a phosphorothioate internucleoside linkage between positions 1 and 2, between positions 2 and 3, and between positions 3 and 4, counting from the 3′-end of the strand, and a phosphorothioate internucleoside linkage between positions 1 and 2, counting from the 5′-end of the strand.

63. The dsRNA of any one of the preceding claims, wherein the antisense strand comprises a phosphorothioate internucleoside linkage between positions 1 and 2, counting from the 3′-end of the strand, and a phosphorothioate internucleoside linkage between positions 1 and 2, and between positions 2 and 3, counting from the 5′-end of the strand.

64. The dsRNA of any one of the preceding claims, wherein the antisense strand comprises a phosphorothioate internucleoside linkage between positions 1 and 2, counting from the 3′-end of the strand, and a phosphorothioate internucleoside linkage between positions 1 and 2, between positions 2 and 3, and between positions 3 and 4, counting from the 5′-end of the strand.

65. The dsRNA of any one of the preceding claims, wherein the sense strand comprises at least one, e.g., two, three, four or more phosphorothioate internucleoside linkages.

66. The dsRNA of any one of the preceding claims, wherein the sense strand comprises a phosphorothioate internucleoside linkage between positions 1 and 2, counting from 5′-end of the strand.

67. The dsRNA of any one of the preceding claims, wherein the sense strand comprises a phosphorothioate internucleoside linkage between positions 1 and 2, counting from 5′-end of the strand, and between positions 1 and 2, counting from 3′-end of the strand.

68. The dsRNA any one of the preceding claims, wherein the sense strand comprises a phosphorothioate internucleoside linkage between positions 1 and 2, and between positions 2 and 3, counting from 5′-end of the strand.

69. The dsRNA any one of the preceding claims, wherein the sense strand comprises a phosphorothioate internucleoside linkage between positions 1 and 2, and between positions 2 and 3, counting from 5′-end of the strand, and between positions 1 and 2, and between positions 2 and 3, counting from 3′-end of the strand.

70. The dsRNA of any one of the preceding claims, wherein the dsRNA comprises a ligand.

71. The dsRNA of claim 70, wherein the ligand is linked to the sense strand.

72. The dsRNA of claim 70 or 71, wherein the ligand is linked to 3′-end of the sense strand.

73. The dsRNA of claim 70 or 71, wherein the ligand is linked to 5′-end of the sense strand.

74. The dsRNA of any one of claims 70-73, wherein the ligand is selected from the group consisting of peptides, centyrins, antibodies (e.g., antiCD-4 antibodies and antiCD-117 antibodies), antibody fragments, T-cell targeting ligands, B-cell targeting ligands, cancer cell targeting ligands (e.g., DUPA, folate, and RGD), spleen targeting functionalities, lung targeting functionalities, bone marrow targeting functionalities, phage display peptides, cell permeation peptides (CPPs), integrin ligands, multianionic ligands, multicationic ligands, monovalent and multivalent carbohydrates (e.g., GalNAc, mannose, mannose-6 phosphate, mucose, and mlucose), kidney targeting ligands, BBB penetration ligands, lipids, and amino acids (e.g., L-amino acids, D-amino acids, ando-amino acids).

75. The dsRNA of any one of claims 70-74, wherein the ligand comprises GalNAc.

76. The dsRNA of any one of claims 70-75, wherein the ligand is

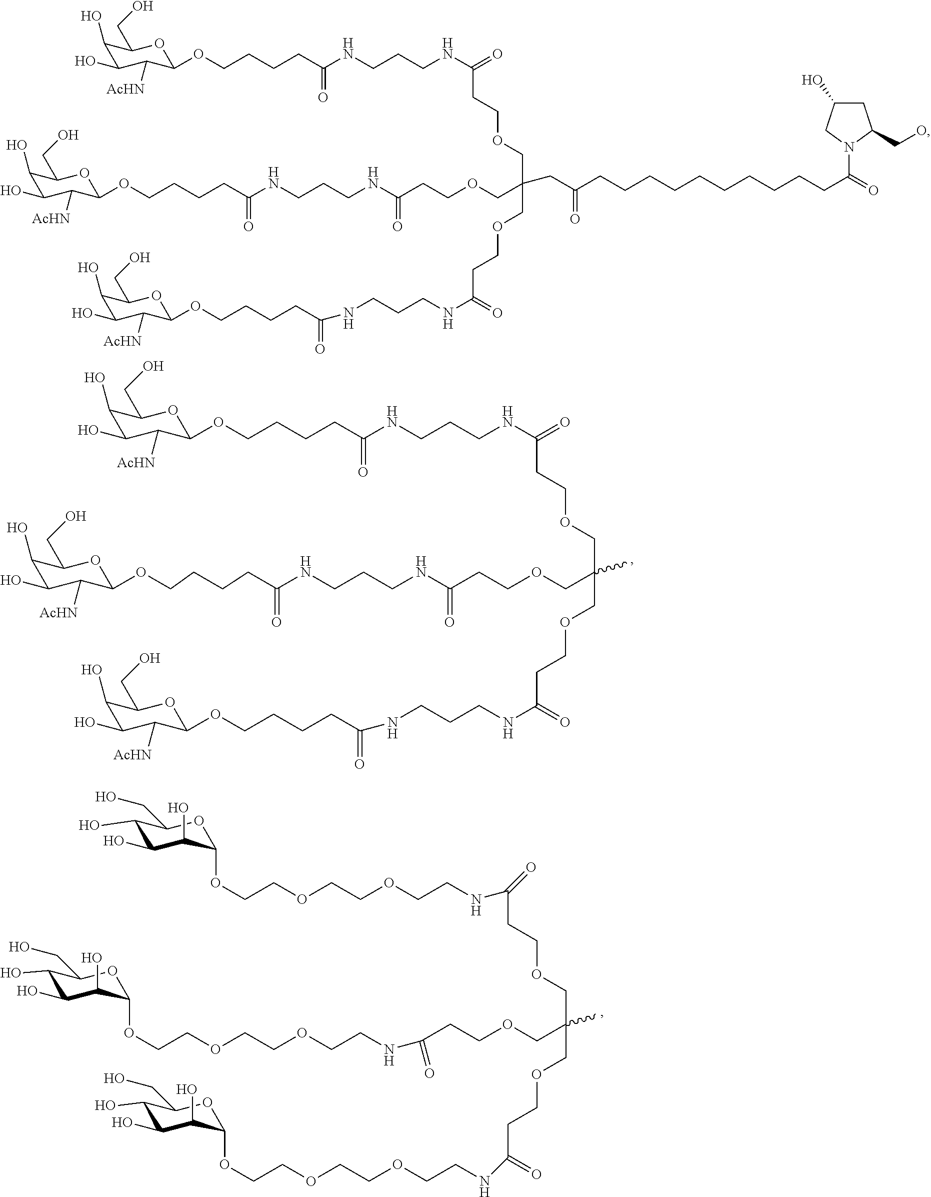

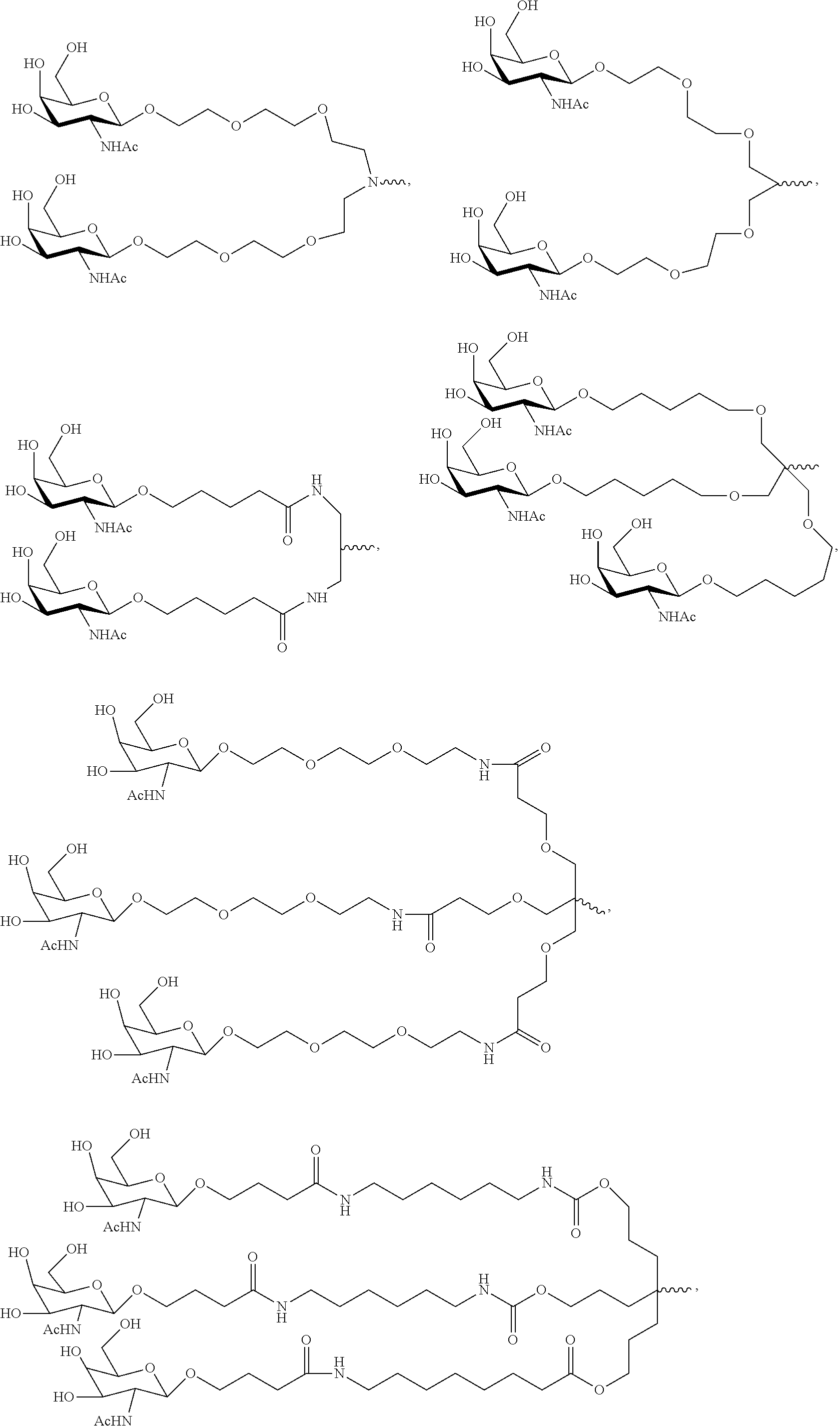

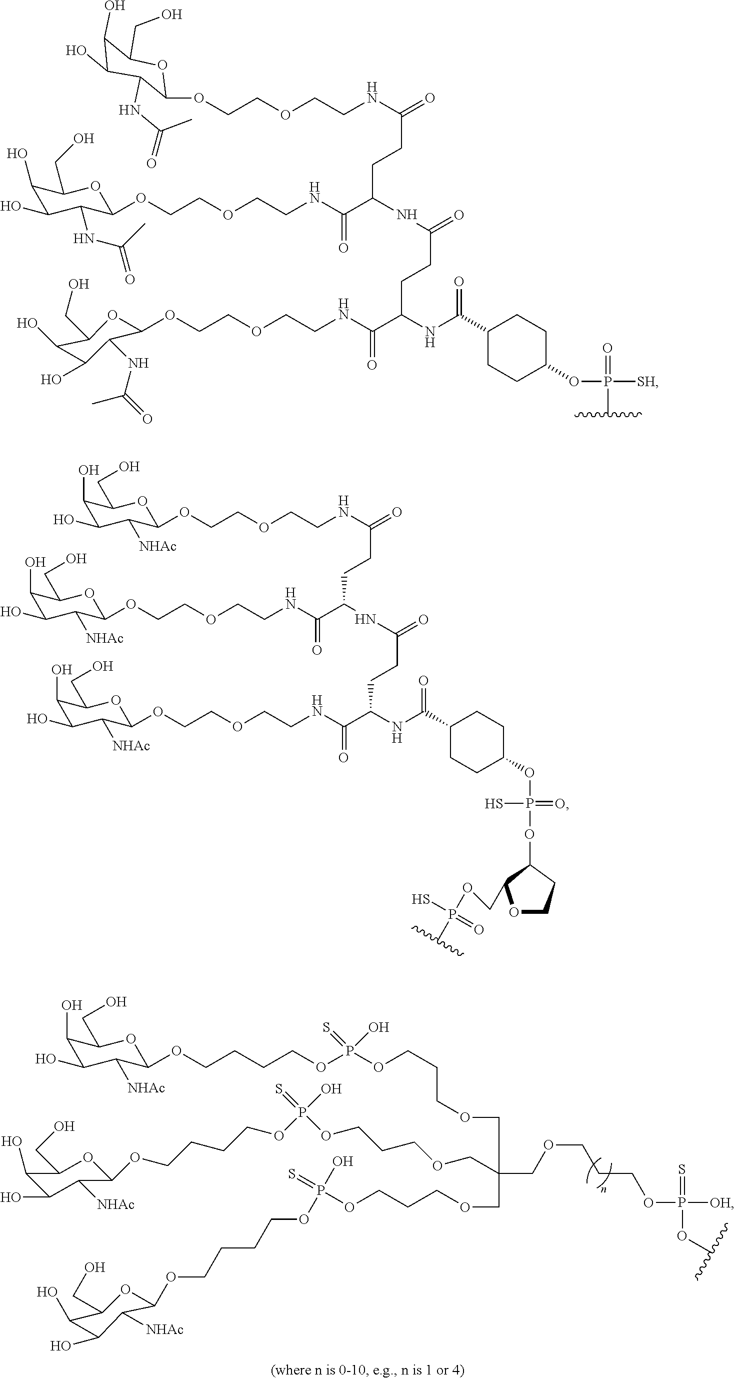

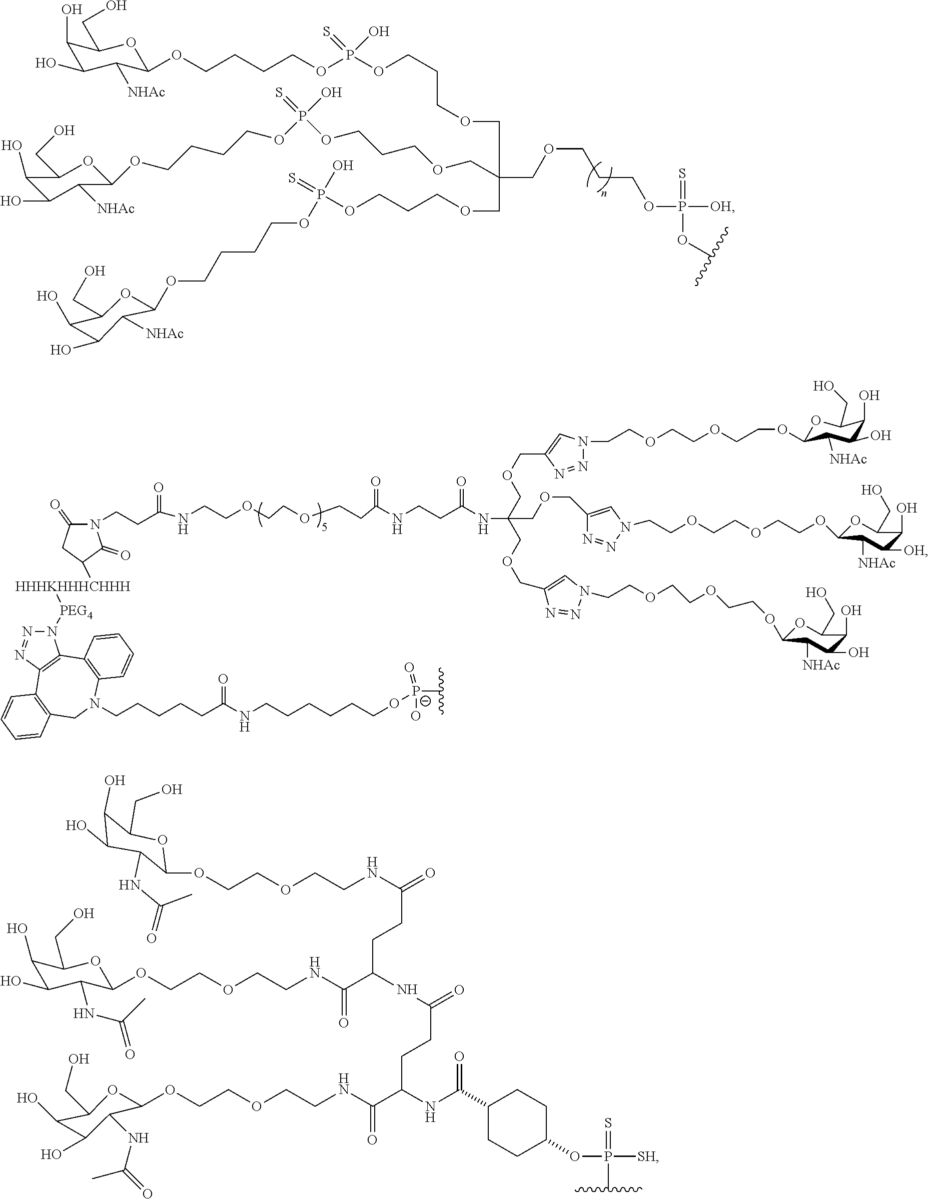

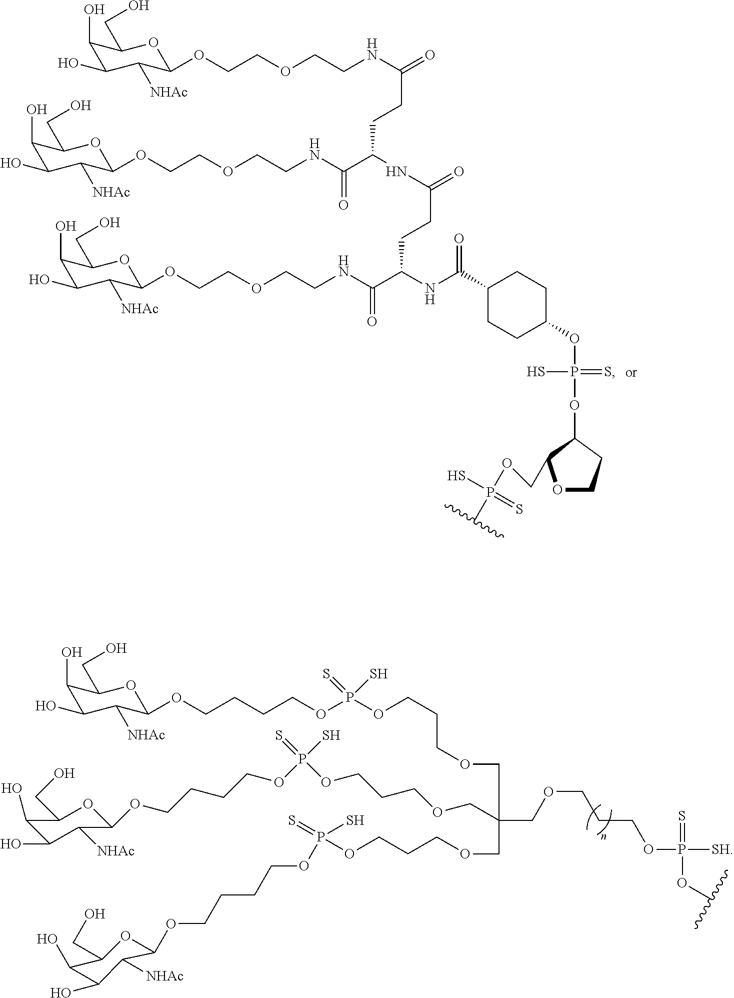

77. The dsRNA of any one of the preceding claims, wherein the dsRNA comprises at least one, e.g., 2, 3, 4, 5, 6, 7, 8, 9, 10 or more 2′-fluoro nucleotide.

78. The dsRNA of any one of the preceding claims, wherein the antisense strand comprises at least one, e.g., 2, 3, 4, 5 or more 2′-fluoro nucleotides.

79. The dsRNA of any one of the preceding claims, wherein the antisense strand comprises a 2′-fluoro nucleotide at positions 2, 14 and 16, counting from the 5′-end of the antisense strand.

80. The dsRNA of any one of the preceding claims, wherein the antisense strand comprises a 2′-fluoro nucleotide at positions 2, 6, 14 and 16, counting from the 5′-end of the antisense strand.

81. The dsRNA of any one of the preceding claims, wherein the antisense strand comprises a 2′-fluoro nucleotide at positions 2, 6, 9, 14 and 16, counting from the 5′-end of the antisense strand.

82. The dsRNA of any one of the preceding claims, wherein the antisense strand comprises a 2′-fluoro nucleotide at positions 2, 6, 8, 9, 14 and 16, counting from the 5′-end of the antisense strand.

83. The dsRNA of any one of the preceding claims, wherein the antisense strand comprises at least one, e.g., 2, 3, 4, 5 or more 2′-fluoro nucleotides.

84. The dsRNA of any one of the preceding claims, wherein the sense strand comprises a 2′-fluoro nucleotide at positions 7, 9 and 11, counting from the 5′-end of the sense strand or at positions 11, 13 and 15, counting from the 3′-end of the sense strand.

85. The dsRNA of any one of the preceding claims, wherein the sense strand comprises a 2′-fluoro nucleotide at positions 7, 9, 10 and 11, counting from the 5′-end of the sense strand or at positions 11, 12, 13 and 15, counting from the 3′-end of the sense strand.

86. The dsRNA of any one of the preceding claims, wherein the sense strand comprises a 2′-fluoro nucleotide at positions 9, 10, and 11, counting from the 5′-end of the sense strand or at positions 11, 12, and 13 counting from the 3′-end of the sense strand.

87. The dsRNA of any one of the preceding claims, the antisense strand comprises at least one, e.g., 2, 3, 4, 5, 6, 7 or more DNA nucleotides.

88. The dsRNA of any one of the preceding claims, wherein the antisense strand comprises a DNA nucleotide at positions 2, 5, 7, and 12, counting from the 5′-end of the antisense strand.

89. The dsRNA of any one of the preceding claims, wherein the antisense strand comprises a DNA nucleotide at positions 2, 5, 7, 12, and 14 counting from the 5′-end of the antisense strand.

90. The dsRNA of any one of the preceding claims, wherein the antisense strand comprises a DNA nucleotide at positions 2, 5, 7, 12, 14 and 16 counting from the 5′-end of the antisense strand.

91. The dsRNA of any one of the preceding claims, wherein the antisense strand comprises a DNA nucleotide at positions 2, 5, 7, and 12 counting from the 5′-end of the antisense strand; and a 2′-fluoro nucleotide at position 14 of the antisense strand.

92. The dsRNA of any one of the preceding claims, wherein the dsRNA comprises at least one, e.g., 2, 3, 4, 5, 6, 7, 8, 9, 10 or more 2′-OMe nucleotides.

93. The dsRNA of any one of the preceding claims, wherein the antisense strand comprises at least one 2′-OMe nucleotide.

94. The dsRNA of anyone of the preceding claims, wherein all remaining nucleotides in the antisense strand are 2′-OMe nucleotides.

95. The dsRNA of any one of the preceding claims, wherein the sense strand comprises at least one 2′-OMe nucleotide.

96. The dsRNA of anyone of the preceding claims, wherein all remaining nucleotides in the sense strand are 2′-OMe nucleotides.

97. The dsRNA of any one of the preceding claims, wherein the dsRNA comprises at least one, e.g., 2, 3, 4, 5, 6, 7, 8, 9, 10 or more locked nucleic acid (LNA) or bridged nucleic acid (BNA) nucleotides.

98. The dsRNA of any one of the preceding claims, wherein the antisense strand comprises at least one, e.g., 2, 3, 4, 5 or more LNA or BNA nucleotides.

99. The dsRNA of any one of the preceding claims, wherein the sense strand comprises at least one, e.g., 2, 3, 4, 5 or more LNA or BNA nucleotides.

100. The dsRNA of any one of the preceding claims, wherein the dsRNA comprises at least one, e.g., 2, 3, 4, 5, 6, 7, 8, 9, 10 or more cyclohexene nucleic acid (CeNA) nucleotides or analogs thereof, optionally the CeNA nucleotide or analog thereof is 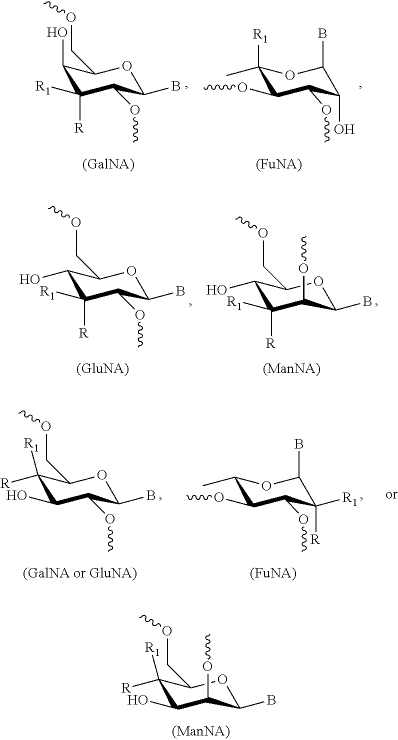
and//or a strand of the dsRNA is prepared using a monomer selected from the group consisting of: 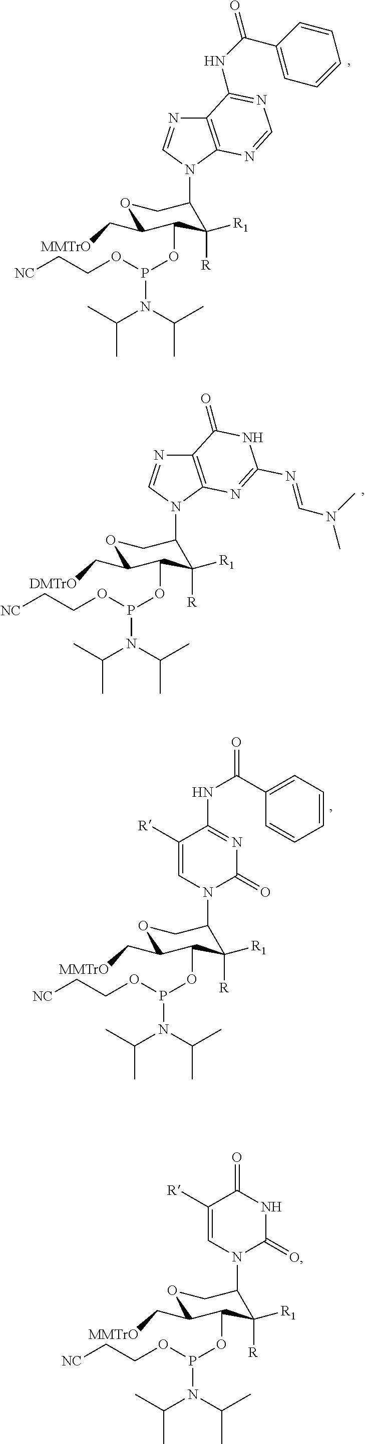 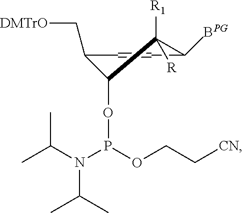 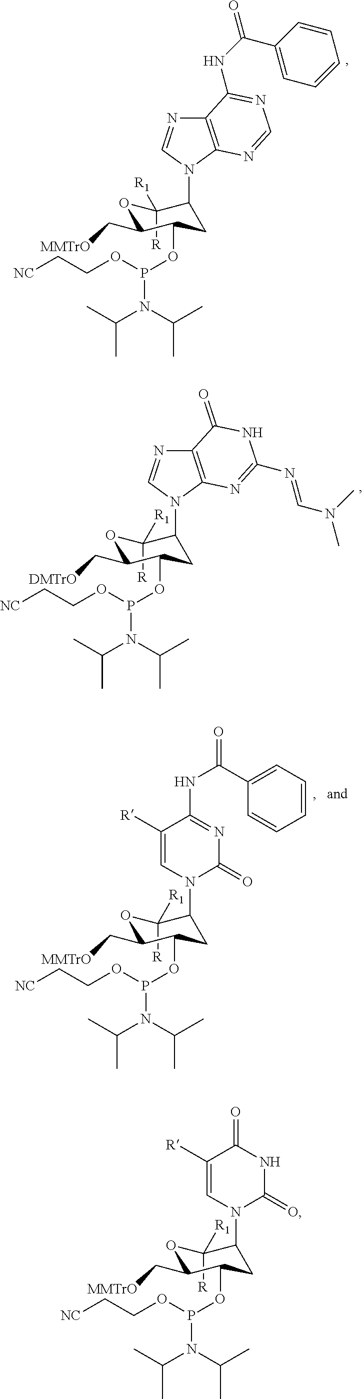
wherein:
B is an optionally modified nucleobase;
R is F, Cl, Br, I, H, protected OH, OMe, F, O-MOE, O-alkyl, O-alkene, O-alkyne, O—C₁₆, branched lipids, or protected aminoalkyl;
R¹ is F, Cl, Br, I, H, protected OH, OMe, F, O-MOE, O-alkyl, O-alkene, O-alkyne, O—C₁₆, branched lipids, protected aminoalkyl;
R¹ is H or CH₃; and
PG is a protecting group.

101. The dsRNA of any one of the preceding claims, wherein the antisense strand comprises at least one, e.g., 2, 3, 4, 5 or more CeNA nucleotides.

102. The dsRNA of any one of the preceding claims, wherein the sense strand comprises at least one, e.g., 2, 3, 4, 5 or more CeNA nucleotides.

103. The dsRNA of any one of the preceding claims, wherein the dsRNA comprises at least one, e.g., 2, 3, 4, 5, 6, 7, 8, 9, 10 or more thermally stabilizing modifications.

104. The dsRNA of any one of the preceding claims, wherein the antisense strand comprises at least one, e.g., 2, 3, 4, 5 or more thermally stabilizing modifications.

105. The dsRNA of any one of the preceding claims, wherein the sense strand comprises at least one, e.g., 2, 3, 4, 5 or more thermally stabilizing modifications.

106. The dsRNA of any one of the preceding claims, wherein the dsRNA comprises at least one, e.g., 2, 3, 4, 5, 6, 7, 8, 9, 10 or more abasic nucleotides.

107. The dsRNA of any one of the preceding claims, wherein the antisense strand comprises at least one, e.g., 2, 3, 4, 5 or more abasic nucleotides.

108. The dsRNA of any one of the preceding claims, wherein the sense strand comprises at least one, e.g., 2, 3, 4, 5 or more abasic nucleotides.

109. The dsRNA of any one of the preceding claims, wherein the dsRNA comprises at least one, e.g., 2, 3, 4, 5, 6, 7, 8, 9, 10 or more 2′-deoxy nucleotides.

110. The dsRNA of any one of the preceding claims, wherein the antisense strand comprises at least one, e.g., 2, 3, 4, 5 or more 2′-deoxy nucleotides.

111. The dsRNA of any one of the preceding claims, wherein the sense strand comprises at least one, e.g., 2, 3, 4, 5 or more 2′-deoxy nucleotides.

112. The dsRNA of any one of the preceding claims, wherein the dsRNA comprises at least one, e.g., 2, 3, 4, 5, 6, 7, 8, 9, 10 or more acyclic (e.g., unlocked nucleic acid (UNA) or glycol nucleic acid (GNA)) nucleotides.

113. The dsRNA of any one of the preceding claims, wherein the antisense strand comprises at least one, e.g., 2, 3, 4, 5 or more acyclic (e.g., unlocked nucleic acid (UNA) or glycol nucleic acid (GNA)) nucleotides.

114. The dsRNA of any one of the preceding claims, wherein the sense strand comprises at least one, e.g., 2, 3, 4, 5 or more acyclic (e.g., unlocked nucleic acid (UNA) or glycol nucleic acid (GNA)) nucleotides.

115. The dsRNA of any one of the preceding claims, wherein the dsRNA comprises at least one thermally destabilizing modification (e.g., is an abasic nucleotide, 2′-deoxy nucleotides, acyclic nucleotide (e.g., unlocked nucleic acid (UNA), glycol nucleic acid (GNA) or (S)-glycol nucleic acid (S-GNA)), a 2′-5′ linked nucleotide (3′-RNA), threose nucleotide (TNA), 2′ gem Me/F nucleotide or mismatch with an opposing nucleotide in the other strand).

116. The dsRNA of any one of the preceding claims, wherein the antisense strand comprises at least one thermally destabilizing modification.

117. The dsRNA of any one of the preceding claims, wherein the antisense strand comprises at least one thermally destabilizing modification in the seed region (i.e., positions 2-9 from the 5′-end) of the antisense strand.

118. The dsRNA of any one of the preceding claims, wherein the antisense strand comprises a thermally destabilizing modification at least at one of positions 6, 7 or 8, counting from the 5′-end of the strand.

119. The dsRNA of any one of the preceding claims, wherein the antisense strand comprises a thermally destabilizing modification at position 7, counting from the 5′-end of the strand.

120. An oligonucleotide comprising one or both of (a) and (b):
(b) a 5′-terminal nucleoside that is a 2′-geminal-substituted nucleoside of formula (II): 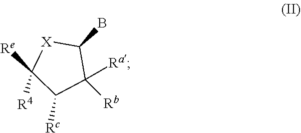
and
(b) at least one 2′-geminal-substituted nucleoside according to formula (I): 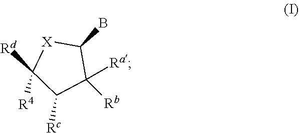
wherein in formulae (I) and (II):
X is O, S, C(R^{X})₂, or N(R^{XN});
each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{N}), or =CH₂;
R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
B is an optionally modified nucleobase;
R^{a′} is halogen, hydrogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a bond to an internucleoside linkage to a subsequent nucleoside;
R^{a2} is hydrogen or hydroxyl protecting group;
R^{a3} is hydrogen or sulfur protecting group;
R^{a4} is independently for each occurrence H, C₁-C₃₀alkyl, cyclyl, heterocyclyl, aryl, heteroaryl, aralkyl, sugar or R^{a5};
R^{a5} is independently for each occurrence amino (NH₂), alkylamino, dialkylamino, arylamino, diarylamino, heteroarylamino, or diheteroaryl amino;
m is 1-50;
n is 1-50;
R^{b} is optionally substituted C₁₋₃₀alkyl, optionally substituted C₂₋₃₀alkenyl, or optionally substituted C₂₋₃₀alkynyl;
R^{c} is a bond to an internucleoside linkage to a subsequent nucleoside, hydrogen, halogen, —OR^{c2}, —SR^{c3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, or a linker covalently attached to a solid support, and where, optionally, at least R^{c} or R^{a′} is a bond to an internucleoside linkage to a subsequent nucleoside;
R^{c2} is hydrogen or hydroxyl protecting group;
R^{c3} is hydrogen or sulfur protecting group;
R^{c4} is independently for each occurrence H, C₁-C₃₀alkyl, cyclyl, heterocyclyl, aryl, heteroaryl, aralkyl, sugar or R^{c5};
R^{c5} is independently for each occurrence amino (NH₂), alkylamino, dialkylamino, arylamino, diarylamino, heteroarylamino, or diheteroaryl amino;
r is 1-50;
s is 1-50;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R^{a} taken together are 4′-C(R^{a11}R^{a12})ᵥ—Y-2′ or 4′-Y—C(R^{a11}R^{a12})ᵥ-2′;
Y is —O—, —CH₂—, —CH(Me)-, —C(CH₃)₂—, —S—, —N(R^{a13})—, —C(O)—, —C(S)—, —S(O)—, —S(O)₂—, —OC(O)—, —C(O)O—, —N(R^{a13})C(O)—, or —C(O)N(R^{a13})—;
R^{a11} and R^{a12} independently are H, optionally substituted C₁-C₆alkyl, optionally substituted C₂-C₆alkenyl or optionally substituted C₂-C₆alkynyl;
R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
v is 1, 2 or 3;
or R⁴ and R^{c} taken together with the atoms to which they are attached form an optionally substituted C₃₋₈cycloalkyl, optionally substituted C₃₋₈cycloalkenyl, or optionally substituted 3-8 membered heterocyclyl;
R^{d} is —CH(R^{d1})—R^{d2} or —C(R^{d1})=CHR^{d2};
R^{d1} is hydrogen, optionally substituted C₁₋₃₀alkyl, optionally substituted —C₂₋₃₀alkenyl, or optionally substituted —C₂₋₃₀alkynyl;
R^{d2} is a bond to an internucleoside linkage to the preceding nucleoside;
R^{e} is optionally substituted —C₂₋₆alkenyl-R^{e1}, optionally substituted C₁₋₆alkyl-R^{e1}, or optionally substituted —C₂₋₆alkynyl-R^{e1};
R^{e1} is —P(O)(OR^{e4})₂, —OR^{e2}, —SR^{e3}, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂;
R^{e2} is hydrogen or oxygen protecting group;
R^{e3} is hydrogen or sulfur protecting group;
each R^{e4} is independently hydrogen, optionally substituted C₁₋₃₀alkyl, optionally substituted C₂₋₃₀alkenyl, or optionally substituted C₂₋₃₀alkynyl, or an oxygen-protecting group;
and each R^{e5} is independently hydrogen, optionally substituted C₁₋₃₀alkyl, optionally substituted C₂₋₃₀alkenyl, or optionally substituted C₂₋₃₀alkynyl, or a sulfur-protecting group, and
provided that the nucleoside of formula (I) is not of structure 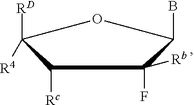
where
R^{b} is hydrogen or a substituted or unsubstituted C₁-C₄ alkyl;
R^{c} is a bond to an internucleotide linkage to a subsequent nucleoside or —OR^{Ix}, where
R_{Ix} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, a ligand carrying monomer, —F, —(CᵣC₆)alkyl, —(C₂-C₆)allyl, —(C(R³)₂)ₙOR³, —(C(R³)₂)ₙSR³, —(C(R³)₂)ₙN(R³)₂, —(C(R³)₂)ₙC(O)N(R³)₂, —(C(R³)₂)ₙO(CᵣC₆)alkyl, —(C(R³)₂)ₙS(CᵣC₆)alkyl, —(C(R³)₂)ₙO(C(R³)₂)ₙN((C₁-C₆)alkyl)₂, —(C(R³)₂)ₙON((C₁-C₆)alkyl)₂, —C(O)R³, —C(O)R³C(O)H, —C(O)R³C(O)OH, —C(O)R³C(O)R³, —C(O)R³C(O)NR³ —PO₂, —P(OR³)₂, —P(N(R³)₂)₂, —P(OR³)N(R³)₂, or a linker;
R⁴ is H;
R^{d} is a bond to an internucleotide linkage to a preceding nucleoside;
M represents independently for each occurrence an alkali metal or a transition metal with an overall charge of +1; and
n is an integer from 1-4, and
(i) the nucleoside of formula (II) is not of structure 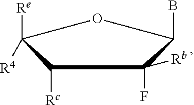
where
R^{b} is hydrogen or a substituted or unsubstituted C₁-C₄ alkyl;
R^{c} is a bond to an internucleotide linkage to a subsequent nucleoside or —OR^{Ix}, where
R^{Ix} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, a ligand carrying monomer, —F, —(CᵣC₆)alkyl, —(C₂-C₆)allyl, —(C(R³)₂)ₙOR³, —(C(R³)₂)ₙSR³, —(C(R³)₂)ₙN(R³)₂, —(C(R³)₂)ₙC(O)N(R³)₂, —(C(R³)₂)ₙO(CᵣC₆)alkyl, —(C(R³)₂)ₙS(CᵣC₆)alkyl, —(C(R³)₂)ₙO(C(R³)₂)ₙN((C₁-C₆)alkyl)₂, —(C(R³)₂)ₙON((C₁-C₆)alkyl)₂, —C(O)R³, —C(O)R³C(O)H, —C(O)R³C(O)OH, —C(O)R³C(O)R³, —C(O)R³C(O)NR³ —PO₂, —P(OR³)₂, —P(N(R³)₂)₂, —P(OR³)N(R³)₂, or a linker;
R⁴ is H;
R^{e} is —CH₂OR^{IIx}, where
R^{IIx} is —H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, or a ligand carrying monomer;
M represents independently for each occurrence an alkali metal or a transition metal with an overall charge of +1; and
n is an integer from 1-4.

121. The oligonucleotide of any one of the preceding claims, wherein the 5′-terminal nucleotide is a 2′-geminal-substituted nucleotide of formula (II) or (II′).

122. The oligonucleotide of any one of the preceding claims, wherein:
(i) the 2′-geminal-substituted nucleoside of formula (II) is according to formula (IIA): 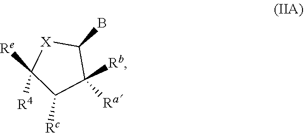
or according to formula (IIB): 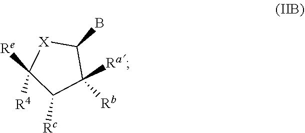
or
(ii) the 2′-geminal-substituted nucleoside of formula (II′) is according to formula (IIA′): 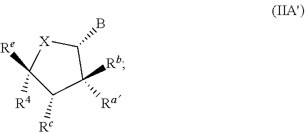
or according to formula (IIB′):

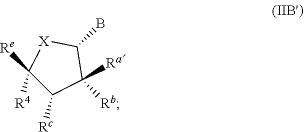

123. The oligonucleotide of any one of the preceding claims, wherein:
(i) the 2′-geminal-substituted nucleoside of formula (I) is according to formula (IA): 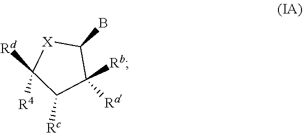
or according to formula (IB): 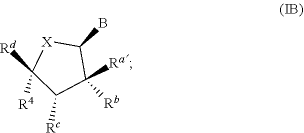
or
(ii) the 2′-geminal-substituted nucleoside of formula (I′) is according to formula (IA′): 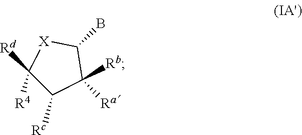
or according to formula IB′):

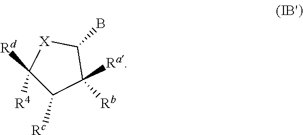

124. The oligonucleotide of any one of the preceding claims, wherein X is O.

125. The oligonucleotide of any one of the preceding claims, wherein R^{a′} is hydrogen, halogen, —OR^{a2}, optionally substituted C₁-C₃₀alkyl, optionally substituted C₁-C₃₀alkoxy, —O(CH₂CH₂O)ₘCH₂CH₂OR^{a4}, or —NH(CH₂CH₂NH)ₙCH₂CH₂—R^{a5}.

126. The oligonucleotide of any one of the preceding claims, wherein R^{a′} is halogen, hydrogen, —OR^{a2}, or optionally substituted C₁-C₃₀alkoxy,

127. The oligonucleotide of any one of the preceding claims, wherein R^{a′} is halogen, —OR^{a2}, or optionally substituted C₁-C₃₀alkoxy.

128. The oligonucleotide of any one of the preceding claims, wherein R^{a′} is F, OH or optionally substituted C₁-C₃₀alkoxy.

129. The oligonucleotide of any one of the preceding claims, wherein R^{a′} is C₁-C₃₀alkoxy optionally substituted with an amino or C₁-C₆alkoxy.

130. The oligonucleotide of any one of the preceding claims, wherein R^{b} is optionally substituted C₁₋₆alkyl, C₁₋₆haloalkyl, optionally substituted C₂₋₆alkenyl, or optionally substituted C₂₋₆alkynyl.

131. The oligonucleotide of any one of the preceding claims, wherein R^{b} is methyl, ethyl, propyl, isopropyl, butyl, iso-butyl, t-butyl, vinyl, ethynyl, allyl or propargyl.

132. The oligonucleotide of any one of the preceding claims, wherein R^{b} is methyl, vinyl, ethynyl, allyl or propargyl.

133. The oligonucleotide of any one of the preceding claims, wherein R⁴ is hydrogen, optionally substituted C₁₋₆alkyl, optionally substituted C₂₋₆alkenyl, optionally substituted C₂₋₆alkynyl, or optionally substituted C₁₋₆alkoxy.

134. The oligonucleotide of any one of the preceding claims, wherein R⁴ is hydrogen.

135. The oligonucleotide of any one of the preceding claims, wherein R^{d} is —CH(R^{d1})—X^{d}-R^{d2}.

136. The oligonucleotide of any one of the preceding claims, wherein X^{d} is O.

137. The oligonucleotide of any one of the preceding claims, wherein R^{d1} is hydrogen or optionally substituted C₁-C₆alkyl.

138. The oligonucleotide of any one of the preceding claims, wherein R^{d6} is hydrogen.

139. The oligonucleotide of any one of the preceding claims, wherein R^{e} is —C₂₋₆alkenyl-R^{e1} or C₁₋₆alkyl-R^{e1}, C₁₋₆alkyl and C₂₋₆alkenyl is optionally substituted.

140. The oligonucleotide of any one of the preceding claims, wherein R is —CH═CHR^{e1}.

141. The oligonucleotide of any one of the preceding claims, wherein R¹ is —P(O)(OR^{e4})₂, —OR^{e2}, or —OP(O)(OR⁴)₂.

142. The oligonucleotide of any one of the preceding claims, wherein R^{e2} is hydrogen or optionally substituted C₁-C₆alkyl.

143. The oligonucleotide of any one of the preceding claims, wherein the 2′-geminal-substituted nucleoside of formula (I) is at least at one of position 2, 3, 4, 5, 6, 7, 8, 9 or 10, counting from the 5′-end of the oligonucleotide, e.g., at least at one of positions 5, 6, 7 and/or 8.

144. The oligonucleotide of any one of the preceding claims, wherein the 2′-geminal-substituted nucleoside of formula (I) is at position 7, counting from the 5′-end of the oligonucleotide.

145. The oligonucleotide of any one of the preceding claims, wherein the oligonucleotide further comprises a ligand linked thereto.

146. The oligonucleotide of any one of the preceding claims, wherein the oligonucleotide solely comprises 2′-geminal-substituted nucleosides of formulae (I), (I′), (II), and (II′).

147. The oligonucleotide of any one of the preceding claims, wherein the oligonucleotide further comprises at least one modified internucleoside linkage.

148. The oligonucleotide of any one of the preceding claims, wherein the oligonucleotide further comprises at least one modified nucleobase.

149. The oligonucleotide of any one of the preceding claims, wherein the wherein the oligonucleotide further comprises at least one nucleoside modified at the 2′-position and wherein the nucleotide modified at the 2′-position is not a 2′-geminal nucleoside.

150. The oligonucleotide of any one of the preceding claims, wherein the at least one nucleoside modified at the 2′-position is a 2′-F or 2′-OMe nucleoside.

151. The oligonucleotide of any one of the preceding claims, wherein the oligonucleotide is from 10 to 50 nucleotides in length.

152. The oligonucleotide of any one of the preceding claims, wherein the oligonucleotide is linked to a solid support.

153. The oligonucleotide of any one of the preceding claims, wherein the 2′-geminal-substituted nucleoside of formula (II) is of formula (HA): 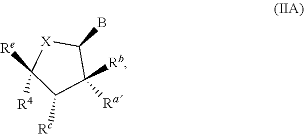
wherein:
X is O;
R^{a′} is halogen (e.g., F, Cl or Br), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl or Br);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R is —CH═CHR^{e1}, where R^{e1} is —P(O)(OR^{e4})₂.

154. The oligonucleotide of any one of claims 120-152, wherein the 2′-geminal-substituted nucleoside of formula (II) is of formula (IIB): 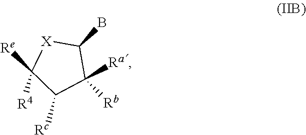
wherein:
X is O;
R^{a′} is halogen (e.g., F), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{e} is —CH═CHR^{e1}, where R^{e1} is —P(O)(OR^{e4})₂.

155. The oligonucleotide of any one of the preceding claims, wherein the 2′-geminal-substituted nucleoside of formula (I) is of formula (IA): 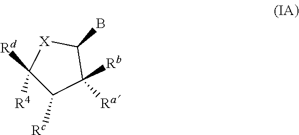
wherein:
X is O;
R^{a′} is halogen (e.g., F, Cl, or Br), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl or Br);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{d} is a bond to an internucleoside linkage to a preceding nucleoside, and
provided that only one of a R^{a′} and R^{c} is a bond to an internucleoside linkage to a subsequent nucleoside.

156. The oligonucleotide of any one of claims 120-154, wherein the 2′-geminal-substituted nucleoside of formula (I) is of formula (IB): 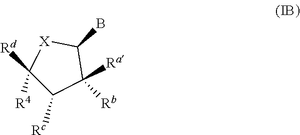
wherein:
X is O;
R^{a′} is halogen (e.g., F, Cl or Br), hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), or a bond to an internucleoside linkage to a subsequent nucleoside;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl or Br);
R^{c} is a bond to an internucleoside linkage to a subsequent nucleoside or hydroxyl, provided that only one of R^{a′} and R^{c} is a bond to an internucleoside linkage to a subsequent nucleoside;
R⁴ is hydrogen; and
R^{d} is a bond to an internucleoside linkage to a preceding nucleoside, and
provided that only one of a R^{a′} and R^{c} is a bond to an internucleoside linkage to a subsequent nucleoside.

157. A double-stranded nucleic acid comprising a first oligonucleotide strand and a second oligonucleotide strand substantially complementary to the first strand, wherein the first strand and/or the second strand is an oligonucleotide of any one of claims 120-156.

158. The double-stranded nucleic acid of claim 157, wherein the first and second strand are independently 15 to 25 nucleotides in length.

159. The double-stranded nucleic acid of any one of claims 157-158, wherein the first and/or the second strand has a 1-5 nucleotide overhang on its respective 5′-end or 3′-end.

160. The double-stranded nucleic acid of any one of claims 157-159, wherein only one of the first or second strand has a 2 nucleotide single-stranded overhang on its 5′-end or 3′-end.

161. The double-stranded nucleic acid of any one of claims 157-160, wherein only one strand has a 2 nucleotide single-stranded overhand on its 3′-end.

162. The double-stranded nucleic acid of any one of claims 157-161, wherein the second strand comprises a ligand linked thereto.

163. The double-stranded nucleic acid of any one of claims 157-162, wherein first strand is substantially complementary to a target nucleic acid and the double-stranded nucleic is capable of inducing RNA interference.

164. A method of reducing the expression of a target gene in a subject, comprising administering to the subject either:
(i) a double-stranded RNA according to any one of claims 1-119, wherein the strand is complementary to a target gene
(ii) a double-stranded RNA according to any one of claims 157-163, wherein the first strand (e.g., antisense strand) is complementary to a target gene; or
(iii) an oligonucleotide according to any one of claims 120-156, wherein the oligonucleotide is complementary to a target gene.

165. A compound of formula (III) or (III′): 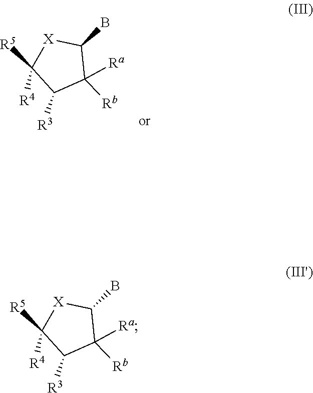
wherein:
X is O, S, C(R^{X})₂, or N(R^{XN});
each R^{X} is independently hydrogen, halogen, optionally substituted C₁₋₄alkyl, C₁₋₄haloalkyl, optionally substituted C₂₋₄alkenyl, or optionally substituted C₂₋₄alkynyl, or both R^{X} taken together form ═O, ═S, ═N(R^{N}), or =CH₂;
R^{XN} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
B is an optionally modified nucleobase;
R^{a} is halogen, hydrogen, —OR^{a2}, —SR^{a3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ₘCH₂CH₂OR^{a4}, cyano, alkyl-thio-alkyl, thioalkoxy, cycloalkyl, aryl, heteroaryl, —NH(CH₂CH₂NH)ₙCH₂CH₂—R^{a5}, NHC(O)R^{a4}, a lipid, a linker covalently attached to a lipid, a ligand, a linker covalently attached to a ligand, a solid support, a linker covalently attached to a solid support, or a reactive phosphorus group;
R^{a2} is hydrogen or hydroxyl protecting group;
R^{a3} is hydrogen or sulfur protecting group;
R^{a4} is independently for each occurrence H, C₁-C₃₀alkyl, cyclyl, heterocyclyl, aryl, heteroaryl, aralkyl, sugar or R^{a5};
R^{a5} is independently for each occurrence amino (NH₂), alkylamino, dialkylamino, arylamino, diarylamino, heteroarylamino, or diheteroaryl amino;
m is 1-50;
n is 1-50;
R^{b} is optionally substituted C₁₋₃₀alkyl, optionally substituted C₂₋₃₀alkenyl, optionally substituted C₂₋₃₀alkynyl, or halogen;
R³ is hydrogen, halogen, —OR^{e2}, —SR^{e3}, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR^{c4}, cyano, alkyl-thio-alkyl, thioalkoxy, cycloalkyl, aryl, heteroaryl, —NH(CH₂CH₂NH)ₛCH₂CH₂—R^{c5}, NHC(O)R^{c4}, a lipid, a linker covalently attached to a lipid, a ligand, a linker covalently attached to a ligand, a solid support, a linker covalently attached to a solid support, or a reactive phosphorus group;
R^{c2} is hydrogen or hydroxyl protecting group;
R^{c3} is hydrogen or sulfur protecting group;
R^{c4} is independently for each occurrence H, C₁-C₃₀alkyl, cyclyl, heterocyclyl, aryl, heteroaryl, aralkyl, sugar or R^{c5};
R^{c5} is independently for each occurrence amino (NH₂), alkylamino, dialkylamino, arylamino, diarylamino, heteroarylamino, or diheteroaryl amino;
r is 1-50;
s is 1-50;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R^{a} taken together are 4′-C(R^{a11}R^{a12})ᵥ—Y-2′ or 4′-Y—C(R^{a11}R^{a12})ᵥ-2′;
Y is —O—, —CH₂—, —CH(Me)-, —C(CH₃)₂—, —S—, —N(R^{a13})—, —C(O)—, —C(S)—, —S(O)—, —S(O)₂—, —OC(O)—, —C(O)O—, —N(R^{a13})C(O)—, or —C(O)N(R^{a13})—;
R^{a11} and R^{a12} independently are H, optionally substituted C₁-C₆alkyl, optionally substituted C₂-C₆alkenyl or optionally substituted C₂-C₆alkynyl;
R^{a13} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
v is 1, 2 or 3;
or R⁴ and R^{c}taken together with the atoms to which they are attached form an optionally substituted C₃₋₈cycloalkyl, optionally substituted C₃₋₈cycloalkenyl, or optionally substituted 3-8 membered heterocyclyl;
R⁵ is optionally substituted —C₂₋₆alkenyl-R^{5a}, optionally substituted C₁₋₆alkyl-R^{5a}, or optionally substituted —C₂₋₆alkynyl-R^{5a};
R^{5a} is a phosphorus group, —OR^{5b}, —SR^{5c}, hydrogen, a protected phosphorous group, a solid support or a linker to a solid support, provided that only one of R^{3a}
R³ and R⁵ is a linkage to a solid support;
R^{5b} is H or hydroxyl protecting group; and
R^{5c} is H or sulfur protecting group, and
provided that only one of R^{a}, R³, and R^{5a} is a solid support or linkage to a solid support;
provided that only one of R^{a}, R³ and R^{5a} is a reactive phosphorous, and
provided that the compound is not of structure 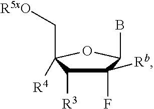
where:
R^{b} is hydrogen or a substituted or unsubstituted C₁-C₄ alkyl;
R^{c} is —OR^{Ix}, where:
R^{Ix} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, a ligand carrying monomer, —F, —(CᵣC₆)alkyl, —(C₂-C₆)allyl, —(C(R³)₂)ₙOR³, —(C(R³)₂)ₙSR³, —(C(R³)₂)ₙN(R³)₂, —(C(R³)₂)ₙC(O)N(R³)₂, —(C(R³)₂)ₙO(CᵣC₆)alkyl, —(C(R³)₂)ₙS(CᵣC₆)alkyl, —(C(R³)₂)ₙO(C(R³)₂)ₙN((C₁-C₆)alkyl)₂, —(C(R³)₂)ₙON((C₁-C₆)alkyl)₂, —C(O)R³, —C(O)R³C(O)H, —C(O)R³C(O)OH, —C(O)R³C(O)R³, —C(O)R³C(O)NR³ —PO₂, —P(OR³)₂, —P(N(R³)₂)₂, —P(OR³)N(R³)₂, or a linker;
R⁴ is H;
R^{5X} is H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, or a ligand carrying monomer;
M represents independently for each occurrence an alkali metal or a transition metal with an overall charge of +1; and
n is an integer from 1-4.

166. The compound of claim 165, wherein
a. the compound of formula (III) is of formula (IIIA): 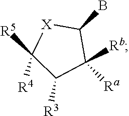
or
b. the compound of formula (III′) is of formula (IIIA′):

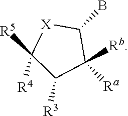

167. The compound of claim 165, wherein:
a. the compound formula (III) is of formula (IIIB): 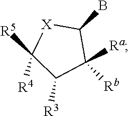
or
b. the compound of formula (III′) is of formula (IIIB′):

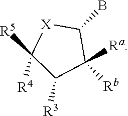

168. The compound of any one of the preceding claims, wherein the reactive phosphorous group is phosphoramidite, H-phosphonate, alkyl-phosphonate, or phosphate triester.

169. The compound of any one of the preceding claims, wherein the reactive phosphorous group is —OP(O^{P})(NR^{P2})₂, —OP(SR^{P})(NR^{P2})₂, —OP(O)(OR^{P})(NR^{P2})₂, —OP(S)(OR^{P})(NR^{P2})₂, —OP(O)(SR^{P})(NR^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}, wherein
R^{P} is an optionally substituted C₁₋₆alkyl; and
each R^{P2} is independently optionally substituted C₁₋₆alkyl;
or both R^{P2} taken together with the nitrogen atom to which they are attached form an optionally substituted 3-8 membered heterocyclyl;
or R^{P} and one of R^{P2} taken together with the atoms to which they are attached form an optionally substituted 4-8 membered heterocyclyl;
and
R^{P3} is an optionally substituted C₁-C₃₀alkyl, optionally substituted C₂-C₃₀alkenyl, or optionally substituted C₂-C₃₀alkynyl.

170. The compound of any one of the preceding claims, wherein the reactive phosphorous group is —OP(OR^{P})(NR^{P2})₂.

171. The compound of claim 169 or 170, wherein R^{P} is C₁₋₆alkyl substituted with cyano or —SC(O)Ph.

172. The compound of any one of claims 169-171, wherein R^{P} is —CH₂CH₂CN.

173. The compound of any one of claims 169-172, wherein each R^{P2} is independently methyl, ethyl, propyl, or isopropyl.

174. The compound of any one of claims 169-173, wherein each R^{P2} is isopropyl.

175. The compound of any one of claims 169-174, wherein R^{P3} is an optionally substituted C₁-C₆alkyl.

176. The compound of anyone of the preceding claims, wherein X is O.

177. The compound of anyone of the preceding claims, wherein R^{a} is halogen, hydrogen, —OR^{a2}, or optionally substituted C₁-C₃₀alkoxy.

178. The compound of anyone of the preceding claims, wherein R^{a} is halogen, —OR^{a2}, or optionally substituted C₁-C₃₀alkoxy.

179. The compound of anyone of the preceding claims, wherein R^{a} is F, Cl, OH or optionally substituted C₁-C₃₀alkoxy.

180. The compound of anyone of the preceding claims, wherein R^{a} is C₁-C₃₀alkoxy optionally substituted with an amino or C₁-C₆alkoxy.

181. The compound of anyone of the preceding claims, wherein R^{b} is optionally substituted C₁₋₆alkyl, C₁₋₆haloalkyl, optionally substituted C₂₋₆alkenyl, optionally substituted C₂₋₆alkynyl, or halogen.

182. The compound of anyone of the preceding claims, wherein R^{b} is methyl, ethyl, propyl, isopropyl, butyl, iso-butyl, t-butyl, vinyl, ethynyl, allyl or propargyl.

183. The compound of anyone of the preceding claims, wherein R^{b} is methyl, vinyl, ethynyl, allyl or propargyl.

184. The compound of anyone of the preceding claims, wherein R³ is H, halogen, OR^{c2}, a reactive phosphorus group, or a linkage to a solid support.

185. The compound of anyone of the preceding claims, wherein R³ is a reactive phosphorus group, or a linkage to a solid support.

186. The compound of anyone of the preceding claims, wherein R³ is a reactive phosphorous group.

187. The compound of anyone of the preceding claims, wherein R⁴ is hydrogen, optionally substituted C₁₋₆alkyl, optionally substituted C₂₋₆alkenyl, optionally substituted C₂₋₆alkynyl, or optionally substituted C₁₋₆alkoxy.

188. The compound of anyone of the preceding claims, wherein R⁴ is hydrogen.

189. The compound of anyone of the preceding claims, wherein R⁵ is optionally substituted optionally substituted —C₂₋₆alkenyl-R^{5a} or C₁₋₆alkyl-R^{5a}.

190. The compound of anyone of the preceding claims, wherein R⁵ is —CH═CHR^{5a}.

191. The compound of anyone of the preceding claims, wherein R^{5a} is a phosphorous group or —OR^{5b}.

192. The compound of anyone of the preceding claims, wherein R^{5a} is a phosphorous group.

193. The compound of anyone of the preceding claims, wherein R^{5a} is —P(O)(OR^{e4})₂, —P(S)(OR^{e4})₂, —P(S)(SR^{e5})(OR^{e4}), —P(S)(SR^{e5})₂, —OP(O)(OR^{e4})₂, —OP(S)(OR^{e4})₂, —OP(S)(SR^{e5})(OR^{e4}), —OP(S)(SR^{e5})₂, —SP(O)(OR^{e4})₂, —SP(S)(OR^{e4})₂, —SP(S)(SR^{e5})(OR^{e4}), or —SP(S)(SR^{e5})₂;
R^{e2} is hydrogen or oxygen protecting group;
R^{e3} is hydrogen or sulfur protecting group;
each R^{e4} is independently hydrogen, optionally substituted C₁₋₃₀alkyl, optionally substituted C₂₋₃₀alkenyl, or optionally substituted C₂₋₃₀alkynyl, or an oxygen-protecting group; and
each R^{e5} is independently hydrogen, optionally substituted C₁₋₃₀alkyl, optionally substituted C₂₋₃₀alkenyl, or optionally substituted C₂₋₃₀alkynyl, or a sulfur-protecting group.

194. The compound of claim 170, wherein the compound is of Formula (IIIa): 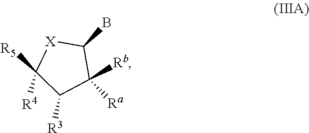
wherein:
X is O;
R^{a} is halogen (e.g., F), hydroxyl, protected hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), a reactive phosphorous group, a solid support, or a linker covalently attached to a solid support;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl);
R⁴ is hydrogen;
R³ is a reactive phosphorous group, a solid support, a linker covalently attached to a solid support, hydroxyl, or protected hydroxyl, provided that only one of R^{a} and R³ is a reactive phosphorous group, a solid support, or a linker covalently attached to a solid support; and
R⁵ is —CH═CHR^{5a}, where R^{5a} is —P(O)(OR^{5e})₂ and each R^{Se} is independently hydrogen, or optionally substituted C₁₋₃₀alkyl.

195. The compound of claim 165, wherein the compound for formula (III) is of Formula (IIIB): 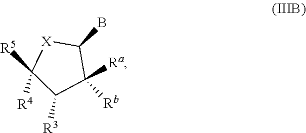
wherein:
X is O;
R^{a} is halogen (e.g., F, Cl, Br), hydroxyl, protected hydroxyl, optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2), a reactive phosphorous group, a solid support, or a linker covalently attached to a solid support;
R^{b} is optionally substituted C₁₋₆alkyl (e.g., methyl, ethyl, propyl, isopropyl, butyl, iso-butyl, t-butyl, vinyl, ethynyl, allyl or propargyl, preferably methyl) or halogen (e.g., F, Cl, Br);
R⁴ is hydrogen;
R³ is a reactive phosphorous group, a solid support, a linker covalently attached to a solid support, hydroxyl, or protected hydroxyl, provided that only one of R^{a} and R³ is a reactive phosphorous group, a solid support, or a linker covalently attached to a solid support; and
R⁵ is —CH═CHR^{5a}, where R^{5a} is —P(O)(OR^{5e})₂ and each R^{5e} is independently hydrogen, or optionally substituted C₁₋₃₀alkyl.

196. The compound of claim 194 or 195, wherein R³ is a reactive phosphorous group.

197. The compound of any one of claims 194-196, wherein R³ is —OP(OR^{P})N(R^{P2})₂, —OP(SR^{P}) N(R^{P2})₂, —OP(O)(OR^{P})N(R^{P2})₂, —OP(S)(OR^{P})N(R^{P2})₂, —OP(O)(SR^{P})N(R^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}

198. The compound of any one of claims 194-197, wherein R³ is —OP(OCH₂CH₂CN)N(iPr)₂.

199. The compound of claim 194 or 195, wherein R³ is hydroxyl or protected hydroxyl.

200. The compound of claim 194 or 195, wherein R³ is a solid support or a linker covalently attached to a solid support.

201. The compound of any one of claims 194-200, wherein R^{a} is F, hydroxyl, protected hydroxyl, or optionally substituted C₁₋₃₀alkoxy (e.g., —(CH2₂)ₙCH₃, where n is 1-21, e.g., 1, 16; or —(CH2₂)ₘ—NH₂, where m is 2-10, e.g., 3 or 6; or —(CH2₂)ₚ—OMe, where p is 1 to 21, e.g., 1 or 2).

202. The compound of claim 165, wherein the compound is selected from the group consisting of: 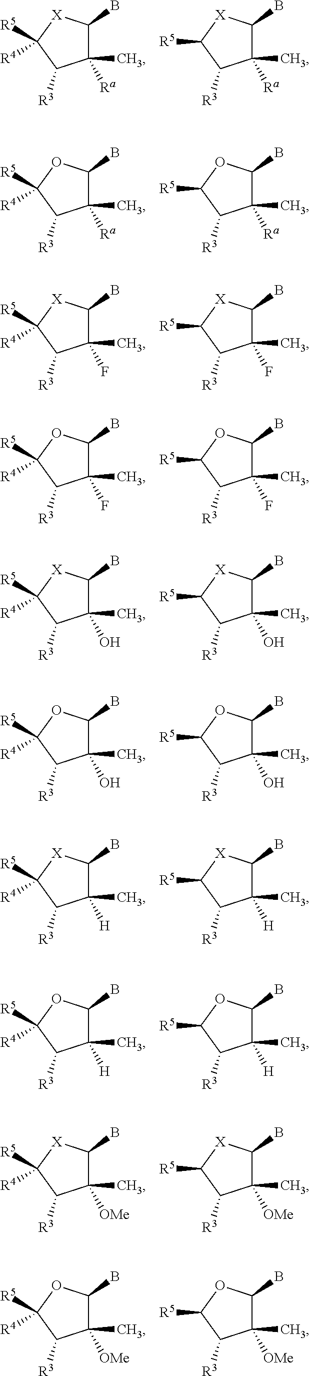 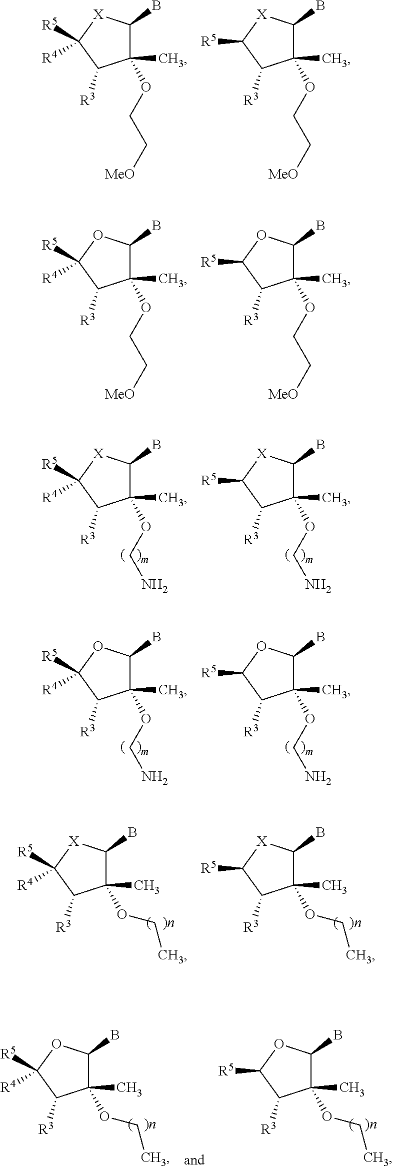
where m is 2-10 (e.g., 3, 6) and n is 1-21, (e.g., 1, 16).

203. The compound of claim 165, wherein the compound is selected from the group consisting of:

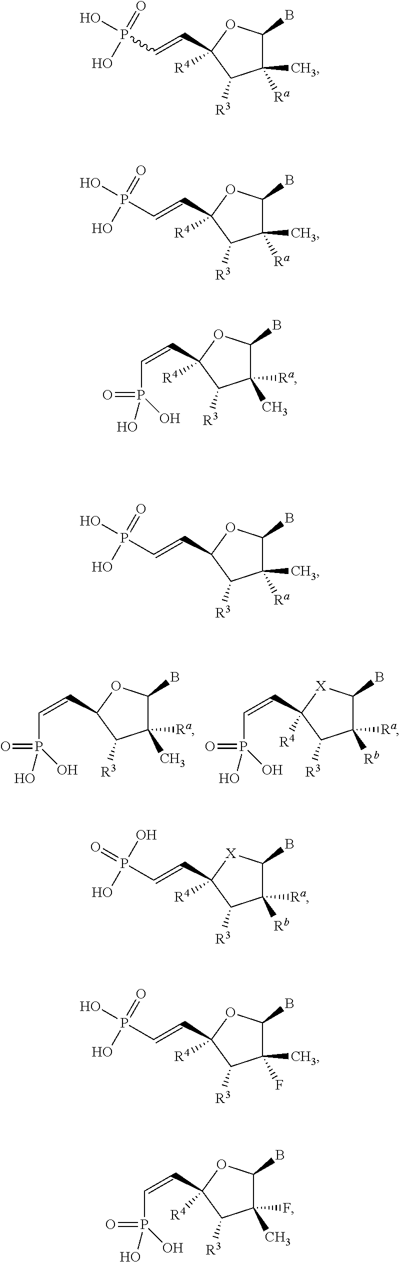

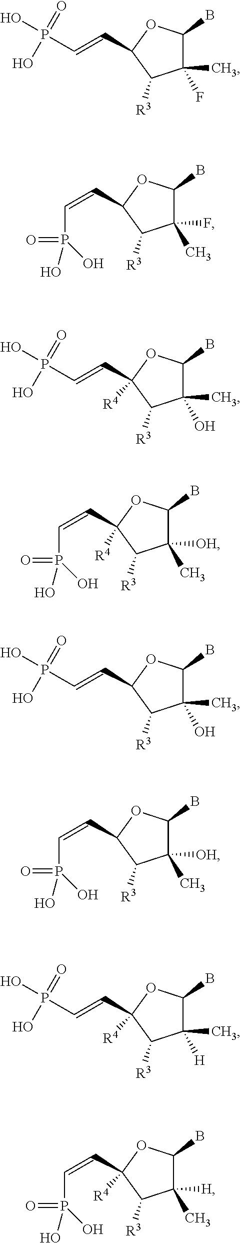

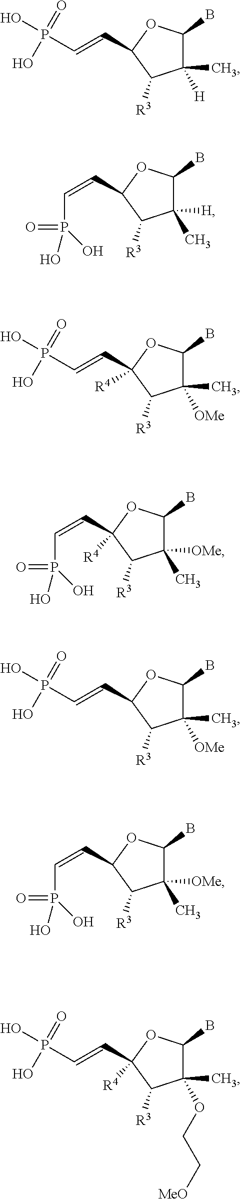

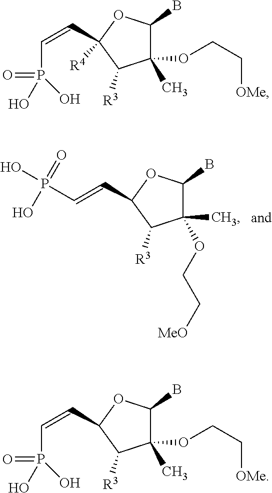

204. The compound of claim 165, wherein the compound is selected from the group consisting of: 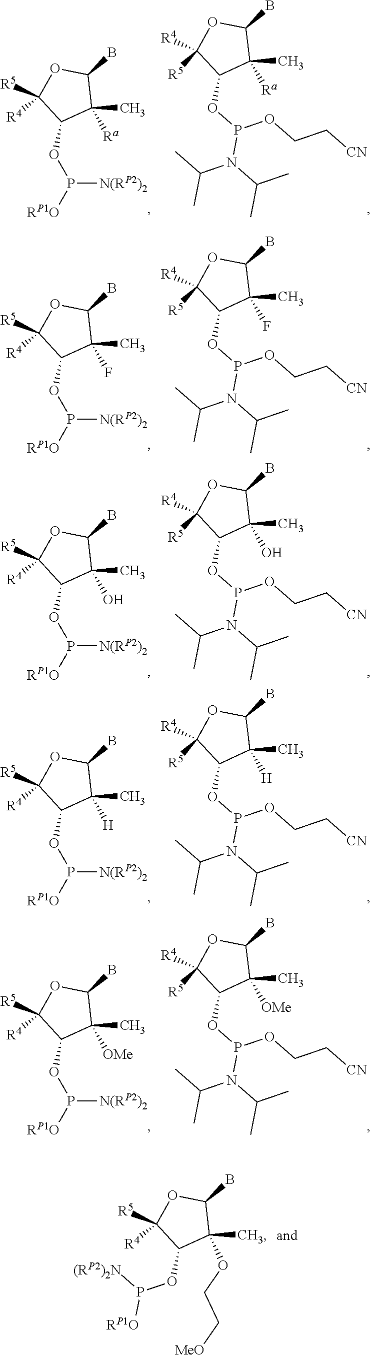 [chemical structure]
